(19) United States
(12) Patent Application Publication
(10) Pub. No.: US 2025/0090663 A1
(43) Pub. Date: Mar. 20, 2025

(54) BINDING DOMAINS AND METHODS OF USE THEREOF

(71) Applicant: Myeloid Therapeutics, Inc., Cambridge, MA (US)

(72) Inventors: Daniel Getts, Medfield, MA (US); Yuxiao Wang, Belmont, MA (US); Vania Emilova Ashminova, North Billerica, MA (US); Thach Chu, Dedham, MA (US)

(21) Appl. No.: 18/884,235

(22) Filed: Sep. 13, 2024

Related U.S. Application Data
(63) Continuation of application No. PCT/US2023/015415, filed on Mar. 16, 2023.
(60) Provisional application No. 63/320,574, filed on Mar. 16, 2022.
(60) Provisional application No. 63/357,484, filed on Jun. 30, 2022.
(60) Provisional application No. 63/422,184, filed on Nov. 3, 2022.

(51) Int. Cl. A61K 39/00; C07K 14/725; C07K 16/28; C07K 16/30

(52) U.S. Cl. CPC A61K 40/4261; A61K 40/31; A61K 40/4217; A61K 40/4232; C07K 14/7051; C07K 16/2866; C07K 16/2875; C07K 16/303; C07K 2317/569; C07K 2317/92

(57) ABSTRACT

Compositions and methods for binding to target antigens. Compositions comprising variable heavy chain domains with binding specificity to CD70, GPC3, IL13Ralpha2 and Cadherin CDH17 antigens.

### CROSS REFERENCE

This application is a continuation of PCT application No. PCT/US2023/015415, filed on Mar. 16, 2023 which claims the benefit of U.S. Provisional Application No. 63/320,574 filed on Mar. 16, 2022, U.S. Provisional Application No. 63/357,484, filed on Jun. 30, 2022; and U.S. Provisional Application No. 63/422,184, filed on Nov. 3, 2022, and each of which is incorporated herein by reference in its entirety.

### SEQUENCE LISTING

The instant application contains a Sequence Listing which has been filed electronically in XML format and is hereby incorporated by reference in its entirety. Said XML copy, created on Apr. 24, 2023 is named 56371-735_601_SL.xml and is 344,462 bytes in size.

### BACKGROUND

Myeloid cells are recently identified as safe and effective cells for developing novel cell therapy. Myeloid cells are present in abundance at the site of disease, inflammation or infection. Efforts are under way to develop myeloid cells as therapeutic tools.

Cancer is a multifactorial disease. Myeloid cells have access to tumors and can be harnessed for developing novel cancer therapeutics.

### SUMMARY

Provided herein are binding domains to target proteins. In some embodiments the target protein is CD70. In some embodiments the target protein is GPC3. In some embodiments the target protein is IL13 receptor alpha 2 (IL13Rα2).

In one aspect, provided herein is a composition comprising a construct comprising an antigen binding domain comprising an anti-CD70 antibody or binding fragment thereof, or a recombinant nucleic acid encoding the antigen binding domain, wherein the antigen binding domain comprises a heavy chain variable domain (VH) comprising a heavy chain complementarity determining region 3 (HC CDR3) that has a sequence selected from the group consisting of ATGPYGLDNALDA (SEQ ID NO: 4) and AKSLRSSPSSRWFGS (SEQ ID NO: 14).

In some embodiments, the VH of the anti-CD70 antibody or binding fragment thereof further comprises a heavy chain complementarity determining region 1 (HC CDR1) that has a sequence selected from the group consisting of RSIFSINA (SEQ ID NO: 2) and GFTFDDYA (SEQ ID NO: 12).

In some embodiments, the VH of the anti-CD70 antibody or binding fragment thereof further comprises a heavy chain complementarity determining region 2 (HC CDR2) that has a sequence selected from the group consisting of ITSGGSP (SEQ ID NO: 3) and ISWSGGTT (SEQ ID NO: 13).

In some embodiments, the VH of the anti-CD70 antibody or binding fragment thereof comprises with 70-100% sequence identity to any one of the sequences selected from the group consisting of

(SEQ ID NO: 1)
QVQLQESGGGLVQAGGSLRLSCAAPRSIFSINAMGWYRQAPGKQRELVA
AITSGGSPTYADSVKGRFTISRDNAKNTVYLQMNSLKAEDTAVYYCATG
PYGLDNALDAWGQGTQVTVSS
and
(SEQ ID NO: 11)
QVQLQESGGGLVQTGGSLRLACTASGFTFDDYAIAWFRQAPGKEREFVA
AISWSGGTTHYADSVKGRFTISRDNAKNTLYLQMSSLKPEDTAVYFCAK
SLRSSPSSRWFGSRGQGTQVTVSS.

In some embodiments, the VH of the anti-CD70 antibody or binding fragment thereof is encoded by a sequence with 70-100% sequence identity to any one of the sequences selected from the group consisting of

(SEQ ID NO: 5)
CAAGTTCAGCTCCAGGAATCAGGTGGGGGACTCGTACAGGCTGGAGGAAGCCTGCGAT
TGAGCTGTGCCGCTCCACGATCTATCTTCTCAATAAATGCTATGGGTTGGTACAGGCAG
GCCCCAGGCAAACAAAGAGAACTTGTAGCAGCCATTACTTCAGGGGGGAGCCCAACCT
ATGCTGACTCAGTAAAGGGGCGGTTCACTATTAGTAGGGATAACGCGAAGAATACGGT
CTACTTGCAGATGAACAGTCTGAAGGCCGAAGATACGGCCGTGTATTATTGTGCCACAG
GTCCCTATGGGCTGGACAATGCGTTGGATGCGTGGGGTCAGGGCACGCAGGTCACTGTG
AGTAGT
and
(SEQ ID NO: 15)
CAGGTGCAGTTGCAGGAGAGTGGCGGTGGCCTGGTGCAGACCGGTGGCAGTCTGAGGC
TGGCCTGCACTGCCAGCGGCTTCACCTTCGACGACTATGCCATCGCGTGGTTCAGGCAG
GCCCCTGGCAAGGAGAGGGAGTTTGTCGCAGCCATCAGCTGGAGCGGAGGCACCACCC
ACTACGCTGACAGCGTGAAGGGCAGGTTCACCATTAGCAGGGACAATGCCAAGAACAC
CCTGTACCTGCAGATGAGCAGTCTCAAGCCCGAAGATACCGCCGTGTACTTCTGCGCCA
AGAGCCTGAGGAGCAGCCCCAGCAGCAGGTGGTTCGGCAGCCGCGGTCAAGGCACCCA
GGTGACCGTGAGCTCT.

In some embodiments, the VH is a single domain antibody domain.

In some embodiments, the VH is a VHH.

In one aspect, provided herein is a composition comprising a construct comprising an antigen binding domain comprising an anti-GPC3 antibody or binding fragment thereof, or a recombinant nucleic acid encoding the antigen binding domain, wherein the antigen binding domain comprises a heavy chain variable domain (VH) comprising a heavy chain complementarity determining region 3 (HC CDR3) of any one of the sequences selected from the group consisting of

(SEQ ID NO: 113)
ATACADTTQYAYDY,
(SEQ ID NO: 114 or SEQ ID NO: 317)
ATACADTTLYEYDY,
(SEQ ID NO: 115)
ATACVDTTQYEYDY,
(SEQ ID NO: 116)
ATACADATQHEYDY,
(SEQ ID NO: 117)
ATACADTTQYDYDY,
(SEQ ID NO: 118)
ATACADTTQYEYDY,
(SEQ ID NO: 119)
ATACADTTHYEYDY,
(SEQ ID NO: 120)
ATACVITTLYEYDY,
(SEQ ID NO: 121)
ATACAETTLYEYDY,
(SEQ ID NO: 122)
ATACADTTQHEYDY,
(SEQ ID NO: 123)
ATACVDTTHYEYDY,
(SEQ ID NO: 124)
ATACASTTLYEYDY,
(SEQ ID NO: 125)
ATACVVTTLYEYDY,
(SEQ ID NO: 126)
ATACGGATGPYDY,
(SEQ ID NO: 127)
ATACAGAIGPYDY,
(SEQ ID NO: 128)
ATACVVVGDQNDY,
(SEQ ID NO: 129)
ATACVVVGDRNDY,
(SEQ ID NO: 130)
ATDCAGGTSTPYDY,
(SEQ ID NO: 131)
ATDCAGGTATPYDY,
(SEQ ID NO: 132)
ATACVVADRNEYDY,
(SEQ ID NO: 133)
ATSCVVVTKNEYDY,
(SEQ ID NO: 134)
ATACSGLTHEYDY,
(SEQ ID NO: 135)
ATTCSGLTHEYDY,
(SEQ ID NO: 136)
ATACANWSSLGPYDY,
(SEQ ID NO: 137)
ATACANWSTLGPYDY,
(SEQ ID NO: 138)
ATACSDPRVYEYDY,
(SEQ ID NO: 139)
ATTCASPEKYEYDY,
(SEQ ID NO: 140)
ATHCGGTSWGTSYDY,
(SEQ ID NO: 141)
ATHCGGSSWSNEYDY,
(SEQ ID NO: 142)
YARYSGRTY,
(SEQ ID NO: 143)
ASSAWPAGPKHQVEYDY,
(SEQ ID NO: 144)
ATACGSLVGMYDY,
(SEQ ID NO: 145)
ATACGSAVHEYDY,
(SEQ ID NO: 146)
ATDCVGFGSNWFDY,
(SEQ ID NO: 147)
ATACASPVIYEYDY,
(SEQ ID NO: 148)
ATDCAGGVGHEYDY,
(SEQ ID NO: 149)
ATDCSLHGSDYPYDY
and
(SEQ ID NO: 150)
AVRIYSGSFDNTLAYDY.

In some embodiments, the VH of the anti-GPC3 antibody or binding fragment thereof further comprises a heavy chain complementarity determining region 1 (HC CDR1) of any one of the sequences selected from the group consisting of sequences: GFPLAYYA (SEQ ID NO: 151), GFSLDYYA (SEQ ID NO: 152), GFPLDYYA (SEQ ID NO: 153), GFTLDYYA (SEQ ID NO: 154), GFSLNYYA (SEQ ID NO: 155), GFTLAYYA (SEQ ID NO: 156), GFTLGYYA (SEQ ID NO: 157 or SEQ ID NO: 320), GFPLNYYA (SEQ ID NO: 158), GFPLHYYA (SEQ ID NO: 159), GFSLGYYA (SEQ ID NO: 160), GFPLGYYA (SEQ ID NO: 161), GFPLEYYA (SEQ ID NO: 162), GSDFRADA (SEQ ID NO: 163), GRTFSSYG (SEQ ID NO: 164), GFSLAYYA (SEQ ID NO: 165) and GLTFRSVG (SEQ ID NO: 166).

In some embodiments, the VH of the anti-GPC3 antibody or binding fragment thereof further comprises a heavy chain complementarity determining region 2 (HC CDR2) of any one of the sequences selected from the group consisting of sequences: ISNSDGST (SEQ ID NO: 167), ISASDGST (SEQ ID NO: 168 or SEQ ID NO: 318), ISSSDGST (SEQ ID NO: 169 or SEQ ID NO: 321), ISSSDGNT (SEQ ID NO: 170), ISSADGST (SEQ ID NO: 171), ISSSGGST (SEQ ID NO: 172), ISSGDGST (SEQ ID NO: 173), ISAGDGNT (SEQ ID NO: 174), ISSSDDST (SEQ ID NO: 175), ISSNDGST (SEQ ID NO: 176), ISSPDGST (SEQ ID NO: 177), ISSRTGGT (SEQ ID NO: 178), ISAGDGSST (SEQ ID NO: 179), ISSSDGSSSDGNT (SEQ ID NO: 180), ISSGDGNT (SEQ ID NO: 181), ISSGDGKT (SEQ ID NO: 182), ISSSDGGT (SEQ ID NO: 183), ISSRTGST (SEQ ID NO: 184), ISSRTGNT (SEQ ID NO: 185), ISSSDGHSST (SEQ ID NO: 186), ISSSSDGNT (SEQ ID NO: 187), ISASNGNT (SEQ ID NO: 188), ISSGSDGNT (SEQ ID NO: 189), ISASDGNT (SEQ ID NO: 190 or SEQ ID NO: 323), IDSITSI (SEQ ID NO: 191), ISWSGGSTIAASVGST (SEQ ID NO: 192), ISSSDGSDGNT (SEQ ID NO: 193 or SEQ ID NO: 329) and ASPSGVIT (SEQ ID NO: 194).

In some embodiments, the VH of the anti-GPC3 antibody or binding fragment thereof comprises with 70-100% sequence identity to any one of the sequences selected from the group consisting of

(SEQ ID NO: 22)
QVQLQESGGGLVHSGGSLRLSCAASGFPLAYYAIGWFRQAPGKEREGVSCISSSDGNTYYA
DAVKGRFTISRDNAKNTVYLQMNSLKPEDTAVYYCATACADTTQHEYDYWGQGTQVTVS
S,
(SEQ ID NO: 23)
QVQLQESGGGLVHSGGSLRLSCAASGFPLDYYAIGWFRQAPGKEREGVSCISSADGSTYYA
DSVKGRFTISRDNAKNTVYLQMNSLGPEDTAVYYCATACADTTQYDYDYWGQGTQVTVS
S,
(SEQ ID NO: 24)
QVQLQESGGGLVHSGGSLRLSCAASGFTLDYYAIGWFRRAPGKEREGVSCISSGDGKTYYA
DSVKGRFTISRDNAKNTVYLQMNSLKPEDTAVYYCATACAGAIGPYDYWGQGTQVTVSS,
(SEQ ID NO: 25)
QVQLQESGGGLVPPGGSLRLSCAASGFPLDYYAIGWFRQAPGKEREGVSCISSADGSTYYA
DSVKGRFTISRDNAKNTVYLQMNSLGPEDTAVYYCATACADTTQYDYDYWGQGTQVTVS
S,
(SEQ ID NO: 26)
QVQLQESGGGLVQAGGSLRLSCAASGFSLGYYAIGWFRQAPGKEREGVSCISSSDGHSSTY
YADSVKGRFTISRDNAKNTVYLQMNNLKPEDTAVYYCATDCAGGTATPYDYWGQGTQVT
VSS,
(SEQ ID NO: 27)
QVQLQESGGGLVQAGGSLRLSCAASGRTFSSYGMGWFRQAPGKEREFVAAISWSGGSTYY
ADSVKGRFTISRDNAKNTVYLQMNSLKPEDTAVYYCASSAWPAGPKHQVEYDYWGQGTQ
VTVSS,
(SEQ ID NO: 28)
QVQLQESGGGLVQAGGSLRLSCTASGFSLDYYAIGWFRQAPGKEREGVACISSRTGSTYYA
DSVKGRFTISRDNAKNTVALQMNSLKPEDTAVYYCATACVVVGDQNDYWGQGTQVTVSS,
(SEQ ID NO: 29)
QVQLQESGGGLVQDGGSLRLSCAASGFPLAYYAIGWFRQAPGKEREGVSCISASDGSTYYA
DSVKGRFTISRDNAKNTVYLQMNSLKPEDTAVYYCATACAETTLYEYDYWGQGTQVTVSS,
(SEQ ID NO: 30)
QVQLQESGGGLVQPGESLRLSCAASGFPLAYYAIGWFRQAPGKEREGVSCISSSDGSTYYA
DSVKGRFTISRDNAKNTVYLQMNSLKPEDTAVYYCATACANWSTLGPYDYWGQGTQVTV
SS,
(SEQ ID NO: 31)
QVQLQESGGGLVQPGESLRLSCAASGFTLAYYAIGWFRQAPGKEREGVSCISSSDGNTYYA
DSVKGRFTISRDNAKNTVYLQMNRLKPEDTAVYYCATACADTTQYEYDYWGQGTQVTVS
S,
(SEQ ID NO: 32)
QVQLQESGGGLVQPGGSLKLSCAASGSDFRADAMGWYRQAPGKEREPVAIDSITSIYYVDS
VEGRFTISRDNTKNTVYLQMTSLKPEDTAVYYCYARYSGRTYWGRGTQVTVSS,
(SEQ ID NO: 33)
QVQLQESGGGLVQPGGSLRLSCAASGFPLAYYAIGWFRQAPGKEREGVSCISASDGSTYYA
DSVKGRFTISRDNAKNTVYLQMNSLRPEDTAVYYCATACADTTLYEYDYWGQGTQVTVSS,
(SEQ ID NO: 34)
QVQLQESGGGLVQPGGSLRLSCAASGFPLAYYAIGWFRQAPGKEREGVSCISSSDGNTYYA
DAVKGRFAISRDNAKNTVYLQMNSLKPEDTAVYYCATACSDPRVYEYDYWGQGTQVTVS
S,
(SEQ ID NO: 35)
QVQLQESGGGLVQPGGSLRLSCAASGFPLAYYAIGWFRQAPGKEREGVSCISSSDGNTYYA
DAVKGRFTISRDNAKNTVYLQMNSLKPEDTAVYYCATACADTTQHEYDYWGQGTQVTVS
S,
(SEQ ID NO: 36)
QVQLQESGGGLVQPGGSLRLSCAASGFPLAYYAIGWFRQAPGKEREGVSCISSSDGNTYYA
DAVKGRFTISRDNAKNTVYLQMNSLKPEDTAVYYCATACVDTTHYEYDYWGQGTQVTVS
S,
(SEQ ID NO: 37)
QVQLQESGGGLVQPGGSLRLSCAASGFPLAYYAIGWFRQAPGKEREGVSCISSSDGNTYYA
DSVKGRFTISRDNAKNTVYLQMNSLKPEDTAVYYCATACADATQHEYDYWGQGTQVTVS
S,
(SEQ ID NO: 38)
QVQLQESGGGLVQPGGSLRLSCAASGFPLAYYAIGWFRQAPGKEREGVSCISSSDGSTYYA
DSVKGRFTISRDNAKNTVYLQMNSLGPEDTAVYYCATACADTTQYDYDYWGQGTQVTVS
S,
(SEQ ID NO: 39)
QVQLQESGGGLVQPGGSLRLSCAASGFPLAYYAIGWFRQAPGKEREGVSCISSSDGSTYYA
DSVKGRFTISRDNAKNTVYLQMNSLKPEDTAVYYCATACADTTQYEYDYWGQGTQVTVS
S,
(SEQ ID NO: 40)
QVQLQESGGGLVQPGGSLRLSCAASGFPLAYYAIGWFRQAPGKEREGVSCISSSDGSTYYA
DSVKGRFTISRDNAKNTVYLQMNSLKPEDTAVYYCATACGGATGPYDYWGQGTQVTVSS,
(SEQ ID NO: 41)
QVQLQESGGGLVQPGGSLRLSCAASGFPLAYYAIGWFRRAPGKEREGVSCISSSDGNTYYA
DAVKGRFTISRDNAKNTVYLQMNSLKPEDTAVYYCATACADTTQHEYDYWGQGTQVTVS
S,
(SEQ ID NO: 42)
QVQLQESGGGLVQPGGSLRLSCAASGFPLDYYAIGWFRQAPGKEREGVSCISAGDGSSTYY
ADSVKGRFTISRDNAKNTVYLQMNSLKPEDTAVYYCATACASTTLYEYDYWGQGTQVTVS
S,
(SEQ ID NO: 43)
QVQLQESGGGLVQPGGSLRLSCAASGFPLDYYAIGWFRQAPGKEREGVSCISSADGSTYYA
DSVKGRFTISRDNAKNAVYLQMNSLGPEDTAVYYCATACADTTQYDYDYWGQGTQVTVS
S,
(SEQ ID NO: 44)
QVQLQESGGGLVQPGGSLRLSCAASGFPLDYYAIGWFRQAPGKEREGVSCISSADGSTYYA
DSVKGRFTISRDNAKNTVYLQMNSLGPEDTAVYYCATACADTTQYDYDYWGQGTQVTVS
S,
(SEQ ID NO: 45)
QVQLQESGGGLVQPGGSLRLSCAASGFPLDYYAIGWFRQAPGKEREGVSCISSADGSTYYA
DSVKGRFTISRDNAKNTVYLQMNSLKPEDTAVYYCATACVDTTQYEYDYWGQGTQVTVS
S,
(SEQ ID NO: 46)
QVQLQESGGGLVQPGGSLRLSCAASGFPLDYYAIGWFRQAPGKEREGVSCISSPDGSTYYA
DSVKGRFTISRDNAKNTVYLQMNSLKPEDTAVYYCATACVDTTQYEYDYWGQGTQVTVS
S,
(SEQ ID NO: 47)
QVQLQESGGGLVQPGGSLRLSCAASGFPLDYYAIGWFRQAPGKEREGVSCISSSDGSDGNT
YYADSVKGRFTISRDNAKNTVYLQMNSLKPEDTAVYYCATDCSLHGSDYPYDYWGQGTQ
VTVSS,
(SEQ ID NO: 48)
QVQLQESGGGLVQPGGSLRLSCAASGFPLDYYAIGWFRQAPGKEREGVSCISSSDGSTYYA
DSVKGRFTISRDNAKNTVYLQMNSLKPEDTAVYYCATACADTTQYEYDYWGQGTQVTVS
S,
(SEQ ID NO: 49)
QVQLQESGGGLVQPGGSLRLSCAASGFPLEYYAIGWFRQAPGKEREGVSCISSSDGSTYYA
DSVKGRFTISRDNAKNTVYLQMNSLKPEDTAVYYCATACSDPRVYEYDYWGQGTQVTVSS,
(SEQ ID NO: 50)
QVQLQESGGGLVQPGGSLRLSCAASGFPLGYYAIGWFRQAPGKEREGVSCISSSDDSTYYA
DSVKGRFTISRDNDKNTVYLQMNSLKPEDTAVYYCATDCAGGTSTPYDYWGQGTQVTVSS,
(SEQ ID NO: 51)
QVQLQESGGGLVQPGGSLRLSCAASGFPLHYYAIGWFRQAPGKEREGVSCISSGDGSTYYA
DSVKGRFTISRDNAKNTVYLQMNSLKPEDTAVYYCATSCVVVTKNEYDYWGQGTQVTVS
S,
(SEQ ID NO: 52)
QVQLQESGGGLVQPGGSLRLSCAASGFPLHYYAIGWFRQAPGKEREGVSCISSSDGSTYYA
DSVKGRFTISRDNAKNTVYLQMNSLKPEDTAVYYCATACGGATGPYDYWGQGTQVTVSS,
(SEQ ID NO: 53)
QVQLQESGGGLVQPGGSLRLSCAASGFPLHYYAIGWFRQAPGKEREGVSCISSSDGSTYYA
DSVKGRFTISRDNAKNTVYLQMNSLKPEDTAVYYCATACVVADRNEYDYWGQGTQVTVS
S,
(SEQ ID NO: 54)
QVQLQESGGGLVQPGGSLRLSCAASGFPLHYYAIGWFRQAPGKEREGVSCISSSDGSTYYA
DSVKGRFTISRDNAKNTVYLQMNSLRPEDTAVYYCATACVVADRNEYDYWGQGTQVTVS
S,
(SEQ ID NO: 55)
QVQLQESGGGLVQPGGSLRLSCAASGFPLNYYAIGWFRQAPGKEREGVSCISASDGNTYYA
DSVKGRFTISRDNAKNTVYLQMNSLKPEDTAVYYCATTCASPEKYEYDYWGQGTQVTVSS,
(SEQ ID NO: 56)
QVQLQESGGGLVQPGGSLRLSCAASGFPLNYYAIGWFRQAPGKEREGVSCISSSDGSTYYA
DSVKGRFIISRDNAKNTVYLQMNSLKPEDTAVYYCATACGGATGPYDYWGQGTQVTVSS,
(SEQ ID NO: 57)
QVQLQESGGGLVQPGGSLRLSCAASGFPLNYYAIGWFRQAPGKEREGVSCISSSDGSTYYA
DSVKGRFTISRDNAKNTVYLQMNSLKPEDTAVYYCATACGSAVHEYDYWGQGTQVTVSS,
(SEQ ID NO: 58)
QVQLQESGGGLVQPGGSLRLSCAASGFSLAYYAIGWFRQAPGKEREGVSCIAASVGSTYYA
DSVKGRFTISRDDAKNTVYLQMNSLKPEDTAVYYCATDCAGGVGHEYDYWGQGTQVTVS
S,
(SEQ ID NO: 59)
QVQLQESGGGLVQPGGSLRLSCAASGFSLDYYAIGWFRQAPGKEREGVSCISSSDGSTYYA
DSVKGRFTISRDNAKNAVYLQMNSLKPEDTAVYYCATACGGATGPYDYWGQGTQVTVSS,
(SEQ ID NO: 60)
QVQLQESGGGLVQPGGSLRLSCAASGFSLDYYAIGWFRQAPGKEREGVSCISSSDGSTYYA
DSVKGRFTISRDNAKNAVYLQMNSLKPEDTAVYYCATACVDTTQYEYDYWGQGTQVTVS
S,
(SEQ ID NO: 61)
QVQLQESGGGLVQPGGSLRLSCAASGFSLDYYAIGWFRQAPGKEREGVSCISSSDGSTYYA
DSVKGRFTISRDNAKNTVYLQMNSLKPEDTAVYYCATDCAGGTSTPYDYWGQGTQVTVSS,
(SEQ ID NO: 62)
QVQLQESGGGLVQPGGSLRLSCAASGFSLNYYAIGWFRQAPGKEREGVSCISAGDGNTYYA
DSVKGRFTISRDNAANTVSLQMDSLKPEDTAVYYCATACVITTLYEYDYWGQGTQVTVSS,
(SEQ ID NO: 63)
QVQLQESGGGLVQPGGSLRLSCAASGFTLAYYAIGWFRQAPGKEREGVSCISSSDGSTYYA
DSVKGRFTISRDNAKNTVYLQMNSLKPEDTAVYYCATACADTTQHEYDYWGQGTQVTVS
S,
(SEQ ID NO: 64)
QVQLQESGGGLVQPGGSLRLSCAASGFTLAYYAIGWFRQAPGKEREGVSCISSSDGSTYYA
DSVKGRFTISRDNAKNTVYLQMNSLKPEDTAVYYCATACADTTQYEYDYWGQGTQVTVS
S,
(SEQ ID NO: 65)
QVQLQESGGGLVQPGGSLRLSCAASGFTLDYYAIGWFRQAPGKEREGVACISSSDGSTYYA
DSVKGRFTISRDNAKNTVYLQMNSLKPEDTAVYYCATACGGATGPYDYWGQGTQVTVSS,
(SEQ ID NO: 66)
QVQLQESGGGLVQPGGSLRLSCAASGFTLDYYAIGWFRQAPGKEREGVACISSSDGSTYYA
DSVKGRFTISRDNAKNTVYLQMNSLKPQDTAVYYCATACGSLVGMYDYWGQGTQVTVSP,
(SEQ ID NO: 67)
QVQLQESGGGLVQPGGSLRLSCAASGFTLDYYAIGWFRQAPGKEREGVSCISASDGNTYYA
DSVKGRFTISRDNAKNTVYLQMNSLKPEDTAVYYCATTCASPEKYEYDYWGQGTQVTVSS,
(SEQ ID NO: 68)
QVQLQESGGGLVQPGGSLRLSCAASGFTLDYYAIGWFRQAPGKEREGVSCISASNGNTYYA
DSVKGRFTISRDSAKNTVYLQMNSLKPEDTAVYYCATTCSGLTHEYDYWGQGTQVTVSS,
(SEQ ID NO: 69)
QVQLQESGGGLVQPGGSLRLSCAASGFTLDYYAIGWFRQAPGKEREGVSCISSGDGNTYYA
DSVKGRFTISRDNAKNTVYLQMNSLKPEDTAVYYCATACGGATGPYDYWGQGTQVTVSS,
(SEQ ID NO: 70)
QVQLQESGGGLVQPGGSLRLSCAASGFTLDYYAIGWFRQAPGKEREGVSCISSGDGSTYYA
DSVKGRFTISRDNAKNTVYLQMNSLKPEDTAVYYCATHCGGSSWSNEYDYWGQGTQVTV
SS,
(SEQ ID NO: 71)
QVQLQESGGGLVQPGGSLRLSCAASGFTLDYYAIGWFRQAPGKEREGVSCISSNDGSTYYA
DSVKGRFTISRDNAKNTVYLQMNSLKPEDTAVYYCATACADTTQHEYDYWGQGTQVTVS
S,
(SEQ ID NO: 72)
QVQLQESGGGLVQPGGSLRLSCAASGFTLDYYAIGWFRQAPGKEREGVSCISSSDGGTYYA
DSVKGRFTISRDNAKNTVYLQMNSLKPEDTAVYYCATACGGATGPYDYWGQGTQVTVSS,
(SEQ ID NO: 73)
QVQLQESGGGLVQPGGSLRLSCAASGFTLDYYAIGWFRQAPGKEREGVSCISSSDGSSSDGN
TYYADSVKGRFTISRDNAKNTVYLQMNNLKPEDTAVYYCATACVVTTLYEYDYWGQGTQ
VTVSS,
(SEQ ID NO: 74)
QVQLQESGGGLVQPGGSLRLSCAASGFTLDYYAIGWFRQAPGKEREGVSCISSSDGSTYYA
DSVKGRFTISRDNAKNTVYLQMNSLKPEDTAVYYCATACADTTQYEYDYWGQGTQVTVS
P,
(SEQ ID NO: 75)
QVQLQESGGGLVQPGGSLRLSCAASGFTLDYYAIGWFRQAPGKEREGVSCISSSDGSTYYA
DSVKGRFTISRDNAKNTVYLQMNSLKPEDTAVYYCATACGGATGPYDYWGQGTQVTVSS,
(SEQ ID NO: 76)
QVQLQESGGGLVQPGGSLRLSCAASGFTLDYYAIGWFRQAPGKEREGVSCISSSGGSTYYA
DSVKGRFTISRDNAKNTVYLQMNMLKPEDTAVYYCATACADTTQYEYDYWGQGTQVTVS
S,
(SEQ ID NO: 77)
QVQLQESGGGLVQPGGSLRLSCAASGFTLDYYAIGWFRQAPGKEREGVSCISSSGGSTYYA
DSVKGRFTISRDNAKNTVYLQMNSLKPEDTAVYYCATACASPVIYEYDYWGQGTQVTVSS,
(SEQ ID NO: 78)
QVQLQESGGGLVQPGGSLRLSCAASGFTLDYYAIGWFRQAPGKEREGVSCISSSGGSTYYA
DSVKGRFTISRDNAKNTVYLQMNSLKPEDTAVYYCATDCAGGTSTPYDYWGQGTQVTVSS,
(SEQ ID NO: 79)
QVQLQESGGGLVQPGGSLRLSCAASGFTLGYYAIGWFRQAPGKEREGVSCISSSDGSTYYA
DSVKGRFTISRDNAKNTVYLQMNSLKPEDTAVYYCATACADTTQYEYDYWGQGTQVTVS
S,
(SEQ ID NO: 80)
QVQLQESGGGLVQPGGSLRLSCAASGFTLGYYAIGWFRQAPGKEREGVSCISSSDGSTYYA
DSVKGRFTISRDNAKNTVYLQMNSLKPEDTAVYYCATACANWSSLGPYDYWGQGTQVTV
SS,
(SEQ ID NO: 81)
QVQLQESGGGLVQPGGSLRLSCAASGFTLGYYAIGWFRQAPGKEREGVSCISSSDGSTYYA
DSVKGRFTISRDNAKNTVYLQMNSLKPEDTGVYYCATACGGATGPYDYWGQGTQVTVSS,
(SEQ ID NO: 82)
QVQLQESGGGLVQPGGSLRLSCEGSGFSLDYYAIGWFRQAPGKEREGVSCISSGDGNTYYA
DSVKGRFTISRDNAKNTVYLQMNSLKPEDTAVYYCATDCVGFGSNWFDYWGQGTQVTVS
S,
(SEQ ID NO: 83)
QVQLQESGGGLVQPGGSLRLSCTASGFSLDYYAIGWFRQAPGKEREGVACISSRTGSTYYA
DSVKGRFTISRDNAKNTVALQMNSLKPEDTAVYYCATACVVVGDQNDYWGQGTQVTVSS,
(SEQ ID NO: 84)
QVQLQESGGGLVQPGGSLRLSCTASGFSLDYYAIGWFRQAPGKEREGVSCISSRTGGTYYA
DSVKGRFTISRDDAKNTVYLQMNSLKPEDTAVYYCATACVVVGDRNDYWGQGTQVTVSS,
(SEQ ID NO: 85)
QVQLQESGGGLVQPGGSLRLSCTASGFSLDYYAIGWFRQAPGKEREGVSCISSRTGGTYYA
DSVKGRFTISRDNAKNTVYLQMNSLKPEDTAVYYCATACVDTTQYEYDYWGQGTQVTVS
S,
(SEQ ID NO: 86)
QVQLQESGGGLVQPGGSLRLSCTASGFSLDYYAIGWFRQAPGKEREGVSCISSRTGGTYYA
DSVKGRFTISRDNAKNTVYLQMNSLKPEDTAVYYCATACVVVGDQNDYWGQGTQVTVSS,
(SEQ ID NO: 87)
QVQLQESGGGLVQPGGSLRLSCTASGFSLDYYAIGWFRQAPGKEREGVSCISSRTGNTYYA
DSVKGRFTISRDDAKNMVYLQMNSLKPEDTAVYYCATACVVVGDQNDYWGQGTQVTVS
S,
(SEQ ID NO: 88)
QVQLQESGGGLVQPGGSLRLSCTASGFSLDYYAIGWFRQAPGKEREGVSCISSRTGSTYYA
DSVKGRFTISRDDAKNTVYLQMNSLKPEDTAVYYCATACVVVGDQNDYWGQGTQVTVSS,
(SEQ ID NO: 89)
QVQLQESGGGLVQPGGSLRLSCTASGFSLGYYAIGWFRQALGKEREGVSCISSRTGSTYYA
DSVKGRFTVSRDDAKNTVYLQMNSLKPEDTAVYYCATACVVVGDQNDYWGQGTQVTVS
S,
(SEQ ID NO: 90)
QVQLQESGGGLVQPGGSLRLSCTASGFSLGYYAIGWFRQAPGKEREGVSCISSRTGSTYYA
DSVKGRFAISRDDAKNTVYLQMNSLKPEDTAVYYCATACVVVGDQNDYWGQGTQVTVSS,
(SEQ ID NO: 91)
QVQLQESGGGLVQPGGSLRLSCTASGFSLGYYAIGWFRQAPGKEREGVSCISSRTGSTYYA
DSVKGRFTISRDDAKNTVYLQMNSLKPEDTAVYYCATACVVVGDQNDYWGQGTQVTVSS,
(SEQ ID NO: 92)
QVQLQESGGGLVQPGGSLRLSCTASGFSLGYYAIGWFRQAPGKEREGVSCISSRTGSTYYA
DSVKGRFTVSRDDAKNTVYLQMNSLKPEDTAVYYCATACVVVGDQNDYWGQGTQVTVS
S,
(SEQ ID NO: 93)
QVQLQESGGGLVQPGGSLRLSCVASGFPLDYYAIGWFRQAPGKEREGVSCISSSDGSTYYA
DSVKGRFTISRDNAKNTVYLQMNSLKPEDTAVYYCATACGGATGPYDYWGQGTQVTVSS,
(SEQ ID NO: 94)
QVQLQESGGGLVQPGGSLRLSCVASGFSLDYYAIGWFRQAPGKEREGVSCISNSDGSTYYA
DSVKGRFTISRDNAKNTVYLQMNSLKPEDTAVYYCATACADTTQYAYDYWGQGTQVTVS
S,
(SEQ ID NO: 95)
QVQLQESGGGLVQPGGSLRLSCVASGFTLDYYAIGWFRQAPGKEREGVSCISSGSDGNTYY
ADSVKGRFTISRDNAKNTVYLQMNSLKPEDTAVYYCATACSGLTHEYDYWGQGTQVTVSS,
(SEQ ID NO: 96)
QVQLQESGGGLVQPGGSLRLSCVASGFTLDYYAIGWFRQAPGKEREGVSCISSSDDSTYYA
DSVKGRFTISRDNAKNTVYLQMNSLKPEDTAVYYCATACADTTQYEYDYWGQGTQVTVS
S,
(SEQ ID NO 97)
QVQLQESGGGLVQPGGSLRLSCVASGFTLDYYAIGWFRQAPGKEREGVSCISSSSDGNTYY
ADSVKGRFTISRDNAKNTVYLQMNSLKPEDTAVYYCATTCSGLTHEYDYWGQGTQVTVSS,
(SEQ ID NO: 98)
QVQLQESGGGLVQPGGSLRLSCVASGFTLGYYAIGWFRQAPGKEREGVSCISSSDGSTYYA
DSVKGRFTISRDNAKNTVYLQMNSLKPEDTAVYYCATACADTTQYDYDYWGQGTQVTVS
S,
(SEQ ID NO: 99)
QVQLQESGGGLVQPGGSLRLSCVGSGFTLDYYAIGWFRQAPGKEREGVSCISSNDGSTYYA
DSVKGRFTISRDNAKNTVYLQMNSLKPEDTAVYYCATACGGATGPYDYWGQGTQVTVSS,
(SEQ ID NO: 100)
QVQLQESGGGLVQSGGSLRLSCAASGFPLAYYAIGWFRQAPGKEREGVSCISASDGSTYYA
DSVKGRFTISRDNAKNTVYLQMNSLKPEDTAVYYCATACAETTLYEYDYWGQGTQVTVSS,
(SEQ ID NO: 101)
QVQLQESGGGLVQTGGSLRLSCAASGFTLDYYAIGWFRQAPGKEREGVSCISSSDGSTYYA
DSVKGRFTISRDNAKNTVYLQMNSLKPEDTAVYYCATACGGATGPYDYWGQGTQVTVSS,
(SEQ ID NO: 102)
QVQLQESGGGMVQAGESLRLSCAASGFPLAYYAIGWFRQAPGKEREGVSCISSSDGNTYYA
DSVKGRFTISRDNAKNTVYLQMNSLKPEDTAVYYCATACADATQHEYDYWGQGTQVTVS
S,
(SEQ ID NO: 103)
QVQLQESGGGSVQPGESLRLSCAASGFPLDYYAIGWFRQAPGKEREGVSCISASDGSTYYA
DSVKGRFTISRDNAKNTVYLQMNSLKPEDTAVYYCATACADTTLYEYDYWGQGTQVTVSS,
(SEQ ID NO: 104)
QVQLQESGGGSVQPGGSLRLSCAASGFTLDYYAIGWFRQAPGKEREGVSCISSGDGSTYYA
DSVKGRFTISRDNAKNTVYLQMNSLKPEDTAVYYCATACADTTHYEYDYWGQGTQVTVS
S,
(SEQ ID NO: 105)
QVQLQESGGGSVQSGGSLRLSCTASGFSLGYYAIGWFRQAPGKEREGVSCISSRTGSTYYAD
SVKGRFTVSRDDAKNTVYLQMNSLKPEDTAVYYCATACVVVGDQNDYWGQGTQVTVSS,
(SEQ ID NO: 106)
QVQLQESGGGSVRPGGSLRLSCAASGFPLAYYAIGWFRQAPGKEREGVSCISSSDGNTYYA
DAVKGRFTISRDNAKNAVYLQMNSLKPEDTAVYYCATACADTTQHEYDYWGQGTQVTVS
S,
(SEQ ID NO: 107)
QVQLQESGGGVAQPGGSLRLSCAASGFPLDYYAIGWFRQAPGKEREGVSCISASDGSTYYA
DSVKGRFTISRDNAKNTVYLQMNSLKPEDTAVYYCATACADTTLYEYDYWGQGTQVTVSS,
(SEQ ID NO: 108)
QVQLQESGGGVVQAGGSLKLSCAASGSDFRADAMGWYRQAPGKEREPVAIDSITSIYYVDS
VEGRFTISRDNTKNTVYLQMTSLKPEDTAVYYCYARYSGRTYWGRGTQVTVSS,
(SEQ ID NO: 109)
QVQLQESGGGVVQPGGSLRLSCAASGFSLDYYAIGWFRQAPGKEREGVSCISSGDGSTYYA
DSVKGRFTISRDNAKNTVYLQMNSLKPEDTAVYYCATHCGGTSWGTSYDYWGQGTQVTV
SS,
(SEQ ID NO: 110)
QVQLQESGGGVVQPGGSLRLSCAASGLTFRSVGMGWFRRAPGKEREFVATASPSGVITYYA
DSVKGRFTISRDNAKNTVYLEMNSLKPEDTAVYYCAVRIYSGSFDNTLAYDYWGQGTQVT
VSS,
(SEQ ID NO: 111)
QVQLQESGGGVVQPGGSLRLSCTASGFSLGYYAIGWFRQAPGKEREGVSCISSRTGSTYYA
DSVKGRFTVSRDDAKNTVYLQMNSLKPEDTAVYYCATACVVVGDQNDYWGQGTQVTVS
S,
and
(SEQ ID NO: 112)
QVQLQESGGGVVQSGGSLRLSCTASGFSLDYYAIGWFRQAPGKEREGVSCISSRTGSTYYA
DSVKGRFTISRDDAKNTVYLQMNSLKPEDTAVYYCATACVVVGDQNDYWGQGTQVTVSS.

In some embodiments, the VH is a single domain antibody domain.

In some embodiments, the VH is a VHH.

In one aspect, provided herein is a composition comprising a construct comprising an antigen binding domain comprising an anti-IL13Rα2 antibody or binding fragment thereof, or a recombinant nucleic acid encoding the antigen binding domain, wherein the antigen binding domain comprises a heavy chain variable domain (VH) comprising a heavy chain complementarity determining region 3 (HC CDR3) sequence selected from the group consisting of ALRRGARIL (SEQ ID NO: 253), AARDRLLNTVTNIDYDY (SEQ ID NO: 254), GADAVFYSGGYYSDS (SEQ ID NO: 255), RVAGRQDDY (SEQ ID NO: 256), AARRASTIA (SEQ ID NO: 257), NVVGVLHTGHYENSF (SEQ ID NO: 258), NARRGARVL (SEQ ID NO: 259), NARPGLRSY (SEQ ID NO: 260), NIVGVLTTGHYEDSF (SEQ ID NO: 261), NIVGVLTTGHYENSF (SEQ ID NO: 262), ALRRGGRIL (SEQ ID NO: 263), NARPGLQSY (SEQ ID NO: 264), NARPGLQSF (SEQ ID NO: 265).

In some embodiments, the VH of the anti-IL13Rα2 antibody or binding fragment thereof further comprises a heavy chain complementarity determining region 1 (HC CDR1) that is the HC CDR1 of any one of the VH sequences selected from the group consisting of GINISSDV (SEQ ID NO: 266), GSSYSTSD (SEQ ID NO: 267), GSIFSINR (SEQ ID NO: 268), ASIFSISA (SEQ ID NO: 269), QTITRLST (SEQ ID NO: 270), GSIFNTNG (SEQ ID NO: 271), GSISSINR (SEQ ID NO: 272), GSIRDIGY (SEQ ID NO: 273), GSIRGIGY (SEQ ID NO: 274 or SEQ ID NO: 334), GIFVSANS (SEQ ID NO: 275), GINASGDV (SEQ ID NO: 276), GLTFSNYD (SEQ ID NO: 277), GGIFTVND (SEQ ID NO: 278), GINISRDV (SEQ ID NO: 279), GIRISSDV (SEQ ID NO: 280), GIFVSANT (SEQ ID NO: 281), GVTGEIEA (SEQ ID NO: 282), GINISNDV (SEQ ID NO: 283), GTNISSDV (SEQ ID NO: 284), GDIYRINT (SEQ ID NO: 285), GIALDYYA (SEQ ID NO: 286).

In some embodiments, the VH of the anti-IL13Rα2 antibody or binding fragment thereof further comprises a heavy chain complementarity determining region 2 (HC CDR2) that is the HC CDR2 of any one of the VH sequences selected from the group consisting of HVTTDSHTE (SEQ ID NO: 287), AISPAGATN (SEQ ID NO: 288), HITTDSRTN (SEQ ID NO: 289), SITSSGDTM (SEQ ID NO: 290), MLGSTTD (SEQ ID NO: 291), HITTYSRTD (SEQ ID NO: 292), VIRRGGNTN (SEQ ID NO: 293), HVSTDSHTE (SEQ ID NO: 294), HVTADSHTE (SEQ ID NO: 295), SITSSGDTT (SEQ ID NO: 296), HITTNSRTE (SEQ ID NO: 297), TINSAGDTN (SEQ ID NO: 298), TINSAGNTN (SEQ ID NO: 299), TINSAGATN (SEQ ID NO: 300), TLNSAGNTN (SEQ ID NO: 301), QITYGDTIK (SEQ ID NO: 302), LMDTAYITD (SEQ ID NO: 303), GISSGGNTV (SEQ ID NO: 304), SMTGNGDTM (SEQ ID NO: 305).

In some embodiments, the VH of the anti-IL13Rα2 antibody or binding fragment thereof comprises with 70-100% sequence identity to any one of the sequences selected from the group consisting of

(SEQ ID NO: 195)
QVQLQESGGGLVQAGGSLRLSCAASGINISSDVMGWYRQGPDNERELVAHVTTDSHTEYA
DSVKGRFTISRDNAKNTVYLQMNDLKPEDTAPYYCALRRGARILWGQGTQVTVSS,
(SEQ ID NO: 196)
QVQLQESGGGLVQPGESLRLSCAASGINISSDVMGWYRQGPDNERELVAHVTTDSHTEYA
DSVKGRFTISRDNAKNTVYLQMNDLKPEDTAPYYCALRRGARILWGQGTQVTVSS,
(SEQ ID NO: 197)
QVQLQESGGGSVQPGGSLRLSCAASGINISSDVMGWYRQGPGNERELVAHVTTDSHTEYA
DSVKGRFTISRDNAKNTVYLQMNDLKPEDTAPYYCALRRGARILWGQGTQVTVSS,
(SEQ ID NO: 198)
QVQLQESGGGSVQPGESLRLSCAASGINISSDVMGWYRQGPDNERELVAHVTTDSHTEYAD
SVKGRFTISRDNAKNTVYLQMNDLKPEDTAPYYCALRRGARILWGQGTQVTVSS,
(SEQ ID NO: 199)
QVQLQESGGGLVQPGGSLRLSCATSGSSYSTSDMGWYRQAPGKQRELVAAISPAGATNYA
DFAQGRFAASRDNAKNTVYLQMNDLKPEDTAPYYCALRRGARILWGQGTQVTVSS,
(SEQ ID NO: 200)
QVQLQESGGGSVRPGGSLRLSCAASGINASGDVMGWYRQAPGNERELVAHITTDSRTNYA
DSVKGRFTISRDNAKNTVYLQMNDLKPEDTAPYYCALRRGARILWGQGTQVTVSS,
(SEQ ID NO: 201)
QVQLQESGGGSVQHGGSLRLSCTASGLTFSNYDMGWYRQGPDNERELVAHVTTDSHTEYA
DSVKGRFTISRDNAKNTVYLEMNDLKPEDTAPYYCALRRGARILWAQGTQVTVSS,
(SEQ ID NO: 202)
QVQLQESGGGSVQPGESLRLSCAASGINISSDVMGWYRQGPDNERELVAHVTTDSHTEYAD
SVKGRFTISRDNAKNMVYLQMNDLKPEDTAPYYCALRRGARILWGQGTQVTVSS,
(SEQ ID NO: 203)
QVQLQESGGGLVQAGESLRLSCAASGINISSDVMGWYRQGPDNERELVAHVTTDSHTEYA
DSVKGRFTISRDNAKNTVYLQMNDLKPEDTAPYYCALRRGARILWGQGTQVTVSS,
(SEQ ID NO: 204)
QVQLQESGGGLVQPGGSLRLSCAASGIFVSANSMGWYRQGPEDARELVASITSSGDTMYG
DSWKGRFAISRDNAKNTLYLQMNLLKSDDTAVYYCNARRGARVLWGKGTQVTVSS,
(SEQ ID NO: 205)
QVQLQESGGGSVQPGGSLRLSCAASGINISSDVMGWYRQGPDNERELVAHVTTDSHTEYA
DSVKGRFTISRDNAKNTVYLQMNDLKPEDTAPYYCALRRGARILWGQGTQVTVSS,
(SEQ ID NO: 206)
QVQLQESGGGLVQPGGSLRLSCAASGINISSDVMGWYRQGPDNERELVAHVTTDSHTEYA
DSVKGRFTISRDNAKNTVYLQMNDLKPEDTAPYYCALRRGARILWGQGTQVTVSS,
(SEQ ID NO: 207)
QVQLQESGGGLVHSGGSLRLSCAASGINISSDVMGWYRQGPDNERELVAHVTTDSHTEYA
DSVKGRFTISRDNAKNTVYLQMNDLKPEDTAPYYCALRRGARILWGQGTQVTVSS,
(SEQ ID NO: 208)
QVQLQESGGGSVQPGESLRLSCAASGINISSDVMGWYRQGPDNERELVAHVTTDSHTEYAD
SVKGRFTISRDNAKNTVYLEMNDLKPEDTAPYYCALRRGARILWGQGTQVTVSS,
(SEQ ID NO: 209)
QVQLQESGGGVAQPGESLRLSCKVSGIALDYYAIGWLRQAPGKEREGVAMLGSTTDYSAS
VKGRFTISRGDAKNTVYLDMNNLKPEDTATYYCALRRGARILWGQGTQVTVSS,
(SEQ ID BO: 210)
QVQLQESGGGSVQPGGSLRLSCVASGINISSDVMEWYRQAPGEERELVAHITTYSRTDYSDS
VKGRFTISRANAKNTLYLQMNGLKPEDTALYYCALRRGARILWGQGTQVTVSS,
(SEQ ID NO: 211)
QVQLQESGGGSVQSGESLKLSCVGSGDIYRINTMAWYRQTPGKEREMVAVIRRGGNTNYA
DSVKGRFTISRDDSKNTMSLQMNSLRPEDTATYYCALRRGARILWGQGTQVTVSS,
(SEQ ID NO: 212)
QVQLQESGGGSVQPGESLRLSCAASGINISSDVMGWYRQGPDNERELVAHVTTDSHTEYAD
SVKGRFTISRDNAKNTVYLQMNDLKPEDTAPYYCALRRGARILWGPGTQVTVSS,
(SEQ ID NO: 213)
QVQLQESGGGSVQPGESLRLSCAASGINISSDVMGWYRQGPDNERELVAHVTTDSHTEYAD
SVKGRFTISRDNAKNTVYLQMNDLKPEDTAPYYCALRRGARILWSQGTQVTVSS,
(SEQ ID NO: 214)
QVQLQESGGGLVEPGGSLRLSCQISGGIFTVNDMGWYRRSPGTDRELVAHVTTDSHTEYAD
SVKGRFTISRDNAKNTVYLQMNDLKPEDTAPYYCALRRGARILWGQGTQVTVSS,
(SEQ ID NO: 215)
QVQLQESGGGLVKPGGSLRLSCAASGIFVSANSMGWYRQGPEDARELVASITSSGDTMYG
DSWKGRFAISRDNAKNTLYLQMNLLKSDDTAVYYCNARRGARVLWGKGTQVTVSS,
(SEQ ID NO: 216)
QVQLQESGGGSVQPGGSLRLSCAASGINISRDVMGWYRQGPVNERELVAHVTTDSHTEYA
DSVKGRFTISRDNAKNTVYLEMNDLKPEDTAPYYCALRRGARILWGQGTQVTVSS,
(SEQ ID NO: 217)
QVQLQESGGGLVQPGGSLRLSCAASGINISSDVMGWYRQGPDNERELVAHVTTDSHTEYA
DSVKGRFTISRDNAKNTVYLQMNDLRPEDTAPYYCALRRGARILWGQGTQVTVSS,
(SEQ ID NO: 218)
QVQLQESGGGLVQPGESLRLSCAASGINISSDVMGWYRQGPDNERELVAHVSTDSHTEYAD
SVKGRFTISRDNAKNTVYLQMNDLKPEDTAPYYCALRRGARILWGQGTQVTVSS,
(SEQ ID NO: 219)
QVQLQESGGGLVHSGESLRLSCAASGINISSDVMGWYRQGPDNERELVAHVTTDSHTEYA
DSVKGRFTISRDNAKNTVYLQMNDLKPEDTAPYYCALRRGARILWGQGTQVTVSS,
(SEQ ID NO: 220)
QVQLQESGGGSVQPGGSLRLSCAASGINISRDVMGWYRQGPGNERELVAHVTTDSHTEYA
DSVKGRFTISRDNAKNTVYLEMNDLKPEDTAPYYCALRRGARILWGQGTQVTVSS,
(SEQ ID NO: 221)
QVQLQESGGGSVQPGESLRLSCAASGINISSDVMGWYRQGPDNEHELVAHVTTDSHTEYA
DSVKGRFTISRDNAKNTVYLQMNDLKPEDTAPYYCALRRGARILWGQGTQVTVSS,
(SEQ ID NO: 222)
QVQLQESGGGSVQPGESLRLSCAASGINISSDVMGWYRQGPDNERELVAHVTTDSHTEYAD
SVKGRFTISRDNAKNTVYLQTNDLKPEDTAPYYCALRRGARILWGQGTQVTVSS,
(SEQ ID NO: 223)
QVQLQESGGGSVQPGGSLRLSCAASGIRISSDVMGWYRQGPGNDRELVAHVTADSHTEYA
DSAKGRFTISRDNAKNTVYLQMNDLKPEDTAPYYCALRRGARILWGQGTQVTVSS,
(SEQ ID NO: 224)
QVQLQESGGGSVQPGESLRLSCAAPGINISSDVMGWYRQGPDNERELVAHVTTDSHTEYAD
SVKGRFTISRDNAKNTVYLQMNDLKPEDTAPYYCALRRGARILWGQGTQVTVSS,
(SEQ ID NO: 225)
QVQLQESGGGSVQPGESLRLSCAASGINISSDVMGWYRQGPGNERELVAHVTTDSHTEYAD
SVKGRFTISRDNAKNTVYLQMNDLKPEDTAPYYCALRRGARILWGQGTQVTVSS,
(SEQ ID NO: 226)
QVQLQESGGGSVQPGESLRLSCAASGINISSDVMGWYRQGPDNERELVAHVTTDSHTEYAD
SVKGRFTISRDNASNTVYLQMNDLKPEDTAPYYCALRRGARILWGQGTQVTVSS,
(SEQ ID NO: 227)
QVQLQESGGGLVQPGGSLRLSCAASGIFVSANTMGWYRQGPEDARELVASITSSGDTTYAD
SWKGRFAISRDNAKNTLYLQMNLLKSDDTAVYYCNARRGARVLWGKGTQVTVSS,
(SEQ ID NO: 228)
QVQLQESGGGLVQPGGSLRLSCAASGINISSDVMGWYRQGPDDERELVAHVTTDSHTEYA
DSVKGRFTISRDNAKNTVYLQMNDLKPEDTAPYYCALRRGARILWGQGTQVTVSS,
(SEQ ID NO: 229)
QVQLQESGGGMVQPGESLRLSCVASGVTGEIEAMGWYRQGPEDARELVASITSSGDTTYG
DSWKGRFAISRDNAKNTLYLQMNLLKSDDTAVYYCNARRGARVLWGKGTQVTVSS,
(SEQ ID NO: 230)
QVQLQESGGGSVQPGGSLRLSCAASGINISSDVMEWFRQGPGEQRELVAHITTNSRTEYADF
VKGRFTISRANAKNTLYLQMNGLKPEDTALYYCALRRGGRILWGQGTQVTVSS,
(SEQ ID NO: 231)
QVQLQESGGGLVQPGESLRLSCAASGINISSDVMGWYRQGPDNERELVAHVTTDSHTEYA
DSVKGRLTISRDNAKNTVYLQMNDLKPEDTAPYYCALRRGARILWGQGTQVTVSS,
(SEQ ID NO: 232)
QVQLQESGGGSVQPGESLRLSCAASGTNISSDVMGWYRQGPDNERELVAHVTTDSHTEYA
DSVKGRFTISRDNAKNTVYLQMNDLKPEDTAPYYCALRRGARILWGQGTQVTVSS,
(SEQ IS NO: 233)
QVQLQESGGGLVTPGDSLRLSCAASGINISSDVMGWYRQGPDNERELVAHVTTDSHTEYA
DSVKGRFTISRDNAKNTVYLQMNDLKPEDTAPYYCALRRGARILWGQGTQVTVSS,
(SEQ ID NO: 234)
QVQLQESGGGLVQPGGSLSVSCAASGINISSDVMGWYRQGPDNERELVAHVTTDSHTEYA
DSVKGRFTISRDNAKNTVYLQMNDLKPEDTAPYYCALRRGARILWGQGTQVTVSS,
(SEQ ID NO: 235)
QVQLQESGGGSVQPGESLRLSCAASGINISNDVMGWYRQGPDNERELVAHVTTDSHTEYA
DSVKGRFTISRDNAKNTVYLQMNDLKPEDTAPYYCALRRGARILWGQGTQVTVSS,
(SEQ ID NO: 236)
QVQLQESGGGSVQPGESLRLSCAASGINISSDVMGWYRQGPDNERELVAHVTTDSHTEYAD
SVKGRFTISRDNAKNTVYLQMNDLKPEDAAPYYCALRRGARILWGQGTQVTVSS,
(SEQ ID NO: 237)
QVQLQESGGGLVQPGGSLRLSCTASGSIRGIGYMAWGRQGPGKPRELIATINSAGDTNYAES
VKGRFTVSRDNAENTLYLQMNNLKSEDTAVYYCNARPGLQSYWGQGTQVTVSS,
(SEQ ID NO: 238)
QVQLQESGGGLVQPGGSLRLSCTASGSIRDIGYMAWGRQAPGKQRELIATINSAGNTNYAE
SVKGRFTVSKDNAENTLYLQMNNLKSEDTAVYYCNARPGLRSYWGQGTQVTVSS,
(SEQ ID NO: 239)
QVQLQESGGGSAQSGGSLRLSCTASGSIRGIGYMAWGRQAPGKQRELIATINSAGDTNYAE
SVKGRFTVSRDNAENTLYLQMNNLKSEDTAVYYCNARPGLRSYWGQGTQVTVSS,
(SEQ ID NO: 240)
QVQLQESGGGLVQPGGSLRLSCTASGSIRGIGYMAWGRQAPDKPRELIATINSAGATNYAES
VKGRFTVSRDNAANTLYLQMNNLKSEDTAVYYCNARPGLQSFWGQGTQVTVSS,
(SEQ ID NO: 241)
QVQLQESGGGLVQPGGSLRLSCTASGSIRGIGYMAWGRQAPGKQRELIATINSAGDTNYAE
SVKGRFTVSRDNAENTLYLQMNNLKSEDTAVYYCNARPGLRSYWGQGTQVTVSS,
(SEQ ID NO: 242)
QVQLQESGGGSVQAGDSLRLSCTASGSIRGIGYMAWGRQAPGKQRELIATINSAGDTNYAE
SVKGRFTVSRDNAENTLYLQMNNLKSEDTAVYYCNARPGLRSYWGQGTQVTVSS,
(SEQ ID NO: 243)
QVQLQESGGGLVHSGGSLTLSCTASGSIRDIGYMAWGRQAPGKQRELIATINSAGNTNYAE
SVKGRFTVSKDNAENTLYLQMNNLKSEDTAVYYCNARPGLRSYWGQGTQVTVSS,
(SEQ ID NO: 244)
QVQLQESGGGLVQPGGSLRLSCTASGSIRDIGYMAWGRQAPGEPRELIATLNSAGNTNYAE
SVKGRFTVSKDNAENTLYLQMNNLKSEDTAVYYCNARPGLRSYWGQGTQVTVSS,
(SEQ ID NO: 245)
QVQLQESGGGLVQPGGSLRLSCEVSGSIFSINRMAWYRQVPEKQREMVAQITYGDTIKYYG
SAKGRFTISRDSAKNAVYLQMNSLKPEDTAVYRCNIVGVLTTGHYEDSFWGQGTQVTVPS,
(SEQ ID NO: 246)
QVQLQESGGGLVQPGGSLRLSCEVSGSIFSINRMAWYRQVPEKEREMVAQITYGDTIKYYG
SAKGRFTISRDSAKNAVYLQMNSLKPEDTAVYRCNIVGVLTTGHYEDSFWGQGTQVTVPS,
(SEQ ID NO: 247)
QVQLQESGGGLVQPGGSLRLSCEVSGSISSINRMAWYRQVPGKEREMVAQITYGDTIKYYG
SAKGRFTISKDSAKNAVYLQMNSLKPEDTAVYRCNIVGVLTTGHYENSFWGQGTQVTVSS,
(SEQ ID NO: 249)
QVQLQESGGGLVQPGGSLRLSCEVSGSIFSINRMAWYRQVPGKQREMVAQITYGDTIKYYG
SAKGRFTISRDSAKNAVYLQMNSLKPEDTAVYRCNVVGVLHTGHYENSFWGQGTQVTVSS,
(SEQ ID NO: 250)
QVQLQESGGGLVQPGGSLRLSCATSASIFSISAMGWYRQTSGSERELVALMDTAYITDYAD
TVKGRFTISRDNAKNTVNLQMNSLKPEDTAVYYCAARRASTIAWGQGTQVTVSS,
(SEQ ID NO: 251)
QVQLQESGGGLVQPGGSLRLSCAASGSIFNTNGMGWYRQAPGKQRERVAGISSGGNTVYA
ASVEGRFTISRDNAKNTGYLQMNNLKPEDTAVYYCRVAGRQDDYWGQGTQVTVSS,
(SEQ ID NO: 252)
QVQLQESGGGSVQAGGSLRLSCAASQTITRLSTIGWYRQSAGKGRDLVASMTGNGDTMYA
DSVRGRFTISRDNAKNTVYLQMDSLKPEDTAVYYCGADAVFYSGGYYSDSWGQGTQVTV
SS,
and
(SEQ ID NO: 248)
QVQLQESGGGSVQPGESLRLSCAASGINISSDVMGWYRQGPDNERELVAHVTTDSHTEYAD
SVKGRFTISRPSARNTVWLQMNSLKPEDTAVYYCAARDRLLNTVTNIDYDYWGQGTQVTV
SS.

In some embodiments, the VH is a single domain antibody domain.

In some embodiments, the VH is a VHH.

In some embodiments, the construct comprises an extracellular domain comprising the antigen binding domain, wherein extracellular domain is operably linked to a transmembrane domain.

In some embodiments, the transmembrane domain is operably linked to a intracellular domain comprising an intracellular signaling domain.

In some embodiments, the intracellular signaling domain is derived from an intracellular PI3-kinase recruitment domain, a phagocytosis receptor intracellular domain, a pattern recognition receptor intracellular domain, a CD40 intracellular domain, an FcR intracellular domain, a cytokine receptor intracellular domain, a chemokine receptor intracellular domain.

In some embodiments, the intracellular domain comprises at least two intracellular signaling domains.

In some embodiments, the extracellular domain comprises a hinge domain connecting the antigen binding domain and the transmembrane domain.

In some embodiments, the recombinant nucleic acid is an RNA.

In some embodiments, the recombinant nucleic acid is an mRNA.

In some embodiments, the recombinant nucleic acid is associated with one or more lipids.

In some embodiments, the recombinant nucleic acid is encapsulated in a liposome.

In some embodiments, the liposome is a lipid nanoparticle.

In some embodiments, the recombinant nucleic acid is a vector.

In some embodiments, the recombinant nucleic acid encodes a chimeric antigen receptor (CAR) comprising a VH domain described herein.

In some embodiments, the CAR comprises an extracellular domain, a transmembrane domain and an intracellular domain. In some embodiments, the CAR comprises a leader sequence. In some embodiments, the leader sequence comprises a sequence of MWLQSLLLLGTVACSIS (SEQ ID NO: 306). In some embodiments, extracellular domain comprises a hinge. In some embodiments the hinge is operatively connected to the transmembrane domain. In some embodiments, the hinge is a CD8alpha hinge. In some embodiments, the hinge has a sequence of ALSNSIMYFSHFVPVFLPAKPTTTPAPRPPTPAPTIASQPLSLRPEACRPAAGGAVHTRGLD (SEQ ID NO: 307). In some embodiments the extracellular domain comprises a linker. In some embodiments, the linker is operatively linked to the VH and the hinge. In some embodiments, the linker is operatively linked to the VH and the transmembrane domain. In some embodiments, the linker has a sequence of SGGGSG (SEQ ID NO: 308). In some embodiments, the transmembrane domain is a CD8 transmembrane domain. In some embodiments, the transmembrane domain has a sequence of IYIWAPLAGTCGVLLLSLVITLYC (SEQ ID NO: 309). In some embodiments, the intracellular domain comprises an intracellular signaling domain from FcεR. In some embodiments, the intracellular domain comprises a sequence of

(SEQ ID NO: 310)
RRLKIQVRKAAITSYEKSDGVYTGLSTRNQETYETLKHEKPPQ.

In some embodiments, the intracellular domain comprises at least two intracellular signaling domains. In some embodiments, the least two intracellular signaling domains are separated by a linker. In some embodiments the linker has a sequence of GSGS (SEQ ID NO: 311). In some embodiments, the intracellular domain comprises an intracellular signaling domain that that is the PI3K recruitment domain. In some embodiments, the PI3K recruitment domain has a sequence of

(SEQ ID NO: 314)
YEDMRGILYAAPQLRSIRGQPGPNHEEDADSYENM.

In some embodiments, the CAR comprises a sequence with 70-100% sequence identity to

(SEQ ID NO: 316)
MWLQSLLLLGTVACSISQVQLQESGGGLVQAGGSLRLSCAAPRSIFSIN
AMGWYRQAPGKQRELVAAITSGGSPTYADSVKGRFTISRDNAKNTVYLQ
MNSLKAEDTAVYYCATGPYGLDNALDAWGQGTQVTVSSSGGGGSGALSN
SIMYFSHFVPVFLPAKPTTTPAPRPPTPAPTIASQPLSLRPEACRPAAG
GAVHTRGLDIYIWAPLAGTCGVLLLSLVITLYCRRLKIQVRKAAITSYE
KSDGVYTGLSTRNQETYETLKHEKPPQGSGSYEDMRGILYAAPQLRSIR
GQPGPNHEEDADSYENM.

In some embodiments, the CAR comprises a sequence with 70-100% sequence identity to

(SEQ ID NO: 315)
QVQLQESGGGLVQAGGSLRLSCAAPRSIFSINAMGWYRQAPGKQRELVA
AITSGGSPTYADSVKGRFTISRDNAKNTVYLQMNSLKAEDTAVYYCATG
PYGLDNALDAWGQGTQVTVSSSGGGGSGALSNSIMYFSHFVPVFLPAKP
TTTPAPRPPTPAPTIASQPLSLRPEACRPAAGGAVHTRGLDIYIWAPLA
GTCGVLLLSLVITLYCRRLKIQVRKAAITSYEKSDGVYTGLSTRNQETY
ETLKHEKPPQGSGSYEDMRGILYAAPQLRSIRGQPGPNHEEDADSYEN
M.

In some embodiments, the CAR comprises a sequence with 70-100% sequence identity to

(SEQ ID NO: 346)
MWLQSLLLLGTVACSISQVQLQESGGGLVQTGGSLRLACTASGFTFDDY
AIAWFRQAPGKEREFVAAISWSGGTTHYADSVKGRFTISRDNAKNTLYL
QMSSLKPEDTAVYFCAKSLRSSPSSRWFGSRGQGTQVTVSSSGGGGSGA
LSNSIMYFSHFVPVFLPAKPTTTPAPRPPTPAPTIASQPLSLRPEACRP
AAGGAVHTRGLDIYIWAPLAGTCGVLLLSLVITLYCRRLKIQVRKAAIT
SYEKSDGVYTGLSTRNQETYETLKHEKPPQGSGSYEDMRGILYAAPQLR
SIRGQPGPNHEEDADSYENM.

In some embodiments, the CAR comprises a sequence with 70-100% sequence identity to

(SEQ ID NO: 347)
QVQLQESGGGLVQTGGSLRLACTASGFTFDDYAIAWFRQAPGKEREFVA
AISWSGGTTHYADSVKGRFTISRDNAKNTLYLQMSSLKPEDTAVYFCAK
SLRSSPSSRWFGSRGQGTQVTVSSSGGGGSGALSNSIMYFSHFVPVFLP
AKPTTTPAPRPPTPAPTIASQPLSLRPEACRPAAGGAVHTRGLDIYIWA
PLAGTCGVLLLSLVITLYCRRLKIQVRKAAITSYEKSDGVYTGLSTRNQ
ETYETLKHEKPPQGSGSYEDMRGILYAAPQLRSIRGQPGPNHEEDADSY
ENM.

In some embodiments, the CAR is encoded by a sequence with 70-100% sequence identity to

(SEQ ID NO: 348)
ATGTGGCTGCAGTCTCTGCTGCTGCTGGGAACAGTGGCCTGCAGCATCAG
CCAGGTGCAGTTGCAGGAGAGTGGCGGTGGCCTGGTGCAGACCGGTGGCA
GTCTGAGGCTGGCCTGCACTGCCAGCGGCTTCACCTTCGACGACTATGCC
ATCGCGTGGTTCAGGCAGGCCCCTGGCAAGGAGAGGGAGTTTGTCGCAGC
CATCAGCTGGAGCGGAGGCACCACCCACTACGCTGACAGCGTGAAGGGCA
GGTTCACCATTAGCAGGGACAATGCCAAGAACACCCTGTACCTGCAGATG
AGCAGTCTCAAGCCCGAAGATACCGCCGTGTACTTCTGCGCCAAGAGCCT
GAGGAGCAGCCCCAGCAGCAGGTGGTTCGGCAGCCGCGGTCAAGGCACCC
AGGTGACCGTGAGCTCTAGCGGAGGCGGAGGATCCGGAGCCCTGAGCAAT
AGCATCATGTACTTCAGCCACTTCGTGCCCGTGTTTCTGCCCGCCAAGCC
TACAACAACACCCGCTCCTAGACCACCTACACCAGCTCCTACAATCGCCA
GCCAGCCTCTGTCTCTCAGACCTGAAGCCTGTAGACCTGCAGCTGGCGGA
GCTGTGCATACCAGAGGCCTGGATATCTACATTTGGGCCCCTCTGGCTGG
CACATGTGGCGTTCTGCTGCTCTCTCTGGTCATCACCCTGTACTGCAGAC
GGCTGAAGATCCAAGTGCGGAAGGCCGCCATCACCAGCTACGAGAAATCT
GATGGCGTGTACACCGGCCTGAGCACCCGGAATCAAGAAACCTACGAGAC
ACTGAAGCACGAGAAGCCTCCACAAGCAGCGGCTCCTATGAGGACATGAG
AGGCATTCTGTACGCCGCTCCACAGCTGAGATCTATCAGAGGCCAGCCTG
GACCTAACCACGAGGAAGATGCCGACTCCTACGAGAACATG.

In some embodiments, the CAR is encoded by a sequence with 70-100% sequence identity to

(SEQ ID NO: 349)
CAGGTGCAGTTGCAGGAGAGTGGCGGTGGCCTGGTGCAGACCGGTGGCAG
TCTGAGGCTGGCCTGCACTGCCAGCGGCTTCACCTTCGACGACTATGCCA
TCGCGTGGTTCAGGCAGGCCCCTGGCAAGGAGAGGGAGTTTGTCGCAGCC
ATCAGCTGGAGCGGAGGCACCACCCACTACGCTGACAGCGTGAAGGGCAG
GTTCACCATTAGCAGGGACAATGCCAAGAACACCCTGTACCTGCAGATGA
GCAGTCTCAAGCCCGAAGATACCGCCGTGTACTTCTGCGCCAAGAGCCTG
AGGAGCAGCCCCAGCAGCAGGTGGTTCGGCAGCCGCGGTCAAGGCACCCA
GGTGACCGTGAGCTCTAGCGGAGGCGGAGGATCCGGAGCCCTGAGCAATA
GCATCATGTACTTCAGCCACTTCGTGCCCGTGTTTCTGCCCGCCAAGCCT
ACAACAACACCCGCTCCTAGACCACCTACACCAGCTCCTACAATCGCCAG
CCAGCCTCTGTCTCTCAGACCTGAAGCCTGTAGACCTGCAGCTGGCGGAG
CTGTGCATACCAGAGGCCTGGATATCTACATTTGGGCCCCTCTGGCTGGC
ACATGTGGCGTTCTGCTGCTCTCTCTGGTCATCACCCTGTACTGCAGACG
GCTGAAGATCCAAGTGCGGAAGGCCGCCATCACCAGCTACGAGAAATCTG
ATGGCGTGTACACCGGCCTGAGCACCCGGAATCAAGAAACCTACGAGACA
CTGAAGCACGAGAAGCCTCCACAAGCAGCGGCTCCTATGAGGACATGAGA
GGCATTCTGTACGCCGCTCCACAGCTGAGATCTATCAGAGGCCAGCCTGG
ACCTAACCACGAGGAAGATGCCGACTCCTACGAGAACATG.

In some embodiments, the CAR is encoded by a sequence with 70-100% sequence identity to

(SEQ ID NO: 350)
ATGTGGCTGCAGTCTCTGCTGCTGCTGGGAACAGTGGCCTGCAGCATCAG
CCAAGTTCAGCTCCAGGAATCAGGTGGGGGACTCGTACAGGCTGGAGGAA
GCCTGCGATTGAGCTGTGCCGCTCCACGATCTATCTTCTCAATAAATGCT
ATGGGTTGGTACAGGCAGGCCCCAGGCAAACAAAGAGAACTTGTAGCAGC
CATTACTTCAGGGGGGAGCCCAACCTATGCTGACTCAGTAAAGGGGCGGT
TCACTATTAGTAGGGATAACGCGAAGAATACGGTCTACTTGCAGATGAAC
AGTCTGAAGGCCGAAGATACGGCCGTGTATTATTGTGCCACAGGTCCCTA
TGGGCTGGACAATGCGTTGGATGCGTGGGGTCAGGGCACGCAGGTCACTG
TGAGTAGTAGCGGAGGCGGAGGATCCGGAGCCCTGAGCAATAGCATCATG
TACTTCAGCCACTTCGTGCCCGTGTTTCTGCCCGCCAAGCCTACAACAAC
ACCCGCTCCTAGACCACCTACACCAGCTCCTACAATCGCCAGCCAGCCTC
TGTCTCTCAGACCTGAAGCCTGTAGACCTGCAGCTGGCGGAGCTGTGCAT
ACCAGAGGCCTGGATATCTACATTTGGGCCCCTCTGGCTGGCACATGTGG
CGTTCTGCTGCTCTCTCTGGTCATCACCCTGTACTGCAGACGGCTGAAGA
TCCAAGTGCGGAAGGCCGCCATCACCAGCTACGAGAAATCTGATGGCGTG
TACACCGGCCTGAGCACCCGGAATCAAGAAACCTACGAGACACTGAAGCA
CGAGAAGCCTCCACAAGCAGCGGCTCCTATGAGGACATGAGAGGCATTCT
GTACGCCGCTCCACAGCTGAGATCTATCAGAGGCCAGCCTGGACCTAACC
ACGAGGAAGATGCCGACTCCTACGAGAACATG.

In some embodiments, the CAR is encoded by a sequence with 70-100% sequence identity to

(SEQ ID NO: 351)
CAAGTTCAGCTCCAGGAATCAGGTGGGGGACTCGTACAGGCTGGAGGAAG
CCTGCGATTGAGCTGTGCCGCTCCACGATCTATCTTCTCAATAAATGCTA
TGGGTTGGTACAGGCAGGCCCCAGGCAAACAAAGAGAACTTGTAGCAGCC
ATTACTTCAGGGGGGAGCCCAACCTATGCTGACTCAGTAAAGGGGCGGTT
CACTATTAGTAGGGATAACGCGAAGAATACGGTCTACTTGCAGATGAACA
GTCTGAAGGCCGAAGATACGGCCGTGTATTATTGTGCCACAGGTCCCTAT
GGGCTGGACAATGCGTTGGATGCGTGGGGTCAGGGCACGCAGGTCACTGT
GAGTAGTAGCGGAGGCGGAGGATCCGGAGCCCTGAGCAATAGCATCATGT
ACTTCAGCCACTTCGTGCCCGTGTTTCTGCCCGCCAAGCCTACAACAACA
CCCGCTCCTAGACCACCTACACCAGCTCCTACAATCGCCAGCCAGCCTCT
GTCTCTCAGACCTGAAGCCTGTAGACCTGCAGCTGGCGGAGCTGTGCATA
CCAGAGGCCTGGATATCTACATTTGGGCCCCTCTGGCTGGCACATGTGGC
GTTCTGCTGCTCTCTCTGGTCATCACCCTGTACTGCAGACGGCTGAAGAT
CCAAGTGCGGAAGGCCGCCATCACCAGCTACGAGAAATCTGATGGCGTGT
ACACCGGCCTGAGCACCCGGAATCAAGAAACCTACGAGACACTGAAGCAC
GAGAAGCCTCCACAAGCAGCGGCTCCTATGAGGACATGAGAGGCATTCTG
TACGCCGCTCCACAGCTGAGATCTATCAGAGGCCAGCCTGGACCTAACCA
CGAGGAAGATGCCGACTCCTACGAGAACATG.

Also provided herein is a cell comprising the recombinant nucleic acid of any of the compositions described herein.

In some embodiments, the cell is an immune cell.

In some embodiments, the cell is a myeloid cell, a lymphoid cell, a precursor cell, a stem cell or an induced pluripotent cell.

In some embodiments, the cell is CD14+/CD16−.

Also provided herein is a chimeric antigen receptor (CAR) comprising a VH domain described herein.

In some embodiments, the CAR comprises an extracellular domain, a transmembrane domain and an intracellular domain. In some embodiments, the CAR comprises a leader sequence. In some embodiments, the leader sequence comprises a sequence of MWLQSLLLLGTVACSIS (SEQ ID NO: 306). In some embodiments, extracellular domain comprises a hinge. In some embodiments the hinge is operatively connected to the transmembrane domain. In some embodiments, the hinge is a CD8alpha hinge. In some embodiments, the hinge has a sequence of ALSNSIMYFSHFVPVFLPAKPTTTPAPRPPTPAPTIASQPLSLRPEACRPAAGGAVHTRGLD (SEQ ID NO: 307). In some embodiments the extracellular domain comprises a linker. In some embodiments, the linker is operatively linked to the VH and the hinge. In some embodiments, the linker is operatively linked to the VH and the transmembrane domain. In some embodiments, the linker has a sequence of SGGGSG (SEQ ID NO: 308). In some embodiments, the transmembrane domain is a CD8 transmembrane domain. In some embodiments, the transmembrane domain has a sequence of IYIWAPLAGTCGVLLLSLVITLYC (SEQ ID NO: 309). In some embodiments, the intracellular domain comprises an intracellular signaling domain from FcεR. In some embodiments, the intracellular domain comprises a sequence of

(SEQ ID NO: 310)
RRLKIQVRKAAITSYEKSDGVYTGLSTRNQETYETLKHEKPPQ.

In some embodiments, the intracellular domain comprises at least two intracellular signaling domains. In some embodiments, the least two intracellular signaling domains are separated by a linker. In some embodiments the linker has a sequence of GSGS (SEQ ID NO: 311). In some embodiments, the intracellular domain comprises an intracellular signaling domain that that is the PI3K recruitment domain. In some embodiments the PI3K recruitment domain has a sequence of

(SEQ ID NO: 314)
YEDMRGILYAAPQLRSIRGQPGPNHEEDADSYENM.

In some embodiments, the CAR comprises a sequence with 70-100% sequence identity to

(SEQ ID NO: 316)
MWLQSLLLLGTVACSISQVQLQESGGGLVQAGGSLRLSCAAPRSIFSINA
MGWYRQAPGKQRELVAAITSGGSPTYADSVKGRFTISRDNAKNTVYLQMN
SLKAEDTAVYYCATGPYGLDNALDAWGQGTQVTVSSSGGGGSGALSNSIM
YFSHFVPVFLPAKPTTTPAPRPPTPAPTIASQPLSLRPEACRPAAGGAVH
TRGLDIYIWAPLAGTCGVLLLSLVITLYCRRLKIQVRKAAITSYEKSDGV
YTGLSTRNQETYETLKHEKPPQGSGSYEDMRGILYAAPQLRSIRGQPGPN
HEEDADSYENM.

In some embodiments, the CAR comprises a sequence with 70-100% sequence identity to

(SEQ ID NO: 315)
QVQLQESGGGLVQAGGSLRLSCAAPRSIFSINAMGWYRQAPGKQRELVAA
ITSGGSPTYADSVKGRFTISRDNAKNTVYLQMNSLKAEDTAVYYCATGPY
GLDNALDAWGQGTQVTVSSSGGGGSGALSNSIMYFSHFVPVFLPAKPTTT
PAPRPPTPAPTIASQPLSLRPEACRPAAGGAVHTRGLDIYIWAPLAGTCG
VLLLSLVITLYCRRLKIQVRKAAITSYEKSDGVYTGLSTRNQETYETLKH
EKPPQGSGSYEDMRGILYAAPQLRSIRGQPGPNHEEDADSYENM.

In some embodiments, the CAR comprises a sequence with 70-100% sequence identity to

(SEQ ID NO: 346)
MWLQSLLLLGTVACSISQVQLQESGGGLVQTGGSLRLACTASGFTFDDYA
IAWFRQAPGKEREFVAAISWSGGTTHYADSVKGRFTISRDNAKNTLYLQM
SSLKPEDTAVYFCAKSLRSSPSSRWFGSRGQGTQVTVSSSGGGGSGALSN
SIMYFSHFVPVFLPAKPTTTPAPRPPTPAPTIASQPLSLRPEACRPAAGG
AVHTRGLDIYIWAPLAGTCGVLLLSLVITLYCRRLKIQVRKAAITSYEKS
DGVYTGLSTRNQETYETLKHEKPPQGSGSYEDMRGILYAAPQLRSIRGQP
GPNHEEDADSYENM.

In some embodiments, the CAR comprises a sequence with 70-100% sequence identity to

(SEQ ID NO: 347)
QVQLQESGGGLVQTGGSLRLACTASGFTFDDYAIAWFRQAPGKEREFVAA
ISWSGGTTHYADSVKGRFTISRDNAKNTLYLQMSSLKPEDTAVYFCAKSL
RSSPSSRWFGSRGQGTQVTVSSSGGGGSGALSNSIMYFSHFVPVFLPAKP
TTTPAPRPPTPAPTIASQPLSLRPEACRPAAGGAVHTRGLDIYIWAPLAG
TCGVLLLSLVITLYCRRLKIQVRKAAITSYEKSDGVYTGLSTRNQETYET
LKHEKPPQGSGSYEDMRGILYAAPQLRSIRGQPGPNHEEDADSYENM.

In some embodiments, the CAR is encoded by a sequence with 70-100% sequence identity to

(SEQ ID NO: 352)
ATGTGGCTGCAGTCTCTGCTGCTGCTGGGAACAGTGGCCTGCAGCATCAG
CCAGGTGCAGTTGCAGGAGAGTGGCGGTGGCCTGGTGCAGACCGGTGGCA
GTCTGAGGCTGGCCTGCACTGCCAGCGGCTTCACCTTCGACGACTATGCC
ATCGCGTGGTTCAGGCAGGCCCCTGGCAAGGAGAGGGAGTTTGTCGCAGC
CATCAGCTGGAGCGGAGGCACCACCCACTACGCTGACAGCGTGAAGGGCA
GGTTCACCATTAGCAGGGACAATGCCAAGAACACCCTGTACCTGCAGATG
AGCAGTCTCAAGCCCGAAGATACCGCCGTGTACTTCTGCGCCAAGAGCCT
GAGGAGCAGCCCCAGCAGCAGGTGGTTCGGCAGCCGCGGTCAAGGCACCC
AGGTGACCGTGAGCTCTAGCGGAGGCGGAGGATCCGGAGCCCTGAGCAAT
AGCATCATGTACTTCAGCCACTTCGTGCCCGTGTTTCTGCCCGCCAAGCC
TACAACAACACCCGCTCCTAGACCACCTACACCAGCTCCTACAATCGCCA
GCCAGCCTCTGTCTCTCAGACCTGAAGCCTGTAGACCTGCAGCTGGCGGA
GCTGTGCATACCAGAGGCCTGGATATCTACATTTGGGCCCCTCTGGCTGG
CACATGTGGCGTTCTGCTGCTCTCTCTGGTCATCACCCTGTACTGCAGAC
GGCTGAAGATCCAAGTGCGGAAGGCCGCCATCACCAGCTACGAGAAATCT
GATGGCGTGTACACCGGCCTGAGCACCCGGAATCAAGAAACCTACGAGAC
ACTGAAGCACGAGAAGCCTCCACAAGGCAGCGGCTCCTATGAGGACATGA
GAGGCATTCTGTACGCCGCTCCACAGCTGAGATCTATCAGAGGCCAGCCT
GGACCTAACCACGAGGAAGATGCCGACTCCTACGAGAACATG.

In some embodiments, the CAR is encoded by a sequence with 70-100% sequence identity to

(SEQ ID NO: 353)
CAGGTGCAGTTGCAGGAGAGTGGCGGTGGCCTGGTGCAGACCGGTGGCAG
TCTGAGGCTGGCCTGCACTGCCAGCGGCTTCACCTTCGACGACTATGCCA
TCGCGTGGTTCAGGCAGGCCCCTGGCAAGGAGAGGGAGTTTGTCGCAGCC
ATCAGCTGGAGCGGAGGCACCACCCACTACGCTGACAGCGTGAAGGGCAG
GTTCACCATTAGCAGGGACAATGCCAAGAACACCCTGTACCTGCAGATGA
GCAGTCTCAAGCCCGAAGATACCGCCGTGTACTTCTGCGCCAAGAGCCTG
AGGAGCAGCCCCAGCAGCAGGTGGTTCGGCAGCCGCGGTCAAGGCACCCA
GGTGACCGTGAGCTCTAGCGGAGGCGGAGGATCCGGAGCCCTGAGCAATA
GCATCATGTACTTCAGCCACTTCGTGCCCGTGTTTCTGCCCGCCAAGCCT
ACAACAACACCCGCTCCTAGACCACCTACACCAGCTCCTACAATCGCCAG
CCAGCCTCTGTCTCTCAGACCTGAAGCCTGTAGACCTGCAGCTGGCGGAG
CTGTGCATACCAGAGGCCTGGATATCTACATTTGGGCCCCTCTGGCTGGC
ACATGTGGCGTTCTGCTGCTCTCTCTGGTCATCACCCTGTACTGCAGACG
GCTGAAGATCCAAGTGCGGAAGGCCGCCATCACCAGCTACGAGAAATCTG
ATGGCGTGTACACCGGCCTGAGCACCCGGAATCAAGAAACCTACGAGACA
CTGAAGCACGAGAAGCCTCCACAAGGCAGCGGCTCCTATGAGGACATGAG
AGGCATTCTGTACGCCGCTCCACAGCTGAGATCTATCAGAGGCCAGCCTG
GACCTAACCACGAGGAAGATGCCGACTCCTACGAGAACATG.

In some embodiments, the CAR is encoded by a sequence with 70-100% sequence identity to

(SEQ ID NO: 354)
ATGTGGCTGCAGTCTCTGCTGCTGCTGGGAACAGTGGCCTGCAGCATCAG
CCAAGTTCAGCTCCAGGAATCAGGTGGGGGACTCGTACAGGCTGGAGGAA
GCCTGCGATTGAGCTGTGCCGCTCCACGATCTATCTTCTCAATAAATGCT
ATGGGTTGGTACAGGCAGGCCCCAGGCAAACAAAGAGAACTTGTAGCAGC
CATTACTTCAGGGGGGAGCCCAACCTATGCTGACTCAGTAAAGGGGCGGT
TCACTATTAGTAGGGATAACGCGAAGAATACGGTCTACTTGCAGATGAAC
AGTCTGAAGGCCGAAGATACGGCCGTGTATTATTGTGCCACAGGTCCCTA
TGGGCTGGACAATGCGTTGGATGCGTGGGGTCAGGGCACGCAGGTCACTG
TGAGTAGTAGCGGAGGCGGAGGATCCGGAGCCCTGAGCAATAGCATCATG
TACTTCAGCCACTTCGTGCCCGTGTTTCTGCCCGCCAAGCCTACAACAAC
ACCCGCTCCTAGACCACCTACACCAGCTCCTACAATCGCCAGCCAGCCTC
TGTCTCTCAGACCTGAAGCCTGTAGACCTGCAGCTGGCGGAGCTGTGCAT
ACCAGAGGCCTGGATATCTACATTTGGGCCCCTCTGGCTGGCACATGTGG
CGTTCTGCTGCTCTCTCTGGTCATCACCCTGTACTGCAGACGGCTGAAGA
TCCAAGTGCGGAAGGCCGCCATCACCAGCTACGAGAAATCTGATGGCGTG
TACACCGGCCTGAGCACCCGGAATCAAGAAACCTACGAGACACTGAAGCA
CGAGAAGCCTCCACAAGGCAGCGGCTCCTATGAGGACATGAGAGGCATTC
TGTACGCCGCTCCACAGCTGAGATCTATCAGAGGCCAGCCTGGACCTAAC
CACGAGGAAGATGCCGACTCCTACGAGAACATG.

In some embodiments, the CAR is encoded by a sequence with 70-100% sequence identity to

(SEQ ID NO: 355)
CAAGTTCAGCTCCAGGAATCAGGTGGGGGACTCGTACAGGCTGGAGGAAG
CCTGCGATTGAGCTGTGCCGCTCCACGATCTATCTTCTCAATAAATGCTA
TGGGTTGGTACAGGCAGGCCCCAGGCAAACAAAGAGAACTTGTAGCAGCC
ATTACTTCAGGGGGGAGCCCAACCTATGCTGACTCAGTAAAGGGGCGGTT
CACTATTAGTAGGGATAACGCGAAGAATACGGTCTACTTGCAGATGAACA
GTCTGAAGGCCGAAGATACGGCCGTGTATTATTGTGCCACAGGTCCCTAT
GGGCTGGACAATGCGTTGGATGCGTGGGGTCAGGGCACGCAGGTCACTGT
GAGTAGTAGCGGAGGCGGAGGATCCGGAGCCCTGAGCAATAGCATCATGT
ACTTCAGCCACTTCGTGCCCGTGTTTCTGCCCGCCAAGCCTACAACAACA
CCCGCTCCTAGACCACCTACACCAGCTCCTACAATCGCCAGCCAGCCTCT
GTCTCTCAGACCTGAAGCCTGTAGACCTGCAGCTGGCGGAGCTGTGCATA
CCAGAGGCCTGGATATCTACATTTGGGCCCCTCTGGCTGGCACATGTGGC
GTTCTGCTGCTCTCTCTGGTCATCACCCTGTACTGCAGACGGCTGAAGAT
CCAAGTGCGGAAGGCCGCCATCACCAGCTACGAGAAATCTGATGGCGTGT
ACACCGGCCTGAGCACCCGGAATCAAGAAACCTACGAGACACTGAAGCAC
GAGAAGCCTCCACAAGGCAGCGGCTCCTATGAGGACATGAGAGGCATTCT
GTACGCCGCTCCACAGCTGAGATCTATCAGAGGCCAGCCTGGACCTAACC
ACGAGGAAGATGCCGACTCCTACGAGAACATG.

Provided herein are chimeric fusion proteins (CFPs) and recombinant nucleic acids encoding CFPs that target a cadherin target protein. In some embodiments the target protein is CDH17.

In one aspect, provided herein is a composition comprising a recombinant nucleic acid comprising a sequence encoding a chimeric fusion protein (CFP), the CFP comprising: an extracellular domain comprising an antigen binding domain that targets a cadherin; a transmembrane domain operatively linked to the extracellular domain; and an intracellular domain comprising an intracellular signaling domain operatively linked to the transmembrane domain.

In some embodiments, the cadherin is CDH17.

In some embodiments, the intracellular domain comprises an intracellular signaling domain from a phagocytosis receptor, a pattern recognition receptor, CD40, an Fc receptor, a cytokine receptor, and/or a chemokine receptor.

In some embodiments, the intracellular domain comprises at least two intracellular signaling domains or at least three two intracellular signaling domains.

In some embodiments, the intracellular domain comprises an intracellular signaling domain from CD40.

In some embodiments, the intracellular domain comprises an intracellular signaling domain from Fc epsilon receptor Ig (FCER1G).

In some embodiments, the intracellular domain comprises an intracellular signaling domain comprising a PI3K recruitment domain.

In some embodiments, the intracellular domain comprises (A) a first intracellular signaling domain from Fc epsilon receptor Ig (FCER1G) and (B) a second intracellular signaling domain comprising a PI3K recruitment domain.

In some embodiments, the intracellular domain comprises (A) a first intracellular signaling domain from CD40 and (B) a second intracellular signaling domain from Fc epsilon receptor Ig (FCER1G) and (C) a third intracellular signaling domain comprising a PI3K recruitment domain.

In some embodiments, the antigen binding domain is an antibody domain or antigen binding fragment thereof.

In some embodiments, the antigen binding domain is an scFv or a single domain antibody domain or a nanobody.

In some embodiments, the antigen binding domain is a VHH domain.

In some embodiments, the extracellular domain comprises a hinge domain.

In some embodiments, the extracellular domain comprises a CD8 hinge domain.

In some embodiments, extracellular domain comprises a hinge domain having the sequence

(SEQ ID NO: 307)
ALSNSIMYFSHFVPVFLPAKPTTTPAPRPPTPAPTIASQPLSLRPEACRP
AAGGAVHTRGLD.

In some embodiments, the CFP comprises a leader sequence.

In some embodiments, the CFP comprises a leader sequence that has the sequence

(SEQ ID NO: 306)
MWLQSLLLLGTVACSIS.

In some embodiments, the extracellular domain comprises a hinge domain and a linker between the hinge domain and the antigen binding domain.

In some embodiments, the extracellular domain comprises a linker between the antigen binding domain and the transmembrane domain.

In some embodiments, the linker has a sequence of (GxS)n, wherein x is an integer of from 1 to 4 and n is an integer of from 1 to 4 (SEQ ID NO: 356).

In some embodiments, the linker has a sequence SGGGSG (SEQ ID NO: 308).

In some embodiments, the transmembrane domain is a CD8 transmembrane domain.

In some embodiments, the transmembrane domain has a sequence of

(SEQ ID NO: 309)
IYIWAPLAGTCGVLLLSLVITLYC.

In some embodiments, the intracellular domain comprises a sequence of

(SEQ ID NO: 310)
RRLKIQVRKAAITSYEKSDGVYTGLSTRNQETYETLKHEKPPQ.

In some embodiments, the intracellular domain comprises PI3K recruitment domain that has a sequence of YEDMRGILYAAPQLRSIRGQPGPNHEEDADSYENM (SEQ ID NO: 314).

In some embodiments, the recombinant nucleic acid is an RNA.

In some embodiments, the recombinant nucleic acid is an mRNA.

In some embodiments, the recombinant nucleic acid is associated with one or more lipids.

In some embodiments, the recombinant nucleic acid is encapsulated in a liposome.

In some embodiments, the liposome is a lipid nanoparticle.

In some embodiments, the recombinant nucleic acid is a vector.

Also provided herein is a composition comprising a cell comprising a recombinant nucleic acid of a composition described herein.

In some embodiments, the cell is an immune cell.

In some embodiments, the cell is a myeloid cell, a lymphoid cell, a precursor cell, a stem cell or an induced pluripotent cell.

In some embodiments, the cell is CD14+/CD16−.

In some embodiments, the cell is a population of cells.

In some embodiments, the cell is a population of at least 1×10\hat{ }5 cells.

Also provided herein is a pharmaceutical composition comprising a composition described herein.

Also provided herein is a method of treating a disease or condition in a subject in need thereof comprising administering to the subject a pharmaceutical composition described herein.

In some embodiments, the disease or condition is a cancer.

In some embodiments, the disease or condition a gastrointestinal cancer or a neuroendocrine cancer.

In some embodiments provided herein is an use of a composition comprising (i) a binding domain for an CD70 antigen, a GPC3 antigen, an IL13Rα2 antigen or a CDH17 antigen in the manufacture of a formulation for treating a cancer in a human subject, wherein the binding domain comprises a VHH, an scFv or a CAR comprising any one of the HC CDR3 of any one of the preceding claims, or selected from the group consisting of: SEQ ID NOs: 4, 14, 113-150 and 253-265, 343; or (ii) a recombinant nucleic acid encoding the binding domain. In some embodiments, the cancer is an AML, CLL, NSCLC, RCC, HCC, melanoma, breast cancer MCL, CTCL, DLBCL, a lymphoma or a hematological cancer.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A depicts exemplary data of the binding kinetics of the shown anti-CD70 binding domains binding kinetics. A snapshot of the affinity measurement method is shown in the graphical illustration presented in the left bottom corner.

FIG. 1B depicts exemplary binding data of the shown anti-CD70 binding domains to H9 parental.

FIG. 1C depicts exemplary titration data of the shown anti-CD70 binding domains.

FIG. 1D depicts exemplary anti-CD70 CAR binding and expression data.

FIG. 1E depicts exemplary data showing phagocytosis of cancer cells with the shown anti-CD70 CAR monocyte constructs.

FIG. 2 depicts exemplary method and workflow of llama immunization and phage library construction for panning of antigen specific sequences. VHH selection is done by phage display and subsequent analyses including determining VHH binding specificity, sequencing and CDR3 family analysis. Figure discloses “6xHis” as SEQ ID NO: 364.

FIG. 3 depicts a schematic representation of llama immunization timeframe. First immunization was performed by injecting a total of 10\hat{ }8 cells from cancer cell lines H9, HEPG2, A375, SK-OV3 and HL60.

FIG. 4 depicts a schematic overview of results of immunization screening. Binding domains are identified for CD70, GPC3 and IL13R alpha 2 (IL13Rα2).

FIG. 5 depicts an image of a SDS-PAGE electrophoresis showing migrating bands of VHH-Fc fusion polypeptide constructs having CD70 binding domains: VHH-Fc fusion polypeptides from previously reported and commercial antibodies (27B3, 1C8 and 8G1); and VHH-Fc fusion polypeptides of currently identified VH domains of 3HCS92 (SEQ ID NO: 1, referred to as H1 elsewhere in the document) and 3CEK186 (SEQ ID NO: 11, referred to as C1 elsewhere in the document), in reduced and non-reduced forms.

FIG. 6A depicts binding data of the anti-CD70 VHH-Fc fusion polypeptide constructs having CD70 binding domains to parental H9 cells. A commercial anti-CD70 antibody from BioLegend, San Diego, CA (BL) was used as a positive control. VHH-Fc fusion polypeptides from previously reported and commercial antibodies were used as positive control: 27B3. Gating strategy is shown in the top left and top middle flow cytometry scan images of unstained cells.

FIG. 6B is a continuation of the data from the analysis run as shown in FIG. 6A, showing binding data of the anti-CD70 VHH-Fc fusion polypeptide constructs to parental H9 cells, positive VHH-Fc controls 1C8 and 8G1; and VHH-Fc fusion polypeptides having currently identified VH domains, H1-Fc (VHH domain, SEQ ID NO: 1), and C1-Fc (VHH domain, SEQ ID NO: 11). Lower right, a is a graphic illustration showing VHH-Fc fusion construct polypeptides in dimeric form as is expressed. The Fc chains are from human IgG1 and comprises the Fc constant domains CH1 and CH2, which help dimerize the VHH-Fc chains as shown. A disulfide bridge is formed in between the two chains as shown graphically.

FIG. 7 depicts binding titration data of the constructs described in FIGS. 6A and 6B.

FIG. 8 depicts data from Incucyte based PBMC killing assay, showing effect of anti-CD70 VHH-Fc fusion polypeptide constructs on human donor PBMC mediated killing of target H9-GFP cells. Proliferation of H9-GFP cells (Y-axis) were plotted against time shown in the X-axis. PBMC: target cell ratio was 5:1. Antibody concentration, 10 nM.

FIG. 9 depicts data showing expression of the anti-CD70 VHH constructs. Anti-VHH (Genescript) was used to stain expression of the VHHs, with no cross-reactivity to scFv. Anti-scFv has no cross reactivity to VHH. VHH construct 3HCS344 was used as VHH expression control.

FIG. 10 depicts data showing results from testing soluble CD70 for CAR-binding assays. CD70 molecules are labeled with Biotin (left) and stained with Streptavidin (Strep) APC-AF750, or HIS-tag (middle) and stained with APC tagged anti-HIS; or FITC tag (right) (fluorescent). Anti-CD70 (8G1)-CAR bound to soluble CD70 as shown in the lowest row. Flow cytometry data indicate Streptavidin-biotin detection yields the best signal for detecting soluble CD70.

FIG. 11 depicts data showing expression and function of anti-CD70 binding VHH-CARs in monocytes. mRNA electroporated monocytes were tested for the CAR expression (left) by anti-VHH staining; and testing binding of soluble CD70 and staining of CAR-expressing monocytes. No electroporation control was used for background. Results indicate that only H1 CAR is detectable by VHH staining as well as binding with soluble CD70.

FIG. 12 depicts data showing phagocytosis assay results for anti-CD70 binders. Human donor monocytes were transfected with CAR expressing MT101 (anti CD5-CAR) as positive control for phagocytosis; mock transfected set was used as negative control. Anti-CD70-ScFv 8G1 (commercial) was used for anti-CD70 positive control. Target cells for phagocytosis were H9 cancer cell line. Effector: Target ratio was 1:1, and assay was performed at 4 hours of incubation.

FIG. 13 shows graph of results from the phagocytosis assay described for FIG. 12.

FIG. 14 shows cytokine analysis data of electroporated cells expressing CAR at 24 hours. Cytokines depicted are GMCSF and IL-10. Data indicate values in presence of target H9 cells (SD values with target) (solid squares) or in absence (SD values with solid circles) of H9 target cells.

FIG. 15 shows data from chemokine analysis of electroporated cells expressing CAR at 24 hours in the study described in FIG. 14. Chemokines depicted are CCL5 and CXCL10.

FIG. 16 shows data from chemokine and cytokine analysis of electroporated cells expressing CAR at 24 hours in the study described in FIG. 14. Chemokines depicted are CCL2, CXCL10 and CXCL1; and cytokine depicted is IL-lb.

FIG. 17 shows cytokine analysis data of electroporated cells expressing CAR at 24 hours for IFN-alpha, IL-6, and TNF-alpha.

FIG. 18 shows expression of CD70 in cancer cell lines A498, which is an exemplary renal cell carcinoma cell line (adherent), (left); and Burkitts lymphoma cell line Raji (right), which is a suspended cell line.

FIG. 19 shows data demonstrating expression and purity of six exemplary GPC3 VHH binder constructs having binding domains listed in Table 3 run on SDS PAGE. For each strip—a marker row is shown on the left.

FIG. 20 shows data demonstrating flow cytometry detection of GPC3 binders by staining various cell lines with GPC3-A647 conjugated binders. CHO cells (control) CHO-GPC3 cells transfected with GPC3 encoding sequence and stained for binding with GPC3 binders indicated on the top. HepG2, HepB2, and Huh7 cells lines are various cancer cell lines expressing GPC3.

FIG. 21A shows a graphical illustration of the affinity assay method.

FIG. 21B illustrates GPC3 binning method and data.

FIG. 22 shows graphical representation of binning of GPC3 VHHs.

FIG. 23 shows expression of six exemplary chimeric antigen receptors (CARs) comprising extracellular VHH-GPC3 binders from Table 3 on monocytes—by electroporating monocytes with respective CAR constructs. The top-most row shows a VHH negative control, and the lowest is mock transfected negative control. All six VHH-CARs were detected on the surface of primary monocytes.

FIG. 24 shows data demonstrating expression and purity of exemplary IL13Rα2 binder constructs having binding domains listed in Table 5 run on SDS PAGE. For each strip—a marker row is shown on the left.

FIG. 25 shows data demonstrating exemplary IL13Rα2 soluble binders to various antigen positive cancer cell lines.

FIG. 26 shows IL13Rα2 binder binning method and data.

FIG. 27 shows binning the IL13Rα2 binders into four separate communities.

FIG. 28 illustrates graphically the different chimeric receptor modalities that are to be generated using the identified antigen binding domains.

### DETAILED DESCRIPTION

Engineered myeloid cells can also be short-lived in vivo, phenotypically diverse, sensitive, plastic, and are often found to be difficult to manipulate in vitro. For example, exogenous gene expression in monocytes has been difficult compared to exogenous gene expression in non-hematopoietic cells. There are significant technical difficulties associated with transfecting myeloid cells (e.g., monocytes/macrophages). As professional phagocytes, myeloid cells, such as monocytes/macrophages, comprise many potent degradative enzymes that can disrupt nucleic acid integrity and make gene transfer into these cells an inefficient process. This is especially true of activated macrophages which undergo a dramatic change in their physiology following exposure to immune or inflammatory stimuli. Viral transduction of these cells has been hampered because macrophages are end-stage cells that generally do not divide; therefore, some of the vectors that depend on integration into a replicative genome have met with limited success. Furthermore, macrophages are quite responsive to “danger signals,” and therefore several of the original viral vectors that were used for gene transfer induced potent anti-viral responses in these cells making these vectors inappropriate for gene delivery. The present disclosure provides innovative methods and compositions that can successfully transfect or transduce a myeloid cell, or otherwise induce a genetic modification in a myeloid cell, with the purpose of augmenting a functional aspect of a myeloid cell, additionally, without compromising the cell's differentiation capability, maturation potential, and/or its plasticity.

Provided herein are binding domains to target proteins. In some embodiments the target protein is CD70. In some embodiments the target protein is GPC3. In some embodiments the target protein is IL13 receptor alpha 2 (IL13Rα2). Detailed description of each embodiment follows in the forthcoming section of this disclosure.

For the description, terms used herein have simple meanings as understood by one of common knowledge in the art. For example, an “agent” can refer to any cell, small molecule chemical compound, antibody or fragment thereof, nucleic acid molecule, or polypeptide.

An “alteration” or “change” can refer to an increase or decrease. For example, an alteration can be an increase or decrease of 1%, 2%, 3%, 4%, 5%, 10%, 20%, 30%, or by 40%, 50%, 60%, or even by as much as 70%, 75%, 80%, 90%, or 100%. For example, an alteration can be an increase or decrease of 1-fold, 2-fold, 3-fold, 4-fold, 5-fold, 10-fold, 20-fold, 30-fold, or by 40-fold, 50-fold, 60-fold, or even by as much as 70-fold, 75-fold, 80-fold, 90-fold, or 100-fold.

An “antigen presenting cell” or “APC” as used herein includes professional antigen presenting cells (e.g., B lymphocytes, macrophages, monocytes, dendritic cells, Langerhans cells), as well as other antigen presenting cells (e.g., keratinocytes, endothelial cells, astrocytes, fibroblasts, oligodendrocytes, thymic epithelial cells, thyroid epithelial cells, glial cells (brain), pancreatic beta cells, and vascular endothelial cells). An APC can express Major Histocompatibility complex (MHC) molecules and can display antigens complexed with MHC on its surface which can be recognized by T cells and trigger T cell activation and an immune response. Professional antigen-presenting cells, notably dendritic cells, play a key role in stimulating naive T cells. Nonprofessional antigen-presenting cells, such as fibroblasts, may also contribute to this process. APCs can also cross-present peptide antigens by processing exogenous antigens and presenting the processed antigens on class I MHC molecules. Antigens that give rise to proteins that are recognized in association with class I MHC molecules are generally proteins that are produced within the cells, and these antigens are processed and associate with class I MHC molecules.

A “biological sample” can refer to any tissue, cell, fluid, or other material derived from an organism.

Antibody may refer to a protein that has the structural ability to bind to a specific antigen. “Antibody” generally refers to a class of proteins that are generally known as immunoglobulins, including, but not limited to IgG1, IgG2, IgG3, and IgG4), IgA (including IgA1 and IgA2), IgD, IgE, IgM, and IgY, The term “antibody” may include, but is not limited to, full length antibodies, single-chain antibodies, single domain antibodies (sdAb) and antigen-binding fragments thereof. Antigen-binding antibody fragments include, but are not limited to, Fab, Fab′ and F(ab′)2, Fd (consisting of V_{H} and C_{H}1), single-chain variable fragment (scFv), single-chain antibodies, disulfide-linked variable fragment (dsFv) and fragments comprising a VL and/or a VH domain. Antibodies can be from any animal origin. Antigen-binding antibody fragments, including single-chain antibodies, can comprise variable region(s) alone or in combination with one or more of a hinge region, a CH1 domain, a CH2 domain, and a CH3 domain. Also included are any combinations of variable region(s) and hinge region, CH1, CH2, and CH3 domains. Antibodies can be monoclonal, polyclonal, chimeric, humanized, and human monoclonal and polyclonal antibodies which, e.g., specifically bind an HLA-associated polypeptide or an HLA-peptide complex. Antibodies may be thought of as glycoproteins and may be made by plasma cells in vivo. Antibodies may be engineered, artificially generated. Antibodies can be monoclonal, polyclonal, chimeric, humanized, and human monoclonal and polyclonal antibodies which, e.g., specifically bind an HLA-associated polypeptide or an HLA-peptide complex.

An antibody may be monospecific, e.g., binds to one specific target antigen, or multispecific, e.g., binds to multiple targets; may be monoclonal, or polyclonal, may be natural or artificial (e.g., recombinant), and may be generated in a host species, e.g. mouse, rat, rabbit, goat cynomolgous (cyno) monkey, or a llama or any other species by antigen injection, a process generally termed as immunization. An antibody may be a primary antibody that binds to a target antigen; a secondary antibody that can bind to a primary antibody, a secondary antibody is often used in the detection of a primary antibody that binds to an antigen. In some examples, and antibody may be a blocking antibody. A fragment thereof of an antibody may be a protein or glycoprotein, comprising a heavy chain or a light chain, or fragments of either or both, and may be an scFv (a single chain variable fragment), a single domain antibody (sdAb), a variable heavy chain (VHH), a nanobody, a VNAR domain, a bispecific antibody, a diabody, or a nanobody. Antibody specificity towards a target antigen may be improved by various means. For example, antibodies may be modified to gain increased affinity, avidity, etc., by a process call affinity maturation. Affinity maturation is a result of somatic hypermutation of immunoglobulin genes in B cells, coupled to selection for antigen binding. This reiterative process occurs in germinal centers and structures within secondary lymphoid organs. For developing antibodies for therapeutic or prophylactic use, one recognizes that the process may be time consuming and requires in vivo work in animal models. Other antibody or binding domain improvement methods may include introducing mutations to the framework regions (FWR) of a wildtype (WT) antibody, e.g. the antibody isolated from an immunized animal, such as to humanize an antibody or a fragment thereof, e.g., a VHH or a scFv. There may be 1, 2, 3, 4, 5, 6 or more critical residues which when mutated can result in decrease in antibody activity, or loss of activity. Such residues when known or encountered through study should not be further mutated for a given framework region. Another method for humanizing antibodies or functional fragments thereof include, for example replacing (e.g. grafting) non-human framework regions of a variable domain with human sequences, allowing rapid and more extensive humanizations, obtaining near human sequences, aimed at avoiding immune reaction in a recipient human.

A binding domain may refer to a structural domain of a protein that is capable of interacting with another biomolecule, typically thought of as an engagement domain. The subject protein is a binder, whereas the entity to which it binds may often be referred to a “target” for the binder, or a ligand, e.g., for a receptor. The target may be a target antigen expressed on the surface of a cell, wherein the cell expressing the target may be referred to as a target cell. For example, a cancer antigen, CD5 is a target of an anti-CD5 binder CFP molecule, and the CD5+ cancer cell is a target cell. A binding domain may be assessed for its quality by determining its binding affinity to a specific target. Binding affinity may be the strength of binding interaction between a single biomolecule (e.g. a protein) to its ligand or partner or target, e.g., binding affinity of a receptor for its target antigen. A binder may be used interchangeably to refer to a binding domain. A binder may refer to a biomolecule with a binding domain. For example, a binder may be an antibody, an scFv, a VHH, a receptor, a chimeric receptor, or a soluble antibody or a domain thereof, e.g. soluble bispecific and trispecific engagers.

A chimeric antigen receptor for phagocytosis is often referred to here as a chimeric fusion protein (CFP). A recombinant nucleic acid is described encoding a chimeric fusion protein (CFP), such as a phagocytic receptor (PR) fusion protein (PFP), a scavenger receptor (SR) fusion protein (SFP), an integrin receptor (IR) fusion protein (IFP) or a caspase-recruiting receptor (caspase-CAR) fusion protein. A CFP encoded by the recombinant nucleic acid can comprise an extracellular domain (ECD) comprising an antigen binding domain that binds to an antigen of a target cell. The extracellular domain can be fused to a hinge domain or an extracellular domain derived from a receptor, such as CD2, CD8, CD28, CD68, a phagocytic receptor, a scavenger receptor or an integrin receptor. The CFP encoded by the recombinant nucleic acid can further comprise a transmembrane domain, such as a transmembrane domain derived from CD2, CD8, CD28, CD68, a phagocytic receptor, a scavenger receptor or an integrin receptor. In some embodiments, a CFP encoded by the recombinant nucleic acid further comprises an intracellular domain comprising an intracellular signaling domain, such as an intracellular signaling domain derived from a phagocytic receptor, a scavenger receptor or an integrin receptor. For example, the intracellular domain can comprise one or more intracellular signaling domains derived from a phagocytic receptor, a scavenger receptor or an integrin receptor. For example, the intracellular domain can comprise one or more intracellular signaling domains that promote phagocytic activity, inflammatory response, nitric oxide production, integrin activation, enhanced effector cell migration (e.g., via chemokine receptor expression), antigen presentation, and/or enhanced cross presentation. In some embodiments, the CFP is a phagocytic receptor fusion protein (PFP). In some embodiments, the CFP is a phagocytic scavenger receptor fusion protein (PFP). In some embodiments, the CFP is an integrin receptor fusion protein (IFP). In some embodiments, the CFP is an inflammatory receptor fusion protein. In some embodiments, a CFP encoded by the recombinant nucleic acid further comprises an intracellular domain comprising a recruitment domain. For example, the intracellular domain can comprise one or more PI3K recruitment domains, caspase recruitment domains or caspase activation and recruitment domains (CARDs). A CAR expression in a myeloid cell may lead in activation of the myeloid cell and increase phagocytosis.

The term “epitope” can refer to any protein determinant, such as a sequence or structure or amino acid residues, capable of binding to an antibody or binding fragment thereof, a T cell receptor, and/or an antibody-like molecule. Epitopic determinants typically consist of chemically active surface groups of molecules such as amino acids or sugar side chains and generally have specific three-dimensional structural characteristics as well as specific charge characteristics. A “T cell epitope” can refer to peptide or peptide-MHC complex recognized by a T cell receptor. Binding affinity may be measured by determination of binding characteristics, e.g. binding kinetics. Binding affinity may be a bioanalytic assay, that determines the equilibrium constant and dissociation kinetics.

An engineered cell, such as an engineered myeloid cell, can refer to a cell that has at least one exogenous nucleic acid sequence in the cell, even if transiently expressed. Expressing an exogenous nucleic acid may be performed by various methods described elsewhere, and encompasses methods known in the art. The present disclosure relates to preparing and using engineered cells, for example, engineered myeloid cells, such as engineered phagocytic cells. The present disclosure relates to, inter alia, an engineered cell comprising an exogenous nucleic acid encoding, for example, a chimeric fusion protein (CFP).

A “fragment” can refer to a portion of a protein or nucleic acid. In some embodiments, a fragment retains at least 50%, 75%, or 80%, or 90%, 95%, or even 99% of the biological activity of a reference protein or nucleic acid.

The term “immune response” includes, but is not limited to, macrophage, monocytes, dendritic cell mediated, or myeloid cell mediated, or T cell mediated, or NK cell mediated and/or B cell mediated immune responses. Exemplary immune responses of a myeloid cell responses include, for example, cytokine production, and phagocytosis. In addition, immune responses include immune responses that are indirectly affected by NK cell activation, B cell activation and/or T cell activation, e.g., antibody production (humoral responses) and activation of cytokine responsive cells, e.g., macrophages. Immune responses include innate and adaptive immune responses. The adaptive immune system can react to foreign molecular structures, such as antigens of an intruding organism. Adaptive immune reactions include humoral immune reactions and cell-mediated immune reactions. In humoral immune reactions, antibodies secreted by B cells into bodily fluids bind to pathogen-derived antigens leading to elimination of the pathogen through a variety of mechanisms, e.g. complement-mediated lysis. In cell-mediated immune reactions, T cells capable of destroying other cells are activated. For example, if proteins associated with a disease are present in a cell, they can be fragmented proteolytically to peptides within the cell. Specific cell proteins can then attach themselves to the antigen or a peptide formed in this manner, and transport them to the surface of the cell, where they can be presented to molecular defense mechanisms, such as T cells. Cytotoxic T cells can recognize these antigens and kill cells that harbor these antigens. The term “immune cell” includes immune effector cells.

“Effector cell,” or immune effector cell may refer to a cell that is involved in an immune response, e.g., in the promotion of an immune effector response. Examples of immune effector cells include, but are not limited to, macrophages, monocytes, dendritic cells, and other myeloid cells.

The term “effector function” or “effector response” refers to a specialized function of a cell. Effector function of a myeloid cell, for example, may be phagocytic activity or helper activity including the secretion of cytokines.

The compositions and methods of the present invention encompass polypeptides and nucleic acids having the sequences specified, or sequences substantially identical or similar thereto, e.g., sequences at least 80%, 85%, 90%, 95% identical or higher to the sequence specified. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 80%, 85%, 90%, 91%, 92%, 93%, 94%, 95%, 96%, 97%, 98% or 99% identity to a reference sequence, e.g., a sequence provided herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity or encode a common structural polypeptide domain or a common functional polypeptide activity. For example, nucleotide sequences having at least about 80%, 85%, 90%, 91%, 92%, 93%, 94%, 95%, 96%, 97%, 98% or 99% identity to a reference sequence, e.g., a sequence provided herein.

The term “variant” may refer to a polypeptide that has a substantially identical amino acid sequence to a reference amino acid sequence or is encoded by a substantially identical nucleotide sequence. In some embodiments, the variant is a functional variant.

A “functional variant” may refer to a polypeptide that has a substantially identical amino acid sequence to a reference amino acid sequence, or is encoded by a substantially identical nucleotide sequence, and is capable of having one or more activities of the reference amino acid sequence.

The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the V_{L} and V_{H} variable regions in either order, e.g., with respect to the N-terminal and C-terminal ends of the polypeptide, the scFv may comprise VL-linker-VH or may comprise VH-linker-V_{L}.

The terms “complementarity determining region” or “CDR,” are used interchangeably herein and refer to the sequences of amino acids within antibody variable regions which confer antigen specificity and binding affinity. For example, in general, there are three CDRs in each heavy chain variable region (e.g., HCDR1, HCDR2, and HCDR3) and three CDRs in each light chain variable region (LCDR1, LCDR2, and LCDR3). For most purposes discussed in this disclosure the CDRs are from the heavy chain, (HC CDR), and unless designated otherwise, a CDR described herein may be from a heavy chain. The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), A1-Lazikani et al., (1997) JMB 273,927-948 (“Chothia” numbering scheme), or a combination thereof. Under the Kabat numbering scheme, in some embodiments, the CDR amino acid residues in the heavy chain variable domain (VH) are numbered 31-35 (HCDR1), 50-65 (HCDR2), and 95-102 (HCDR3); and the CDR amino acid residues in the light chain variable domain (V_{L}) are numbered 24-34 (LCDR1), 50-56 (LCDR2), and 89-97 (LCDR3). Under the Chothia numbering scheme, in some embodiments, the CDR amino acids in the V_{H} are numbered 26-32 (HCDR1), 52-56 (HCDR2), and 95-102 (HCDR3); and the CDR amino acid residues in the V_{L} are numbered 26-32 (LCDR1), 50-52 (LCDR2), and 91-96 (LCDR3). In a combined Kabat and Chothia numbering scheme, in some embodiments, the CDRs correspond to the amino acid residues that are part of a Kabat CDR, a Chothia CDR, or both. For instance, in some embodiments, the CDRs correspond to amino acid residues 26-35 (HCDR1), 50-65 (HCDR2), and 95-102 (HCDR3) in a V_{H}, e.g., a mammalian V_{H}, e.g., a human V_{H}; and amino acid residues 24-34 (LCDR1), 50-56 (LCDR2), and 89-97 (LCDR3) in a V_{L}, e.g., a mammalian V_{L}, e.g., a human VL.

The numbering system followed in this disclosure is IMGT system, unless mentioned otherwise. The IMGT unique numbering has been defined to compare the variable domains whatever the antigen receptor, the chain type, or the species. The IMGT unique numbering provides a standardized delimitation of the framework regions and of the complementarity determining regions. As gaps represent unoccupied positions, the CDR-IMGT lengths become crucial information. The IMGT unique numbering is used in 2D graphical representations, designated as IMGT Colliers de Perles and in 3D structures in IMGT/3Dstructure-DB. (Lefranc M.-P., “Unique database numbering system for immunogenetic analysis” Immunology Today, 18, 509 (1997); Ruiz, M. and Lefranc, M.-P. “IMGT gene identification and Colliers de Perles of human immunoglobulin with known 3D structures” Immunogenetics, 53, 857-883 (2002)).

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Fully human” refers to an immunoglobulin, such as an antibody or antibody fragment, where the whole molecule is of human origin or consists of an amino acid sequence identical to a human form of the antibody or immunoglobulin.

The term “specifically binds,” refers to an antibody, or a ligand, which recognizes and binds with a cognate binding partner (e.g., a stimulatory and/or costimulatory molecule present on a T cell) protein present in a sample, but which antibody or ligand does not substantially recognize or bind other molecules in the sample.

A “ligand” can refer to a molecule which is capable of binding or forming a complex with another molecule, such as a receptor. A ligand can include, but is not limited to, a protein, a glycoprotein, a carbohydrate, a lipoprotein, a hormone, a fatty acid, a phospholipid, or any component that binds to a receptor. In some embodiments, a receptor has a specific ligand. In some embodiments, a receptor may have promiscuous binding to a ligand, in which case it can bind to several ligands that share at least a similarity in structural configuration, charge distribution or any other physicochemical characteristic. A ligand may be a biomolecule. A ligand may be an abiotic material. For example, a ligand may be a negative charged particle that is a ligand for scavenger receptor MARCO. For example, a ligand may be TiO₂, which is a ligand for the scavenger receptor SRA1.

A myeloid cell may be a cell that broadly encompass a cell having a myeloid lineage of cellular differentiation. For example, a myeloid cell may be a CD14+ cell, a CD14^{high} or a CD14a^{dim}cell. A myeloid cell may be a CD14+/CD16− cell, a CD14+/CD16+ cell, a CD14—/CD16+ cell, CD14−/CD16− cell, a dendritic cell, an M0 macrophage, an M2 macrophage, an M1 macrophage or a mosaic myeloid cell/macrophage/dendritic cell. A myeloid cell may be a phagocytic cell, or exhibit low phagocytosis, may exhibit immune response, may produce a cytokine. Engineered myeloid cells, such as macrophages and other phagocytic cells, can be prepared by incorporating nucleic acid sequences (e.g., mRNA, plasmids, viral constructs) encoding a chimeric fusion protein (CFP), that has an extracellular binding domain specific to disease associated antigens (e.g., cancer antigens), into the cells using, for example, recombinant nucleic acid technology, synthetic nucleic acids, gene editing techniques (e.g., CRISPR), transduction (e.g., using viral constructs), electroporation, or nucleofection. It has been found that myeloid cells can be engineered to have a broad and diverse range of activities. For example, it has been found that myeloid cells can be engineered to express a chimeric fusion protein (CFP) containing an antigen binding domain to have a broad and diverse range of activities. For example, it has been found that myeloid cells can be engineered to have enhanced phagocytic activity such that upon binding of the CFP to an antigen on a target cell, the cell exhibits increased phagocytosis of the target cell. It has also been found that myeloid cells can be engineered to promote T cell activation such that upon binding of the CFP to an antigen on a target cell, the cell promotes activation of T cells, such as T cells in the tumor microenvironment. The engineered myeloid cells can be engineered to promote secretion of tumoricidal molecules such that upon binding of the CFP to an antigen on a target cell, the cell promotes secretion of tumoricidal molecules from nearby cells. The engineered myeloid cells can be engineered to promote recruitment and trafficking of immune cells and molecules such that upon binding of the CFP to an antigen on a target cell, the cell promotes recruitment and trafficking of immune cells and molecules to the target cell or a tumor microenvironment.

The present disclosure is based on the important finding that engineered myeloid cells overcome at least some of the limitations of CAR-T cells, including being readily recruited to solid tumors; having an engineerable duration of survival, therefore lowering the risk of prolonged persistence resulting in aplasia and immunodeficiency; myeloid cells cannot be contaminated with T cells; myeloid cells can avoidance of fratricide, for example because they do not express the same antigens as malignant T cells; and myeloid cells have a plethora of anti-tumor functions that can be deployed. In some respects, engineered myeloid derived cells can be safer immunotherapy tools to target and destroy diseased cells.

Moreover, myeloid cells, such as macrophages, have been ubiquitously found in the tumor environment (TME) and are notably the most abundant cells in some tumor types. As part of their role in the immune system, myeloid cells, such as macrophages, are naturally engaged in clearing diseased cells. The present disclosure relates to harnessing myeloid cell function and specifically for targeting, killing and directly and/or indirectly clearing diseased cells as well as the delivery payloads such as antigens and cytokines.

“Phagocytosis” is used interchangeably with “engulfment” and can refer to a process by which a cell engulfs a particle, such as a cancer cell or an infected cell. This process can give rise to an internal compartment (phagosome) containing the particle. This process can be used to ingest and or remove a particle, such as a cancer cell or an infected cell from the body. A phagocytic receptor may be involved in the process of phagocytosis. The process of phagocytosis can be closely coupled with an immune response and antigen presentation. The processing of exogenous antigens follows their uptake into professional antigen presenting cells by some type of endocytic event. Phagocytosis can also facilitate antigen presentation. For example, antigens from phagocytosed cells or pathogens, including cancer antigens, can be processed and presented on the cell surface of antigen presenting cells (APCs).

A polypeptide can refer to a molecule containing amino acids linked together via a peptide bond, such as a glycoprotein, a lipoprotein, a cellular protein or a membrane protein. A polypeptide may comprise one or more subunits of a protein. A polypeptide may be encoded by a recombinant nucleic acid. In some embodiments, polypeptide may comprise more than one peptide sequence in a single amino acid chain, which may be separated by a spacer, a linker or peptide cleavage sequence. A polypeptide may be a fused polypeptide. A polypeptide may comprise one or more domains, modules or moieties. The terms “polypeptide”, “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures. A “polypeptide” can refer to a molecule containing amino acids linked together via a peptide bond, such as a glycoprotein, a lipoprotein, a cellular protein or a membrane protein. A polypeptide may comprise one or more subunits of a protein. A polypeptide may be encoded by a recombinant nucleic acid. In some embodiments, polypeptide may comprise more than one peptide sequence in a single amino acid chain, which may be separated by a spacer, a linker or peptide cleavage sequence. A polypeptide may be a fused polypeptide. A polypeptide may comprise one or more domains, modules or moieties.

The terms “nucleic acid,” “nucleic acid sequence,” “nucleotide sequence,” or “polynucleotide sequence,” and “polynucleotide” are used interchangeably. They refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant nucleic acid, or a polynucleotide of genomic, cDNA, semisynthetic, or synthetic origin which either does not occur in nature or is linked to another polynucleotide in a non-natural arrangement.

A “receptor” can refer to a chemical structure composed of a polypeptide, which transduces a signal, such as a polypeptide that transduces an extracellular signal to a cell. A receptor can serve to transmit information in a cell, a cell formation or an organism. A receptor comprises at least one receptor unit and can contain two or more receptor units, where each receptor unit comprises a protein molecule, e.g., a glycoprotein molecule. A receptor can contain a structure that binds to a ligand and can form a complex with the ligand. Signaling information can be transmitted by a conformational change of the receptor following binding with the ligand on the surface of a cell.

The term “recombinant nucleic acid” refers a nucleic acid prepared, expressed, created or isolated by recombinant means. A recombinant nucleic acid can contain a nucleotide sequence that is not naturally occurring. A recombinant nucleic acid may be synthesized in the laboratory. A recombinant nucleic acid may be prepared by using recombinant DNA technology, for example, enzymatic modification of DNA, such as enzymatic restriction digestion, ligation, and DNA cloning. A recombinant nucleic acid can be DNA, RNA, analogues thereof, or a combination thereof. A recombinant DNA may be transcribed ex vivo or in vitro, such as to generate a messenger RNA (mRNA). A recombinant mRNA may be isolated, purified and used to transfect a cell. A recombinant nucleic acid may encode a protein or a polypeptide.

The term “vector” can refer to a nucleic acid molecule capable of autonomous replication in a host cell, and which allow for cloning of nucleic acid molecules. As known to those skilled in the art, a vector includes, but is not limited to, a plasmid, cosmid, phagemid, viral vectors, phage vectors, yeast vectors, mammalian vectors and the like. For example, a vector for exogenous gene transformation may be a plasmid. In certain embodiments, a vector comprises a nucleic acid sequence containing an origin of replication and other elements necessary for replication and/or maintenance of the nucleic acid sequence in a host cell. In some embodiments, a vector or a plasmid provided herein is an expression vector. Expression vectors are capable of directing the expression of genes and/or nucleic acid sequence to which they are operatively linked. In some embodiments, an expression vector or plasmid is in the form of circular double stranded DNA molecules. A vector or plasmid may or may not be integrated into the genome of a host cell. In some embodiments, nucleic acid sequences of a plasmid are not integrated in a genome or chromosome of the host cell after introduction. For example, the plasmid may comprise elements for transient expression or stable expression of the nucleic acid sequences, e.g. genes or open reading frames harbored by the plasmid, in a host cell. In some embodiments, a vector is a transient expression vector. In some embodiments, a vector is a stably expressed vector that replicates autonomously in a host cell. In some embodiments, nucleic acid sequences of a plasmid are integrated into a genome or chromosome of a host cell upon introduction into the host cell. Expression vectors that can be used in the methods as disclosed herein include, but are not limited to, plasmids, episomes, bacterial artificial chromosomes, yeast artificial chromosomes, bacteriophages or viral vectors. A vector can be a DNA or RNA vector. In some embodiments, a vector provide herein is a RNA vector that is capable of integrating into a host cell's genome upon introduction into the host cell (e.g., via reverse transcription), for example, a retroviral vector or a lentiviral vector. Other forms of expression vectors known by those skilled in the art which serve the equivalent functions can also be used, for example, self-replicating extrachromosomal vectors or vectors capable of integrating into a host genome. Exemplary vectors are those capable of autonomous replication and/or expression of nucleic acids to which they are linked.

The terms “spacer” or “linker” as used in reference to a fusion protein refers to a peptide sequence that joins two other peptide sequences of the fusion protein. In some embodiments, a linker or spacer has no specific biological activity other than to join or to preserve some minimum distance or other spatial relationship between the proteins or RNA sequences. In some embodiments, the constituent amino acids of a spacer can be selected to influence some property of the molecule such as the folding, flexibility, net charge, or hydrophobicity of the molecule. Suitable linkers for use in an embodiment of the present disclosure are well known to those of skill in the art and include, but are not limited to, straight or branched-chain carbon linkers, heterocyclic carbon linkers, or peptide linkers. In some embodiments, a linker is used to separate two or more polypeptides, e.g. two antigenic peptides by a distance sufficient to ensure that each antigenic peptide properly folds. Exemplary peptide linker sequences adopt a flexible extended conformation and do not exhibit a propensity for developing an ordered secondary structure. Amino acids in flexible linker protein region may include Gly, Asn and Ser, or any permutation of amino acid sequences containing Gly, Asn and Ser. Other near neutral amino acids, such as Thr and Ala, also can be used in the linker sequence.

The terms “treat,” “treated,” “treating,” “treatment,” and the like are meant to refer to reducing, preventing, or ameliorating a disorder and/or symptoms associated therewith (e.g., a neoplasia or tumor or infectious agent or an autoimmune disease). “Treating” can refer to administration of the therapy to a subject after the onset, or suspected onset, of a disease (e.g., cancer or infection by an infectious agent or an autoimmune disease). “Treating” includes the concepts of “alleviating”, which can refer to lessening the frequency of occurrence or recurrence, or the severity, of any symptoms or other ill effects related to the disease and/or the side effects associated with therapy. The term “treating” also encompasses the concept of “managing” which refers to reducing the severity of a disease or disorder in a patient, e.g., extending the life or prolonging the survivability of a patient with the disease, or delaying its recurrence, e.g., lengthening the period of remission in a patient who had suffered from the disease. It is appreciated that, although not precluded, treating a disorder or condition does not require that the disorder, condition, or symptoms associated therewith be completely eliminated. The term “prevent”, “preventing”, “prevention” and their grammatical equivalents as used herein, can refer to avoiding or delaying the onset of symptoms associated with a disease or condition in a subject that has not developed such symptoms at the time the administering of an agent or compound commences. In certain embodiments, treating a subject or a patient as described herein comprises administering a therapeutic composition, such as a drug, a metabolite, a preventive component, a nucleic acid, a peptide, or a protein that encodes or otherwise forms a drug, a metabolite or a preventive component. In some embodiments, treating comprises administering a cell or a population of cells to a subject in need thereof. In some embodiments, treating comprises administering to the subject one or more of engineered cells described herein, e.g. one or more engineered myeloid cells, such as phagocytic cells. Treating comprises treating a disease or a condition or a syndrome, which may be a pathological disease, condition or syndrome, or a latent disease, condition or syndrome. In some cases, treating, as used herein may comprise administering a therapeutic vaccine. In some embodiments, the engineered phagocytic cell is administered to a patient or a subject. In some embodiments, a cell administered to a human subject results in reduced immunogenicity. For example, an engineered phagocytic cell may lead to no or reduced graft versus host disease (GVHD) or fratricide effect. In some embodiments, an engineered cell administered to a human subject is immunocompatible to the subject (i.e. having a matching HLA subtype that is naturally expressed in the subject). Subject specific HLA alleles or HLA genotype of a subject can be determined by any method known in the art. In exemplary embodiments, the methods include determining polymorphic gene types that can comprise generating an alignment of reads extracted from a sequencing data set to a gene reference set comprising allele variants of the polymorphic gene, determining a first posterior probability or a posterior probability derived score for each allele variant in the alignment, identifying the allele variant with a maximum first posterior probability or posterior probability derived score as a first allele variant, identifying one or more overlapping reads that aligned with the first allele variant and one or more other allele variants, determining a second posterior probability or posterior probability derived score for the one or more other allele variants using a weighting factor, identifying a second allele variant by selecting the allele variant with a maximum second posterior probability or posterior probability derived score, the first and second allele variant defining the gene type for the polymorphic gene, and providing an output of the first and second allele variant.

The terms “isolated,” “purified”, “biologically pure” and their grammatical equivalents refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of the present disclosure is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications can give rise to different isolated proteins, which can be separately purified.

Nucleic acid molecules useful in the methods of the disclosure include, but are not limited to, any nucleic acid molecule with activity or that encodes a polypeptide. Polynucleotides having substantial identity to an endogenous sequence are typically capable of hybridizing with at least one strand of a double-stranded nucleic acid molecule. % Identity may be used to describe that the sequence of a nucleic acid molecule may be within a certain range of differentiation from a reference nucleic acid molecule. For example, a certain nucleic acid molecule having at least 90% identity to a reference nucleic acid R, whose sequence is known or is disclosed herein, it may refer to the understanding that the certain nucleic acid molecule has a sequence that is may be 90% identical base by base to the reference or 91% identical, or 92% identical . . . . 99% or 100% identity to the reference nucleic acid.

### Anti-CD70 Binding Domains and Compositions

CD70 is a member of the TNF superfamily, whose expression in tissues are tightly regulated, and are found on activated T- and B- cells, thymic epithelial cells, and some dendritic cells. CD70 binds to CD27 on T cells, which is recognized as the costimulatory pathway. CD70 is expressed in a variety of hematological cancers and solid tumors. CD70 increases the frequency of Tregs in tumor microenvironment. Expression of CD70 appears to be low in normal tissue. This in addition to its role in cancer, makes it an attractive candidate for targeting.

In one aspect, provided herein is a myeloid cell therapeutic, wherein CD70 specific chimeric receptor is expressed in a myeloid cell comprising a VHH or scFv having an anti-CD70 binding domain, which when expressed in a myeloid cell in vivo, activates the myeloid cell and drives the cell to kill its target that expresses CD70. Additionally, bispecific myeloid cell activator (BIME) and trispecific myeloid cell activator (TRiME) polypeptide compositions are designed using the composition and methods described that target CD70 expressing cells and kill the cells. In one aspect, a pharmaceutical composition is described herein, comprising a polynucleotide encoding any one of the above, and also comprising a suitable delivery vehicle for administering the polynucleotide. For example, the polynucleotide is an mRNA. For example, the delivery vehicle is a lipid nanoparticle.

In one aspect, provided herein is a composition comprising a construct comprising an antigen binding domain comprising an anti-CD70 antibody or binding fragment thereof, or a recombinant nucleic acid encoding the antigen binding domain, wherein the antigen binding domain comprises a heavy chain variable domain (VH) comprising a heavy chain complementarity determining region 3 (HC CDR3) that has a sequence selected from the group consisting of ATGPYGLDNALDA (SEQ ID NO: 4). In some embodiments, the VH of the anti-CD70 antibody or binding fragment thereof further comprises a heavy chain complementarity determining region 1 (HC CDR1) that has a selected from the group consisting of RSIFSINA (SEQ ID NO: 2) and GFTFDDYA (SEQ ID NO: 12).

In some embodiments, the VH of the anti-CD70 antibody or binding fragment thereof further comprises a heavy chain complementarity determining region 2 (HC CDR2) that is selected from the group consisting of ITSGGSP (SEQ ID NO: 3) and ISWSGGTT (SEQ ID NO: 13).

In one embodiment, provided herein is an anti-CD70 binding domain, e.g., a variable heavy chain (VH), comprising an anti-CDR3 having a sequence ATGPYGLDNALDA (SEQ ID NO: 4) and a CDR1 having a sequence RSIFSINA (SEQ ID NO: 2), and a CDR2 having a sequence

(SEQ ID NO: 3)
ITSGGSP.

In one embodiment, provided herein is an anti-CD70 binding domain, e.g., a variable heavy chain (VH), comprising an anti-CDR3 having a sequence ATGPYGLDNALDA (SEQ ID NO: 4) and a CDR1 having a sequence RSIFSINA (SEQ ID NO: 2), and a CDR2 having a sequence

(SEQ ID NO: 13)
ISWSGGTT.

In one embodiment, provided herein is an anti-CD70 binding domain, e.g., a variable heavy chain (VH), comprising an anti-CDR3 having a sequence ATGPYGLDNALDA (SEQ ID NO: 4) and a CDR1 having a sequence GFTFDDYA (SEQ ID NO: 12), and a CDR2 having a sequence ITSGGSP (SEQ ID NO: 3).

(SEQ ID NO: 3)
ITSGGSP.

In one embodiment, provided herein is an anti-CD70 binding domain, e.g., a variable heavy chain (VH), comprising an anti-CDR3 having a sequence ATGPYGLDNALDA (SEQ ID NO: 4) and a CDR1 having a sequence GFTFDDYA (SEQ ID NO: 12), and a CDR2 having a sequence

(SEQ ID NO: 13)
ISWSGGTT.

In one aspect, provided herein is a composition comprising a construct comprising an antigen binding domain comprising an anti-CD70 antibody or binding fragment thereof, or a recombinant nucleic acid encoding the antigen binding domain, wherein the antigen binding domain comprises a heavy chain variable domain (VH) comprising a heavy chain complementarity determining region 3 (HC CDR3) that has a sequence selected from the group consisting of

(SEQ ID NO: 14)
AKSLRSSPSSRWFGS.

In some embodiments, the VH of the anti-CD70 antibody or binding fragment thereof further comprises a heavy chain complementarity determining region 1 (HC CDR1) that has a selected from the group consisting of RSIFSINA (SEQ ID NO: 2) and GFTFDDYA (SEQ ID NO: 12).

In some embodiments, the VH of the anti-CD70 antibody or binding fragment thereof further comprises a heavy chain complementarity determining region 2 (HC CDR2) that is selected from the group consisting of ITSGGSP (SEQ ID NO: 3) and ISWSGGTT (SEQ ID NO: 13).

In one embodiment, provided herein is an anti-CD70 binding domain, e.g., a variable heavy chain (VH), comprising an anti-CDR3 having a sequence AKSLRSSPSSRWFGS (SEQ ID NO: 14) and a CDR1 having a sequence RSIFSINA (SEQ ID NO: 2), and a CDR2 having a sequence ITSGGSP (SEQ ID NO: 3).

(SEQ ID NO: 3)
ITSGGSP.

In one embodiment, provided herein is an anti-CD70 binding domain, e.g., a variable heavy chain (VH), comprising an anti-CDR3 having a sequence AKSLRSSPSSRWFGS (SEQ ID NO: 14) and a CDR1 having a sequence RSIFSINA (SEQ ID NO: 2), and a CDR2 having a sequence

(SEQ ID NO: 13)
ISWSGGTT

In one embodiment, provided herein is an anti-CD70 binding domain, e.g., a variable heavy chain (VH), comprising an anti-CDR3 having a sequence AKSLRSSPSSRWFGS (SEQ ID NO: 14) and a CDR1 having a sequence GFTFDDYA (SEQ ID NO: 12), and a CDR2 having a sequence

(SEQ ID NO: 3)
ITSGGSP.

In one embodiment, provided herein is an anti-CD70 binding domain, e.g., a variable heavy chain (VH), comprising an anti-CDR3 having a sequence AKSLRSSPSSRWFGS (SEQ ID NO: 14) and a CDR1 having a sequence GFTFDDYA (SEQ ID NO: 12), and a CDR2 having a sequence

(SEQ ID NO: 13)
ISWSGGTT.

In some embodiments, the anti-CD70 binding domain comprises a framework region 1 (FWR1), a framework region 2 (FWR2), a framework region 3 (FWR3) and a framework region 4 (FWR4). In some embodiments, an anti-CD70 binding domain comprises in tandem: FWR1, CDR1, FWR2, CDR2 and FWR3, CDR3 and FWR4.

In some embodiments, humanization of an antibody or fragment thereof involves, in one method, altering one or more amino acids within the framework region of an original antibody (e.g. an antibody originating from an animal) with an amino acid located in a human counterpart antibody (immunoglobulin molecule). In some embodiments, the altering involves replacing 1, 2, 3, 4, 5, 6, 7, 8, 9, 10 or more amino acids in the original antibody with an amino acid located in a human counterpart antibody. Unless otherwise mentioned, the antibody numbering scheme followed in this disclosure is the IMGT numbering scheme.

In some embodiments, the anti-CD70 binding domain comprises a FWR1 comprising a sequence QVQLVESGGGLVQPGGSLRLSC (SEQ ID NO: 357) or a sequence that has 1, 2, 3, 4, 5, 6, 7 or more amino acid replacements relative to the sequence QVQLVESGGGLVQPGGSLRLSC (SEQ ID NO: 357).

In some embodiments, the anti-CD70 binding domain comprises a FWR2 comprising a sequence WFRQAPGQGLEAV (SEQ ID NO: 358) or a sequence that has 1, 2, 3, 4, 5, 6, 7 or more amino acid replacements relative to the sequence WFRQAPGQGLEAV (SEQ ID NO: 358).

In some embodiments, the anti-CD70 binding domain comprises a FWR3 comprising a sequence RFTISRDNSKNTLYLQMNSLRAEDTAVYYC (SEQ ID NO: 359) or a sequence that has 1, 2, 3, 4, 5, 6, 7 or more amino acid replacements relative to the sequence

(SEQ ID NO: 359)
RFTISRDNSKNTLYLQMNSLRAEDTAVYYC.

In some embodiments, the anti-CD70 binding domain comprises a FWR4 comprising a sequence YWGQGTLVTVSS (SEQ ID NO: 360) or a sequence that has 1, 2, 3, 4, 5, 6, 7 or more amino acid replacements relative to the sequence YWGQGTLVTVSS (SEQ ID NO: 360).

In some embodiments, humanization of an antibody or fragment thereof involves grafting the selected CDRs, CDR1, 2 and 3 into a human immunoglobulin variable domain, comprising human immunoglobulin framework 1, 2, 3 and 4. In some embodiments, an anti-CD70 binding domain comprises a CDR1, CDR2 and CDR3 described as above, grafted into human immunoglobulin heavy chain comprising a framework region 1 (FWR1), a framework region 2 (FWR2), a framework region 3 (FWR3) and a framework region 4 (FWR4), wherein the FWR1 has an amino acid sequence EVQLVESGGGLVQPGGSLRLSC (SEQ ID NO: 361); the FWR2 has an amino acid sequence WVRQAPGKGLEWV (SEQ ID NO: 362); the FWR3 has an amino acid sequence FTISRDNSKNTLYLQMNSLRAEDTAVYYC (SEQ ID NO: 363); and a FWR4 having a sequence YWGQGTLVTVSS (SEQ ID NO: 360). In some embodiments the one or more framework regions described above may further comprise one or more amino acid replacements.

An antibody may be further developed via affinity maturation, a process by which antibodies gain increased affinity, avidity, and anti-pathogen activity and is the result of somatic hypennutation (SHM) of immunoglobulin genes in B cells, coupled to selection for anigen binding.

In some embodiments, the anti-CD70 antibody or a fragment thereof is a partially humanized antibody. In some embodiments, the anti-CD70 antibody or a fragment thereof is a humanized antibody. In some embodiments, the anti-CD70 antibody or a fragment thereof comprises one or more framework regions that comprise one or more amino acid alterations, e.g. replacements of one or more amino acids in the framework amino acid sequence of the original antibody or fragment thereof raised that was in an animal to incorporate amino acid from a human framework region.

In some embodiments, the anti-CD70 antibody or a fragment thereof comprises a sequence that is about 84.6% human, for example, the anti-CD70 antibody or a fragment thereof has a sequence of QVQLQESGGGLVQAGGSLRLSCAAPRSIFSINAMGWYRQAPGKQRELVAAITSGGSPTYAD SVKGRFTISRDNAKNTVYLQMNSLKAEDTAVYYCATGPYGLDNALDAWGQGTQVTVSS (SEQ ID NO: 1). In some embodiments, the sequence provided above is subjected to at least 5 FWR mutations. In some embodiments, the sequence provided above is subjected to at least 6 FWR mutations. In some embodiments, the sequence provided above is subjected to at least 7 FWR mutations. In some embodiments, the sequence provided above is subjected to at least 8 FWR mutations. In some embodiments, the sequence provided above is subjected to at least 9 FWR mutations. In some embodiments, the sequence provided above is subjected to at least 10 FWR mutations. In some embodiments, the sequence provided above is subjected to at least 11 FWR mutations. In some embodiments, the sequence provided above is subjected to at least 12 FWR mutations. In some embodiments, the sequence provided above is subjected to at least 13 FWR mutations. In some embodiments, the sequence provided above is subjected to at least 14 FWR mutations, e.g. 14 alterations in the framework region to obtain a 100% humanized antibody.

In some embodiments, the anti-CD70 antibody or a fragment thereof has a sequence that is about 91.2% human, e.g. clone h6 (SEQ ID NO: 6 in Table 1). In some embodiments, the anti-CD70 antibody or a fragment thereof has a sequence that is about 92.3% human, e.g. clone h7 in Table 1 (SEQ ID NO: 7 in Table 1). In some embodiments, the anti-CD70 antibody or a fragment thereof has a sequence that is about 93.4% human, e.g. clone h8 in Table 1 (SEQ ID NO: 8). In some embodiments, the anti-CD70 antibody or a fragment thereof has a sequence that is about 94.5% human, e.g. clone h9 in (SEQ ID NO: 9). In some embodiments, the anti-CD70 antibody or a fragment thereof has a sequence that is about 95.6% human, e.g. clone h10 in Table 1 (SEQ ID NO: 10).

In some embodiments, the anti-CD70 antibody variable heavy chain comprises a sequence having at least 70% sequence identity to QVQLQESGGGLVQAGGSLRLSCAAPRSIFSINAMGWYRQAPGKQRELVAAITSGGSPTYAD SVKGRFTISRDNAKNTVYLQMNSLKAEDTAVYYCATGPYGLDNALDAWGQGTQVTVSS (SEQ ID NO: 1). In some embodiments, the anti-CD70 antibody V_{H} comprises at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of the sequences selected from the group consisting of SEQ ID NOs: 1, 6-9. In some embodiments, the anti-CD70 V_{H} domain comprises a sequence that is at least 90% identical to EVQLLESGGGLVQPGGSLRLSCAASRSIFSINAMSWYRQAPGKQRELVSAITSGGSPTYADS VKGRFTISRDNSKNTLYLQMNSLRAEDTAVYYCATGPYGLDNALDAWGQGTLVTVSS (SEQ ID NO: 10). In some embodiments, the anti-CD70 V_{H} domain comprises a sequence that is at least 91%, 92%, 93%, 94%, 95%, 96%, 97%, 98%, 99%, or 100% sequence identity to SEQ ID NO: 10.

In some embodiments, the anti-CD70 antibody variable heavy chain comprises a sequence having at least 70% sequence identity to QVQLQESGGGLVQTGGSLRLACTASGFTFDDYAIAWFRQAPGKEREFVAAISWSGGTTHY ADSVKGRFTISRDNAKNTLYLQMSSLKPEDTAVYFCAKSLRSSPSSRWFGSRGQGTQVTVS S (SEQ ID NO: 11), which comprises about 84.6% humanized sequence. In some embodiments, the anti-CD70 antibody or a fragment thereof has a sequence that is about 90.1% human, e.g., clone h5 (SEQ ID NO: 16). In some embodiments, the anti-CD70 antibody or a fragment thereof has a sequence that is about 91.2% human, e.g., clone h6 (SEQ ID NO: 17 in Table 1). In some embodiments, the anti-CD70 antibody or a fragment thereof has a sequence that is about 92.3% human, e.g., clone h7 (SEQ ID NO: 18 in Table 1). In some embodiments, the anti-CD70 antibody or a fragment thereof has a sequence that is about 93.4% human, e.g., clone h8 in Table 1 (SEQ ID NO: 19). In some embodiments, the anti-CD70 antibody or a fragment thereof has a sequence that is about 94.5% human, e.g., clone h9 in (SEQ ID NO: 20). In some embodiments, the anti-CD70 antibody or a fragment thereof has a sequence that is about 95.6% human, e.g., clone h10 in Table 1 (SEQ ID NO: 342).

In some embodiments, the anti-CD70 antibody V_{H} comprises at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of the sequences selected from the group consisting of SEQ ID NOs: 11, 16-20. In some embodiments, the anti-CD70 VH domain comprises a sequence that is at least 90% identical to EVQLLESGGGLVQPGGSLRLSCAASRSIFSINAMSWYRQAPGKQRELVSAITSGGSPTYADS VKGRFTISRDNSKNTLYLQMNSLRAEDTAVYYCATGPYGLDNALDAWGQGTLVTVSS (SEQ ID NO: 21). In some embodiments, the anti-CD70 VH domain comprises a sequence that is at least 91%, 92%, 93%, 94%, 95%, 96%, 97%, 98%, 99%, or 100% sequence identity to SEQ ID NO: 21.

In some embodiments, the anti-CD70 antibody VH comprises at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of the sequences selected from the group consisting of SEQ ID NO: 11, 16-20.

In some embodiments, the VH of the anti-CD70 antibody or binding fragment thereof is encoded by a sequence with 70-100% sequence identity to

(SEQ ID NO: 5)
CAAGTTCAGCTCCAGGAATCAGGTGGGGGACTCGTACAGGCTGGAGGAAG
CCTGCGATTGAGCTGTGCCGCTCCACGATCTATCTTCTCAATAAATGCTA
TGGGTTGGTACAGGCAGGCCCCAGGCAAACAAAGAGAACTTGTAGCAGCC
ATTACTTCAGGGGGGAGCCCAACCTATGCTGACTCAGTAAAGGGGCGGTT
CACTATTAGTAGGGATAACGCGAAGAATACGGTCTACTTGCAGATGAACA
GTCTGAAGGCCGAAGATACGGCCGTGTATTATTGTGCCACAGGTCCCTAT
GGGCTGGACAATGCGTTGGATGCGTGGGGTCAGGGCACGCAGGTCACTGT
GAGTAGT

In some embodiments, the VH of the anti-CD70 antibody or binding fragment thereof is encoded by a sequence with 70-100% sequence identity to

(SEQ ID NO: 15)
CAGGTGCAGTTGCAGGAGAGTGGCGGTGGCCTGGTGCAGACCGGTGGCAG
TCTGAGGCTGGCCTGCACTGCCAGCGGCTTCACCTTCGACGACTATGCCA
TCGCGTGGTTCAGGCAGGCCCCTGGCAAGGAGAGGGAGTTTGTCGCAGCC
ATCAGCTGGAGCGGAGGCACCACCCACTACGCTGACAGCGTGAAGGGCAG
GTTCACCATTAGCAGGGACAATGCCAAGAACACCCTGTACCTGCAGATGA
GCAGTCTCAAGCCCGAAGATACCGCCGTGTACTTCTGCGCCAAGAGCCTG
AGGAGCAGCCCCAGCAGCAGGTGGTTCGGCAGCCGCGGTCAAGGCACCCA
GGTGACCGTGAGCTCT

In some embodiments, the VH is a single domain antibody domain.

In some embodiments, the VH is a VHH.

TABLE 1

| | | SEQ |
|---|---|---|
| Anti-CD70 VHH | | ID |
| Anti-CD70 | QVQLQESGGGLVQAGGSLRLSCAAPRSIFSINAMGWYRQAPGKQ | 1 |
| VHH, | RELVAAITSGGSPTYADSVKGRFTISRDNAKNTVYLQMNSLKAED | |
| 3HCS92 | TAVYYCATGPYGLDNALDAWGQGTQVTVSS | |
| CDR1 | RSIFSINA | 2 |
| CDR2 | ITSGGSP | 3 |
| CDR3 | ATGPYGLDNALDA | 4 |
| Anti-CD70 | CAAGTTCAGCTCCAGGAATCAGGTGGGGGACTCGTACAGGCT | 5 |
| VH | GGAGGAAGCCTGCGATTGAGCTGTGCCGCTCCACGATCTATCT | |
| (3HCS92- | TCTCAATAAATGCTATGGGTTGGTACAGGCAGGCCCCAGGCAA | |
| coding | ACAAAGAGAACTTGTAGCAGCCATTACTTCAGGGGGGAGCCC | |
| sequence) | AACCTATGCTGACTCAGTAAAGGGGCGGTTCACTATTAGTAGG | |
| | GATAACGCGAAGAATACGGTCTACTTGCAGATGAACAGTCTG | |
| | AAGGCCGAAGATACGGCCGTGTATTATTGTGCCACAGGTCCCT | |
| | ATGGGCTGGACAATGCGTTGGATGCGTGGGGTCAGGGCACGC | |
| | AGGTCACTGTGAGTAGT | |
| 3HCS92 | EVQLLESGGGLVQPGGSLRLSCAAPRSIFSINAMGWYRQAPGKQR | 6 |
| clone h6 | ELVAAITSGGSPTYADSVKGRFTISRDNSKNTLYLQMNSLKAEDT | |
| | AVYYCATGPYGLDNALDAWGQGTLVTVSS | |
| 3HCS92 | EVQLLESGGGLVQPGGSLRLSCAAPRSIFSINAMGWYRQAPGKQR | 7 |
| clone h7 | ELVAAITSGGSPTYADSVKGRFTISRDNSKNTLYLQMNSLRAEDT | |
| | AVYYCATGPYGLDNALDAWGQGTLVTVSS | |
| 3HCS92 | EVQLLESGGGLVQPGGSLRLSCAAPRSIFSINAMGWYRQAPGKQR | 8 |
| clone h8 | ELVSAITSGGSPTYADSVKGRFTISRDNSKNTLYLQMNSLRAEDT | |
| | AVYYCATGPYGLDNALDAWGQGTLVTVSS | |
| 3HCS92 | EVQLLESGGGLVQPGGSLRLSCAAPRSIFSINAMSWYRQAPGKQR | 9 |
| clone h9 | ELVSAITSGGSPTYADSVKGRFTISRDNSKNTLYLQMNSLRAEDT | |
| | AVYYCATGPYGLDNALDAWGQGTLVTVSS | |
| 3HCS92 | EVQLLESGGGLVQPGGSLRLSCAASRSIFSINAMSWYRQAPGKQR | 10 |
| clone h10 | ELVSAITSGGSPTYADSVKGRFTISRDNSKNTLYLQMNSLRAEDT | |
| | AVYYCATGPYGLDNALDAWGQGTLVTVSS | |
| Anti-CD70 | QVQLQESGGGLVQTGGSLRLACTASGFTFDDYAIAWFRQAPGKE | 11 |
| VHH clone | REFVAAISWSGGTTHYADSVKGRFTISRDNAKNTLYLQMSSLKPE | |
| 3CEK186 | DTAVYFCAKSLRSSPSSRWFGSRGQGTQVTVSS | |
| CDR1 | GFTFDDYA | 12 |
| CDR2 | ISWSGGTT | 13 |
| CDR3 | AKSLRSSPSSRWFGS | 14 |
| Anti-CD70 | CAGGTGCAGTTGCAGGAGAGTGGCGGTGGCCTGGTGCAGACC | 15 |
| VH clone | GGTGGCAGTCTGAGGCTGGCCTGCACTGCCAGCGGCTTCACCT | |
| 3CEK186 | TCGACGACTATGCCATCGCGTGGTTCAGGCAGGCCCCTGGCAA | |
| coding | GGAGAGGGAGTTTGTCGCAGCCATCAGCTGGAGCGGAGGCAC | |
| sequence | CACCCACTACGCTGACAGCGTGAAGGGCAGGTTCACCATTAGC | |
| | AGGGACAATGCCAAGAACACCCTGTACCTGCAGATGAGCAGT | |
| | CTCAAGCCCGAAGATACCGCCGTGTACTTCTGCGCCAAGAGCC | |
| | TGAGGAGCAGCCCCAGCAGCAGGTGGTTCGGCAGCCGCGGTC | |
| | AAGGCACCCAGGTGACCGTGAGCTCT | |
| 3CEK186h5 | EVQLQESGGGLVQPGGSLRLSCAASGFTFDDYAIAWFRQAPGKE | 16 |
| | REFVSAISWSGGTTHYADSVKGRFTISRDNAKNTLYLQMSSLKPE | |
| | DTAVYFCAKSLRSSPSSRWFGSRGQGTQVTVS | |
| 3CEK186h6 | EVQLQESGGGLVQPGGSLRLSCAASGFTFDDYAIAWFRQAPGKE | 17 |
| | REFVSAISWSGGTTHYADSVKGRFTISRDNAKNTLYLQMSSLKPE | |
| | DTAVYFCAKSLRSSPSSRWFGSWGQGTQVTVS | |
| 3CEK186h7 | EVQLQESGGGLVQPGGSLRLSCAASGFTFDDYAIAWFRQAPGKE | 18 |
| | REFVSTISWSGGTTHYADSVKGRFTISRDNAKNTLYLQMSSLKPE | |
| | DTAVYFCAKSLRSSPSSRWFGSWGQGTQVTVS | |
| 3CEK186h8 | EVQLQESGGGLVQPGGSLRLSCAASGFTFDDYAIAWFRQAPGKE | 19 |
| | REFVSTISWSGGTTHYADSVKGRFTISRDNAKNTLYLQMSSLKPE | |
| | DTAVYYCAKSLRSSPSSRWFGSWGQGTQVTVS | |
| 3CEK186h9 | EVQLQESGGGLVQPGGSLRLSCAASGFTFDDYAITWFRQAPGKE | 20 |
| | REFVSTISWSGGTTHYADSVKGRFTISRDNAKNTLYLQMSSLKPE | |
| | DTAVYYCAKSLRSSPSSRWFGSWGQGTQVTVS | |
| 3CEK186h10 | EVQLQESGGGLVQPGGSLRLSCAASGFTFDDYAMTWFRQAPGKE | 342 |
| | REFVSTISWSGGTTHYADSVKGRFTISRDNAKNTLYLQMSSLKPE | |
| | DTAVYYCAKSLRSSPSSRWFGSWGQGTQVTVSS | |

In one aspect, provided herein is an scFV having a variable heavy chain comprising a sequence having at least 70% sequence identity to SEQ ID NO: 1. In some embodiments, an scFV as used herein comprises a variable heavy chain (VH) comprising a sequence having at least 80% sequence identity to SEQ ID NO: 1. In some embodiments, the scFV comprises a VH comprising a sequence having at least 85% sequence identity to SEQ ID NO: 1. In some embodiments, the scFV comprises a VH comprising a sequence having at least 90% sequence identity to SEQ ID NO: 1. In some embodiments, the scFV comprises a VH comprising a sequence having at least 91% sequence identity to SEQ ID NO: 1. In some embodiments, the scFV comprises a VH comprising a sequence having at least 92% sequence identity to SEQ ID NO: 1. In some embodiments, the scFV comprises a VH comprising a sequence having at least 93% sequence identity to SEQ ID NO: 1. In some embodiments, the scFV comprises a VH comprising a sequence having at least 94% sequence identity to SEQ ID NO: 1. In some embodiments, the scFV comprises a VH comprising a sequence having at least 95% sequence identity to SEQ ID NO: 1. In some embodiments, the scFV comprises a VH comprising a sequence having at least 96% sequence identity to SEQ ID NO: 1. In some embodiments, the scFV comprises a VH comprising a sequence having at least 97% sequence identity to SEQ ID NO: 1. In some embodiments, the scFV comprises a VH comprising a sequence having at least 98% sequence identity to SEQ ID NO: 1. In some embodiments, the scFV comprises a VH comprising a sequence having at least 99% sequence identity to SEQ ID NO: 1. In some embodiments, the scFV comprises a VH comprising a sequence having a sequence of SEQ ID NO: 1.

In some embodiments, the scFV comprises a VH comprising a sequence having at least 70% sequence identity to SEQ ID NO: 11. In some embodiments, the scFV comprises a VH comprising a sequence having at least 75% sequence identity to SEQ ID NO: 11. In some embodiments, the scFV comprises a VH comprising a sequence having at least 85% sequence identity to SEQ ID NO: 11. In some embodiments, the scFV comprises a VH comprising a sequence having at least 90% sequence identity to SEQ ID NO: 11. In some embodiments, the scFV comprises a VH comprising a sequence having at least 91% sequence identity to SEQ ID NO: 11. In some embodiments, the scFV comprises a VH comprising a sequence having at least 92% sequence identity to SEQ ID NO: 11. In some embodiments, the scFV comprises a VH comprising a sequence having at least 93% sequence identity to SEQ ID NO: 11. In some embodiments, the scFV comprises a VH comprising a sequence having at least 94% sequence identity to SEQ ID NO: 11. In some embodiments, the scFV comprises a VH comprising a sequence having at least 95% sequence identity to SEQ ID NO: 11. In some embodiments, the scFV comprises a VH comprising a sequence having at least 96% sequence identity to SEQ ID NO: 11. In some embodiments, the scFV comprises a VH comprising a sequence having at least 97% sequence identity to SEQ ID NO: 11. In some embodiments, the scFV comprises a VH comprising a sequence having at least 98% sequence identity to SEQ ID NO: 11. In some embodiments, the scFV comprises a VH comprising a sequence having at least 99% sequence identity to SEQ ID NO: 11. In some embodiments, the scFV comprises a VH comprising a sequence having a sequence of SEQ ID NO: 11. In some embodiments, the scFv comprises a variable light chain (VL) domain, that is linked to the N-terminus or the C terminus of the VH domain via a linker peptide comprising 5, 6, 7, 8, 9, 10, 11, 12, 13, 14, 15, 16, 17, 18 or more amino acids. In some embodiments the amino acids in the linker peptide are G, or S or various combinations comprising G and S residues.

In another aspect, provided herein are bispecific or trispecific engagers comprising anti CD70 binding domains. Bispecific or trispecific engagers are secreted molecules having binding domains for two or more extracellular biomolecular, e.g., antigens. Provided herein is a bispecific or trispecific engager having at least one binding domain that binds to CD70. In some embodiments, provided herein is a bispecific engager having an anti-CD70 binder variable domain (V_{H}) having a sequence that is at least 70% identical to SEQ ID NO: 1. In some embodiments, provided herein is a bispecific engager having an anti-CD70 binder variable domain having a sequence that is at least 80% identical to SEQ ID NO: 1. In some embodiments, provided herein is a bispecific engager having an anti-CD70 binder variable domain having a sequence that is at least 85% identical to SEQ ID NO: 1. In some embodiments, provided herein is a bispecific engager having an anti-CD70 binder variable domain having a sequence that is at least 90% identical to SEQ ID NO: 1. In some embodiments, provided herein is a bispecific engager having an anti-CD70 binder variable domain having a sequence that is at least 91% identical to SEQ ID NO: 1. In some embodiments, provided herein is a bispecific engager having an anti-CD70 binder variable domain having a sequence that is at least 92% identical to SEQ ID NO: 1. In some embodiments, provided herein is a bispecific engager having an anti-CD70 binder variable domain having a sequence that is at least 93% identical to SEQ ID NO: 1. In some embodiments, provided herein is a bispecific engager having an anti-CD70 binder variable domain having a sequence that is at least 94% identical to SEQ ID NO: 1. In some embodiments, provided herein is a bispecific engager having an anti-CD70 binder variable domain having a sequence that is at least 95% identical to SEQ ID NO: 1. In some embodiments, provided herein is a bispecific engager having an anti-CD70 binder variable domain having a sequence that is at least 96% identical to SEQ ID NO: 1. In some embodiments, provided herein is a bispecific engager having an anti-CD70 binder variable domain having a sequence that is at least 97% identical to SEQ ID NO: 1. In some embodiments, provided herein is a bispecific engager having an anti-CD70 binder variable domain having a sequence that is at least 98% identical to SEQ ID NO: 1. In some embodiments, provided herein is a bispecific engager having an anti-CD70 binder variable domain having a sequence that is at least 99% identical to SEQ ID NO: 1. In some embodiments, provided herein is a bispecific engager having an anti-CD70 binder variable domain having a sequence that is at least 90%, 91%, 92%, 93%, 94%, 95%, 96%, 97%, 98%, 99%, or 100% identical to a humanized version SEQ ID NO: 1, e.g., SEQ ID NO: 10.

In some embodiments, provided herein is a bispecific engager having an anti-CD70 binder variable domain (VH) having a sequence that is at least 70% identical to SEQ ID NO: 11. In some embodiments, provided herein is a bispecific engager having an anti-CD70 binder variable domain having a sequence that is at least 80% identical to SEQ ID NO: 11. In some embodiments, provided herein is a bispecific engager having an anti-CD70 binder variable domain having a sequence that is at least 85% identical to SEQ ID NO: 11. In some embodiments, provided herein is a bispecific engager having an anti-CD70 binder variable domain having a sequence that is at least 90% identical to SEQ ID NO: 11. In some embodiments, provided herein is a bispecific engager having an anti-CD70 binder variable domain having a sequence that is at least 91% identical to SEQ ID NO: 11. In some embodiments, provided herein is a bispecific engager having an anti-CD70 binder variable domain having a sequence that is at least 92% identical to SEQ ID NO: 11. In some embodiments, provided herein is a bispecific engager having an anti-CD70 binder variable domain having a sequence that is at least 93% identical to SEQ ID NO: 11. In some embodiments, provided herein is a bispecific engager having an anti-CD70 binder variable domain having a sequence that is at least 94% identical to SEQ ID NO: 11. In some embodiments, provided herein is a bispecific engager having an anti-CD70 binder variable domain having a sequence that is at least 95% identical to SEQ ID NO: 11. In some embodiments, provided herein is a bispecific engager having an anti-CD70 binder variable domain having a sequence that is at least 96% identical to SEQ ID NO: 11. In some embodiments, provided herein is a bispecific engager having an anti-CD70 binder variable domain having a sequence that is at least 97% identical to SEQ ID NO: 11. In some embodiments, provided herein is a bispecific engager having an anti-CD70 binder variable domain having a sequence that is at least 98% identical to SEQ ID NO: 11. In some embodiments, provided herein is a bispecific engager having an anti-CD70 binder variable domain having a sequence that is at least 99% identical to SEQ ID NO: 11. In some embodiments, provided herein is a bispecific engager having an anti-CD70 binder variable domain having a sequence that is at least 90%, 91%, 92%, 93%, 94%, 95%, 96%, 97%, 98%, 99%, or 100% identical to a humanized version SEQ ID NO: 11, e.g., SEQ ID NO: 21.

In some embodiments, provided herein is a trispecific engager having an anti-CD70 binder variable domain (VH) having a sequence that is at least 70% identical to SEQ ID NO: 1. In some embodiments, the trispecific engager having an anti-CD70 binder variable domain having a sequence that is at least 80% identical to SEQ ID NO: 1. In some embodiments, the trispecific engager having an anti-CD70 binder variable domain having a sequence that is at least 85% identical to SEQ ID NO: 1. In some embodiments, the trispecific engager having an anti-CD70 binder variable domain having a sequence that is at least 90% identical to SEQ ID NO: 1. In some embodiments, the trispecific engager having an anti-CD70 binder variable domain having a sequence that is at least 91% identical to SEQ ID NO: 1. In some embodiments, the trispecific engager having an anti-CD70 binder variable domain having a sequence that is at least 92% identical to SEQ ID NO: 1. In some embodiments, the trispecific engager having an anti-CD70 binder variable domain having a sequence that is at least 93% identical to SEQ ID NO: 1. In some embodiments, the trispecific engager having an anti-CD70 binder variable domain having a sequence that is at least 94% identical to SEQ ID NO: 1. In some embodiments, the trispecific engager having an anti-CD70 binder variable domain having a sequence that is at least 95% identical to SEQ ID NO: 1. In some embodiments, the trispecific engager having an anti-CD70 binder variable domain having a sequence that is at least 96% identical to SEQ ID NO: 1. In some embodiments, the trispecific engager having an anti-CD70 binder variable domain having a sequence that is at least 97% identical to SEQ ID NO: 1. In some embodiments, the trispecific engager having an anti-CD70 binder variable domain having a sequence that is at least 98% identical to SEQ ID NO: 1. In some embodiments, the trispecific engager having an anti-CD70 binder variable domain having a sequence that is at least 99% identical to SEQ ID NO: 1. In some embodiments, provided herein is a trispecific engager having an anti-CD70 binder variable domain having a sequence that is at least 90%, 91%, 92%, 93%, 94%, 95%, 96%, 97%, 98%, 99%, or 100% identical to a humanized version SEQ ID NO: 1, e.g., SEQ ID NO: 10.

In some embodiments, the trispecific engager having an anti-CD70 binder variable domain (V_{H}) having a sequence that is at least 70% identical to SEQ ID NO: 11. In some embodiments, the trispecific engager having an anti-CD70 binder variable domain having a sequence that is at least 80% identical to SEQ ID NO: 11. In some embodiments, the trispecific engager having an anti-CD70 binder variable domain having a sequence that is at least 85% identical to SEQ ID NO: 11. In some embodiments, the trispecific engager having an anti-CD70 binder variable domain having a sequence that is at least 90% identical to SEQ ID NO: 11. In some embodiments, the trispecific engager having an anti-CD70 binder variable domain having a sequence that is at least 91% identical to SEQ ID NO: 11. In some embodiments, the trispecific engager having an anti-CD70 binder variable domain having a sequence that is at least 92% identical to SEQ ID NO: 11. In some embodiments, the trispecific engager having an anti-CD70 binder variable domain having a sequence that is at least 93% identical to SEQ ID NO: 11. In some embodiments, the trispecific engager having an anti-CD70 binder variable domain having a sequence that is at least 94% identical to SEQ ID NO: 11. In some embodiments, the trispecific engager having an anti-CD70 binder variable domain having a sequence that is at least 95% identical to SEQ ID NO: 11. In some embodiments, the trispecific engager having an anti-CD70 binder variable domain having a sequence that is at least 96% identical to SEQ ID NO: 11. In some embodiments, the trispecific engager having an anti-CD70 binder variable domain having a sequence that is at least 97% identical to SEQ ID NO: 11. In some embodiments, the trispecific engager having an anti-CD70 binder variable domain having a sequence that is at least 98% identical to SEQ ID NO: 11. In some embodiments, the trispecific engager having an anti-CD70 binder variable domain having a sequence that is at least 99% identical to SEQ ID NO: 11. In some embodiments, provided herein is a trispecific engager having an anti-CD70 binder variable domain having a sequence that is at least 90%, 91%, 92%, 93%, 94%, 95%, 96%, 97%, 98%, 99%, or 100% identical to a humanized version SEQ ID NO: 11, e.g., SEQ ID NO: 21.

In one aspect, the variable domain is a component of a chimeric antigen receptor (CAR). In some embodiments contemplated herein is an anti-CD70 CAR, comprising an antigen binding domain capable of binding to CD70, the antigen binding domain being a single-chain variable fragment (scFv), a nanobody, a V_{H} domain, a single domain antibody (sdAb), a VNAR domain, and a VHH domain, a bispecific antibody, a diabody, or a functional fragment of any thereof. In some embodiments, the antigen-binding domain comprises an antibody or an antigen binding thereof, e.g., an Fab, a single-chain variable fragment (scFv), a nanobody, a VH domain, a VL domain, a single domain antibody (sdAb), a VNAR domain, and a VHH domain, a bispecific antibody, a diabody, or a functional fragment thereof that specifically binds to one or more antigens, at least one of which is CD70, and the antigen binding domain comprising the anti-CD70 variable heavy chain domain (VH) described herein.

In some embodiments, the anti-CD70-CAR comprises a VHH antigen binding domain, comprising a sequence that has at least 80%, 90%, 91%, 92%, 93%, 94%, 95%, 96%, 97%, 98%, 99%, or 100% sequence identity to SEQ ID NO: 1, or a humanized or partially humanized version of the VHH, SEQ ID NO: 10. In some embodiments the anti-CD70 CAR comprises an antigen binding domain that is a VHH, having at least 90%, 91%, 92%, 93%, 94%, 95%, 96%, 97%, 98%, 99%, sequence identity to SEQ ID NO: 10 or is 100% identical to a humanized version SEQ ID NO: 1, namely SEQ ID NO: 10. In some embodiments, the anti-CD70-CAR comprises a VHH antigen binding domain, comprising a sequence that has at least 80%, 90%, 91%, 92%, 93%, 94%, 95%, 96%, 97%, 98%, 99%, or 100% sequence identity to SEQ ID NO: 11, or a humanized or partially humanized version of the VHH, SEQ ID NO: 21. In some embodiments the anti-CD70 CAR comprises an antigen binding domain that is a VHH, having at least 90%, 91%, 92%, 93%, 94%, 95%, 96%, 97%, 98%, 99%, sequence identity to SEQ ID NO: 10 or is 100% identical to a humanized version SEQ ID NO: 11, namely SEQ ID NO: 21.

In some embodiments contemplated herein is a CAR that can be expressed in a myeloid cell, e.g., a monocyte, macrophage, dendritic cell, such that the CAR enhances a function of the myeloid cell that expresses the CAR. In some embodiments, a cell therapy is contemplated herein wherein the CAR is expressed in a cell that is introduced into an organism. In some embodiments, the cell is a myeloid cell. In some embodiments, the cell is a CD14+ cell. In some embodiments, the cell is a CD14+ and CD16+ cell. In some embodiments, the cell is a CD14+ and CD16−cell. In some embodiments, the organism is a human.

In some embodiments, a CAR designed for expressing in a myeloid cell comprises an extracellular domain; the extracellular domain comprises an anti-CD70 antigen binding domain, wherein the antigen binding domain is an scFv or a VHH domain; a transmembrane domain that oligomerizes with an endogenous receptor on a myeloid cell and expresses on the membrane of a myeloid cell. In some embodiments, the extracellular antigen binding domain comprises an scFv or a VHH domain that binds to CD70, wherein the scFV or the VHH comprises an amino acid sequence having at least 70% sequence identity to SEQ ID NO: 1 or having at least 70% sequence identity to SEQ ID NO: 11.

In some embodiments, the CAR comprises an antigen binding domain comprising a variable domain heavy chain (VH); the VH having a sequence that is at least 70% identical to SEQ ID NO: 1. In some embodiments, the antigen binding domain comprises a variable domain heavy chain having a sequence that is at least 75% identical to SEQ ID NO: 1. In some embodiments, the antigen binding domain comprises a variable domain heavy chain having a sequence that is at least 80% identical to SEQ ID NO: 1. In some embodiments, the antigen binding domain comprises a variable domain heavy chain having a sequence that is at least 85% identical to SEQ ID NO: 1. In some embodiments, the antigen binding domain comprises a variable domain heavy chain having a sequence that is at least 90% identical to SEQ ID NO: 1. In some embodiments, the antigen binding domain comprises a variable domain heavy chain having a sequence that is at least 95% identical to SEQ ID NO: 1. In some embodiments, the antigen binding domain comprises a variable domain heavy chain having a sequence that is at least 96% identical to SEQ ID NO: 1. In some embodiments, the antigen binding domain comprises a variable domain heavy chain having a sequence that is at least 97% identical to SEQ ID NO: 1. In some embodiments, the antigen binding domain comprises a variable domain heavy chain having a sequence that is at least 98% identical to SEQ ID NO: 1. In some embodiments, the antigen binding domain comprises a variable domain heavy chain having a sequence that is at least 99% identical to SEQ ID NO: 1 In some embodiments, the antigen binding domain comprises a variable domain heavy chain having a sequence that is at least 100% identical to SEQ ID NO: 1.

In some embodiments, the CAR comprises a leader sequence. In some embodiments, the leader sequence (signal sequence) comprises a sequence of MWLQSLLLLGTVACSIS (SEQ ID NO: 306). In some embodiments, extracellular domain comprises a hinge. In some embodiments the hinge is operatively connected to the transmembrane domain. In some embodiments, the hinge is a CD8alpha hinge. In some embodiments, the hinge has a sequence of ALSNSIMYFSHFVPVFLPAKPTTTPAPRPPTPAPTIASQPLSLRPEACRPAAGGAVHTRGLD (SEQ ID NO: 307). In some embodiments the extracellular domain comprises a linker. In some embodiments, the linker is operatively linked to the V_{H} and the hinge. In some embodiments, the linker is operatively linked to the V_{H} and the transmembrane domain. In some embodiments, the linker has a sequence of SGGGSG (SEQ ID NO: 308). In some embodiments, the transmembrane domain is a CD8 transmembrane domain. In some embodiments, the transmembrane domain has a sequence of IYIWAPLAGTCGVLLLSLVITLYC (SEQ ID NO: 309). In some embodiments, the intracellular domain comprises an intracellular signaling domain from FcεR. In some embodiments, the intracellular domain comprises a sequence of

(SEQ ID NO: 310)
RRLKIQVRKAAITSYEKSDGVYTGLSTRNQETYETLKHEKPPQ.

In some embodiments, the intracellular domain comprises at least two intracellular signaling domains. In some embodiments, the least two intracellular signaling domains are separated by a linker. In some embodiments the linker has a sequence of GSGS (SEQ ID NO: 311). In some embodiments, for a cell specific CAR, e.g., a myeloid cell specific CAR is designed, wherein the transmembrane domain is capable of dimerizing or multimerizing with an endogenous protein in a myeloid cell and this dimerizing or multimerizing of the CAR domain with an endogenous protein allows expression and function of the CAR on myeloid cell surface. In some embodiments, the myeloid cell specific CAR comprises a TM domain of a CD16 protein (e.g., a human CD16), having a sequence GLAVSTISSFFPPGYQVSFCLVMVLLFAVDTGLYFSV (SEQ ID NO: 312), which comprises a short extracellular region and the transmembrane region. In some embodiments, the myeloid cell specific CAR comprises a human CD89 domain, having a sequence IHQDYTTQNLIRMAVAGLVLVALLAILV (SEQ ID NO: 313). Incorporating a TM domain of SEQ ID NO: 312 and 313 in the CAR allows oligomerization with a myeloid cell endogenous protein FcR-γ which allows the CAR to be expressed selectively in the cell that expresses the endogenous protein FcR-γ. In some embodiments, the intracellular domain comprises an intracellular signaling domain that that is the PI3K recruitment domain. In some embodiments, the PI3K recruitment domain has a sequence of YEDMRGILYAAPQLRSIRGQPGPNHEEDADSYENM (SEQ ID NO: 314).

In some embodiments, the anti-CD70 antigen binding domain of a CAR comprises an amino acid sequence:

(SEQ ID NO: 315)
QVQLQESGGGLVQAGGSLRLSCAAPRSIFSINAMGWYRQAPGKQRELVA
AITSGGSPTYADSVKGRFTISRDNAKNTVYLQMNSLKAEDTAVYYCATG
PYGLDNALDAWGQGTQVTVSSSGGGGSGALSNSIMYFSHFVPVFLPAKP
TTTPAPRPPTPAPTIASQPLSLRPEACRPAAGGAVHTRGLDIYIWAPLA
GTCGVLLLSLVITLYCRRLKIQVRKAAITSYEKSDGVYTGLSTRNQETY
ETLKHEKPPQGSGSYEDMRGILYAAPQLRSIRGQPGPNHEEDADSYEN
M.

In some embodiments, the CAR comprising the anti-CD70 antigen binding domain comprises a sequence with 70-100% sequence identity to

(SEQ ID NO: 316)
MWLQSLLLLGTVACSISQVQLQESGGGLVQAGGSLRLSCAAPRSIFSIN
AMGWYRQAPGKQRELVAAITSGGSPTYADSVKGRFTISRDNAKNTVYLQ
MNSLKAEDTAVYYCATGPYGLDNALDAWGQGTQVTVSSSGGGGSGALSN
SIMYFSHFVPVFLPAKPTTTPAPRPPTPAPTIASQPLSLRPEACRPAAG
GAVHTRGLDIYIWAPLAGTCGVLLLSLVITLYCRRLKIQVRKAAITSYE
KSDGVYTGLSTRNQETYETLKHEKPPQGSGSYEDMRGILYAAPQLRSIR
GQPGPNHEEDADSYENM.

TABLE 2
Various domains of an exemplary anti-CD70 CAR (SEQ ID NO: 316)

| Domain | Sequence | SEQ ID NO |
|---|---|---|
| Signal peptide | MWLQSLLLLGTVACSIS | SEQ ID NO: 306 |
| leader sequence | | |
| Extracellular | QVQLQESGGGLVQAGGSLRLSCAAPRSIFSINAMG | SEQ ID NO: 1 |
| domain (Anti- | WYRQAPGKQRELVAAITSGGSPTYADSVKGRFTIS | |
| CD70 VHH) | RDNAKNTVYLQMNSLKAEDTAVYYCATGPYGLD | |
| | NALDAWGQGTQVTVSS | |
| Linker | SGGGGSG | SEQ ID NO: 345 |
| CD8 alpha hinge | ALSNSIMYFSHFVPVFLPAKPTTTPAPRPPTPAPTIA | SEQ ID NO: 307 |
| | SQPLSLRPEACRPAAGGAVHTRGLD | |
| CD8 alpha TMD | IYIWAPLAGTCGVLLLSLVITLYC | SEQ ID NO: 309 |
| FCϵR ICD | RRLKIQVRKAAITSYEKSDGVYTGLSTRNQETYETL | SEQ ID NO: 310 |
| | KHEKPPQ | |
| Short linker | GSGS | SEQ ID NO: 311 |
| PI3-K ICD | YEDMRGILYAAPQLRSIRGQPGPNHEEDADSYENM | SEQ ID NO: 314 |

In some embodiments, the CAR comprises an amino acid sequence having at least 80% sequence identity to SEQ ID NO: 316. In some embodiments, the CAR comprises an amino acid sequence having at least 82% sequence identity to SEQ ID NO: 316. In some embodiments, the CAR comprises an amino acid sequence having at least 83% sequence identity to SEQ ID NO: 316. In some embodiments, the CAR comprises an amino acid sequence having at least 84% sequence identity to SEQ ID NO: 316. In some embodiments, the CAR comprises an amino acid sequence having at least 85% sequence identity to SEQ ID NO: 316. In some embodiments, the CAR comprises an amino acid sequence having at least 86% sequence identity to SEQ ID NO: 316. In some embodiments, the CAR comprises an amino acid sequence having at least 87% sequence identity to SEQ ID NO: 316. In some embodiments, the CAR comprises an amino acid sequence having at least 88% sequence identity to SEQ ID NO: 316. In some embodiments, the CAR comprises an amino acid sequence having at least 89% sequence identity to SEQ ID NO: 316. In some embodiments, the CAR comprises an amino acid sequence having at least 90% sequence identity to SEQ ID NO: 316. In some embodiments, the CAR comprises an amino acid sequence having at least 91% sequence identity to SEQ ID NO: 316. In some embodiments, the CAR comprises an amino acid sequence having at least 92% sequence identity to SEQ ID NO: 316. In some embodiments, the CAR comprises an amino acid sequence having at least 93% sequence identity to SEQ ID NO: 316. In some embodiments, the CAR comprises an amino acid sequence having at least 94% sequence identity to SEQ ID NO: 316. In some embodiments, the CAR comprises an amino acid sequence having at least 95% sequence identity to SEQ ID NO: 316. In some embodiments, the CAR comprises an amino acid sequence having at least 96% sequence identity to SEQ ID NO: 316. In some embodiments, the CAR comprises an amino acid sequence having at least 97% sequence identity to SEQ ID NO: 316. In some embodiments, the CAR comprises an amino acid sequence having at least 98% sequence identity to SEQ ID NO: 316. In some embodiments, the CAR comprises an amino acid sequence having at least 99% sequence identity to SEQ ID NO: 316. In some embodiments, the CAR comprises an amino acid sequence of SEQ ID NO: 316.

In some embodiments, the CAR comprises a sequence with 70-100% sequence identity to

(SEQ ID NO: 316)
MWLQSLLLLGTVACSISQVQLQESGGGLVQAGGSLRLSCAAPRSIFSIN
AMGWYRQAPGKQRELVAAITSGGSPTYADSVKGRFTISRDNAKNTVYLQ
MNSLKAEDTAVYYCATGPYGLDNALDAWGQGTQVTVSSSGGGGSGALSN
SIMYFSHFVPVFLPAKPTTTPAPRPPTPAPTIASQPLSLRPEACRPAAG
GAVHTRGLDIYIWAPLAGTCGVLLLSLVITLYCRRLKIQVRKAAITSYE
KSDGVYTGLSTRNQETYETLKHEKPPQGSGSYEDMRGILYAAPQLRSIR
GQPGPNHEEDADSYENM.

In some embodiments, the CAR comprises a sequence with 70-100% sequence identity to

(SEQ ID NO: 315)
QVQLQESGGGLVQAGGSLRLSCAAPRSIFSINAMGWYRQAPGKQRELVA
AITSGGSPTYADSVKGRFTISRDNAKNTVYLQMNSLKAEDTAVYYCATG
PYGLDNALDAWGQGTQVTVSSSGGGGSGALSNSIMYFSHFVPVFLPAKP
TTTPAPRPPTPAPTIASQPLSLRPEACRPAAGGAVHTRGLDIYIWAPLA
GTCGVLLLSLVITLYCRRLKIQVRKAAITSYEKSDGVYTGLSTRNQETY
ETLKHEKPPQGSGSYEDMRGILYAAPQLRSIRGQPGPNHEEDADSYEN
M.

In some embodiments, the CAR comprises a sequence with 70-100% sequence identity to

(SEQ ID NO: 346)
MWLQSLLLLGTVACSISQVQLQESGGGLVQTGGSLRLACTASGFTFDDY
AIAWFRQAPGKEREFVAAISWSGGTTHYADSVKGRFTISRDNAKNTLYL
QMSSLKPEDTAVYFCAKSLRSSPSSRWFGSRGQGTQVTVSSSGGGGSGA
LSNSIMYFSHFVPVFLPAKPTTTPAPRPPTPAPTIASQPLSLRPEACRP
AAGGAVHTRGLDIYIWAPLAGTCGVLLLSLVITLYCRRLKIQVRKAAIT
SYEKSDGVYTGLSTRNQETYETLKHEKPPQGSGSYEDMRGILYAAPQLR
SIRGQPGPNHEEDADSYENM.

In some embodiments, the CAR comprises a sequence with 70-100% sequence identity to

(SEQ ID NO: 347)
QVQLQESGGGLVQTGGSLRLACTASGFTFDDYAIAWFRQAPGKEREFVA
AISWSGGTTHYADSVKGRFTISRDNAKNTLYLQMSSLKPEDTAVYFCAK
SLRSSPSSRWFGSRGQGTQVTVSSSGGGGSGALSNSIMYFSHFVPVFLP
AKPTTTPAPRPPTPAPTIASQPLSLRPEACRPAAGGAVHTRGLDIYIWA
PLAGTCGVLLLSLVITLYCRRLKIQVRKAAITSYEKSDGVYTGLSTRNQ
ETYETLKHEKPPQGSGSYEDMRGILYAAPQLRSIRGQPGPNHEEDADSY
ENM.

In some embodiments, the CAR is encoded by a sequence with 70-100% sequence identity to

(SEQ ID NO: 352)
ATGTGGCTGCAGTCTCTGCTGCTGCTGGGAACAGTGGCCTGCAGCATCAGCCAGGTGCA
GTTGCAGGAGAGTGGCGGTGGCCTGGTGCAGACCGGTGGCAGTCTGAGGCTGGCCTGC
ACTGCCAGCGGCTTCACCTTCGACGACTATGCCATCGCGTGGTTCAGGCAGGCCCCTGG
CAAGGAGAGGGAGTTTGTCGCAGCCATCAGCTGGAGCGGAGGCACCACCCACTACGCT
GACAGCGTGAAGGGCAGGTTCACCATTAGCAGGGACAATGCCAAGAACACCCTGTACC
TGCAGATGAGCAGTCTCAAGCCCGAAGATACCGCCGTGTACTTCTGCGCCAAGAGCCTG
AGGAGCAGCCCCAGCAGCAGGTGGTTCGGCAGCCGCGGTCAAGGCACCCAGGTGACCG
TGAGCTCTAGCGGAGGCGGAGGATCCGGAGCCCTGAGCAATAGCATCATGTACTTCAG
CCACTTCGTGCCCGTGTTTCTGCCCGCCAAGCCTACAACAACACCCGCTCCTAGACCAC
CTACACCAGCTCCTACAATCGCCAGCCAGCCTCTGTCTCTCAGACCTGAAGCCTGTAGA
CCTGCAGCTGGCGGAGCTGTGCATACCAGAGGCCTGGATATCTACATTTGGGCCCCTCT
GGCTGGCACATGTGGCGTTCTGCTGCTCTCTCTGGTCATCACCCTGTACTGCAGACGGCT
GAAGATCCAAGTGCGGAAGGCCGCCATCACCAGCTACGAGAAATCTGATGGCGTGTAC
ACCGGCCTGAGCACCCGGAATCAAGAAACCTACGAGACACTGAAGCACGAGAAGCCTC
CACAAGGCAGCGGCTCCTATGAGGACATGAGAGGCATTCTGTACGCCGCTCCACAGCT
GAGATCTATCAGAGGCCAGCCTGGACCTAACCACGAGGAAGATGCCGACTCCTACGAG
AACATG.

In some embodiments, the CAR is encoded by a sequence with 70-100% sequence identity to

(SEQ ID NO: 353)
CAGGTGCAGTTGCAGGAGAGTGGCGGTGGCCTGGTGCAGACCGGTGGCAGTCTGAGGC
TGGCCTGCACTGCCAGCGGCTTCACCTTCGACGACTATGCCATCGCGTGGTTCAGGCAG
GCCCCTGGCAAGGAGAGGGAGTTTGTCGCAGCCATCAGCTGGAGCGGAGGCACCACCC
ACTACGCTGACAGCGTGAAGGGCAGGTTCACCATTAGCAGGGACAATGCCAAGAACAC
CCTGTACCTGCAGATGAGCAGTCTCAAGCCCGAAGATACCGCCGTGTACTTCTGCGCCA
AGAGCCTGAGGAGCAGCCCCAGCAGCAGGTGGTTCGGCAGCCGCGGTCAAGGCACCCA
GGTGACCGTGAGCTCTAGCGGAGGCGGAGGATCCGGAGCCCTGAGCAATAGCATCATG
TACTTCAGCCACTTCGTGCCCGTGTTTCTGCCCGCCAAGCCTACAACAACACCCGCTCCT
AGACCACCTACACCAGCTCCTACAATCGCCAGCCAGCCTCTGTCTCTCAGACCTGAAGC
CTGTAGACCTGCAGCTGGCGGAGCTGTGCATACCAGAGGCCTGGATATCTACATTTGGG
CCCCTCTGGCTGGCACATGTGGCGTTCTGCTGCTCTCTCTGGTCATCACCCTGTACTGCA
GACGGCTGAAGATCCAAGTGCGGAAGGCCGCCATCACCAGCTACGAGAAATCTGATGG
CGTGTACACCGGCCTGAGCACCCGGAATCAAGAAACCTACGAGACACTGAAGCACGAG
AAGCCTCCACAAGGCAGCGGCTCCTATGAGGACATGAGAGGCATTCTGTACGCCGCTCC
ACAGCTGAGATCTATCAGAGGCCAGCCTGGACCTAACCACGAGGAAGATGCCGACTCC
TACGAGAACATG.

In some embodiments, the CAR is encoded by a sequence with 70-100% sequence identity to

(SEQ ID NO: 354)
ATGTGGCTGCAGTCTCTGCTGCTGCTGGGAACAGTGGCCTGCAGCATCAGCCAAGTTCA
GCTCCAGGAATCAGGTGGGGGACTCGTACAGGCTGGAGGAAGCCTGCGATTGAGCTGT
GCCGCTCCACGATCTATCTTCTCAATAAATGCTATGGGTTGGTACAGGCAGGCCCCAGG
CAAACAAAGAGAACTTGTAGCAGCCATTACTTCAGGGGGGAGCCCAACCTATGCTGAC
TCAGTAAAGGGGCGGTTCACTATTAGTAGGGATAACGCGAAGAATACGGTCTACTTGCA
GATGAACAGTCTGAAGGCCGAAGATACGGCCGTGTATTATTGTGCCACAGGTCCCTATG
GGCTGGACAATGCGTTGGATGCGTGGGGTCAGGGCACGCAGGTCACTGTGAGTAGTAG
CGGAGGCGGAGGATCCGGAGCCCTGAGCAATAGCATCATGTACTTCAGCCACTTCGTGC
CCGTGTTTCTGCCCGCCAAGCCTACAACAACACCCGCTCCTAGACCACCTACACCAGCT
CCTACAATCGCCAGCCAGCCTCTGTCTCTCAGACCTGAAGCCTGTAGACCTGCAGCTGG
CGGAGCTGTGCATACCAGAGGCCTGGATATCTACATTTGGGCCCCTCTGGCTGGCACAT
GTGGCGTTCTGCTGCTCTCTCTGGTCATCACCCTGTACTGCAGACGGCTGAAGATCCAA
GTGCGGAAGGCCGCCATCACCAGCTACGAGAAATCTGATGGCGTGTACACCGGCCTGA
GCACCCGGAATCAAGAAACCTACGAGACACTGAAGCACGAGAAGCCTCCACAAGGCAG
CGGCTCCTATGAGGACATGAGAGGCATTCTGTACGCCGCTCCACAGCTGAGATCTATCA
GAGGCCAGCCTGGACCTAACCACGAGGAAGATGCCGACTCCTACGAGAACATG.

In some embodiments, the CAR is encoded by a sequence with 70-100% sequence identity to

(SEQ ID NO: 355)
CAAGTTCAGCTCCAGGAATCAGGTGGGGGACTCGTACAGGCTGGAGGAAGCCTGCGAT
TGAGCTGTGCCGCTCCACGATCTATCTTCTCAATAAATGCTATGGGTTGGTACAGGCAG
GCCCCAGGCAAACAAAGAGAACTTGTAGCAGCCATTACTTCAGGGGGGAGCCCAACCT
ATGCTGACTCAGTAAAGGGGCGGTTCACTATTAGTAGGGATAACGCGAAGAATACGGT
CTACTTGCAGATGAACAGTCTGAAGGCCGAAGATACGGCCGTGTATTATTGTGCCACAG
GTCCCTATGGGCTGGACAATGCGTTGGATGCGTGGGGTCAGGGCACGCAGGTCACTGTG
AGTAGTAGCGGAGGCGGAGGATCCGGAGCCCTGAGCAATAGCATCATGTACTTCAGCC
ACTTCGTGCCCGTGTTTCTGCCCGCCAAGCCTACAACAACACCCGCTCCTAGACCACCT
ACACCAGCTCCTACAATCGCCAGCCAGCCTCTGTCTCTCAGACCTGAAGCCTGTAGACC
TGCAGCTGGCGGAGCTGTGCATACCAGAGGCCTGGATATCTACATTTGGGCCCCTCTGG
CTGGCACATGTGGCGTTCTGCTGCTCTCTCTGGTCATCACCCTGTACTGCAGACGGCTGA
AGATCCAAGTGCGGAAGGCCGCCATCACCAGCTACGAGAAATCTGATGGCGTGTACAC
CGGCCTGAGCACCCGGAATCAAGAAACCTACGAGACACTGAAGCACGAGAAGCCTCCA
CAAGGCAGCGGCTCCTATGAGGACATGAGAGGCATTCTGTACGCCGCTCCACAGCTGA
GATCTATCAGAGGCCAGCCTGGACCTAACCACGAGGAAGATGCCGACTCCTACGAGAA
CATG.

### Methods of Use

In one aspect, provided herein is a composition for treating cancer in a subject, comprising a polynucleotide sequence encoding a VHH selected from a group consisting of SEQ ID NO: 1-4, 6-14, 16-21, 315 and 316. In some embodiments, the cancer is a solid tumor. In some embodiments, the cancer is a melanoma. In some embodiments, the cancer is a hematological cancer. In one embodiment, the cancer is RCC, melanoma, breast cancer, MCL, CTCL, DLBCL, an AML, thymic carcinoma, nasophryngeal carcinoma, mesothelioma, CLL, NSCLC or GBM. In some embodiments, the therapeutic composition comprises a polypeptide from Table 1, comprising a sequence of SEQ ID NOs 1-20, and 342. In some embodiments, a human subject with the cancer is treated with a composition comprising a polypeptide comprising any one or more of the sequences of SEQ ID NO: 1-4, 6-14, 16-21, or a recombinant polynucleic acid encoding any one or more of the sequences of SEQ ID NO: 1-4, 6-14, 16-21. In some embodiments, the polynucleotide may comprise a sequence of SEQ ID NO: 5, or SEQ ID NO: 15. In some embodiments, the polynucleotide is an mRNA. In some embodiments, the composition comprises a delivery vehicle, e.g., a nanoparticle. In some embodiments, the nanoparticle is a lipid nanoparticle. In some embodiments, the nanoparticle is a non-lipid nanoparticle. In some embodiments the composition is formulated for systemic administration to the subject, e.g., via intravenous injection, intramuscular injection, subcutaneous injection, intraocular injection and so on.

In one aspect, provided herein is a composition for treating cancer in a subject, comprising a polynucleotide sequence encoding a polypeptide comprising a CDR1, CDR2 and a CDR3 sequence, wherein the CDR3 is SEQ ID NO: 4. In one aspect, provided herein is a composition for treating cancer in a subject, comprising a polynucleotide sequence encoding a polypeptide comprising a CDR1, CDR2 and a CDR3 sequence, wherein the CDR3 is SEQ ID NO: 14. In some embodiments, the polypeptide is a chimeric antigen receptor (CAR). In some embodiments, the CAR comprises a binding domain having a CDR3 of SEQ ID NO: 4, with a CDR1 of SEQ ID NO: 2 and a CDR2 of SEQ ID NO: 3. In some embodiments, the CAR comprises a binding domain having a CDR3 of SEQ ID NO: 14, with a CDR1 of SEQ ID NO: 12 and a CDR2 of SEQ ID NO: 13.

In some embodiments, the recombinant nucleic acid is a vector. Provided herein is a composition comprising a cell comprising the recombinant nucleic acid of the composition described above. In some embodiments, the cell is an immune cell, wherein the cell is a myeloid cell, a lymphoid cell, a precursor cell, a stem cell or an induced pluripotent cell. In some embodiments, the cell is CD14+/CD16−. In some embodiment, the cell is a population of cells, comprising at least 1×10\hat{ }5 cells.

Provided herein is a pharmaceutical composition comprising the composition described above.

Provided herein is a method of treating a disease or condition in a subject in need thereof comprising administering to the subject the pharmaceutical composition described above, wherein the disease or condition is a cancer.

### Anti-GPC3 Binding Domains and Compositions

Glypican-3 (GPC3) is highly expressed in hepatocellular carcinoma (HCC) and usually is associated with poor prognosis. GPC3 is overexpressed in the liver of HCC patients but is not in healthy adult liver. Therefore, GPC3 is a promising candidate for therapeutic targets.

In one aspect, provided herein is a composition comprising a construct comprising an antigen binding domain comprising an anti-GPC3 antibody or binding fragment thereof, or a recombinant nucleic acid encoding the antigen binding domain, wherein the antigen binding domain comprises a heavy chain variable domain (VH) comprising a heavy chain complementarity determining region 3 (HC CDR3) of any one of the sequences selected from the group consisting of

(SEQ ID NO: 113)
ATACADTTQYAYDY,
(SEQ ID NO: 114)
ATACADTTLYEYDY,
(SEQ ID NO: 115)
ATACVDTTQYEYDY,
(SEQ ID NO: 116)
ATACADATQHEYDY,
(SEQ ID NO: 117)
ATACADTTQYDYDY,
(SEQ ID NO: 118)
ATACADTTQYEYDY,
(SEQ ID NO: 119)
ATACADTTHYEYDY,
(SEQ ID NO: 120)
ATACVITTLYEYDY,
(SEQ ID NO: 121)
ATACAETTLYEYDY,
(SEQ ID NO: 122)
ATACADTTQHEYDY,
SEQ ID NO: 123)
ATACVDTTHYEYDY,
SEQ ID NO: 124)
ATACASTTLYEYDY,
(SEQ ID NO: 125)
ATACVVTTLYEYDY,
(SEQ ID NO: 126)
ATACGGATGPYDY,
(SEQ ID NO: 127)
ATACAGAIGPYDY,
(SEQ ID NO: 128)
ATACVVVGDQNDY,
(SEQ ID NO: 129)
ATACVVVGDRNDY,
(SEQ ID NO: 130)
ATDCAGGTSTPYDY,
(SEQ ID NO: 131)
ATDCAGGTATPYDY,
(SEQ ID NO: 132)
ATACVVADRNEYDY,
(SEQ ID NO: 133)
ATSCVVVTKNEYDY,
(SEQ ID NO: 134)
ATACSGLTHEYDY,
(SEQ ID NO: 135)
ATTCSGLTHEYDY,
(SEQ ID NO: 136)
ATACANWSSLGPYDY,
(SEQ ID NO: 137)
ATACANWSTLGPYDY,
(SEQ ID NO: 138)
ATACSDPRVYEYDY,
(SEQ ID NO: 139)
ATTCASPEKYEYDY,
(SEQ ID NO: 140)
ATHCGGTSWGTSYDY,
(SEQ ID NO: 141)
ATHCGGSSWSNEYD,
(SEQ ID NO: 142)
YARYSGRTY,
(SEQ ID NO: 143)
ASSAWPAGPKHQVEYDY,
(SEQ ID NO: 144)
ATACGSLVGMYDY,
(SEQ ID NO: 145)
ATACGSAVHEYDY,
(SEQ ID NO: 146)
ATDCVGFGSNWFDY,
(SEQ ID NO: 147)
ATACASPVIYEYDY,
(SEQ ID NO: 148)
ATDCAGGVGHEYDY,
(SEQ ID NO: 149)
ATDCSLHGSDYPYDY
and
(SEQ ID NO: 150)
AVRIYSGSFDNTLAYDY.

In some embodiments, the VH of the anti-GPC3 antibody or binding fragment thereof further comprises a heavy chain complementarity determining region 1 (HC CDR1) of any one of the sequences selected from the group consisting of SEQ ID NOs: GFPLAYYA (SEQ ID NO: 151), GFSLDYYA (SEQ ID NO: 152), GFPLDYYA (SEQ ID NO: 153), GFTLDYYA (SEQ ID NO: 154), GFSLNYYA (SEQ ID NO: 155), GFTLAYYA (SEQ ID NO: 156), GFTLGYYA (SEQ ID NO: 157), GFPLNYYA (SEQ ID NO: 158), GFPLHYYA (SEQ ID NO: 159), GFSLGYYA (SEQ ID NO: 160), GFPLGYYA (SEQ ID NO: 161), GFPLEYYA (SEQ ID NO: 162), GSDFRADA (SEQ ID NO: 163), GRTFSSYG (SEQ ID NO: 164), GFSLAYYA (SEQ ID NO: 165) and GLTFRSVG (SEQ ID NO: 166).

In some embodiments, the VH of the anti-GPC3 antibody or binding fragment thereof further comprises a heavy chain complementarity determining region 2 (HC CDR2) of any one of the sequences selected from the group consisting of SEQ ID NOs: ISNSDGST (SEQ ID NO: 167), ISASDGST (SEQ ID NO: 168), ISSSDGST (SEQ ID NO: 169), ISSSDGNT (SEQ ID NO: 170), ISSADGST (SEQ ID NO: 171), ISSSGGST (SEQ ID NO: 172), ISSGDGST (SEQ ID NO: 173), ISAGDGNT (SEQ ID NO: 174), ISSSDDST (SEQ ID NO: 175), ISSNDGST (SEQ ID NO: 176), ISSPDGST (SEQ ID NO: 177), ISSRTGGT (SEQ ID NO: 178), ISAGDGSST (SEQ ID NO: 179), ISSSDGSSSDGNT (SEQ ID NO: 180), ISSGDGNT (SEQ ID NO: 181), ISSGDGKT (SEQ ID NO: 182), ISSSDGGT (SEQ ID NO: 183), ISSRTGST (SEQ ID NO: 184), ISSRTGNT (SEQ ID NO: 185), ISSSDGHSST (SEQ ID NO: 186), ISSSSDGNT (SEQ ID NO: 187), ISASNGNT (SEQ ID NO: 188), ISSGSDGNT (SEQ ID NO: 189), ISASDGNT (SEQ ID NO: 190), IDSITSI (SEQ ID NO: 191), ISWSGGSTIAASVGST (SEQ ID NO: 192), ISSSDGSDGNT (SEQ ID NO: 193) and ASPSGVIT (SEQ ID NO: 194).

TABLE 3

| | | SEQ |
|---|---|---|
| Anti-GPC3 VH domains | | ID |
| Anti-GPC3 VH-1 | QVQLQESGGGLVHSGGSLRLSCAASGFPLAYYAIGWFRQAPG | 22 |
| | KEREGVSCISSSDGNTYYADAVKGRFTISRDNAKNTVYLQMN | |
| | SLKPEDTAVYYCATACADTTQHEYDYWGQGTQVTVSS | |
| Anti-GPC3 VH-2 | QVQLQESGGGLVHSGGSLRLSCAASGFPLDYYAIGWFRQAPG | 23 |
| | KEREGVSCISSADGSTYYADSVKGRFTISRDNAKNTVYLQMN | |
| | SLGPEDTAVYYCATACADTTQYDYDYWGQGTQVTVSS | |
| Anti-GPC3 VH-3 | QVQLQESGGGLVHSGGSLRLSCAASGFTLDYYAIGWFRRAPG | 24 |
| | KEREGVSCISSGDGKTYYADSVKGRFTISRDNAKNTVYLQMN | |
| | SLKPEDTAVYYCATACAGAIGPYDYWGQGTQVTVSS | |
| Anti-GPC3 VH-4 | QVQLQESGGGLVPPGGSLRLSCAASGFPLDYYAIGWFRQAPG | 25 |
| | KEREGVSCISSADGSTYYADSVKGRFTISRDNAKNTVYLQMN | |
| | SLGPEDTAVYYCATACADTTQYDYDYWGQGTQVTVSS | |
| Anti-GPC3 VH-5 | QVQLQESGGGLVQAGGSLRLSCAASGFSLGYYAIGWFRQAPG | 26 |
| | KEREGVSCISSSDGHSSTYYADSVKGRFTISRDNAKNTVYLQ | |
| | MNNLKPEDTAVYYCATDCAGGTATPYDYWGQGTQVTVSS | |
| Anti-GPC3 VH-6 | QVQLQESGGGLVQAGGSLRLSCAASGRTFSSYGMGWFRQAP | 27 |
| | GKEREFVAAISWSGGSTYYADSVKGRFTISRDNAKNTVYLQM | |
| | NSLKPEDTAVYYCASSAWPAGPKHQVEYDYWGQGTQVTVSS | |
| Anti-GPC3 VH-7 | QVQLQESGGGLVQAGGSLRLSCTASGFSLDYYAIGWFRQAPG | 28 |
| | KEREGVACISSRTGSTYYADSVKGRFTISRDNAKNTVALQMN | |
| | SLKPEDTAVYYCATACVVVGDQNDYWGQGTQVTVSS | |
| Anti-GPC3 VH-8 | QVQLQESGGGLVQDGGSLRLSCAASGFPLAYYAIGWFRQAPG | 29 |
| | KEREGVSCISASDGSTYYADSVKGRFTISRDNAKNTVYLQMN | |
| | SLKPEDTAVYYCATACAETTLYEYDYWGQGTQVTVSS | |
| Anti-GPC3 VH-9 | QVQLQESGGGLVQPGESLRLSCAASGFPLAYYAIGWFRQAPG | 30 |
| | KEREGVSCISSSDGSTYYADSVKGRFTISRDNAKNTVYLQMN | |
| | SLKPEDTAVYYCATACANWSTLGPYDYWGQGTQVTVSS | |
| Anti-GPC3 VH-10 | QVQLQESGGGLVQPGESLRLSCAASGFTLAYYAIGWFRQAPG | 31 |
| | KEREGVSCISSSDGNTYYADSVKGRFTISRDNAKNTVYLQMN | |
| | RLKPEDTAVYYCATACADTTQYEYDYWGQGTQVTVSS | |
| Anti-GPC3 VH-11 | QVQLQESGGGLVQPGGSLKLSCAASGSDFRADAMGWYRQAP | 32 |
| | GKEREPVAIDSITSIYYVDSVEGRFTISRDNTKNTVYLQMTSLK | |
| | PEDTAVYYCYARYSGRTYWGRGTQVTVSS | |
| Anti-GPC3 VH-12 | QVQLQESGGGLVQPGGSLRLSCAASGFPLAYYAIGWFRQAPG | 33 |
| | KEREGVSCISASDGSTYYADSVKGRFTISRDNAKNTVYLQMN | |
| | SLRPEDTAVYYCATACADTTLYEYDYWGQGTQVTVSS | |
| Anti-GPC3 VH-13 | QVQLQESGGGLVQPGGSLRLSCAASGFPLAYYAIGWFRQAPG | 34 |
| | KEREGVSCISSSDGNTYYADAVKGRFAISRDNAKNTVYLQMN | |
| | SLKPEDTAVYYCATACSDPRVYEYDYWGQGTQVTVSS | |
| Anti-GPC3 VH-14 | QVQLQESGGGLVQPGGSLRLSCAASGFPLAYYAIGWFRQAPG | 35 |
| | KEREGVSCISSSDGNTYYADAVKGRFTISRDNAKNTVYLQMN | |
| | SLKPEDTAVYYCATACADTTQHEYDYWGQGTQVTVSS | |
| Anti-GPC3 VH-15 | QVQLQESGGGLVQPGGSLRLSCAASGFPLAYYAIGWFRQAPG | 36 |
| | KEREGVSCISSSDGNTYYADAVKGRFTISRDNAKNTVYLQMN | |
| | SLKPEDTAVYYCATACVDTTHYEYDYWGQGTQVTVSS | |
| Anti-GPC3 VH-16 | QVQLQESGGGLVQPGGSLRLSCAASGFPLAYYAIGWFRQAPG | 37 |
| | KEREGVSCISSSDGNTYYADSVKGRFTISRDNAKNTVYLQMN | |
| | SLKPEDTAVYYCATACADATQHEYDYWGQGTQVTVSS | |
| Anti-GPC3 VH-17 | QVQLQESGGGLVQPGGSLRLSCAASGFPLAYYAIGWFRQAPG | 38 |
| | KEREGVSCISSSDGSTYYADSVKGRFTISRDNAKNTVYLQMN | |
| | SLGPEDTAVYYCATACADTTQYDYDYWGQGTQVTVSS | |
| Anti-GPC3 VH-18 | QVQLQESGGGLVQPGGSLRLSCAASGFPLAYYAIGWFRQAPG | 39 |
| | KEREGVSCISSSDGSTYYADSVKGRFTISRDNAKNTVYLQMN | |
| | SLKPEDTAVYYCATACADTTQYEYDYWGQGTQVTVSS | |
| Anti-GPC3 VH-19 | QVQLQESGGGLVQPGGSLRLSCAASGFPLAYYAIGWFRQAPG | 40 |
| | KEREGVSCISSSDGSTYYADSVKGRFTISRDNAKNTVYLQMN | |
| | SLKPEDTAVYYCATACGGATGPYDYWGQGTQVTVSS | |
| Anti-GPC3 VH-20 | QVQLQESGGGLVQPGGSLRLSCAASGFPLAYYAIGWFRRAPG | 41 |
| | KEREGVSCISSSDGNTYYADAVKGRFTISRDNAKNTVYLQMN | |
| | SLKPEDTAVYYCATACADTTQHEYDYWGQGTQVTVSS | |
| Anti-GPC3 VH-21 | QVQLQESGGGLVQPGGSLRLSCAASGFPLDYYAIGWFRQAPG | 42 |
| | KEREGVSCISAGDGSSTYYADSVKGRFTISRDNAKNTVYLQM | |
| | NSLKPEDTAVYYCATACASTTLYEYDYWGQGTQVTVSS | |
| Anti-GPC3 VH-22 | QVQLQESGGGLVQPGGSLRLSCAASGFPLDYYAIGWFRQAPG | 43 |
| | KEREGVSCISSADGSTYYADSVKGRFTISRDNAKNAVYLQMN | |
| | SLGPEDTAVYYCATACADTTQYDYDYWGQGTQVTVSS | |
| Anti-GPC3 VH-23 | QVQLQESGGGLVQPGGSLRLSCAASGFPLDYYAIGWFRQAPG | 44 |
| | KEREGVSCISSADGSTYYADSVKGRFTISRDNAKNTVYLQMN | |
| | SLGPEDTAVYYCATACADTTQYDYDYWGQGTQVTVSS | |
| Anti-GPC3 VH-24 | QVQLQESGGGLVQPGGSLRLSCAASGFPLDYYAIGWFRQAPG | 45 |
| | KEREGVSCISSADGSTYYADSVKGRFTISRDNAKNTVYLQMN | |
| | SLKPEDTAVYYCATACVDTTQYEYDYWGQGTQVTVSS | |
| Anti-GPC3 VH-25 | QVQLQESGGGLVQPGGSLRLSCAASGFPLDYYAIGWFRQAPG | 46 |
| | KEREGVSCISSPDGSTYYADSVKGRFTISRDNAKNTVYLQMN | |
| | SLKPEDTAVYYCATACVDTTQYEYDYWGQGTQVTVSS | |
| Anti-GPC3 VH-26 | QVQLQESGGGLVQPGGSLRLSCAASGFPLDYYAIGWFRQAPG | 47 |
| | KEREGVSCISSSDGSDGNTYYADSVKGRFTISRDNAKNTVYL | |
| | QMNSLKPEDTAVYYCATDCSLHGSDYPYDYWGQGTQVTVSS | |
| Anti-GPC3 VH-27 | QVQLQESGGGLVQPGGSLRLSCAASGFPLDYYAIGWFRQAPG | 48 |
| | KEREGVSCISSSDGSTYYADSVKGRFTISRDNAKNTVYLQMN | |
| | SLKPEDTAVYYCATACADTTQYEYDYWGQGTQVTVSS | |
| Anti-GPC3 VH-28 | QVQLQESGGGLVQPGGSLRLSCAASGFPLEYYAIGWFRQAPG | 49 |
| | KEREGVSCISSSDGSTYYADSVKGRFTISRDNAKNTVYLQMN | |
| | SLKPEDTAVYYCATACSDPRVYEYDYWGQGTQVTVSS | |
| Anti-GPC3 VH-29 | QVQLQESGGGLVQPGGSLRLSCAASGFPLGYYAIGWFRQAPG | 50 |
| | KEREGVSCISSSDDSTYYADSVKGRFTISRDNDKNTVYLQMN | |
| | SLKPEDTAVYYCATDCAGGTSTPYDYWGQGTQVTVSS | |
| Anti-GPC3 VH-30 | QVQLQESGGGLVQPGGSLRLSCAASGFPLHYYAIGWFRQAPG | 51 |
| | KEREGVSCISSGDGSTYYADSVKGRFTISRDNAKNTVYLQMN | |
| | SLKPEDTAVYYCATSCVVVTKNEYDYWGQGTQVTVSS | |
| Anti-GPC3 VH-31 | QVQLQESGGGLVQPGGSLRLSCAASGFPLHYYAIGWFRQAPG | 52 |
| | KEREGVSCISSSDGSTYYADSVKGRFTISRDNAKNTVYLQMN | |
| | SLKPEDTAVYYCATACGGATGPYDYWGQGTQVTVSS | |
| Anti-GPC3 VH-32 | QVQLQESGGGLVQPGGSLRLSCAASGFPLHYYAIGWFRQAPG | 53 |
| | KEREGVSCISSSDGSTYYADSVKGRFTISRDNAKNTVYLQMN | |
| | SLKPEDTAVYYCATACVVADRNEYDYWGQGTQVTVSS | |
| Anti-GPC3 VH-33 | QVQLQESGGGLVQPGGSLRLSCAASGFPLHYYAIGWFRQAPG | 54 |
| | KEREGVSCISSSDGSTYYADSVKGRFTISRDNAKNTVYLQMN | |
| | SLRPEDTAVYYCATACVVADRNEYDYWGQGTQVTVSS | |
| Anti-GPC3 VH-34 | QVQLQESGGGLVQPGGSLRLSCAASGFPLNYYAIGWFRQAPG | 55 |
| | KEREGVSCISASDGNTYYADSVKGRFTISRDNAKNTVYLQMN | |
| | SLKPEDTAVYYCATTCASPEKYEYDYWGQGTQVTVSS | |
| Anti-GPC3 VH-35 | QVQLQESGGGLVQPGGSLRLSCAASGFPLNYYAIGWFRQAPG | 56 |
| | KEREGVSCISSSDGSTYYADSVKGRFIISRDNAKNTVYLQMNS | |
| | LKPEDTAVYYCATACGGATGPYDYWGQGTQVTVSS, | |
| Anti-GPC3 VH-36 | QVQLQESGGGLVQPGGSLRLSCAASGFPLNYYAIGWFRQAPG | 57 |
| | KEREGVSCISSSDGSTYYADSVKGRFTISRDNAKNTVYLQMN | |
| | SLKPEDTAVYYCATACGSAVHEYDYWGQGTQVTVSS, | |
| Anti-GPC3 VH-37 | QVQLQESGGGLVQPGGSLRLSCAASGFSLAYYAIGWFRQAPG | 58 |
| | KEREGVSCIAASVGSTYYADSVKGRFTISRDDAKNTVYLQMN | |
| | SLKPEDTAVYYCATDCAGGVGHEYDYWGQGTQVTVSS | |
| Anti-GPC3 VH-38 | QVQLQESGGGLVQPGGSLRLSCAASGFSLDYYAIGWFRQAPG | 59 |
| | KEREGVSCISSSDGSTYYADSVKGRFTISRDNAKNAVYLQMN | |
| | SLKPEDTAVYYCATACGGATGPYDYWGQGTQVTVSS | |
| Anti-GPC3 VH-39 | QVQLQESGGGLVQPGGSLRLSCAASGFSLDYYAIGWFRQAPG | 60 |
| | KEREGVSCISSSDGSTYYADSVKGRFTISRDNAKNAVYLQMN | |
| | SLKPEDTAVYYCATACVDTTQYEYDYWGQGTQVTVSS | |
| Anti-GPC3 VH-40 | QVQLQESGGGLVQPGGSLRLSCAASGFSLDYYAIGWFRQAPG | 61 |
| | KEREGVSCISSSDGSTYYADSVKGRFTISRDNAKNTVYLQMN | |
| | SLKPEDTAVYYCATDCAGGTSTPYDYWGQGTQVTVSS | |
| Anti-GPC3 VH-41 | QVQLQESGGGLVQPGGSLRLSCAASGFSLNYYAIGWFRQAPG | 62 |
| | KEREGVSCISAGDGNTYYADSVKGRFTISRDNAANTVSLQMD | |
| | SLKPEDTAVYYCATACVITTLYEYDYWGQGTQVTVSS | |
| Anti-GPC3 VH-42 | QVQLQESGGGLVQPGGSLRLSCAASGFTLAYYAIGWFRQAPG | 63 |
| | KEREGVSCISSSDGSTYYADSVKGRFTISRDNAKNTVYLQMN | |
| | SLKPEDTAVYYCATACADTTQHEYDYWGQGTQVTVSS | |
| Anti-GPC3 VH-43 | QVQLQESGGGLVQPGGSLRLSCAASGFTLAYYAIGWFRQAPG | 64 |
| | KEREGVSCISSSDGSTYYADSVKGRFTISRDNAKNTVYLQMN | |
| | SLKPEDTAVYYCATACADTTQYEYDYWGQGTQVTVSS | |
| Anti-GPC3 VH-44 | QVQLQESGGGLVQPGGSLRLSCAASGFTLDYYAIGWFRQAPG | 65 |
| | KEREGVACISSSDGSTYYADSVKGRFTISRDNAKNTVYLQMN | |
| | SLKPEDTAVYYCATACGGATGPYDYWGQGTQVTVSS | |
| Anti-GPC3 VH-45 | QVQLQESGGGLVQPGGSLRLSCAASGFTLDYYAIGWFRQAPG | 66 |
| | KEREGVACISSSDGSTYYADSVKGRFTISRDNAKNTVYLQMN | |
| | SLKPQDTAVYYCATACGSLVGMYDYWGQGTQVTVSP | |
| Anti-GPC3 VH-46 | QVQLQESGGGLVQPGGSLRLSCAASGFTLDYYAIGWFRQAPG | 67 |
| | KEREGVSCISASDGNTYYADSVKGRFTISRDNAKNTVYLQMN | |
| | SLKPEDTAVYYCATTCASPEKYEYDYWGQGTQVTVSS | |
| Anti-GPC3 VH-47 | QVQLQESGGGLVQPGGSLRLSCAASGFTLDYYAIGWFRQAPG | 68 |
| | KEREGVSCISASNGNTYYADSVKGRFTISRDSAKNTVYLQMN | |
| | SLKPEDTAVYYCATTCSGLTHEYDYWGQGTQVTVSS | |
| Anti-GPC3 VH-48 | QVQLQESGGGLVQPGGSLRLSCAASGFTLDYYAIGWFRQAPG | 69 |
| | KEREGVSCISSGDGNTYYADSVKGRFTISRDNAKNTVYLQMN | |
| | SLKPEDTAVYYCATACGGATGPYDYWGQGTQVTVSS, | |
| Anti-GPC3 VH-49 | QVQLQESGGGLVQPGGSLRLSCAASGFTLDYYAIGWFRQAPG | 70 |
| | KEREGVSCISSGDGSTYYADSVKGRFTISRDNAKNTVYLQMN | |
| | SLKPEDTAVYYCATHCGGSSWSNEYDYWGQGTQVTVSS, | |
| Anti-GPC3 VH-51 | QVQLQESGGGLVQPGGSLRLSCAASGFTLDYYAIGWFRQAPG | 71 |
| | KEREGVSCISSNDGSTYYADSVKGRFTISRDNAKNTVYLQMN | |
| | SLKPEDTAVYYCATACADTTQHEYDYWGQGTQVTVSS, | |
| Anti-GPC3 VH-52 | QVQLQESGGGLVQPGGSLRLSCAASGFTLDYYAIGWFRQAPG | 72 |
| | KEREGVSCISSSDGGTYYADSVKGRFTISRDNAKNTVYLQMN | |
| | SLKPEDTAVYYCATACGGATGPYDYWGQGTQVTVSS, | |
| Anti-GPC3 VH-53 | QVQLQESGGGLVQPGGSLRLSCAASGFTLDYYAIGWFRQAPG | 73 |
| | KEREGVSCISSSDGSSSDGNTYYADSVKGRFTISRDNAKNTVY | |
| | LQMNNLKPEDTAVYYCATACVVTTLYEYDYWGQGTQVTVS | |
| | S, | |
| Anti-GPC3 VH-54 | QVQLQESGGGLVQPGGSLRLSCAASGFTLDYYAIGWFRQAPG | 74 |
| | KEREGVSCISSSDGSTYYADSVKGRFTISRDNAKNTVYLQMN | |
| | SLKPEDTAVYYCATACADTTQYEYDYWGQGTQVTVSP, | |
| Anti-GPC3 VH-55 | QVQLQESGGGLVQPGGSLRLSCAASGFTLDYYAIGWFRQAPG | 75 |
| | KEREGVSCISSSDGSTYYADSVKGRFTISRDNAKNTVYLQMN | |
| | SLKPEDTAVYYCATACGGATGPYDYWGQGTQVTVSS, | |
| Anti-GPC3 VH-56 | QVQLQESGGGLVQPGGSLRLSCAASGFTLDYYAIGWFRQAPG | 76 |
| | KEREGVSCISSSGGSTYYADSVKGRFTISRDNAKNTVYLQMN | |
| | MLKPEDTAVYYCATACADTTQYEYDYWGQGTQVTVSS, | |
| Anti-GPC3 VH-57 | QVQLQESGGGLVQPGGSLRLSCAASGFTLDYYAIGWFRQAPG | 77 |
| | KEREGVSCISSSGGSTYYADSVKGRFTISRDNAKNTVYLQMN | |
| | SLKPEDTAVYYCATACASPVIYEYDYWGQGTQVTVSS | |
| Anti-GPC3 VH-58 | QVQLQESGGGLVQPGGSLRLSCAASGFTLDYYAIGWFRQAPG | 78 |
| | KEREGVSCISSSGGSTYYADSVKGRFTISRDNAKNTVYLQMN | |
| | SLKPEDTAVYYCATDCAGGTSTPYDYWGQGTQVTVSS, | |
| Anti-GPC3 VH-59 | QVQLQESGGGLVQPGGSLRLSCAASGFTLGYYAIGWFRQAPG | 79 |
| | KEREGVSCISSSDGSTYYADSVKGRFTISRDNAKNTVYLQMN | |
| | SLKPEDTAVYYCATACADTTQYEYDYWGQGTQVTVSS, | |
| Anti-GPC3 VH-60 | QVQLQESGGGLVQPGGSLRLSCAASGFTLGYYAIGWFRQAPG | 80 |
| | KEREGVSCISSSDGSTYYADSVKGRFTISRDNAKNTVYLQMN | |
| | SLKPEDTAVYYCATACANWSSLGPYDYWGQGTQVTVSS, | |
| Anti-GPC3 VH-61 | QVQLQESGGGLVQPGGSLRLSCAASGFTLGYYAIGWFRQAPG | 81 |
| | KEREGVSCISSSDGSTYYADSVKGRFTISRDNAKNTVYLQMN | |
| | SLKPEDTGVYYCATACGGATGPYDYWGQGTQVTVSS, | |
| Anti-GPC3 VH-62 | QVQLQESGGGLVQPGGSLRLSCEGSGFSLDYYAIGWFRQAPG | 82 |
| | KEREGVSCISSGDGNTYYADSVKGRFTISRDNAKNTVYLQMN | |
| | SLKPEDTAVYYCATDCVGFGSNWFDYWGQGTQVTVSS, | |
| Anti-GPC3 VH-63 | QVQLQESGGGLVQPGGSLRLSCTASGFSLDYYAIGWFRQAPG | 83 |
| | KEREGVACISSRTGSTYYADSVKGRFTISRDNAKNTVALQMN | |
| | SLKPEDTAVYYCATACVVVGDQNDYWGQGTQVTVSS, | |
| Anti-GPC3 VH-64 | QVQLQESGGGLVQPGGSLRLSCTASGFSLDYYAIGWFRQAPG | 84 |
| | KEREGVSCISSRTGGTYYADSVKGRFTISRDDAKNTVYLQMN | |
| | SLKPEDTAVYYCATACVVVGDRNDYWGQGTQVTVSS | |
| Anti-GPC3 VH-65 | QVQLQESGGGLVQPGGSLRLSCTASGFSLDYYAIGWFRQAPG | 85 |
| | KEREGVSCISSRTGGTYYADSVKGRFTISRDNAKNTVYLQMN | |
| | SLKPEDTAVYYCATACVDTTQYEYDYWGQGTQVTVSS, | |
| Anti-GPC3 VH-66 | QVQLQESGGGLVQPGGSLRLSCTASGFSLDYYAIGWFRQAPG | 86 |
| | KEREGVSCISSRTGGTYYADSVKGRFTISRDNAKNTVYLQMN | |
| | SLKPEDTAVYYCATACVVVGDQNDYWGQGTQVTVSS, | |
| Anti-GPC3 VH-67 | QVQLQESGGGLVQPGGSLRLSCTASGFSLDYYAIGWFRQAPG | 87 |
| | KEREGVSCISSRTGNTYYADSVKGRFTISRDDAKNMVYLQMN | |
| | SLKPEDTAVYYCATACVVVGDQNDYWGQGTQVTVSS, | |
| Anti-GPC3 VH-68 | QVQLQESGGGLVQPGGSLRLSCTASGFSLDYYAIGWFRQAPG | 88 |
| | KEREGVSCISSRTGSTYYADSVKGRFTISRDDAKNTVYLQMN | |
| | SLKPEDTAVYYCATACVVVGDQNDYWGQGTQVTVSS, | |
| Anti-GPC3 VH-69 | QVQLQESGGGLVQPGGSLRLSCTASGFSLGYYAIGWFRQALG | 89 |
| | KEREGVSCISSRTGSTYYADSVKGRFTVSRDDAKNTVYLQMN | |
| | SLKPEDTAVYYCATACVVVGDQNDYWGQGTQVTVSS, | |
| Anti-GPC3 VH-70 | QVQLQESGGGLVQPGGSLRLSCTASGFSLGYYAIGWFRQAPG | 90 |
| | KEREGVSCISSRTGSTYYADSVKGRFAISRDDAKNTVYLQMN | |
| | SLKPEDTAVYYCATACVVVGDQNDYWGQGTQVTVSS, | |
| Anti-GPC3 VH-71 | QVQLQESGGGLVQPGGSLRLSCTASGFSLGYYAIGWFRQAPG | 91 |
| | KEREGVSCISSRTGSTYYADSVKGRFTISRDDAKNTVYLQMN | |
| | SLKPEDTAVYYCATACVVVGDQNDYWGQGTQVTVSS, | |
| Anti-GPC3 VH-72 | QVQLQESGGGLVQPGGSLRLSCTASGFSLGYYAIGWFRQAPG | 92 |
| | KEREGVSCISSRTGSTYYADSVKGRFTVSRDDAKNTVYLQMN | |
| | SLKPEDTAVYYCATACVVVGDQNDYWGQGTQVTVSS, | |
| Anti-GPC3 VH-73 | QVQLQESGGGLVQPGGSLRLSCVASGFPLDYYAIGWFRQAPG | 93 |
| | KEREGVSCISSSDGSTYYADSVKGRFTISRDNAKNTVYLQMN | |
| | SLKPEDTAVYYCATACGGATGPYDYWGQGTQVTVSS, | |
| Anti-GPC3 VH-74 | QVQLQESGGGLVQPGGSLRLSCVASGFSLDYYAIGWFRQAPG | 94 |
| | KEREGVSCISNSDGSTYYADSVKGRFTISRDNAKNTVYLQMN | |
| | SLKPEDTAVYYCATACADTTQYAYDYWGQGTQVTVSS | |
| Anti-GPC3 VH-75 | QVQLQESGGGLVQPGGSLRLSCVASGFTLDYYAIGWFRQAPG | 95 |
| | KEREGVSCISSGSDGNTYYADSVKGRFTISRDNAKNTVYLQM | |
| | NSLKPEDTAVYYCATACSGLTHEYDYWGQGTQVTVSS, | |
| Anti-GPC3 VH-76 | QVQLQESGGGLVQPGGSLRLSCVASGFTLDYYAIGWFRQAPG | 96 |
| | KEREGVSCISSSDDSTYYADSVKGRFTISRDNAKNTVYLQMN | |
| | SLKPEDTAVYYCATACADTTQYEYDYWGQGTQVTVSS, | |
| Anti-GPC3 VH-77 | QVQLQESGGGLVQPGGSLRLSCVASGFTLDYYAIGWFRQAPG | 97 |
| | KEREGVSCISSSSDGNTYYADSVKGRFTISRDNAKNTVYLQM | |
| | NSLKPEDTAVYYCATTCSGLTHEYDYWGQGTQVTVSS, | |
| Anti-GPC3 VH-78 | QVQLQESGGGLVQPGGSLRLSCVASGFTLGYYAIGWFRQAPG | 98 |
| | KEREGVSCISSSDGSTYYADSVKGRFTISRDNAKNTVYLQMN | |
| | SLKPEDTAVYYCATACADTTQYDYDYWGQGTQVTVSS, | |
| Anti-GPC3 VH-79 | QVQLQESGGGLVQPGGSLRLSCVGSGFTLDYYAIGWFRQAPG | 99 |
| | KEREGVSCISSNDGSTYYADSVKGRFTISRDNAKNTVYLQMN | |
| | SLKPEDTAVYYCATACGGATGPYDYWGQGTQVTVSS, | |
| Anti-GPC3 VH-80 | QVQLQESGGGLVQSGGSLRLSCAASGFPLAYYAIGWFRQAPG | 100 |
| | KEREGVSCISASDGSTYYADSVKGRFTISRDNAKNTVYLQMN | |
| | SLKPEDTAVYYCATACAETTLYEYDYWGQGTQVTVSS, | |
| Anti-GPC3 VH-81 | QVQLQESGGGLVQTGGSLRLSCAASGFTLDYYAIGWFRQAPG | 101 |
| | KEREGVSCISSSDGSTYYADSVKGRFTISRDNAKNTVYLQMN | |
| | SLKPEDTAVYYCATACGGATGPYDYWGQGTQVTVSS, | |
| Anti-GPC3 VH-82 | QVQLQESGGGMVQAGESLRLSCAASGFPLAYYAIGWFRQAP | 102 |
| | GKEREGVSCISSSDGNTYYADSVKGRFTISRDNAKNTVYLQM | |
| | NSLKPEDTAVYYCATACADATQHEYDYWGQGTQVTVSS, | |
| Anti-GPC3 VH-83 | QVQLQESGGGSVQPGESLRLSCAASGFPLDYYAIGWFRQAPG | 103 |
| | KEREGVSCISASDGSTYYADSVKGRFTISRDNAKNTVYLQMN | |
| | SLKPEDTAVYYCATACADTTLYEYDYWGQGTQVTVSS | |
| Anti-GPC3 VH-84 | QVQLQESGGGSVQPGGSLRLSCAASGFTLDYYAIGWFRQAPG | 104 |
| | KEREGVSCISSGDGSTYYADSVKGRFTISRDNAKNTVYLQMN | |
| | SLKPEDTAVYYCATACADTTHYEYDYWGQGTQVTVSS | |
| Anti-GPC3 VH-85 | QVQLQESGGGSVQSGGSLRLSCTASGFSLGYYAIGWFRQAPG | 105 |
| | KEREGVSCISSRTGSTYYADSVKGRFTVSRDDAKNTVYLQMN | |
| | SLKPEDTAVYYCATACVVVGDQNDYWGQGTQVTVSS, | |
| Anti-GPC3 VH-86 | QVQLQESGGGSVRPGGSLRLSCAASGFPLAYYAIGWFRQAPG | 106 |
| | KEREGVSCISSSDGNTYYADAVKGRFTISRDNAKNAVYLQMN | |
| | SLKPEDTAVYYCATACADTTQHEYDYWGQGTQVTVSS | |
| Anti-GPC3 VH-87 | QVQLQESGGGVAQPGGSLRLSCAASGFPLDYYAIGWFRQAPG | 107 |
| | KEREGVSCISASDGSTYYADSVKGRFTISRDNAKNTVYLQMN | |
| | SLKPEDTAVYYCATACADTTLYEYDYWGQGTQVTVSS, | |
| Anti-GPC3 VH-88 | QVQLQESGGGVVQAGGSLKLSCAASGSDFRADAMGWYRQA | 108 |
| | PGKEREPVAIDSITSIYYVDSVEGRFTISRDNTKNTVYLQMTSL | |
| | KPEDTAVYYCYARYSGRTYWGRGTQVTVSS, | |
| Anti-GPC3 VH-89 | QVQLQESGGGVVQPGGSLRLSCAASGFSLDYYAIGWFRQAPG | 109 |
| | KEREGVSCISSGDGSTYYADSVKGRFTISRDNAKNTVYLQMN | |
| | SLKPEDTAVYYCATHCGGTSWGTSYDYWGQGTQVTVSS | |
| Anti-GPC3 VH-90 | QVQLQESGGGVVQPGGSLRLSCAASGLTFRSVGMGWFRRAP | 110 |
| | GKEREFVATASPSGVITYYADSVKGRFTISRDNAKNTVYLEM | |
| | NSLKPEDTAVYYCAVRIYSGSFDNTLAYDYWGQGTQVTVSS | |
| Anti-GPC3 VH-91 | QVQLQESGGGVVQPGGSLRLSCTASGFSLGYYAIGWFRQAPG | 111 |
| | KEREGVSCISSRTGSTYYADSVKGRFTVSRDDAKNTVYLQMN | |
| | SLKPEDTAVYYCATACVVVGDQNDYWGQGTQVTVSS | |
| Anti-GPC3 VH-92 | QVQLQESGGGVVQSGGSLRLSCTASGFSLDYYAIGWFRQAPG | 112 |
| | KEREGVSCISSRTGSTYYADSVKGRFTISRDDAKNTVYLQMN | |
| | SLKPEDTAVYYCATACVVVGDQNDYWGQGTQVTVSS | |

In some embodiments, the VH domain of the anti-GPC3 antibody outlined above or the binding fragment thereof comprises with 70-100% sequence identity to any one of the sequences selected from the group consisting of SEQ ID NOs: 22-112. In some embodiments, the VH domain of the anti-GPC3 antibody or binding fragment thereof comprises with at least 7500 sequence identity to any one of the sequences selected from the group consisting of SEQ ID NOs: 22-112. In some embodiments, the VH domain of the anti-GPC3 antibody or binding fragment thereof comprises with at least 8000 sequence identity to any one of the sequences selected from the group consisting of SEQ ID NOs: 22-112. In some embodiments, the VH domain of the anti-GPC3 antibody or binding fragment thereof comprises with at least 85% sequence identity to any one of the sequences selected from the group consisting of SEQ ID NOs: 22-112 In some embodiments, the VH domain of the anti-GPC3 antibody or binding fragment thereof comprises with at least 90% sequence identity to any one of the sequences selected from the group consisting of SEQ ID NOs: 22-112. In some embodiments, the VH domain of the anti-GPC3 antibody or binding fragment thereof comprises with at least 91%, 92%, 93%, 94%, 95%, 96%, 97%, 98%, 99% or 100% sequence identity to any one of the sequences selected from the group consisting of SEQ ID NOs: 22-112.

In one aspect, provided herein is a composition comprising a recombinant nucleic acid, the recombinant nucleic acid comprises a sequence encoding a CAR, wherein the CAR comprises an extracellular anti-GPC3 antigen binding domain having any one of the sequences of SEQ ID NOs: 22-112, or a sequence that is at least 70% identical to any one of the sequences of SEQ ID NOs: 22-112.

In one aspect, provided herein is an anti-GPC3 binder comprising a variable heavy chain that comprises a sequence with at least 70% sequence identity to any one of SEQ ID NO: 33, SEQ ID NO: 47, SEQ ID NO: 55, SEQ ID NO: 58, SEQ ID NO: 80, and SEQ ID NO: 108.

In one embodiment, provided herein is an anti-GPC3 binder having a CDR3 sequence of ATACADTTLYEYDY (SEQ ID NO: 114 or SEQ ID NO: 317). In some embodiments, the anti-GPC3 binder comprises a CDR1 having a sequence GFPLAYYA (SEQ ID NO: 151), and a CDR2 having a sequence of ISASDGST (SEQ ID NO: 168 or SEQ ID NO: 318). In one embodiment, provided herein is a VHH comprising a variable heavy chain, comprising a CDR3, wherein the CDR3 has a sequence of SEQ ID NO: 114 or SEQ ID NO: 343. In some embodiments, the scFv comprises a CDR1 sequence of SEQ ID NO: 151 and a CDR2 of SEQ ID NO: 168. In one embodiment, provided herein is an scFv comprising a variable heavy chain, comprising a CDR3, wherein the CDR3 has a sequence of SEQ ID NO: 114 or SEQ ID NO: 343. In some embodiments, the scFv comprises a CDR1 sequence of SEQ ID NO: 151 and a CDR2 of SEQ ID NO: 168. In one embodiment, the scFv comprises a variable heavy chain that has at least 70% sequence identity to SEQ ID NO: 33. In some embodiments, the scFv comprises a variable heavy chain that has at least 80% sequence identity to SEQ ID NO: 33. In some embodiments, the scFv comprises a variable heavy chain that has at least 85% sequence identity to SEQ ID NO: 33. In some embodiments, the scFv comprises a variable heavy chain that has at least 90% sequence identity to SEQ ID NO: 33. In some embodiments, the scFv comprises a variable heavy chain that has at least 91%, 92%, 93%, 94%, 95%, 96%, 97%, 98%, 99% sequence identity to SEQ ID NO: 33.

In some embodiments, provided herein is a bispecific or a trispecific engager having an anti-GPC3 VH domain, comprising a CDR3 sequence of SEQ ID NO: 114 or SEQ ID NO: 343. In some embodiments, the bispecific or the trispecific engager comprises a CDR1 sequence of SEQ ID NO: 151 and a CDR2 of SEQ ID NO: 168. In one embodiment, the bispecific or a trispecific engager comprises a variable heavy chain that has at least 70% sequence identity to SEQ ID NO: 33. In some embodiments, the bispecific or a trispecific engager comprises a variable heavy chain that has at least 85%, 90%, 91%, 92%, 93%, 94%, 95%, 96%, 97%, 98%, 99% sequence identity to SEQ ID NO: 33, or 100% sequence identity to SEQ ID NO: 33.

In some embodiments, the anti-GPC3 binder is a CAR comprising an extracellular antigen binding domain comprising an antibody or a fragment thereof, having a VH domain, wherein the VH domain comprises a CDR3 having a sequence of SEQ ID NO: 114 or SEQ ID NO: 343. In some embodiments, the anti-GPC3 CAR VH domain comprises a CDR1 sequence of SEQ ID NO: 151 and a CDR2 of SEQ ID NO: 168. In one embodiment, the anti-GPC3 CAR VH domain comprises a variable heavy chain that has at least 70% sequence identity to SEQ ID NO: 33. In some embodiments, the anti-GPC3 CAR VH domain comprises a variable heavy chain that has at least 85%, 90%, 91%, 92%, 93%, 94%, 95%, 96%, 97%, 98%, 99% sequence identity to SEQ ID NO: 33, or 100% sequence identity to SEQ ID NO: 33.

In some embodiments, the anti-GPC3 binder is a VHH, single domain antibody, an scFV or a CAR, having a VH domain comprising a CDR3 having a sequence of ATACANWSSLGPYDY (SEQ ID NO: 136). In some embodiments, the anti-GPC3 binder comprises a CDR1 having the sequence of GFTLGYYA (SEQ ID NO: 157 or SEQ ID NO: 320) and a CDR2 sequence ISSSDGST (SEQ ID NO: 169 or SEQ ID NO: 321). In some embodiments, the anti-GPC3 binder is a VHH, single domain antibody, an scFV or a CAR, having a VH domain comprising a sequence that has at least 70% sequence identity to SEQ ID NO: 80. In some embodiments, the anti-GPC3 CAR VH domain comprises a variable heavy chain that has at least 85%, 90%, 91%, 92%, 93%, 94%, 95%, 96%, 97%, 98%, 99% sequence identity to SEQ ID NO: 80, or 100% sequence identity to SEQ ID NO: 80.

In some embodiments, the anti-GPC3 binder is a VHH, single domain antibody, an scFV or a CAR, having a VH domain comprising a CDR3 having a sequence of ATTCASPEKYEYDY (SEQ ID NO: 139). In some embodiments, the anti-GPC3 binder comprises a CDR1 having the sequence of GFPLNYYA (SEQ ID NO: 158) and a CDR2 sequence ISASDGNT (SEQ ID NO: 190 or SEQ ID NO: 323). In some embodiments, the anti-GPC3 binder is a VHH, single domain antibody, an scFV or a CAR, having a VH domain comprising a sequence that has at least 70% sequence identity to SEQ ID NO: 55. In some embodiments, the anti-GPC3 CAR VH domain comprises a variable heavy chain that has at least 85%, 90%, 91%, 92%, 93%, 94%, 95%, 96%, 97%, 98%, 99% sequence identity to SEQ ID NO: 55, or 100% sequence identity to SEQ ID NO: 55.

In some embodiments, the anti-GPC3 binder is a VHH, single domain antibody, an scFV or a CAR, having a VH domain comprising a CDR3 having a sequence of YARYSGRTY (SEQ ID NO: 142). In some embodiments, the anti-GPC3 binder comprises a CDR1 having the sequence of GSDFRADA (SEQ ID NO: 163) and a CDR2 sequence DSITSI (SEQ ID NO: 325). In some embodiments, the anti-GPC3 binder is a VHH, single domain antibody, an scFV or a CAR, having a VH domain comprising a sequence that has at least 70% sequence identity to SEQ ID NO: 108. In some embodiments, the anti-GPC3 CAR VH domain comprises a variable heavy chain that has at least 85%, 90%, 91%, 92%, 93%, 94%, 95%, 96%, 97%, 98%, 99% sequence identity to SEQ ID NO: 108, or 100% sequence identity to SEQ ID NO: 108.

In some embodiments, the anti-GPC3 binder is a VHH, single domain antibody, an scFV or a CAR, having a VH domain comprising a CDR3 having a sequence of ATDCAGGVGHEYDY (SEQ ID NO: 148). In some embodiments, the anti-GPC3 binder comprises a CDR1 having the sequence of GFSLAYYA (SEQ ID NO: 165) and a CDR2 sequence IAASVGST (SEQ ID NO: 327). In some embodiments, the anti-GPC3 binder is a VHH, single domain antibody, an scFV or a CAR, having a VH domain comprising a sequence that has at least 70% sequence identity to SEQ ID NO: 58. In some embodiments, the anti-GPC3 CAR VH domain comprises a variable heavy chain that has at least 85%, 90%, 91%, 92%, 93%, 94%, 95%, 96%, 97%, 98%, 99% sequence identity to SEQ ID NO: 58, or 100% sequence identity to SEQ ID NO: 58.

In some embodiments, the anti-GPC3 binder is a VHH, single domain antibody, an scFV or a CAR, having a VH domain comprising a CDR3 having a sequence of ATDCSLHGSDYPYDY (SEQ ID NO: 149). In some embodiments, the anti-GPC3 binder comprises a CDR1 having the sequence of GFPLDYYA (SEQ ID NO: 153) and a CDR2 sequence ISSSDGSDGNT (SEQ ID NO: 193 or SEQ ID NO: 329). In some embodiments, the anti-GPC3 binder is a VHH, single domain antibody, an scFV or a CAR, having a VH domain comprising a sequence that has at least 70% sequence identity to SEQ ID NO: 47. In some embodiments, the anti-GPC3 CAR VH domain comprises a variable heavy chain that has at least 85%, 90%, 91%, 92%, 93%, 94%, 95%, 96%, 97%, 98%, 99% sequence identity to SEQ ID NO: 47, or 100% sequence identity to SEQ ID NO: 47.

In some embodiments, the CAR comprises a leader sequence. In some embodiments, the leader sequence (signal sequence) comprises a sequence of MWLQSLLLLGTVACSIS (SEQ ID NO: 306). In some embodiments, extracellular domain comprises a hinge. In some embodiments the hinge is operatively connected to the transmembrane domain. In some embodiments, the hinge is a CD8alpha hinge. In some embodiments, the hinge has a sequence of ALSNSIMYFSHFVPVFLPAKPTTTPAPRPPTPAPTIASQPLSLRPEACRPAAGGAVHTRGLD (SEQ ID NO: 307). In some embodiments the extracellular domain comprises a linker. In some embodiments, the linker is operatively linked to the VH and the hinge. In some embodiments, the linker is operatively linked to the VH and the transmembrane domain. In some embodiments, the linker has a sequence of SGGGSG (SEQ ID NO: 308). In some embodiments, the transmembrane domain is a CD8 transmembrane domain. In some embodiments, the transmembrane domain has a sequence of IYIWAPLAGTCGVLLLSLVITLYC (SEQ ID NO: 309). In some embodiments, the intracellular domain comprises an intracellular signaling domain from FcεR. In some embodiments, the intracellular domain comprises a sequence of

(SEQ ID NO: 310)
RRLKIQVRKAAITSYEKSDGVYTGLSTRNQETYETLKHEKPPQ.

In some embodiments, the intracellular domain comprises at least two intracellular signaling domains. In some embodiments, the least two intracellular signaling domains are separated by a linker. In some embodiments the linker has a sequence of GSGS (SEQ ID NO: 311). In some embodiments, for a cell specific CAR, e.g., a myeloid cell specific CAR is designed, wherein the transmembrane domain is capable of dimerizing or multimerizing with an endogenous protein in a myeloid cell and this dimerizing or multimerizing of the CAR domain with an endogenous protein allows expression and function of the CAR on myeloid cell surface. In some embodiments, the myeloid cell specific CAR comprises a TM domain of a CD16 protein (e.g., a human CD16), having a sequence GLAVSTISSFFPPGYQVSFCLVMVLLFAVDTGLYFSV (SEQ ID NO: 312), which comprises a short extracellular region and the transmembrane region. In some embodiments, the myeloid cell specific CAR comprises a human CD89 domain, having a sequence IHQDYTTQNLIRMAVAGLVLVALLAILV (SEQ ID NO: 313). Incorporating a TM domain of SEQ ID NO: 312 and 313 in the CAR allows oligomerization with a myeloid cell endogenous protein FcR-γ which allows the CAR to be expressed selectively in the cell that expresses the endogenous protein FcR-γ.

In some embodiments, the transmembrane domain and the antigen binding domain are operatively linked through a linker. In some embodiments, the transmembrane domain and the antigen binding domain are operatively linked through a linker such as a hinge region of CD8a, IgG1 or IgG4.

In some embodiments, the extracellular domain comprises a multimerization scaffold.

In some embodiments, the transmembrane domain comprises a CD8 transmembrane domain. In some embodiments, the transmembrane domain comprises a CD28 transmembrane domain. In some embodiments, the transmembrane domain comprises a CD68 transmembrane domain. In some embodiments, the transmembrane domain comprises a CD2 transmembrane domain. In some embodiments, the transmembrane domain comprises an FcR transmembrane domain. In some embodiments, the transmembrane domain comprises an FcRγ transmembrane domain. In some embodiments, the transmembrane domain comprises an FcRα transmembrane domain. In some embodiments, the transmembrane domain comprises an FcRβ transmembrane domain. In some embodiments, the transmembrane domain comprises an FcRε transmembrane domain.

In some embodiments, the intracellular domain comprises an intracellular signaling domain that that is the PI3K recruitment domain. In some embodiments, the PI3K recruitment domain has a sequence of YEDMRGILYAAPQLRSIRGQPGPNHEEDADSYENM (SEQ ID NO: 314).

In some embodiments, the VH is a single domain antibody.

In some embodiments, the VH is a VHH.

TABLE 4
Domains of an exemplary anti-GPC3-CAR.

| Domain | Sequence | SEQ ID NO |
|---|---|---|
| Signal peptide leader | MWLQSLLLLGTVACSIS | SEQ ID NO: 306 |
| sequence | | |
| Extracellular domain | QVQLQESGGGLVHSGGSLRLSCAASGFPLAY | SEQ ID NO: 22 |
| (Anti-GPC3 VHH) | YAIGWFRQAPGKEREGVSCISSSDGNTYYAD | |
| | AVKGRFTISRDNAKNTVYLQMNSLKPEDTAV | |
| | YYCATACADTTQHEYDYWGQGTQVTVSS | |
| Linker | SGGGGSG | SEQ ID NO: 345 |
| CD8 alpha hinge | ALSNSIMYFSHFVPVFLPAKPTTTPAPRPPTPA | SEQ ID NO: 307 |
| | PTIASQPLSLRPEACRPAAGGAVHTRGLD | |
| CD8 alpha TMD | IYIWAPLAGTCGVLLLSLVITLYC | SEQ ID NO: 309 |
| FCϵR ICD | RRLKIQVRKAAITSYEKSDGVYTGLSTRNQET | SEQ ID NO: 310 |
| | YETLKHEKPPQ | |
| Short linker | GSGS | SEQ ID NO: 311 |
| PI3-K ICD | YEDMRGILYAAPQLRSIRGQPGPNHEEDADSY | SEQ ID NO: 314 |
| | ENM | |

In some embodiments, the recombinant nucleic acid is an mRNA. In some embodiments, the recombinant nucleic acid is a vector. Provided herein is a composition comprising a cell comprising the recombinant nucleic acid of the composition described above. In some embodiments, the cell is an immune cell, wherein the cell is a myeloid cell, a lymphoid cell, a precursor cell, a stem cell or an induced pluripotent cell. In some embodiments, the cell is CD14+/CD16−. In some embodiment, the cell is a population of cells, comprising at least 1×10\hat{ }5 cells.

Provided herein is a pharmaceutical composition comprising the composition described above.

Provided herein is a method of treating a disease or condition in a subject in need thereof comprising administering to the subject the pharmaceutical composition described above, wherein the disease or condition is a cancer.

### Anti-IL13RA2 Binding Domain Compositions

In one aspect, provided herein is a composition comprising a construct comprising an antigen binding domain comprising an anti-IL13Rat2 antibody or binding fragment thereof, or a recombinant nucleic acid encoding the antigen binding domain, wherein the antigen binding domain comprises a heavy chain variable domain (VH) comprising a heavy chain complementarity determining region 3 (HC CDR3) sequence selected from the group consisting of ALRRGARIL (SEQ ID NO: 253), AARDRLLNTVTNIDYDY (SEQ ID NO: 254), GADAVFYSGGYYSDS (SEQ ID NO: 255), RVAGRQDDY (SEQ ID NO: 256), AARRASTIA (SEQ ID NO: 257), NVVGVLHTGHYENSF(SEQ ID NO: 258), NARRGARVL (SEQ ID NO: 259), NARPGLRSY (SEQ ID NO: 260), NIVGVLTTGHYEDSF (SEQ ID NO: 261), NIVGVLTTGHYENSF (SEQ ID NO: 262), ALRRGGRIL (SEQ ID NO: 263), NARPGLQSY (SEQ ID NO: 264), NARPGLQSF (SEQ ID NO: 265).

In some embodiments, the VH of the anti-IL13Rα2 antibody or binding fragment thereof further comprises a heavy chain complementarity determining region 1 (HC CDR1) that is the HC CDR1 of any one of the VH sequences selected from the group consisting of GINISSDV (SEQ ID NO: 266), GSSYSTSD (SEQ ID NO: 267), GSIFSINR (SEQ ID NO: 268), ASIFSISA (SEQ ID NO: 269), QTITRLST (SEQ ID NO: 270), GSIFNTNG (SEQ ID NO: 271), GSISSINR (SEQ ID NO: 272), GSIRDIGY (SEQ ID NO: 273), GSIRGIGY (SEQ ID NO: 274), GIFVSANS (SEQ ID NO: 275), GINASGDV (SEQ ID NO: 276), GLTFSNYD (SEQ ID NO: 277), GGIFTVND (SEQ ID NO: 278), GINISRDV (SEQ ID NO: 279), GIRISSDV (SEQ ID NO: 280), GIFVSANT (SEQ ID NO: 281), GVTGEIEA (SEQ ID NO: 282), GINISNDV (SEQ ID NO: 283), GTNISSDV (SEQ ID NO: 284), GDIYRINT (SEQ ID NO: 285), GIALDYYA (SEQ ID NO: 286).

In some embodiments, the VH of the anti-IL13Rα2 antibody or binding fragment thereof further comprises a heavy chain complementarity determining region 2 (HC CDR2) that is the HC CDR2 of any one of the VH sequences selected from the group consisting of HVTTDSHTE (SEQ ID NO: 287), AISPAGATN (SEQ ID NO: 288), HITTDSRTN (SEQ ID NO: 289), SITSSGDTM (SEQ ID NO: 290), MLGSTTD (SEQ ID NO: 291), HITTYSRTD (SEQ ID NO: 292), VIRRGGNTN (SEQ ID NO: 293), HVSTDSHTE (SEQ ID NO: 294), HVTADSHTE (SEQ ID NO: 295), SITSSGDTT (SEQ ID NO: 296), HITTNSRTE (SEQ ID NO: 297), TINSAGDTN (SEQ ID NO: 298), TINSAGNTN (SEQ ID NO: 299), TINSAGATN (SEQ ID NO: 300), TLNSAGNTN(SEQ ID NO: 301), QITYGDTIK (SEQ ID NO: 302), LMDTAYITD (SEQ ID NO: 303), GISSGGNTV (SEQ ID NO: 304), SMTGNGDTM (SEQ ID NO: 305).

In some embodiments, the VH of the anti-IL13Rα2 antibody or binding fragment thereof comprises with 70-100% sequence identity to any one of the sequences selected from the group consisting of SEQ ID NOs. 195-252.

TABLE 5
Anti-IL 13Rα2 VH sequences identified.

| | | SEQ |
|---|---|---|
| Anti-IL13Rα2 VH domains | | ID |
| Anti-IL13Rα2 VH1 | QVQLQESGGGLVQAGGSLRLSCAASGINISSDVMGWYRQGP | 195 |
| | DNERELVAHVTTDSHTEYADSVKGRFTISRDNAKNTVYLQM | |
| | NDLKPEDTAPYYCALRRGARILWGQGTQVTVSS | |
| Anti-IL13Rα2 VH2 | QVQLQESGGGLVQPGESLRLSCAASGINISSDVMGWYRQGP | 196 |
| | DNERELVAHVTTDSHTEYADSVKGRFTISRDNAKNTVYLQM | |
| | NDLKPEDTAPYYCALRRGARILWGQGTQVTVSS | |
| Anti-IL13Rα2 VH3 | QVQLQESGGGSVQPGGSLRLSCAASGINISSDVMGWYRQGP | 197 |
| | GNERELVAHVTTDSHTEYADSVKGRFTISRDNAKNTVYLQM | |
| | NDLKPEDTAPYYCALRRGARILWGQGTQVTVSS | |
| Anti-IL13Rα2 VH4 | QVQLQESGGGSVQPGESLRLSCAASGINISSDVMGWYRQGP | 198 |
| | DNERELVAHVTTDSHTEYADSVKGRFTISRDNAKNTVYLQM | |
| | NDLKPEDTAPYYCALRRGARILWGQGTQVTVSS | |
| Anti-IL13Rα2 VH5 | QVQLQESGGGLVQPGGSLRLSCATSGSSYSTSDMGWYRQAP | 199 |
| | GKQRELVAAISPAGATNYADFAQGRFAASRDNAKNTVYLQ | |
| | MNDLKPEDTAPYYCALRRGARILWGQGTQVTVSS | |
| Anti-IL13Rα2 VH6 | QVQLQESGGGSVRPGGSLRLSCAASGINASGDVMGWYRQAP | 200 |
| | GNERELVAHITTDSRTNYADSVKGRFTISRDNAKNTVYLQM | |
| | NDLKPEDTAPYYCALRRGARILWGQGTQVTVSS | |
| Anti-IL13Rα2 VH7 | QVQLQESGGGSVQHGGSLRLSCTASGLTFSNYDMGWYRQG | 201 |
| | PDNERELVAHVTTDSHTEYADSVKGRFTISRDNAKNTVYLE | |
| | MNDLKPEDTAPYYCALRRGARILWAQGTQVTVSS | |
| Anti-IL13Rα2 VH8 | QVQLQESGGGSVQPGESLRLSCAASGINISSDVMGWYRQGP | 202 |
| | DNERELVAHVTTDSHTEYADSVKGRFTISRDNAKNMVYLQ | |
| | MNDLKPEDTAPYYCALRRGARILWGQGTQVTVSS | |
| Anti-IL13Rα2 VH9 | QVQLQESGGGLVQAGESLRLSCAASGINISSDVMGWYRQGP | 203 |
| | DNERELVAHVTTDSHTEYADSVKGRFTISRDNAKNTVYLQM | |
| | NDLKPEDTAPYYCALRRGARILWGQGTQVTVSS | |
| Anti-IL13Rα2 | QVQLQESGGGLVQPGGSLRLSCAASGIFVSANSMGWYRQGP | 204 |
| VH10 | EDARELVASITSSGDTMYGDSWKGRFAISRDNAKNTLYLQM | |
| | NLLKSDDTAVYYCNARRGARVLWGKGTQVTVSS | |
| Anti-IL13Rα2 | QVQLQESGGGSVQPGGSLRLSCAASGINISSDVMGWYRQGP | 205 |
| VH11 | DNERELVAHVTTDSHTEYADSVKGRFTISRDNAKNTVYLQM | |
| | NDLKPEDTAPYYCALRRGARILWGQGTQVTVSS | |
| Anti-IL13Rα2 | QVQLQESGGGLVQPGGSLRLSCAASGINISSDVMGWYRQGP | 206 |
| VH12 | DNERELVAHVTTDSHTEYADSVKGRFTISRDNAKNTVYLQM | |
| | NDLKPEDTAPYYCALRRGARILWGQGTQVTVSS | |
| Anti-IL13Rα2 | QVQLQESGGGLVHSGGSLRLSCAASGINISSDVMGWYRQGP | 207 |
| VH13 | DNERELVAHVTTDSHTEYADSVKGRFTISRDNAKNTVYLQM | |
| | NDLKPEDTAPYYCALRRGARILWGQGTQVTVSS | |
| Anti-IL13Rα2 | QVQLQESGGGSVQPGESLRLSCAASGINISSDVMGWYRQGP | 208 |
| VH14 | DNERELVAHVTTDSHTEYADSVKGRFTISRDNAKNTVYLEM | |
| | NDLKPEDTAPYYCALRRGARILWGQGTQVTVSS | |
| Anti-IL13Rα2 | QVQLQESGGGVAQPGESLRLSCKVSGIALDYYAIGWLRQAP | 209 |
| VH15 | GKEREGVAMLGSTTDYSASVKGRFTISRGDAKNTVYLDMN | |
| | NLKPEDTATYYCALRRGARILWGQGTQVTVSS | |
| Anti-IL13Rα2 | QVQLQESGGGSVQPGGSLRLSCVASGINISSDVMEWYRQAP | 210 |
| VH16 | GEERELVAHITTYSRTDYSDSVKGRFTISRANAKNTLYLQMN | |
| | GLKPEDTALYYCALRRGARILWGQGTQVTVSS | |
| Anti-IL13Rα2 | QVQLQESGGGSVQSGESLKLSCVGSGDIYRINTMAWYRQTP | 211 |
| VH17 | GKEREMVAVIRRGGNTNYADSVKGRFTISRDDSKNTMSLQM | |
| | NSLRPEDTATYYCALRRGARILWGQGTQVTVSS | |
| Anti-IL13Rα2 | QVQLQESGGGSVQPGESLRLSCAASGINISSDVMGWYRQGP | 212 |
| VH18 | DNERELVAHVTTDSHTEYADSVKGRFTISRDNAKNTVYLQM | |
| | NDLKPEDTAPYYCALRRGARILWGPGTQVTVSS | |
| Anti-IL13Rα2 | QVQLQESGGGSVQPGESLRLSCAASGINISSDVMGWYRQGP | 213 |
| VH19 | DNERELVAHVTTDSHTEYADSVKGRFTISRDNAKNTVYLQM | |
| | NDLKPEDTAPYYCALRRGARILWSQGTQVTVSS | |
| Anti-IL13Rα2 | QVQLQESGGGLVEPGGSLRLSCQISGGIFTVNDMGWYRRSPG | 214 |
| VH20 | TDRELVAHVTTDSHTEYADSVKGRFTISRDNAKNTVYLQMN | |
| | DLKPEDTAPYYCALRRGARILWGQGTQVTVSS | |
| Anti-IL13Rα2 | QVQLQESGGGLVKPGGSLRLSCAASGIFVSANSMGWYRQGP | 215 |
| VH21 | EDARELVASITSSGDTMYGDSWKGRFAISRDNAKNTLYLQM | |
| | NLLKSDDTAVYYCNARRGARVLWGKGTQVTVSS | |
| Anti-IL13Rα2 | QVQLQESGGGSVQPGGSLRLSCAASGINISRDVMGWYRQGP | 216 |
| VH22 | VNERELVAHVTTDSHTEYADSVKGRFTISRDNAKNTVYLEM | |
| | NDLKPEDTAPYYCALRRGARILWGQGTQVTVSS | |
| Anti-IL13Rα2 | QVQLQESGGGLVQPGGSLRLSCAASGINISSDVMGWYRQGP | 217 |
| VH23 | DNERELVAHVTTDSHTEYADSVKGRFTISRDNAKNTVYLQM | |
| | NDLRPEDTAPYYCALRRGARILWGQGTQVTVSS | |
| Anti-IL13Rα2 | QVQLQESGGGLVQPGESLRLSCAASGINISSDVMGWYRQGP | 218 |
| VH24 | DNERELVAHVSTDSHTEYADSVKGRFTISRDNAKNTVYLQM | |
| | NDLKPEDTAPYYCALRRGARILWGQGTQVTVSS | |
| Anti-IL13Rα2 | QVQLQESGGGLVHSGESLRLSCAASGINISSDVMGWYRQGP | 219 |
| VH25 | DNERELVAHVTTDSHTEYADSVKGRFTISRDNAKNTVYLQM | |
| | NDLKPEDTAPYYCALRRGARILWGQGTQVTVSS | |
| Anti-IL13Rα2 | QVQLQESGGGSVQPGGSLRLSCAASGINISRDVMGWYRQGP | 220 |
| VH26 | GNERELVAHVTTDSHTEYADSVKGRFTISRDNAKNTVYLEM | |
| | NDLKPEDTAPYYCALRRGARILWGQGTQVTVSS | |
| Anti-IL13Rα2 | QVQLQESGGGSVQPGESLRLSCAASGINISSDVMGWYRQGP | 221 |
| VH27 | DNEHELVAHVTTDSHTEYADSVKGRFTISRDNAKNTVYLQM | |
| | NDLKPEDTAPYYCALRRGARILWGQGTQVTVSS | |
| Anti-IL13Rα2 | QVQLQESGGGSVQPGESLRLSCAASGINISSDVMGWYRQGP | 222 |
| VH28 | DNERELVAHVTTDSHTEYADSVKGRFTISRDNAKNTVYLQT | |
| | NDLKPEDTAPYYCALRRGARILWGQGTQVTVSS | |
| Anti-IL13Rα2 | QVQLQESGGGSVQPGGSLRLSCAASGIRISSDVMGWYRQGP | 223 |
| VH29 | GNDRELVAHVTADSHTEYADSAKGRFTISRDNAKNTVYLQ | |
| | MNDLKPEDTAPYYCALRRGARILWGQGTQVTVSS | |
| Anti-IL13Rα2 | QVQLQESGGGSVQPGESLRLSCAAPGINISSDVMGWYRQGP | 224 |
| VH30 | DNERELVAHVTTDSHTEYADSVKGRFTISRDNAKNTVYLQM | |
| | NDLKPEDTAPYYCALRRGARILWGQGTQVTVSS | |
| Anti-IL13Rα2 | QVQLQESGGGSVQPGESLRLSCAASGINISSDVMGWYRQGP | 225 |
| VH31 | GNERELVAHVTTDSHTEYADSVKGRFTISRDNAKNTVYLQM | |
| | NDLKPEDTAPYYCALRRGARILWGQGTQVTVSS | |
| Anti-IL13Rα2 | QVQLQESGGGSVQPGESLRLSCAASGINISSDVMGWYRQGP | 226 |
| VH32 | DNERELVAHVTTDSHTEYADSVKGRFTISRDNASNTVYLQM | |
| | NDLKPEDTAPYYCALRRGARILWGQGTQVTVSS | |
| Anti-IL13Rα2 | QVQLQESGGGLVQPGGSLRLSCAASGIFVSANTMGWYRQGP | 227 |
| VH33 | EDARELVASITSSGDTTYADSWKGRFAISRDNAKNTLYLQM | |
| | NLLKSDDTAVYYCNARRGARVLWGKGTQVTVSS | |
| Anti-IL13Rα2 | QVQLQESGGGLVQPGGSLRLSCAASGINISSDVMGWYRQGP | 228 |
| VH34 | DDERELVAHVTTDSHTEYADSVKGRFTISRDNAKNTVYLQM | |
| | NDLKPEDTAPYYCALRRGARILWGQGTQVTVSS | |
| Anti-IL13Rα2 | QVQLQESGGGMVQPGESLRLSCVASGVTGEIEAMGWYRQG | 229 |
| VH35 | PEDARELVASITSSGDTTYGDSWKGRFAISRDNAKNTLYLQM | |
| | NLLKSDDTAVYYCNARRGARVLWGKGTQVTVSS | |
| Anti-IL13Rα2 | QVQLQESGGGSVQPGGSLRLSCAASGINISSDVMEWFRQGPG | 230 |
| VH36 | EQRELVAHITTNSRTEYADFVKGRFTISRANAKNTLYLQMNG | |
| | LKPEDTALYYCALRRGGRILWGQGTQVTVSS | |
| Anti-IL13Rα2 | QVQLQESGGGLVQPGESLRLSCAASGINISSDVMGWYRQGP | 231 |
| VH37 | DNERELVAHVTTDSHTEYADSVKGRLTISRDNAKNTVYLQM | |
| | NDLKPEDTAPYYCALRRGARILWGQGTQVTVSS | |
| Anti-IL13Rα2 | QVQLQESGGGSVQPGESLRLSCAASGTNISSDVMGWYRQGP | 232 |
| VH38 | DNERELVAHVTTDSHTEYADSVKGRFTISRDNAKNTVYLQM | |
| | NDLKPEDTAPYYCALRRGARILWGQGTQVTVSS | |
| Anti-IL13Rα2 | QVQLQESGGGLVTPGDSLRLSCAASGINISSDVMGWYRQGP | 233 |
| VH39 | DNERELVAHVTTDSHTEYADSVKGRFTISRDNAKNTVYLQM | |
| | NDLKPEDTAPYYCALRRGARILWGQGTQVTVSS | |
| Anti-IL13Rα2 | QVQLQESGGGLVQPGGSLSVSCAASGINISSDVMGWYRQGP | 234 |
| VH40 | DNERELVAHVTTDSHTEYADSVKGRFTISRDNAKNTVYLQM | |
| | NDLKPEDTAPYYCALRRGARILWGQGTQVTVSS | |
| Anti-IL13Rα2 | QVQLQESGGGSVQPGESLRLSCAASGINISNDVMGWYRQGP | 235 |
| VH41 | DNERELVAHVTTDSHTEYADSVKGRFTISRDNAKNTVYLQM | |
| | NDLKPEDTAPYYCALRRGARILWGQGTQVTVSS | |
| Anti-IL13Rα2 | QVQLQESGGGSVQPGESLRLSCAASGINISSDVMGWYRQGP | 236 |
| VH42 | DNERELVAHVTTDSHTEYADSVKGRFTISRDNAKNTVYLQM | |
| | NDLKPEDAAPYYCALRRGARILWGQGTQVTVSS | |
| Anti-IL13Rα2 | QVQLQESGGGLVQPGGSLRLSCTASGSIRGIGYMAWGRQGP | 237 |
| VH43 | GKPRELIATINSAGDTNYAESVKGRFTVSRDNAENTLYLQMN | |
| | NLKSEDTAVYYCNARPGLQSYWGQGTQVTVSS | |
| Anti-IL13Rα2 | QVQLQESGGGLVQPGGSLRLSCTASGSIRDIGYMAWGRQAP | 238 |
| VH44 | GKQRELIATINSAGNTNYAESVKGRFTVSKDNAENTLYLQM | |
| | NNLKSEDTAVYYCNARPGLRSYWGQGTQVTVSS | |
| Anti-IL13Rα2 | QVQLQESGGGSAQSGGSLRLSCTASGSIRGIGYMAWGRQAP | 239 |
| VH45 | GKQRELIATINSAGDTNYAESVKGRFTVSRDNAENTLYLQM | |
| | NNLKSEDTAVYYCNARPGLRSYWGQGTQVTVSS | |
| Anti-IL13Rα2 | QVQLQESGGGLVQPGGSLRLSCTASGSIRGIGYMAWGRQAP | 240 |
| VH46 | DKPRELIATINSAGATNYAESVKGRFTVSRDNAANTLYLQM | |
| | NNLKSEDTAVYYCNARPGLQSFWGQGTQVTVSS | |
| Anti-IL13Rα2 | QVQLQESGGGLVQPGGSLRLSCTASGSIRGIGYMAWGRQAP | 241 |
| VH47 | GKQRELIATINSAGDTNYAESVKGRFTVSRDNAENTLYLQM | |
| | NNLKSEDTAVYYCNARPGLRSYWGQGTQVTVSS | |
| Anti-IL13Rα2 | QVQLQESGGGSVQAGDSLRLSCTASGSIRGIGYMAWGRQAP | 242 |
| VH48 | GKQRELIATINSAGDTNYAESVKGRFTVSRDNAENTLYLQM | |
| | NNLKSEDTAVYYCNARPGLRSYWGQGTQVTVSS | |
| Anti-IL13Rα2 | QVQLQESGGGLVHSGGSLTLSCTASGSIRDIGYMAWGRQAP | 243 |
| VH49 | GKQRELIATINSAGNTNYAESVKGRFTVSKDNAENTLYLQM | |
| | NNLKSEDTAVYYCNARPGLRSYWGQGTQVTVSS | |
| Anti-IL13Rα2 | QVQLQESGGGLVQPGGSLRLSCTASGSIRDIGYMAWGRQAP | 244 |
| VH50 | GEPRELIATLNSAGNTNYAESVKGRFTVSKDNAENTLYLQM | |
| | NNLKSEDTAVYYCNARPGLRSYWGQGTQVTVSS | |
| Anti-IL13Rα2 | QVQLQESGGGLVQPGGSLRLSCEVSGSIFSINRMAWYRQVPE | 245 |
| VH51 | KQREMVAQITYGDTIKYYGSAKGRFTISRDSAKNAVYLQMN | |
| | SLKPEDTAVYRCNIVGVLTTGHYEDSFWGQGTQVTVPS | |
| Anti-IL13Rα2 | QVQLQESGGGLVQPGGSLRLSCEVSGSIFSINRMAWYRQVPE | 246 |
| VH52 | KEREMVAQITYGDTIKYYGSAKGRFTISRDSAKNAVYLQMN | |
| | SLKPEDTAVYRCNIVGVLTTGHYEDSFWGQGTQVTVPS | |
| Anti-IL13Rα2 | QVQLQESGGGLVQPGGSLRLSCEVSGSISSINRMAWYRQVPG | 247 |
| VH53 | KEREMVAQITYGDTIKYYGSAKGRFTISKDSAKNAVYLQMN | |
| | SLKPEDTAVYRCNIVGVLTTGHYENSFWGQGTQVTVSS | |
| Anti-IL13Rα2 | QVQLQESGGGSVQPGESLRLSCAASGINISSDVMGWYRQGP | 248 |
| VH54 | DNERELVAHVTTDSHTEYADSVKGRFTISRPSARNTVWLQM | |
| | NSLKPEDTAVYYCAARDRLLNTVTNIDYDYWGQGTQVTVSS | |
| Anti-IL13Rα2 | QVQLQESGGGLVQPGGSLRLSCEVSGSIFSINRMAWYRQVPG | 249 |
| VH55 | KQREMVAQITYGDTIKYYGSAKGRFTISRDSAKNAVYLQMN | |
| | SLKPEDTAVYRCNVVGVLHTGHYENSFWGQGTQVTVSS | |
| Anti-IL13Rα2 | QVQLQESGGGLVQPGGSLRLSCATSASIFSISAMGWYRQTSG | 250 |
| VH56 | SERELVALMDTAYITDYADTVKGRFTISRDNAKNTVNLQMN | |
| | SLKPEDTAVYYCAARRASTIAWGQGTQVTVSS | |
| Anti-IL13Rα2 | QVQLQESGGGLVQPGGSLRLSCAASGSIFNTNGMGWYRQAP | 251 |
| VH57 | GKQRERVAGISSGGNTVYAASVEGRFTISRDNAKNTGYLQM | |
| | NNLKPEDTAVYYCRVAGRQDDYWGQGTQVTVSS | |
| Anti-IL13Rα2 | QVQLQESGGGSVQAGGSLRLSCAASQTITRLSTIGWYRQSAG | 252 |
| VH58 | KGRDLVASMTGNGDTMYADSVRGRFTISRDNAKNTVYLQM | |
| | DSLKPEDTAVYYCGADAVFYSGGYYSDSWGQGTQVTVSS | |

In some embodiments, the VH is a single domain antibody domain.

In some embodiments, the VH is a VHH.

In some embodiments, the construct comprises an extracellular domain comprising the antigen binding domain that binds to IL13Ra2, wherein extracellular domain is operably linked to a transmembrane domain. In some embodiments, the variable domain is a component of a chimeric antigen receptor (CAR). In some embodiments contemplated herein is an anti-IL13Rα2 CAR, comprising an antigen binding domain capable of binding to IL13Rα2, wherein the antigen binding domain being a single-chain variable fragment (scFv), a nanobody, a VH domain, a single domain antibody (sdAb), a VNAR domain, and a VHH domain, a bispecific antibody, a diabody, or a functional fragment of any thereof. In some embodiments, the antigen-binding domain comprises an antibody or an antigen binding thereof, e.g., an Fab, a single-chain variable fragment (scFv), a nanobody, a VH domain, a VL domain, a single domain antibody (sdAb), a VNAR domain, and a VHH domain, a bispecific antibody, a diabody, or a functional fragment thereof that specifically binds to one or more antigens, at least one of which is IL13Rα2, and the antigen binding domain comprising an IL13Rα2 variable heavy chain domain (VH) selected from the domains listed in Table 5.

In some embodiments, provided herein is a composition comprising a recombinant nucleic acid, the recombinant nucleic acid comprises a sequence encoding a CAR, wherein the CAR comprises an extracellular anti-IL13Rα2 antigen binding domain having any one of the sequences of SEQ ID NOs: 195-252, or a sequence that is at least 70% identical to any one of the sequences of SEQ ID NOs: 195-252.

In one embodiment, the composition comprises a recombinant nucleic acid comprising a sequence encoding a CAR comprising an extracellular anti-IL13Rα2 antigen binding domain having any one of the sequences of SEQ ID NOs: 230, 237, 246, 247, 251 or 340, or a sequence that is at least 70% identical to any one of the sequences.

In some embodiments, provided herein is a recombinant nucleic acid comprising a sequence encoding a CAR, the CAR comprising an extracellular anti-IL13Rα2 binding domain, comprising a V_{H} domain, the V_{H} domain comprising a HC CDR3 of any one of ALRRGGRIL (SEQ ID NO: 263), NARPGLQSY (SEQ ID NO: 264), NIVGVLTTGHYEDSF (SEQ ID NO: 261), NIVGVLTTGHYENSF (SEQ ID NO: 262) or RVAGRQDDY (SEQ ID NO: 256).

In some embodiments, the anti-IL13Rα2 binder is a VHH, single domain antibody, an scFV or a CAR with an extracellular an IL13Rα2 V_{H} domain comprising a CDR3 having a sequence of ALRRGGRIL SEQ ID NO: 263). In some embodiments, the anti-IL13RA2 binder comprises a CDR1 having the sequence of GINISSDV (SEQ ID NO: 331) and a CDR2 domain ITTNSRTE (SEQ ID NO: 332). In some embodiments, the anti-IL13RA2 binder is a VHH, single domain antibody, an scFV or a CAR, having a V_{H} domain comprising a sequence that has at least 70% sequence identity to SEQ ID NO: 230. In some embodiments, the anti-IL13RA2 CAR VH domain comprises a variable heavy chain that has at least 85%, 90%, 91%, 92%, 93%, 94%, 95%, 96%, 97%, 98%, 99% sequence identity to SEQ ID NO: 230, or 100% sequence identity to SEQ ID NO: 230.

In some embodiments, the anti-IL13RA2 binder is a VHH, single domain antibody, an scFV or a CAR with an extracellular anti-IL2Rα2 binding domain having a VH domain comprising a CDR3 having a sequence of NARPGLQSY (SEQ ID NO: 264). In some embodiments, the anti-IL13RA2 binder comprises a CDR1 having the sequence of GSIRGIGY (SEQ ID NO: 274 or SEQ ID NO: 334) and a CDR2 domain INSAGDT (SEQ ID NO: 335) or TINSAGDTN (SEQ ID NO: 298). In some embodiments, the anti-IL 13RA2 binder is a VHH, single domain antibody, an scFV or a CAR, having a V_{H} domain comprising a sequence that has at least 70% sequence identity to SEQ ID NO: 237. In some embodiments, the anti-IL13RA2 CAR VH domain comprises a variable heavy chain that has at least 85%, 90%, 91%, 92%, 93%, 94%, 95%, 96%, 97%, 98%, 99% sequence identity to SEQ ID NO: 237, or 100% sequence identity to SEQ ID NO: 237.

In some embodiments, the anti-IL13RA2 binder is a VHH, single domain antibody, an scFV or a CAR, having a V_{H} domain comprising a CDR3 having a sequence of NIVGVLTTGHYEDSF (SEQ ID NO: 261). In some embodiments, the anti-IL13RA2 binder comprises a CDR1 having a sequence of GSIFSINR (SEQ ID NO: 268) and a CDR2 sequence ITYGDTI (SEQ ID NO: 337) or

(SEQ ID NO: 344)
QITYGDTI.

In some embodiments, an anti-IL13RA2 binder comprises a CDR3 sequence of NIVGVLTTGHYENSF (SEQ ID NO: 262). In some embodiments, the anti-IL13RA2 binder comprises a CDR1 having the sequence of GSISSINR (SEQ ID NO: 272) and a CDR2 sequence ITYGDTI (SEQ ID NO: 337) or QITYGDTI (SEQ ID NO: 344). In some embodiments, the anti-IL13Rα2 binder is a VHH, single domain antibody, an scFV or a CAR, having a VH domain comprising a sequence that has at least 70% sequence identity to SEQ ID NO: 247. In some embodiments, the anti-IL2Rα2 CAR VH domain comprises a variable heavy chain that has at least 85%, 90%, 91%, 92%, 93%, 94%, 95%, 96%, 97%, 98%, 99% sequence identity to SEQ ID NO: 247, or 100% sequence identity to SEQ ID NO: 247.

In some embodiments, the anti-IL13RA2 binder is a VHH, single domain antibody, an scFV or a CAR, having a VH domain comprising a CDR3 having a sequence of RVAGRQDDY (SEQ ID NO: 256). In some embodiments, the anti-IL13RA2 binder comprises a CDR1 having the sequence of GSIFNTNG (SEQ ID NO: 271) and a CDR2 sequence ISSGGNT (SEQ ID NO: 339) or GISSGGNTV (SEQ ID NO: 304). In some embodiments, the anti-IL13RA2 binder is a VHH, single domain antibody, an scFV or a CAR, having a VH domain comprising a sequence that has at least 70% sequence identity to SEQ ID NO: 251. In some embodiments, the anti-IL13RA2 CAR V_{H} domain comprises a variable heavy chain that has at least 85%, 90%, 91%, 92%, 93%, 94%, 95%, 96%, 97%, 98%, 99% sequence identity to SEQ ID NO: 251, or 100% sequence identity to SEQ ID NO: 251.

The VHH domains described in this section as well as elsewhere in the disclosure may be made into a nanobody, for example by fusing with an Fc domain. In some embodiments, the VHH-Fc fusion proteins may be used as polypeptides in therapeutic application. In some embodiments the polypeptides in soluble form may enhance phagocytosis and cytokine generation of myeloid cells in proximity. This can also be tested in vitro.

In some embodiments, the CAR comprises an extracellular IL 13Rα2 binding domain of any one of the binder sequences listed in Table 5. In some embodiments, the CAR comprises an extracellular IL13Rα2 binding domain of any one of the binder sequences from SEQ ID NOs: 195-252 or fragments thereof. In some embodiments a leader sequence. In some embodiments, the leader sequence (signal sequence) comprises a sequence of MWLQSLLLLGTVACSIS (SEQ ID NO: 306). In some embodiments, extracellular domain comprises a hinge. In some embodiments the hinge is operatively connected to the transmembrane domain. In some embodiments, the hinge is a CD8alpha hinge. In some embodiments, the hinge has a sequence of ALSNSIMYFSHFVPVFLPAKPTTTPAPRPPTPAPTIASQPLSLRPEACRPAAGGAVHTRGLD (SEQ ID NO: 307). In some embodiments the extracellular domain comprises a linker. In some embodiments, the linker is operatively linked to the V_{H} and the hinge. In some embodiments, the linker is operatively linked to the V_{H} and the transmembrane domain. In some embodiments, the linker has a sequence of SGGGSG (SEQ ID NO: 308). In some embodiments, the transmembrane domain is a CD8 transmembrane domain. In some embodiments, the transmembrane domain has a sequence of IYIWAPLAGTCGVLLLSLVITLYC (SEQ ID NO: 309). In some embodiments, the intracellular domain comprises an intracellular signaling domain from FcsR. In some embodiments, the intracellular domain comprises a sequence of

(SEQ ID NO: 310)
RRLKIQVRKAAITSYEKSDGVYTGLSTRNQETYETLKHEKPPQ.

In some embodiments, the intracellular domain comprises at least two intracellular signaling domains. In some embodiments, the least two intracellular signaling domains are separated by a linker. In some embodiments the linker has a sequence of GSGS (SEQ ID NO: 311). In some embodiments, for a cell specific CAR, e.g., a myeloid cell specific CAR is designed, wherein the transmembrane domain is capable of dimerizing or multimerizing with an endogenous protein in a myeloid cell and this dimerizing or multimerizing of the CAR domain with an endogenous protein allows expression and function of the CAR on myeloid cell surface. In some embodiments, the myeloid cell specific CAR comprises a TM domain of a CD16 protein (e.g., a human CD16), having a sequence GLAVSTISSFFPPGYQVSFCLVMVLLFAVDTGLYFSV (SEQ ID NO: 312), which comprises a short extracellular region and the transmembrane region. In some embodiments, the myeloid cell specific CAR comprises a human CD89 domain, having a sequence IHQDYTTQNLIRMAVAGLVLVALLAILV (SEQ ID NO: 313). Incorporating a TM domain of SEQ ID NO: 312 and 313 in the CAR allows oligomerization with a myeloid cell endogenous protein FcR-γ which allows the CAR to be expressed selectively in the cell that expresses the endogenous protein FcR-γ. In some embodiments, the intracellular domain comprises an intracellular signaling domain that that is the PI3K recruitment domain. In some embodiments, the PI3K recruitment domain has a sequence of YEDMRGILYAAPQLRSIRGQPGPNHEEDADSYENM (SEQ ID NO: 314).

In some embodiments, the transmembrane domain is operably linked to an intracellular domain comprising an intracellular signaling domain.

In some embodiments, the intracellular signaling domain is derived from an intracellular PI3-kinase recruitment domain, a phagocytosis receptor intracellular domain, a pattern recognition receptor intracellular domain, a CD40 intracellular domain, an FcR intracellular domain, a cytokine receptor intracellular domain, a chemokine receptor intracellular domain.

In some embodiments, the intracellular domain comprises at least two intracellular signaling domains.

In some embodiments, the extracellular domain comprises a hinge domain connecting the antigen binding domain and the transmembrane domain.

In some embodiments, the recombinant nucleic acid is an RNA.

In some embodiments, the recombinant nucleic acid is an mRNA.

In some embodiments, the recombinant nucleic acid is associated with one or more lipids.

In some embodiments, the recombinant nucleic acid is encapsulated in a liposome.

In some embodiments, the liposome is a lipid nanoparticle.

In some embodiments, the recombinant nucleic acid is a vector.

In some embodiments, the recombinant nucleic acid encodes a chimeric antigen receptor (CAR) comprising a VH domain described herein.

Also provided herein is a cell comprising the recombinant nucleic acid of any of the compositions described herein.

In some embodiments, the cell is an immune cell.

In some embodiments, the cell is a myeloid cell, a lymphoid cell, a precursor cell, a stem cell or an induced pluripotent cell.

In some embodiments, the cell is CD14+/CD16−.

### Cadherin Binding Domains

Provided herein are chimeric fusion proteins (CFPs) and recombinant nucleic acids encoding CFPs that target a cadherin target protein. In some embodiments the target protein is CDH17.

In one aspect, provided herein is a composition comprising a recombinant nucleic acid comprising a sequence encoding a chimeric fusion protein (CFP), the CFP comprising: an extracellular domain comprising an antigen binding domain that targets a cadherin; a transmembrane domain operatively linked to the extracellular domain; and an intracellular domain comprising an intracellular signaling domain operatively linked to the transmembrane domain.

In some embodiments, the cadherin is CDH17.

In some embodiments, the intracellular domain comprises an intracellular signaling domain from a phagocytosis receptor, a pattern recognition receptor, CD40, an Fc receptor, a cytokine receptor, and/or a chemokine receptor.

In some embodiments, the intracellular domain comprises at least two intracellular signaling domains or at least three two intracellular signaling domains.

In some embodiments, the intracellular domain comprises an intracellular signaling domain from CD40.

In some embodiments, the intracellular domain comprises an intracellular signaling domain from Fc epsilon receptor Ig (FCER1G).

In some embodiments, the intracellular domain comprises an intracellular signaling domain comprising a PI3K recruitment domain.

In some embodiments, the intracellular domain comprises (A) a first intracellular signaling domain from Fc epsilon receptor Ig (FCER1G) and (B) a second intracellular signaling domain comprising a PI3K recruitment domain.

In some embodiments, the intracellular domain comprises (A) a first intracellular signaling domain from CD40 and (B) a second intracellular signaling domain from Fc epsilon receptor Ig (FCER1G) and (C) a third intracellular signaling domain comprising a PI3K recruitment domain.

In some embodiments, the antigen binding domain is an antibody domain or antigen binding fragment thereof.

In some embodiments, the antigen binding domain is an scFv or a single domain antibody domain or a nanobody.

In some embodiments, the antigen binding domain is a VHH domain.

In some embodiments, the extracellular domain comprises a hinge domain.

In some embodiments, the extracellular domain comprises a CD8 hinge domain.

In some embodiments, extracellular domain comprises a hinge domain having the sequence

(SEQ ID NO: 307)
ALSNSIMYFSHFVPVFLPAKPTTTPAPRPPTPAPTIASQPLSLRPEACR
PAAGGAVHTRGLD.

In some embodiments, the CFP comprises a leader sequence.

In some embodiments, the CFP comprises a leader sequence that has the sequence

(SEQ ID NO: 306)
MWLQSLLLLGTVACSIS.

In some embodiments, the extracellular domain comprises a hinge domain and a linker between the hinge domain and the antigen binding domain.

In some embodiments, the extracellular domain comprises a linker between the antigen binding domain and the transmembrane domain.

In some embodiments, the linker has a sequence of (GxS)n, wherein x is an integer of from 1 to 4 and n is an integer of from 1 to 4 (SEQ ID NO: 356).

In some embodiments, the linker has a sequence SGGGSG (SEQ ID NO: 308).

In some embodiments, the transmembrane domain is a CD8 transmembrane domain.

In some embodiments, the transmembrane domain has a sequence of

(SEQ ID NO: 309)
IYIWAPLAGTCGVLLLSLVITLYC.

In some embodiments, the intracellular domain comprises a sequence of

(SEQ ID NO: 310)
RRLKIQVRKAAITSYEKSDGVYTGLSTRNQETYETLKHEKPPQ.

In some embodiments, the intracellular domain comprises PI3K recruitment domain that has a sequence of YEDMRGILYAAPQLRSIRGQPGPNHEEDADSYENM (SEQ ID NO: 314).

In some embodiments, the recombinant nucleic acid is an RNA.

In some embodiments, the recombinant nucleic acid is an mRNA.

In some embodiments, the recombinant nucleic acid is associated with one or more lipids.

In some embodiments, the recombinant nucleic acid is encapsulated in a liposome.

In some embodiments, the liposome is a lipid nanoparticle.

In some embodiments, the recombinant nucleic acid is a vector.

Also provided herein is a composition comprising a cell comprising a recombinant nucleic acid of a composition described herein.

In some embodiments, the cell is an immune cell.

In some embodiments, the cell is a myeloid cell, a lymphoid cell, a precursor cell, a stem cell or an induced pluripotent cell.

In some embodiments, the cell is CD14+/CD16−.

In some embodiments, the cell is a population of cells.

In some embodiments, the cell is a population of at least 1×10\hat{ }5 cells.

Also provided herein is a pharmaceutical composition comprising a composition described herein.

Also provided herein is a method of treating a disease or condition in a subject in need thereof comprising administering to the subject a pharmaceutical composition described herein.

In some embodiments, the disease or condition is a cancer.

In some embodiments, the disease or condition a gastrointestinal cancer or a neuroendocrine cancer.

### Therapeutic Compositions and Methods

Provided herein, in one aspect, is a myeloid cell, such as a CD14+ cell, a CD14+/CD16− cell, a CD14+/CD16+ cell, a CD14−/CD16+ cell, CD14−/CD16—cell, a dendritic cell, an M0 macrophage, an M2 macrophage, an M1 macrophage or a mosaic myeloid cell/macrophage/dendritic cell. In some embodiments, provided herein is a therapeutic composition comprising at least 20%, at least 30%, at least 40% or at least 50% CD14+ cells. In some embodiments, the therapeutic composition comprises at least 20%, at least 30%, at least 40% or at least 50% CD14+/CD16− cells.

In some embodiments, a therapeutic composition comprises at least a polypeptide comprising any one of the sequences listed at least in Tables 1, 3, 5, 7 and 8. In some embodiments, therapeutic composition comprises at least a polypeptide comprising a sequence of SEQ ID NO: 1-4, 6-14, 16-21, 22-112, 195-252, 315, 316. In some embodiments, therapeutic composition comprises at least a polypeptide comprising a sequence of SEQ ID NO: 1-4, 6-14, 16-21, 22-112, 195-252, 315, 316, or a polynucleic acid encoding the same. In some embodiments the polynucleotide is an mRNA. In some embodiments, the mRNA comprises a 5′ UTR and a 3′UTR, wherein the 5′UTR or the 3′UTR may be modified for update, prolonged expression and/or stability. In some embodiments the mRNA may be encapsulated in a nanoparticle. In some embodiments, the nanoparticle may be a lipid nanoparticle, and comprises at least one cationic lipid. In some embodiments the nanoparticle may be a non-lipid nanoparticle.

Provided herein is a therapeutic combination for treating a cancer. Cancers that can be treated include, but are not limited to, T cell lymphoma, cutaneous lymphoma, B cell cancer (e.g., multiple myeloma, Waldenstrom's macroglobulinemia), the heavy chain diseases (such as, for example, alpha chain disease, gamma chain disease, and mu chain disease), benign monoclonal gammopathy, and immunocytic amyloidosis, melanomas, breast cancer, lung cancer, bronchus cancer, colorectal cancer, prostate cancer (e.g., metastatic, hormone refractory prostate cancer), pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, and the like. Other non-limiting examples of types of cancers applicable to the methods encompassed by the present disclosure include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, the cancer is an epithelial cancer such as, but not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers can be characterized in various other ways including, but not limited to, serous, endometrioid, mucinous, clear cell, or undifferentiated. In some embodiments, the present disclosure is used in the treatment, diagnosis, and/or prognosis of lymphoma or its subtypes, including, but not limited to, mantle cell lymphoma. Lymphoproliferative disorders are also considered to be proliferative diseases.

In some embodiments, the cancer may be a hematological cancer. In one embodiment, the cancer is AML, and the therapeutic composition comprises a polypeptide from Table 1, comprising a sequence of SEQ ID NOs 1-20, and 342. In some embodiments, a human subject with AML is treated with a composition comprising a polypeptide comprising any one or more of the sequences of SEQ ID NO: 1-4, 6-14, 16-21, 342, or a polynucleotide encoding any one or more of the sequences of SEQ ID NO: 1-4, 6-14, 16-21, 342. In some embodiments, the polynucleotide may comprise a sequence of SEQ ID NO: 5, or SEQ ID NO: 15.

In one embodiment, the cancer is thymic carcinoma, and the therapeutic composition comprises a polypeptide from Table 1, comprising a sequence of SEQ ID Nos 1-20, and 342 or a polynucleotide sequence encoding one or more polypeptides listed therein. In some embodiments, a human subject with thymic carcinoma is treated with a composition comprising a polypeptide comprising any one or more of the sequences of SEQ ID NO: 1-4, 6-14, 16-21, 342, or a polynucleotide encoding any one or more of the sequences of SEQ ID NO: 1-4, 6-14, 16-21, 342. In some embodiments, the polynucleotide may comprise a sequence of SEQ ID NO: 5, or SEQ ID NO: 15.

In one embodiment, the cancer is nasopharyngeal carcinoma, and the therapeutic composition comprises a polypeptide from Table 1, comprising a sequence of SEQ ID NOs 1-20, and 342 or a polynucleotide sequence encoding one or more polypeptides listed therein. In some embodiments, a human subject with nasopharyngeal carcinoma is treated with a composition comprising a polypeptide comprising any one or more of the sequences of SEQ ID NO: 1-4, 6-14, 16-21, 342, or a polynucleotide encoding any one or more of the sequences of SEQ ID NO: 1-4, 6-14, 16-21, 342. In some embodiments, the polynucleotide may comprise a sequence of SEQ ID NO: 5, or SEQ ID NO: 15. In some embodiments, the cancer is an EBV related cancer.

In one embodiment, the cancer is renal cell carcinoma, and the therapeutic composition comprises a polypeptide from Table 1, comprising a sequence of SEQ ID NOs 1-20, and 342 or a polynucleotide sequence encoding one or more polypeptides listed therein. In some embodiments, a human subject with renal cell carcinoma is treated with a composition comprising a polypeptide comprising any one or more of the sequences of SEQ ID NO: 1-4, 6-14, 16-21, 342, or a polynucleotide encoding any one or more of the sequences of SEQ ID NO: 1-4, 6-14, 16-21, 342. In some embodiments, the polynucleotide may comprise a sequence of SEQ ID NO: 5, or SEQ ID NO: 15.

In one embodiment, the cancer is DLBCL, and the therapeutic composition comprises a polypeptide from Table 1, comprising a sequence of SEQ ID NOs 1-20, and 342 or a polynucleotide sequence encoding one or more polypeptides listed therein. In some embodiments, a human subject with DLBCL is treated with a composition comprising a polypeptide comprising any one or more of the sequences of SEQ ID NO: 1-4, 6-14, 16-21, 342, or a polynucleotide encoding any one or more of the sequences of SEQ ID NO: 1-4, 6-14, 16-21, 342. In some embodiments, the polynucleotide may comprise a sequence of SEQ ID NO: 5, or SEQ ID NO: 15.

In one embodiment, the cancer is mesothelioma, and the therapeutic composition comprises a polypeptide from Table 1, comprising a sequence of SEQ ID NOs 1-20, and 342 or a polynucleotide sequence encoding one or more polypeptides listed therein. In some embodiments, a human subject with mesothelioma is treated with a composition comprising a polypeptide comprising any one or more of the sequences of SEQ ID NO: 1-4, 6-14, 16-21, 342, or a polynucleotide encoding any one or more of the sequences of SEQ ID NO: 1-4, 6-14, 16-21, 342. In some embodiments, the polynucleotide may comprise a sequence of SEQ ID NO: 5, or SEQ ID NO: 15.

In one embodiment, the cancer is CLL, and the therapeutic composition comprises a polypeptide from Table 1, comprising a sequence of SEQ ID NOs 1-20, and 342 or a polynucleotide sequence encoding one or more polypeptides listed therein. In some embodiments, a human subject with CLL is treated with a composition comprising a polypeptide comprising any one or more of the sequences of SEQ ID NO: 1-4, 6-14, 16-21, 342, or a polynucleotide encoding any one or more of the sequences of SEQ ID NO: 1-4, 6-14, 16-21, 342. In some embodiments, the polynucleotide may comprise a sequence of SEQ ID NO: 5, or SEQ ID NO: 15.

In one embodiment, the cancer is NSCLC, and the therapeutic composition comprises a polypeptide from Table 1, comprising a sequence of SEQ ID NOs 1-20, and 342 or a polynucleotide sequence encoding one or more polypeptides listed therein. In some embodiments, a human subject with NSCLC is treated with a composition comprising a polypeptide comprising any one or more of the sequences of SEQ ID NO: 1-4, 6-14, 16-21, 342, or a polynucleotide encoding any one or more of the sequences of SEQ ID NO: 1-4, 6-14, 16-21, 342. In some embodiments, the polynucleotide may comprise a sequence of SEQ ID NO: 5, or SEQ ID NO: 15.

In one embodiment, the cancer is GBM, and the therapeutic composition comprises a polypeptide from Table 1, comprising a sequence of SEQ ID NOs 1-20, and 342 or a polynucleotide sequence encoding one or more polypeptides listed therein. In some embodiments, a human subject with GBM is treated with a composition comprising a polypeptide comprising any one or more of the sequences of SEQ ID NO: 1-4, 6-14, 16-21, 342, or a polynucleotide encoding any one or more of the sequences of SEQ ID NO: 1-4, 6-14, 16-21, 342. In some embodiments, the polynucleotide may comprise a sequence of SEQ ID NO: 5, or SEQ ID NO: 15.

In some embodiments, the cancer is a T cell lymphoma, an RCC, a melanoma, an MCL, CTCL, DLBCL, or breast cancer. A human subject with any one of a T cell lymphoma, an RCC, a melanoma, an MCL, CTCL, DLBCL, or breast cancer is treated with a composition comprising a polypeptide comprising any one or more of the sequences of SEQ ID NO: 1-4, 6-14, 16-21, 342, or a polynucleotide encoding any one or more of the sequences of SEQ ID NO: 1-4, 6-14, 16-21, 342, or a sequence having at least 80% sequence identity to any one of the above. In some embodiments, the polynucleotide may comprise a sequence of SEQ ID NO: 5, or SEQ ID NO: 15.

In some embodiments, provided herein is a therapeutic composition comprising less than 20%, less than 15%, less than 10% or less than 5% dendritic cells. The myeloid cell for the therapeutic composition as described herein, comprises a recombinant nucleic acid that encodes a chimeric fusion protein encoding a CFP receptor protein or an engager protein as described herein. The myeloid cell for the therapeutic composition as described herein, expresses the CFP encoded by the recombinant nucleic acid or expresses an engager protein encoded by the recombinant nucleic acid as described herein.

In some embodiments, provided herein is a recombinant polypeptide for the treatment of a cancer in a subject in need thereof, the recombinant polypeptide comprises a binding domain, having a CDR1, CDR2 and a CDR3 sequence, wherein the CDR3 sequence is selected from SEQ ID NOs: 4, and 14; or a polynucleotide comprising a sequence encoding the polypeptide.

In one aspect provided herein is a recombinant nucleic acid encoding a polypeptide comprising a sequence selected from the group consisting of SEQ ID NOs: 22-112, or a sequence that has at least 80% sequence identity to the sequences of SEQ ID NOs: 22-112. In some embodiments, the polynucleotide is an mRNA. In some embodiments, the composition is a pharmaceutical composition. In some embodiments, the pharmaceutical composition further comprises a delivery vehicle. In some embodiments the delivery vehicle is a nanoparticle, e.g. a lipid nanoparticle. In some embodiments, the nanoparticle is a non-lipid nanoparticle. In some embodiments, the pharmaceutical composition is formulated for in vivo administration. In some embodiments the pharmaceutical composition is formulated for systemic delivery, e.g., via injection, e.g., i.v., i.m., i.e., or subcutaneous. In one aspect, provided herein is a recombinant polypeptide for the treatment of a cancer in a subject in need thereof, the recombinant polypeptide comprises a binding domain, having a CDR1, CDR2 and a CDR3 sequence, wherein the CDR3 sequence is selected from the group consisting of SEQ ID NOs: 113-150, and SEQ ID NO: 343. In one embodiment, the binding domains further comprise a CDR1 sequence selected from SEQ ID NOs: 151-166; and CDR2 sequence selected from SEQ ID NOs: 167-194.

In some embodiments, provided herein is a recombinant nucleic acid for the treatment of a cancer in a subject in need thereof, the recombinant nucleic acid comprising a sequence encoding a polypeptide comprising a binding domain, having a CDR1, a CDR2 and a CDR3 sequence, wherein the CDR3 sequence is selected from SEQ ID NOs: 113-150, and SEQ ID NO: 343, wherein the polynucleotide is an mRNA. In some embodiments, the polypeptide comprises a CDR1 sequence of any one of SEQ ID NOs. 151-166. In some embodiments, the polypeptide further comprises a CDR2 sequence of any one of SEQ ID NOs. 167-194. In some embodiments, the therapeutic polypeptide may be expressed in vivo upon administration of an mRNA encoding it. In some embodiments, the therapeutic polypeptide is engineered for specific uptake and/or expression in myeloid cells, and not substantially expressed in non-myeloid cells e.g., T cells, epithelial cells or hepatocytes or neuronal cells, upon administering in vivo an mRNA comprising a sequence encoding the polypeptide. In some embodiments the polypeptide is a VHH polypeptide. In some embodiments, the cancer is an AML, CLL, NSCLC, RCC, melanoma, breast cancer MCL, CTCL DLBCL or a lymphoma.

In one embodiment, provided herein is a composition comprising a recombinant nucleic acid, comprising a sequence encoding any one of the polypeptides having a sequence of SEQ ID NO: 33, 80, 55, 108, 58 or 47. In one embodiment, provided herein is a composition comprising a polynucleotide, comprising a sequence encoding any one of the polypeptides having a sequence selected from a group consisting of SEQ ID NO: 33, 80, 55, 108, 58 and 47. In some embodiments, provided herein is a recombinant nucleic acid for the treatment of a cancer in a subject in need thereof, the recombinant nucleic acid comprising a sequence encoding a polypeptide comprising a binding domain, having a CDR1, a CDR2 and a CDR3 sequence, wherein the CDR3 sequence is selected from the sequences ACADTTLYEYDY (SEQ ID NO: 343), ACANWSSLGPYDY (SEQ ID NO: 319), TCASPEKYEYDY (SEQ ID NO: 322), RYSGRTY (SEQ ID NO: 324), DCAGGVGHEYDY (SEQ ID NO: 326) and DCSLHGSDYPYD (SEQ ID NO: 328). In some embodiments, provided herein is a recombinant nucleic acid for the treatment of a cancer in a subject in need thereof, the recombinant nucleic acid comprising a sequence encoding a polypeptide comprising a binding domain, having a CDR1, a CDR2 and a CDR3 sequence, wherein the CDR3 sequence comprises any one of the sequences ACADTTLYEYDY (SEQ ID NO: 343), ACANWSSLGPYDY (SEQ ID NO: 319), TCASPEKYEYDY (SEQ ID NO: 322), RYSGRTY (SEQ ID NO: 324), DCAGGVGHEYDY (SEQ ID NO: 326) or DCSLHGSDYPYD (SEQ ID NO: 328). In some embodiments, the therapeutic polypeptide may be expressed in vivo upon administration of an mRNA encoding it. In some embodiments, the therapeutic polypeptide is engineered for specific uptake and/or expression in myeloid cells, and not substantially expressed in non-myeloid cells e.g., T cells, epithelial cells or hepatocytes or neuronal cells, upon administering in vivo an mRNA comprising a sequence encoding the polypeptide. In some embodiments the polypeptide is a VHH polypeptide. In some embodiments, the cancer is an AML, CLL, NSCLC, RCC, melanoma, breast cancer MCL, CTCL DLBCL or a lymphoma.

In one aspect provided herein is a pharmaceutical composition comprising a recombinant nucleic acid encoding a polypeptide comprising a sequence selected from the group consisting of SEQ ID Ns: 195-252. In some embodiments, the polynucleotide is an mRNA. In some embodiments the polypeptide comprises an anti IL13Rα2 binding domain. In some embodiments, the composition is a pharmaceutical composition. In some embodiments, the pharmaceutical composition further comprises a delivery vehicle. In some embodiments the delivery vehicle is a nanoparticle, e.g. a lipid nanoparticle. In some embodiments, the nanoparticle is a non-lipid nanoparticle. In some embodiments, the pharmaceutical composition is formulated for in vivo administration. In some embodiments the pharmaceutical composition is formulated for systemic delivery, e.g., via injection, e.g., i.v., i.m., i.e., or subcutaneous. In one aspect, provided herein is a recombinant polypeptide for the treatment of a cancer in a subject in need thereof, the recombinant polypeptide comprises a binding domain, having a CDR1, CDR2 and a CDR3 sequence, wherein the CDR3 sequence is selected from the group consisting of SEQ ID NOs: 253-265. In one embodiment, the binding domains further comprise a CDR1 sequence selected from SEQ ID NOs: 266-286; and CDR2 sequence selected from SEQ ID NOs: 287-305.

In some embodiments, the pharmaceutical composition comprises a recombinant nucleic acid comprising a sequence encoding polypeptides having a sequence of any one of SEQ ID NOs: 230, 237 246, 247 251 or 340; or a sequence that has at least 80% sequence identity to any one of the sequences of SEQ ID NOs: 230, 237, 246, 247, 251 or 340. In some embodiments, the pharmaceutical composition comprises a recombinant nucleic acid comprising a sequence encoding a polypeptide comprising a sequence selected from the group consisting of SEQ ID NOs: 230, 237, 246, 247, 251 and 340, or a sequence that has at least 80% sequence identity to the sequences of SEQ ID NOs: 230, 237, 246, 247, 251 and 340.

In some embodiments, provided herein is a therapeutic composition comprising a chimeric fusion protein, such as a chimeric fusion receptor protein (CFP), the CFP comprises: (a) an extracellular domain comprising: (i) a scFv that specifically binds any one of the targets disclosed herein, and (ii) a hinge domain derived from CD8, a hinge domain derived from CD28 or at least a portion of an extracellular domain from CD68; (b) a CD8 transmembrane domain, a CD28 transmembrane domain, a CD2 transmembrane domain or a CD68 transmembrane domain; and (c) an intracellular domain comprising at least two intracellular signaling domains, wherein the at least two intracellular signaling domains comprise: (i) a first intracellular signaling domain derived from FcRγ or FcRε, (ii) a second intracellular signaling domain e.g., intracellular signaling domain derived from TLR3, TLR4, TLR7, TLR 9, TRIF, RIG-1, MYD88, MAL, IRAK1, MDA-5, an IFN-receptor, STING, MAVS, TRIF or TASL intracellular domains, an NLRP family member, NLRP1-14, NOD1, NOD2, Pyrin, AIM2, NLRC4, FCGR3A, FCERIG, IL-1, IL3, IL5, IL-6, IL-12, IL-13, IL-23, TNF, IL-18, IL-23, IL-27, CSF, MCSF, GMCSF, IL17, IP-10, or RANTES and a second intracellular signaling domain, that comprises a PI3K recruitment domain, or a domain that is derived from CD40.

In some embodiments, the pharmaceutical composition comprises a population of cells comprising therapeutically effective dose of the myeloid cells. In some embodiments, the population of cells: differentiate into effector cells in the subject after administration; infiltrate into a diseased site of the subject after administration or migrate to a diseased site of the subject after administration; and/or have a life-span of at least 5 days in the subject after administration.

In some embodiments, myeloid cells may be further modified or manipulated to develop a therapeutically effective myeloid cells. Isolated cells can be manipulated by expressing a gene or a fragment thereof in the cell, without altering its functional and developmental plasticity, differential potential and cell viability.

In some embodiments, myeloid cells may be further modified or manipulated to develop a therapeutically effective myeloid cells by expressing a non-endogenous polynucleotide into the cell. A non-endogenous polynucleotide may encode for a protein or a peptide. Alternatively, a non-endogenous polypeptide may be a non-coding sequence, such as an inhibitory RNA, or a morpholino.

In some embodiments, myeloid cells may be further modified or manipulated to develop a therapeutically effective myeloid cells by stably altering the genomic sequence of the cell. In some embodiments, the myeloid cell is manipulated by editing the myeloid cell genome using a CRISPR-CAS system. In some embodiments, one or more genes may be edited to silence the gene expression. In some embodiments, the myeloid cell is manipulated to delete a gene. In some embodiments, one or more genes may be edited to enhance the gene expression. In some embodiments, the genetic material is introduced into a myeloid cell in the form of a messenger RNA, wherein the messenger RNA encodes a protein or a peptide, thereby rendering the myeloid cell therapeutically effective. In some embodiments, naked DNA or messenger RNA (mRNA) may be used to introduce the nucleic acid inside the myeloid cell. In some embodiments, DNA or mRNA encoding the chimeric antigen receptor is introduced into the phagocytic cell by lipid nanoparticle (LNP) encapsulation. mRNA is single stranded and may be codon optimized. In some embodiments the mRNA may comprise one or more modified or unnatural bases such as 5′-Methylcytosine, or Pseudouridine or methyl pseudouridine. In some embodiments greater than or about 50% uridine (‘U’) residues of the mRNA may be converted to methyl-pseudouridine. In some embodiments, the mRNA may be 50-10,000 bases long. In one aspect the transgene is delivered as an mRNA. The mRNA may comprise greater than about 100, 200, 300, 400, 500, 600, 700, 800, 900, 1000, 1100, 1200, 1300, 1400, 1500, 1600, 1700, 1800, 1900, 2000, 3000, 4000, 5000, 6000, 7000, 8000, 9000, 10,000 bases. In some embodiments, the mRNA may be more than 10,000 bases long. In some embodiments, the mRNA may be about 11,000 bases long. In some embodiments, the mRNA may be about 12,000 bases long. In some embodiments, the mRNA comprises a transgene sequence that encodes a fusion protein. LNP encapsulated DNA or RNA can be used for transfecting a macrophage or can be administered to a subject. In some embodiments, the mRNA is incorporated into an effector myeloid cell population by transient transfection. In some embodiments the transient transfection method comprises electroporation of the mRNA. In some embodiments, the transient transfection comprises chemical transfection. In some embodiments, 1-5,000 micrograms/ml of the mRNA may be used for transfection using a suitable protocol for the methods described above. In some embodiments, 1-2,000 micrograms/ml of the mRNA may be used for transfection. In some embodiments, 1-1,000 micrograms/ml of the mRNA may be used for transfection. In some embodiments, 1-1,000 micrograms/ml of the mRNA may be used for transfection. In some embodiments, 1-500 micrograms/ml of the mRNA may be used for transfection. In some embodiments, 1-250 micrograms/ml of the mRNA may be used for transfection. In some embodiments, about 500 micrograms/ml of the mRNA or less may be used for transfection. In some embodiments, about 250 micrograms/ml of the mRNA or less may be used for transfection. In some embodiments, about 10 micrograms/ml of the mRNA is used. In some embodiments, about 20 micrograms/ml of the mRNA is used. In some embodiments, about 30 micrograms/ml of the mRNA is used. In some embodiments, about 40 micrograms/ml of the mRNA is used. In some embodiments, about 50 micrograms/ml of the mRNA is used. In some embodiments, about 60 micrograms/ml of the mRNA is used. In some embodiments, about 80 micrograms/ml of the mRNA is used. In some embodiments, about 100 micrograms/ml of the mRNA is used. In some embodiments, about 150 micrograms/ml of the mRNA is used. In some embodiments, about 200 micrograms/ml of the mRNA is used. In some embodiments, 20, 50, 100, 150, 200, 250, 300, 400, 500 or about 1000 micrograms/ml of the mRNA is used. A suitable cell density is selected for a transfection, based on the method and instrument and/or reagent manufacturer's instructions, or as is well-known to one of skill in the art.

In some embodiments the recombinant nucleic acid is an mRNA. mRNA constructs may be thawed on ice and gently pipetted to monocytes and pre-mixed. In some embodiments, the mRNA is electroporated into the cells. Cells following elutriation may be pooled, centrifuged and may be subjected to electroporation with mRNA using MaxCyte ATX system optimized for the said purpose. In some embodiments, optimized electroporation buffer, cell density, and/or mRNA concentration is used for each protocol for each construct.

In some embodiments, a polynucleotide may be introduced into a myeloid cell in the form of a circular RNA (circRNAs). In circular RNAs (circRNAs) the 3′ and 5′ ends are covalently linked. CircRNA may be delivered inside a cell using LNPs.

In some embodiments, a stable integration of transgenes into macrophages and other phagocytic cells may be accomplished via the use of a transposase and transposable elements, in particular, mRNA-encoded transposase. In one embodiment, Long Interspersed Element-1 (L1) RNAs may be contemplated for retrotransposition of the transgene and stable integration into a macrophage or a phagocytic cell. Retrotransposon may be used for stable integration of a recombinant nucleic acid encoding a phagocytic or tethering receptor (PR) fusion protein (PFP).

In some embodiments, the myeloid cell may be modified by expressing a transgene via incorporation of the transgene in a transient expression vector. In some embodiments expression of the transgene may be temporally regulated by a regulator from outside the cell. Examples include the Tet-on Tet-off system, where the expression of the transgene is regulated via presence or absence of tetracycline.

In some embodiments, the myeloid cell may be modified to develop a therapeutically effective cell by contacting the cell with a compound, which compound may be an inhibitor or an activator of a protein or enzyme within the myeloid cell.

In some embodiments, a polynucleotide encoding a chimeric antigen receptor may be introduced into an isolated myeloid cell that is obtained by the method described in the preceding section, where the chimeric antigen receptor upon expression in the myeloid cell augments an innate immune response function of the myeloid cell. In some embodiments, the chimeric antigen receptor expression can direct a myeloid cell to a specific target in vivo or in vitro. In some embodiments, the chimeric antigen receptor may increase the phagocytic potential of the myeloid cell. In some embodiments, the chimeric antigen receptor increases the immunogenicity of the myeloid cell. In some embodiments, the chimeric antigen receptor may increase augment intracellular signaling. In some embodiments, the chimeric antigen receptor may function cooperatively with one or more proteins within the cell. In some embodiments, the chimeric antigen receptor may dimerize or multimerize with a second receptor or transmembrane protein inside the myeloid cell, where the second receptor or transmembrane protein is an endogenous protein. In some embodiments, the cells are cultured ex vivo briefly after thawing or after incorporation of the nucleic acid. In some embodiments, the ex vivo culture is performed in presence of a suitable medium, that may comprise a regulated serum component, e.g., human serum albumin (HSA). In some embodiments, the ex vivo culture and manipulation may be performed in low serum containing media. In some embodiment, the serum is specifically treated for compliment deactivation. In some embodiments, the myeloid cells may be cultured ex vivo as described above, in the presence of M-CSF. In some embodiments, the myeloid cells may be cultured ex vivo as described above, in the presence of GM-CSF. In some embodiments, the myeloid cells may be cultured in the presence of one or more cytokines. In some embodiments, the myeloid cells may be cultured or manipulated ex vivo in the absence of growth factor or cytokines for a period. In some embodiments, the method provided herein comprises isolation or enrichment and manipulation of a myeloid cell in less than 72 hours, 70 hours, 65 hours, 60 hours, 55 hours, 50 hours, 45 hours, 40 hours, or 35 hours, or 30 hours, or 28 hours, or 26 hours or 24 hours. In some embodiments, the myeloid cell may be culture for less than 24 hours, or less than 20 hours or less than 16 hours, or less than 14 hours, or less than 12 hours, or less than 10 hours, or less than 8 hours, or less than 6 hours or less than about 4 hours. The myeloid cell following isolation or enrichment and manipulation may be cultured briefly and frozen till further use. In some embodiments, the myeloid cell is thawed once or at the most twice.

In some embodiments, the therapeutically competent cells are cells that have been electroporated with a recombinant nucleic acid encoding a polypeptide, frozen and thawed, culture stabilized for less than 24 hours, and wherein the cells in the cell population at the time of administration exhibit (i) greater than at least 70% viability, (ii) greater than at least 50% CD14+ and CD16− cells; and/or greater than 50% CD11b+/CD14+/CD16− cells; (iii) less than 5% CD3+ cells, less than 5% CD19+ cells, less than about 10% CD56+ cells, less than about 10% CD42b+ cells (iv) greater than 50% cells express the polypeptide encoded by the electroporated nucleic acid. In some embodiments, the therapeutically competent cells are cells that have been electroporated with a recombinant nucleic acid encoding a polypeptide, culture stabilized for less than 24 hours, frozen and thawed, and wherein the cells in the cell population at the time of administration exhibit (i) greater than at least 70% viability, (ii) greater than at least 50% CD14+ and CD16− cells; and/or greater than 50% CD11b+/CD14+/CD16− cells; (iii) less than 5% CD3+ cells, less than 5% CD19+ cells, less than about 10% CD56+ cells, less than about 10% CD42b+ cells (iv) greater than 50% cells express the polypeptide encoded by the electroporated nucleic acid. In some embodiments, the therapeutically competent cells are cells that have been culture stabilized for less than 24 hours, that have been electroporated with a recombinant nucleic acid encoding a polypeptide, frozen and thawed, and wherein the cells in the cell population at the time of administration exhibit (i) greater than at least 70% viability, (ii) greater than at least 50% CD14+ and CD16− cells; and/or greater than 50% CD11b+/CD14+/CD16− cells; (iii) less than 5% CD3+ cells, less than 5% CD19+ cells, less than about 10% CD56+ cells, less than about 10% CD42b+ cells (iv) greater than 50% cells express the polypeptide encoded by the electroporated nucleic acid. Cells must be pathogen free. In the above embodiments, the therapeutically competent cells may have been frozen and thawed not more than twice, preferably once, and may be administered within 24 hours of thawing, within 18 hours of thawing, within 8 hours of thawing, or within 2 hours of thawing. Cells are tested for quality assurance to meet the standards as described herein in the disclosure prior to administering.

Provided herein are methods for treating cancer in a subject using a pharmaceutical composition comprising engineered phagocytic cells, particularly macrophages, expressing recombinant nucleic acid encoding a phagocytic receptor (PR) fusion protein (PFP), which is specifically designed to target, attack and kill cancer cells. The PFP is also designated as a chimeric antigenic receptor for phagocytosis (CAR-P), and both the terms may be used interchangeably herein. The engineered phagocytic cells are also designated as CAR-P cells in the descriptions herein.

In general, cellular immunotherapy comprises providing the patient a medicament comprising live cells. In some aspects a patient or a subject having cancer, is treated with autologous cells, the method comprising, isolation or enrichment of PBMC-derived macrophages, modifying the macrophages ex vivo to generate highly phagocytic macrophages capable of tumor lysis by introducing into the macrophages a recombinant nucleic acid encoding chimeric antigenic receptor for phagocytosis which is a phagocytic receptor fusion protein (PFP), and administering the modified macrophages into the patient or the subject.

In one aspect, a subject is administered one or more doses of a pharmaceutical composition comprising therapeutic phagocytic cells, wherein the cells are allogeneic. An HLA may be matched for compatibility with the subject, and such that the cells do not lead to graft versus Host Disease, GVHD. A subject arriving at the clinic is HLA typed for determining the HLA antigens expressed by the subject, prior to determining a therapeutic or therapeutic regimen.

In some embodiments a therapeutically effective dose ranges between 10\hat{ }7 cells to 10\hat{ }12 myeloid cells for one infusion. The cell number may vary according to the age, body weight and other subject-related parameters and can be determined by a medical practitioner. In some embodiments, a therapeutically effective dose is about 10\hat{ }7 myeloid cells. In some embodiments, a therapeutically effective dose is about 2×10\hat{ }7 myeloid cells. In some embodiments, a therapeutically effective dose is about 3×10\hat{ }7 myeloid cells. In some embodiments, a therapeutically effective dose is about 4×10\hat{ }7 myeloid cells. In some embodiments, a therapeutically effective dose is about 5×10\hat{ }7 myeloid cells. In some embodiments, a therapeutically effective dose is about 6×10\hat{ }7 myeloid cells. In some embodiments, a therapeutically effective dose is about 7×10\hat{ }7 myeloid cells. In some embodiments, a therapeutically effective dose is about 8×10\hat{ }7 myeloid cells. In some embodiments, a therapeutically effective dose is about 9×10\hat{ }7 myeloid cells. In some embodiments, a therapeutically effective dose is about 10\hat{ }8 myeloid cells. In some embodiments, a therapeutically effective dose is about 2×10\hat{ }8 myeloid cells. In some embodiments, a therapeutically effective dose is about 3×10\hat{ }8 myeloid cells. In some embodiments, a therapeutically effective dose is about 4×10\hat{ }8 myeloid cells. In some embodiments, a therapeutically effective dose is about 5×10\hat{ }8 myeloid cells. In some embodiments, a therapeutically effective dose is about 6×10\hat{ }8 myeloid cells. In some embodiments, a therapeutically effective dose is about 7×10\hat{ }8 myeloid cells. In some embodiments, a therapeutically effective dose is about 8×10\hat{ }8 myeloid cells. In some embodiments, a therapeutically effective dose is about 9×10\hat{ }8 myeloid cells. In some embodiments, a therapeutically effective dose is about 10\hat{ }9 myeloid cells. In some embodiments, a therapeutically effective dose is about 2×10\hat{ }9 myeloid cells. In some embodiments, a therapeutically effective dose is about 3×10\hat{ }9 myeloid cells. In some embodiments, a therapeutically effective dose is about 4×10\hat{ }9 myeloid cells. In some embodiments, a therapeutically effective dose is about 5×10\hat{ }9 myeloid cells. In some embodiments, a therapeutically effective dose is about 6×10\hat{ }9 myeloid cells. In some embodiments, a therapeutically effective dose is about 7×10\hat{ }9 myeloid cells. In some embodiments, a therapeutically effective dose is about 8×10\hat{ }9 myeloid cells. In some embodiments, a therapeutically effective dose is about 9×10\hat{ }9 myeloid cells. In some embodiments, a therapeutically effective dose is about 10\hat{ }10 myeloid cells. In some embodiments, a therapeutically effective dose is about 5×10\hat{ }10 myeloid cells. In some embodiments a therapeutically effective dose is about 10\hat{ }11 myeloid cells. In some embodiments a therapeutically effective dose is about 5×10\hat{ }11 myeloid cells. In some embodiments a therapeutically effective dose is about 10\hat{ }12 myeloid cells.

Provided herein, in one aspect, one or more recombinant polynucleic acid(s) encoding one or more recombinant proteins that can be a chimeric fusion protein such as a receptor, or an engager as described herein. In some embodiments, the recombinant polynucleic acid(s) is an mRNA. In some embodiments, the recombinant polynucleic acid comprises a circRNA. In some embodiments, the recombinant polynucleic acid is encompassed in a viral vector. In some embodiments, the recombinant polynucleic acid is delivered via a viral vector.

In some embodiments, provided herein is a therapeutic composition comprising a recombinant nucleic acid encoding a chimeric fusion protein, such as a chimeric fusion receptor protein (CFP), the CFP comprises: (a) an extracellular domain comprising: (i) a scFv that specifically binds any one of the targets disclosed herein, and (ii) a hinge domain derived from CD8, a hinge domain derived from CD28 or at least a portion of an extracellular domain from CD68; (b) a CD8 transmembrane domain, a CD28 transmembrane domain, a CD2 transmembrane domain or a CD68 transmembrane domain; and (c) an intracellular domain comprising at least two intracellular signaling domains, wherein the at least two intracellular signaling domains comprise: (i) a first intracellular signaling domain derived from FcRγ or FcRε, an interferon inducing domain, and/or (ii) a third intracellular signaling domain that: (A) comprises a PI3K recruitment domain, or (B) is derived from CD40.

In some embodiments, provided herein is therapeutic composition comprising a recombinant nucleic acid encoding a bispecific or trispecific engager as disclosed herein.

In some embodiments, the therapeutic composition comprises VHH-Fc fusion polypeptides. In some embodiments, the therapeutic composition comprises VHH conjugates that may have functions related to activating macrophages and enhancing myeloid cell mediated phagocytosis directed to the target cells of the VHH binding domains.

### Other Therapeutic Compositions for Co-Administration

In some embodiments, the therapeutic composition further comprises an additional therapeutic agent selected from the group consisting of a CD47 agonist, an agent that inhibits Rac, an agent that inhibits Cdc42, an agent that inhibits a GTPase, an agent that promotes F-actin disassembly, an agent that promotes PI3K recruitment to the PFP, an agent that promotes PI3K activity, an agent that promotes production of phosphatidylinositol 3,4,5-trisphosphate, an agent that promotes ARHGAP12 activity, an agent that promotes ARHGAP25 activity, an agent that promotes SH3BP1 activity, an agent that promotes sequestration of lymphocytes in primary and/or secondary lymphoid organs, an agent that increases concentration of naive T cells and central memory T cells in secondary lymphoid organs, and any combination thereof.

In some embodiments, the myeloid cell further comprises: (a) an endogenous peptide or protein that dimerizes with the CFP, (b) a non-endogenous peptide or protein that dimerizes with the CFP; and/or (c) a second recombinant polynucleic acid sequence, wherein the second recombinant polynucleic acid sequence comprises a sequence encoding a peptide or protein that interacts with the CFP; wherein the dimerization or the interaction potentiates phagocytosis by the myeloid cell expressing the CFP as compared to a myeloid cell that does not express the CFP.

In some embodiments, the myeloid cell exhibits (i) an increase in effector activity, cross-presentation, respiratory burst, ROS production, iNOS production, inflammatory mediators, extra-cellular vesicle production, phosphatidylinositol 3,4,5-trisphosphate production, trogocytosis with the target cell expressing the antigen, resistance to CD47 mediated inhibition of phagocytosis, resistance to LILRB1 mediated inhibition of phagocytosis, or any combination thereof; and/or (ii) an increase in expression of a IL-1, IL3, IL-6, IL-10, IL-12, IL-13, IL-23, TNFα, a TNF family of cytokines, CCL2, CXCL9, CXCL10, CXCL11, IL-18, IL-23, IL-27, CSF, MCSF, GMCSF, IL-17, IP-10, RANTES, an interferon, MHC class I protein, MHC class II protein, CD40, CD48, CD58, CD80, CD86, CD112, CD155, a TRAIL/TNF Family death receptor, TGFβ, B7-DC, B7-H2, LIGHT, HVEM, TL1A, 41BBL, OX40L, GITRL, CD30L, TIM1, TIM4, SLAM, PDL1, an MMP (e.g., MMP2, MMP7 and MMP9) or any combination thereof.

In some embodiments, the intracellular signaling domain is derived from a phagocytic or tethering receptor or wherein the intracellular signaling domain comprises a phagocytosis activation domain. In some embodiments, the intracellular signaling domain is derived from a receptor other than a phagocytic receptor selected from Megf10, MerTk, FcR-alpha, or Bail. In some embodiments, the intracellular signaling domain is derived from a protein, such as receptor (e.g., a phagocytic receptor), selected from the group consisting of TNFR1, MDA5, CD40, lectin, dectin 1, CD206, scavenger receptor A1 (SRA1), MARCO, CD36, CD163, MSR1, SCARA3, COLEC12, SCARA5, SCARB1, SCARB2, CD68, OLR1, SCARF1, SCARF2, CXCL16, STAB1, STAB2, SRCRB4D, SSC5D, CD205, CD207, CD209, RAGE, CD14, CD64, F4/80, CCR2, CX3CR1, CSF1R, Tie2, HuCRIg(L), CD64, CD32a, CD16a, CD89, Fcα receptor I, CR1, CD35, CD3ζ, a complement receptor, CR3, CR4, Tim-1, Tim-4 and CD169. In some embodiments, the intracellular signaling domain comprises a pro-inflammatory signaling domain. In some embodiments, the intracellular signaling domain comprises a pro-inflammatory signaling domain that is not a PI3K recruitment domain.

In some embodiments, the intracellular signaling domain is derived from an ITAM domain containing receptor.

Provided herein is a composition comprising a recombinant nucleic acid encoding a CFP, such as a phagocytic or tethering receptor (PR) fusion protein (PFP), comprising: a PR subunit comprising: a transmembrane domain, and an intracellular domain comprising an intracellular signaling domain; and an extracellular domain comprising an antigen binding domain specific to an antigen of a target cell; wherein the transmembrane domain and the extracellular domain are operatively linked; and wherein the intracellular signaling domain is derived from a phagocytic receptor other than a phagocytic receptor selected from Megf10, MerTk, FcRα, or Bail.

In some embodiments, upon binding of the CFP to the antigen of the target cell, the killing activity of a cell expressing the CFP is increased by at least greater than 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 100%, 150%, 200%, 250%, 300%, 350%, 400%, 450%, 500%, 550%, 600%, 650%, 700%, 750%, 800%, 850%, 900%, 950%, or 1000% compared to a cell not expressing the CFP. In some embodiments, the CFP functionally incorporates into a cell membrane of a cell when the CFP is expressed in the cell. In some embodiments, upon binding of the CFP to the antigen of the target cell, the killing activity of a cell expressing the CFP is increased by at least 1.1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold,-fold, 17-fold, 18-fold, 19-fold, 20-fold, 25-fold, 30-fold, 40-fold, 50-fold, 75-fold, or 100-fold compared to a cell not expressing the CFP.

In some embodiments, the intracellular signaling domain is derived from a receptor, such as a phagocytic receptor, selected from the group consisting of TNFR1, MDA5, CD40, lectin, dectin 1, CD206, scavenger receptor A1 (SRA1), MARCO, CD36, CD163, MSR1, SCARA3, COLEC12, SCARA5, SCARB1, SCARB2, CD68, OLR1, SCARF1, SCARF2, CXCL16, STAB1, STAB2, SRCRB4D, SSC5D, CD205, CD207, CD209, RAGE, CD14, CD64, F4/80, CCR2, CX3CR1, CSF1R, Tie2, HuCRIg(L), CD64, CD32a, CD16a, CD89, Fcα receptor I, CR1, CD35, CD3ζ, CR3, CR4, Tim-1, Tim-4 and CD169. In some embodiments, the intracellular signaling domain comprises a pro-inflammatory signaling domain.

Provided herein is a composition comprising a recombinant nucleic acid encoding a CFP, such as a phagocytic or tethering receptor (PR) fusion protein (PFP), comprising: a PR subunit comprising: a transmembrane domain, and an intracellular domain comprising an intracellular signaling domain; and an extracellular domain comprising an antigen binding domain specific to an antigen of a target cell; wherein the transmembrane domain and the extracellular domain are operatively linked; and wherein the intracellular signaling domain is derived from a receptor, such as a phagocytic receptor, selected from the group consisting of TNFR1, MDA5, CD40, lectin, dectin 1, CD206, scavenger receptor A1 (SRA1), MARCO, CD36, CD163, MSR1, SCARA3, COLEC12, SCARA5, SCARB1, SCARB2, CD68, OLR1, SCARF1, SCARF2, CXCL16, STAB1, STAB2, SRCRB4D, SSC5D, CD205, CD207, CD209, RAGE, CD14, CD64, F4/80, CCR2, CX3CR1, CSF1R, Tie2, HuCRIg(L), CD64, CD32a, CD16a, CD89, Fc receptor I, CR1, CD35, CD3ζ, CR3, CR4, Tim-1, Tim-4 and CD169.

In some embodiments, upon binding of the CFP to the antigen of the target cell, the killing activity of a cell expressing the CFP is increased by at least greater than 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 100%, 150%, 200%, 250%, 300%, 350%, 400%, 450%, 500%, 550%, 600%, 650%, 700%, 750%, 800%, 850%, 900%, 950%, or 1000% compared to a cell not expressing the CFP. In some embodiments, the intracellular signaling domain is derived from a phagocytic receptor other than a phagocytic receptor selected from Megf10, MerTk, FcRα, or Bail. In some embodiments, the intracellular signaling domain comprises a pro-inflammatory signaling domain. In some embodiments, the intracellular signaling domain comprises a PI3K recruitment domain, such as a PI3K recruitment domain derived from CD19. In some embodiments, the intracellular signaling domain comprises a pro-inflammatory signaling domain that is not a PI3K recruitment domain.

In some embodiments, a cell expressing the CFP exhibits an increase in phagocytosis of a target cell expressing the antigen compared to a cell not expressing the CFP. In some embodiments, a cell expressing the CFP exhibits at least a 1.1-fold increase in phagocytosis of a target cell expressing the antigen compared to a cell not expressing the CFP. In some embodiments, a cell expressing the CFP exhibits at least a 2-fold, 3-fold, 4-fold, 5-fold, 6-fold, 7-fold, 8-fold, 9-fold, 10-fold, 20-fold, 30-fold or 50-fold increase in phagocytosis of a target cell expressing the antigen compared to a cell not expressing the CFP. In some embodiments, a cell expressing the CFP exhibits an increase in production of a cytokine compared to a cell not expressing the CFP. In some embodiments, the cytokine is selected from the group consisting of IL-1, IL3, IL-6, IL-12, IL-13, IL-23, TNF, CCL2, CXCL9, CXCL10, CXCL11, IL-18, IL-23, IL-27, CSF, MCSF, GMCSF, IL17, IP-10, RANTES, an interferon and combinations thereof. In some embodiments, a cell expressing the CFP exhibits an increase in effector activity compared to a cell not expressing the CFP. In some embodiments, a cell expressing the CFP exhibits an increase in cross-presentation compared to a cell not expressing the CFP. In some embodiments, a cell expressing the CFP exhibits an increase in expression of an MHC class II protein compared to a cell not expressing the CFP. In some embodiments, a cell expressing the CFP exhibits an increase in expression of CD80 compared to a cell not expressing the CFP. In some embodiments, a cell expressing the CFP exhibits an increase in expression of CD86 compared to a cell not expressing the CFP. In some embodiments, a cell expressing the CFP exhibits an increase in expression of MHC class I protein compared to a cell not expressing the CFP. In some embodiments, a cell expressing the CFP exhibits an increase in expression of TRAIL/TNF Family death receptors compared to a cell not expressing the CFP. In some embodiments, a cell expressing the CFP exhibits an increase in expression of B7-H2 compared to a cell not expressing the CFP. In some embodiments, a cell expressing the CFP exhibits an increase in expression of LIGHT compared to a cell not expressing the CFP. In some embodiments, a cell expressing the CFP exhibits an increase in expression of HVEM compared to a cell not expressing the CFP. In some embodiments, a cell expressing the CFP exhibits an increase in expression of CD40 compared to a cell not expressing the CFP. In some embodiments, a cell expressing the CFP exhibits an increase in expression of TL1A compared to a cell not expressing the CFP. In some embodiments, a cell expressing the CFP exhibits an increase in expression of 41BBL compared to a cell not expressing the CFP. In some embodiments, a cell expressing the CFP exhibits an increase in expression of OX40L compared to a cell not expressing the CFP. In some embodiments, a cell expressing the CFP exhibits an increase in expression of GITRL death receptors compared to a cell not expressing the CFP. In some embodiments, a cell expressing the CFP exhibits an increase in expression of CD30L compared to a cell not expressing the CFP. In some embodiments, a cell expressing the CFP exhibits an increase in expression of TIM4 compared to a cell not expressing the CFP. In some embodiments, a cell expressing the CFP exhibits an increase in expression of TIM1 ligand compared to a cell not expressing the CFP. In some embodiments, a cell expressing the CFP exhibits an increase in expression of SLAM compared to a cell not expressing the CFP. In some embodiments, a cell expressing the CFP exhibits an increase in expression of CD48 compared to a cell not expressing the CFP. In some embodiments, a cell expressing the CFP exhibits an increase in expression of CD58 compared to a cell not expressing the CFP. In some embodiments, a cell expressing the CFP exhibits an increase in expression of CD155 compared to a cell not expressing the CFP. In some embodiments, a cell expressing the CFP exhibits an increase in expression of CD 112 compared to a cell not expressing the CFP. In some embodiments, a cell expressing the CFP exhibits an increase in expression of PDL1 compared to a cell not expressing the CFP. In some embodiments, a cell expressing the CFP exhibits an increase in expression of B7-DC compared to a cell not expressing the CFP. In some embodiments, a cell expressing the CFP exhibits an increase in respiratory burst compared to a cell not expressing the CFP. In some embodiments, a cell expressing the CFP exhibits an increase in ROS production compared to a cell not expressing the CFP. In some embodiments, a cell expressing the CFP exhibits an increase in iNOS production compared to a cell not expressing the CFP. In some embodiments, a cell expressing the CFP exhibits an increase in iNOS production compared to a cell not expressing the CFP. In some embodiments, a cell expressing the CFP exhibits an increase in extra-cellular vesicle production compared to a cell not expressing the CFP. In some embodiments, a cell expressing the CFP exhibits an increase in trogocytosis with a target cell expressing the antigen compared to a cell not expressing the CFP. In some embodiments, a cell expressing the CFP exhibits an increase in resistance to CD47 mediated inhibition of phagocytosis compared to a cell not expressing the CFP. In some embodiments, a cell expressing the CFP exhibits an increase in resistance to LILRB1 mediated inhibition of phagocytosis compared to a cell not expressing the CFP. In some embodiments, a cell expressing the CFP exhibits an increase in phosphatidylinositol 3,4,5-trisphosphate production.

Also provided herein is a pharmaceutical composition comprising a composition described herein, such as a recombinant nucleic acid described herein, a vector described herein, a polypeptide described herein or a cell described herein; and a pharmaceutically acceptable excipient. The engineered cell is a myeloid cell. In one aspect, a pharmaceutical composition is disclosed, comprising a recombinant nucleic acid of encoding or comprising any one of the sequences of SEQ ID NOs: 1-51 and 342, or a cell comprising the recombinant nucleic acid of encoding or comprising any one of the sequences of SEQ ID NOs: 1-51 and 342, or an engineered cell comprising the recombinant nucleic acid of encoding or comprising any one of the sequences of SEQ ID NOs: 1-340, and 342-345; and a pharmaceutically acceptable excipient. In one embodiment the cell is a myeloid cell. In one embodiment, the cell is a mammalian cell. In one embodiment the cell is a primary human cell. In one embodiment, the cell is a human primary immune cell. In some embodiments the cell is a precursor cell or a stem cell, or an undifferentiated cell. In some embodiments the cell is obtained from a biological sample of a human subject. In some embodiments the cell is isolated from a biological sample of a human subject, and is selected for a phenotype, such as expression of cell surface markers. In one embodiment, the isolated cell is a precursor cell, a precursor myeloid cell, a cell characterized as CD14+/CD16−.

In one embodiment, the isolated cell or the engineered cell is CD14+/CD16−.

In one aspect, a pharmaceutical composition comprises an engineered cell, wherein the engineered cell is CD14+/CD16−.

In one aspect the pharmaceutical composition comprises a population of cells wherein at least 50% of the cells are CD14+/CD16−, and less than 10% cells are dendritic cells. In one embodiment, the cells exhibit high expression of CCR2. In one embodiment, the cells do not exhibit tonal signaling and activation de novo, and exhibit M0, M1 or M2 differentiation upon activation.

Provided herein is a method of treating a cancer or a viral infection in a subject comprising: administering to the subject the pharmaceutical composition comprising the recombinant nucleic acid of encoding or comprising any one of the sequences of SEQ ID NOs: 1-340, and 342-345, or a cell comprising the recombinant nucleic acid of encoding or comprising any one of the sequences of SEQ ID NOs: 1-340, and 342-345, or an engineered cell comprising the recombinant nucleic acid of encoding or comprising any one of the sequences of SEQ ID Nos: 1-340, and 342-345; and a pharmaceutically acceptable excipient. In some embodiments, the pharmaceutical composition comprises a population of cells wherein at least 50% of the cells are CD14+/CD16−, with less than 10% cells that are dendritic cells; and the cells exhibit high expression of CCR2. In one embodiment, the cells do not exhibit tonal signaling and activation de novo, and exhibit M0, M1 or M2 differentiation upon activation. In some embodiments, the myeloid cell is a CD14+ cell, a CD14+/CD16− cell, a CD14+/CD16+ cell, a CD14−/CD16+ cell, CD14−/CD16− cell, a dendritic cell, an M0 macrophage, an M2 macrophage, an M1 macrophage or a mosaic myeloid cell/macrophage/dendritic cell.

### EXAMPLES

The present disclosure will be more specifically illustrated by the following Examples. However, it should be understood that the present disclosure is not limited by these examples in any manner.

### Example 1: VHH Binder Selection Process

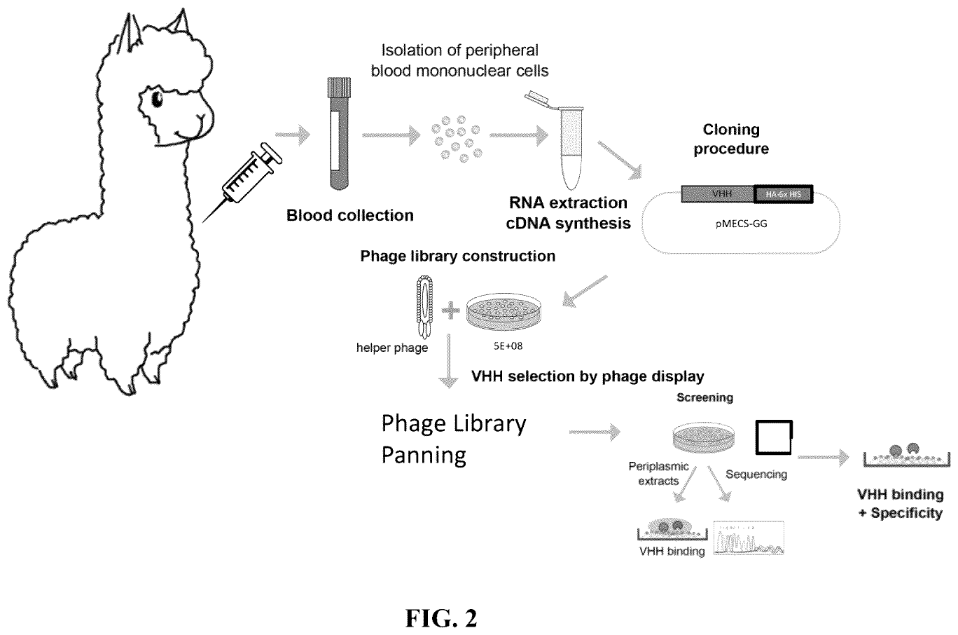

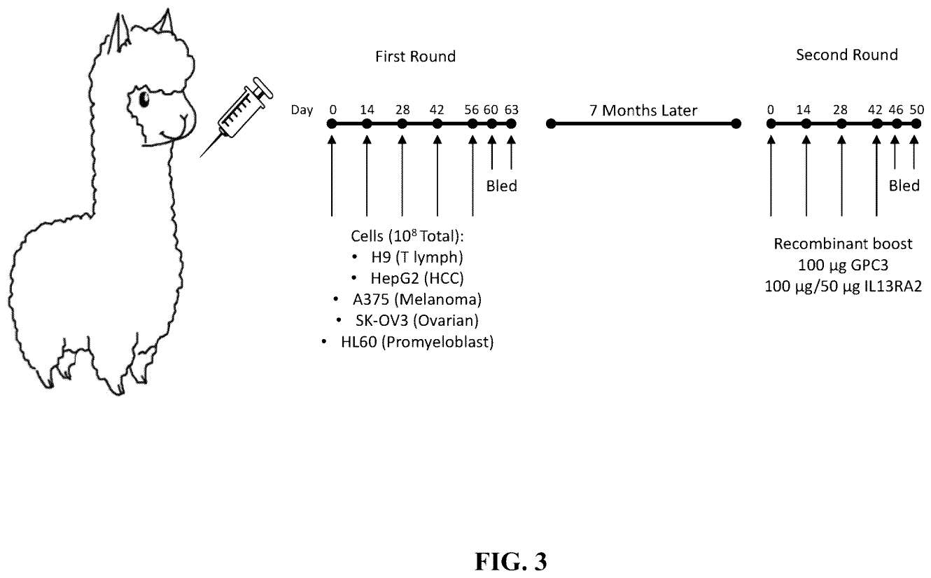

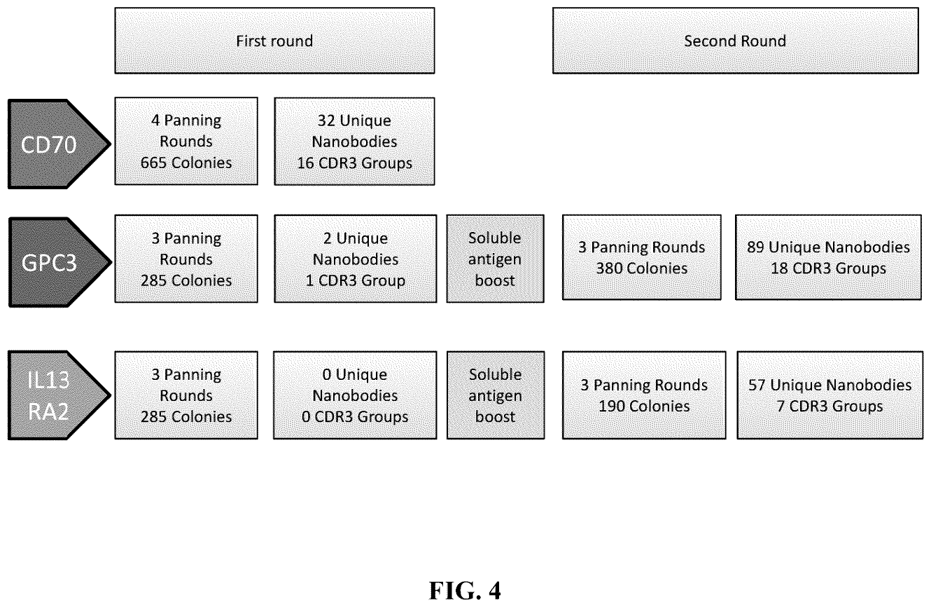

In the exemplary process described herein, identification and generation of specific binders for cancer targets, e.g., CD70 antigen, GPC3 antigen and IL13Rα2. In the method, llama are immunized with relevant cancer cell types that express CD70 antigen, GPC3 antigen and/or IL13Rα2. A total of 10\hat{ }8 cells, having T lymphoma cells from H9 cell line, hepatocarcinoma (HCC) cells from HepG2 cell line, A375 melanoma cells, SK-OV3 ovarian cancer cells and HL60 promyeloblast cells are injected in a llama at a time interval of 2 week for 5 times, at day 0, 14, 28, 42, 56. The animals are bled to collect peripheral blood on days 60 and 63. Seven months after day 56, the animals are given a boost with recombinant antigens, e.g., 100 ug GPC3 antigen or 100 ug (or 50 ug) IL13Rα2, as shown in FIG. 3. Upon collection of peripheral blood, RNA is extracted, and cDNA is synthesized from total blood. VHH sequences were cloned into a pMECS-GG plasmid and a phage library is constructed with a helper phage. VHH selection was done by phage display, screened and sequenced. The overview of this process is represented graphically in FIG. 2. An overview of the screen results is shown in FIG. 4.

Upon receiving the sequences, the binding domains were sought to improve, e.g., by substitutions in the framework regions and CDR analysis. CDR families were generated, and representative candidates were selected. Humanization and functional analysis were carried on. IgG1 fusions were generated and expressed in mammalian cell lines. Protein A mediated purification was performed.

For expressing GPC3 and IL13Rα2 binders, E. coli were transformed and 6×HIS (SEQ ID NO: 364) purified and processed. Affinity measurements and binning was performed.

For CD70 binders, FWR mutations were performed in order to humanize the llama WT sequences for H1 (SEQ ID NO: 1) and C1 (SEQ ID NO: 11). For H1, 14 mutations were necessary to attain 100% humanization of SEQ ID NO: 1. However, 4 were hall mark sequences. Therefore 95.6% was achieved at best with 10 mutations (SEQ ID NO: 10). H1 h10—95.6% human. The other respective clones and % humanization are as follows:
H1 h9—94.5% human
H1 h8—93.4% human
H1 h7—92.3% human
H1 h6—91.2% human
H1WT—84.6% human

For C1 (SEQ ID NO: 11, WT), similarly, due to the 4 hallmark residues, which cannot be mutated the final clone is 95.6% humanized (C1, SEQ ID NO: 342). Intermediate clones are as follows:
C1 h10—95.6% human
C1 h9—94.5% human
C1 h8—93.4% human
C1 h7—92.3% human
C1 h6—91.2% human
C1 h5—90.1% human
C1 WT—84.6% human

### Example 2. Anti-CD70 Binding VH Domain Expression and Binding Kinetics

### Expression Analysis

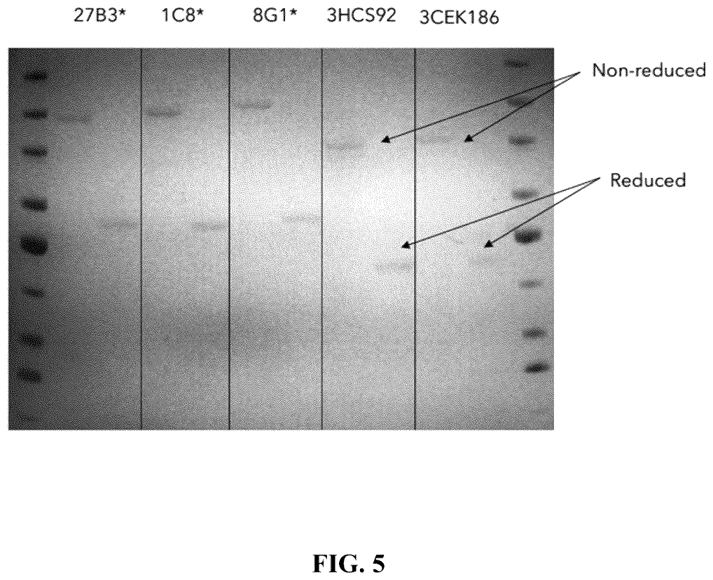

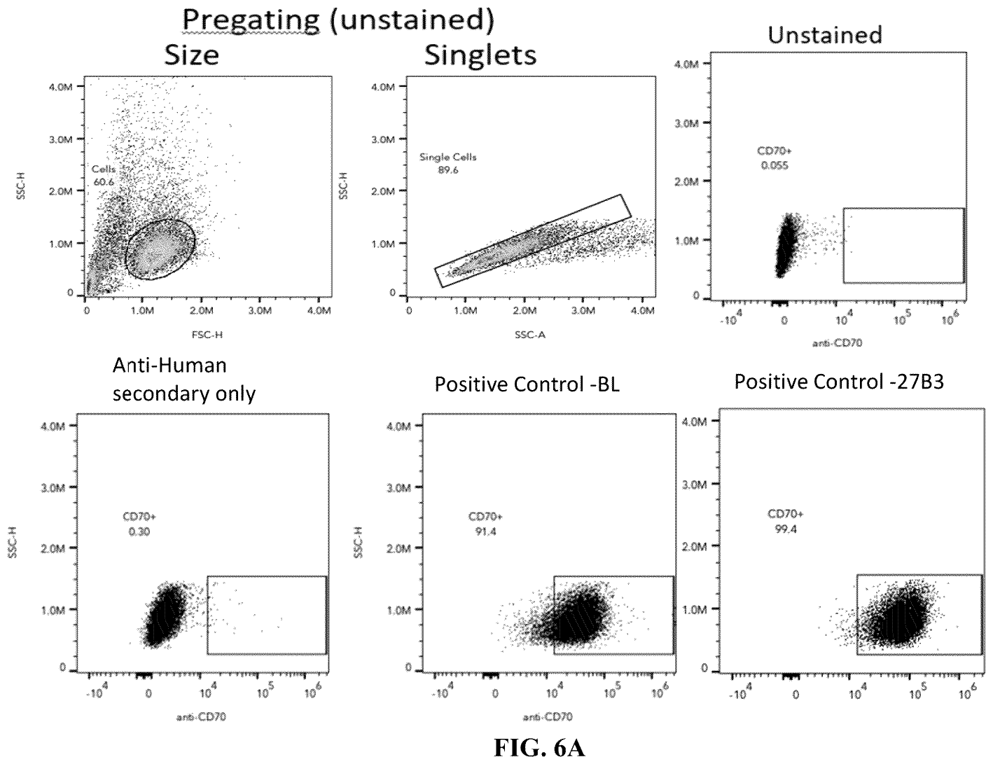

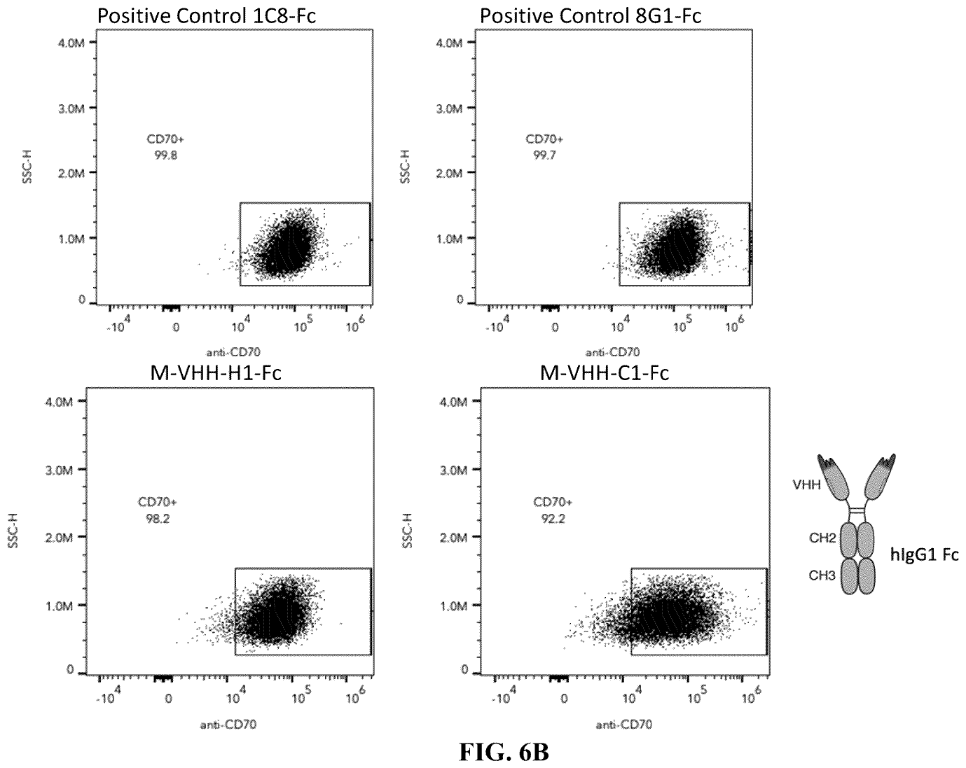

In this example, anti-CD70 binders were generated following the process described above. For some purposes CD70 binders were generated as an Fc fusion construct, exemplified in FIG. 6B (bottom, right). The Fc fusion allows stabilization and dimerization for binding studies. Expression and purification was verified by electrophoresing the purified proteins and as demonstrated in FIG. 5 single bands were obtained for reduced and non-reduced proteins for H1 and C1 constructs (two samples on the right most columns). FIGS. 6A and 6B show flow cytometry data for expression.

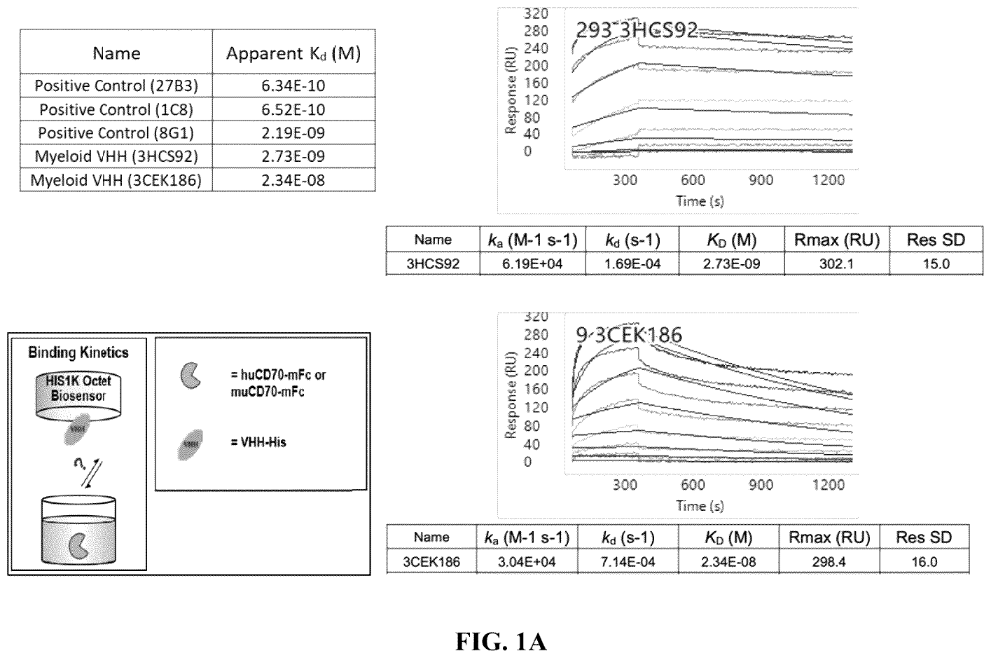

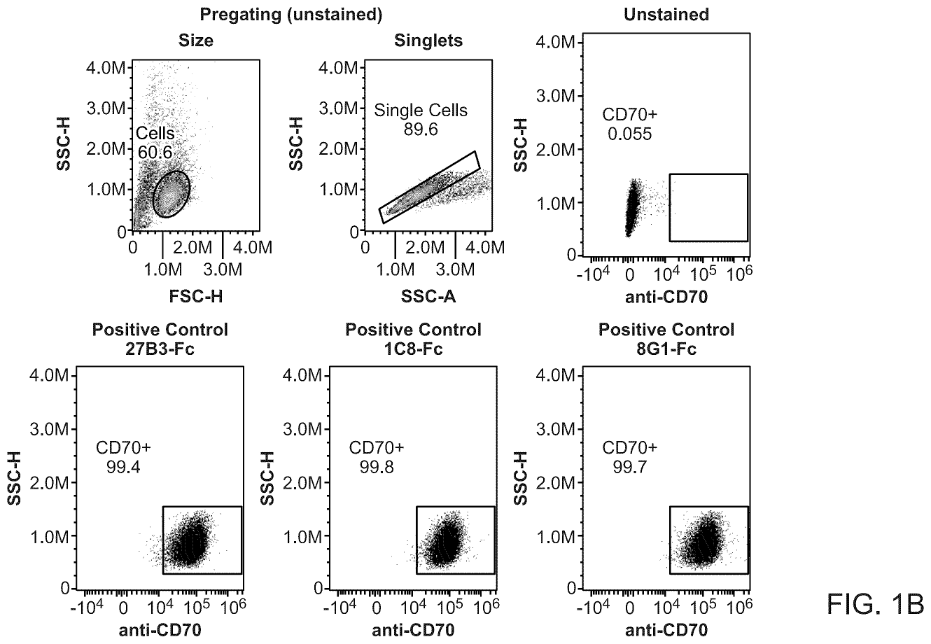

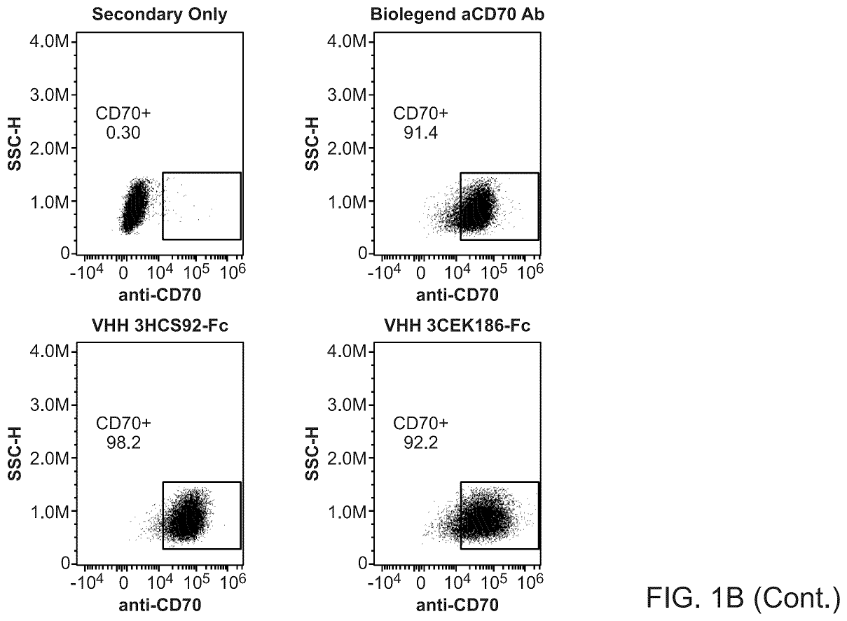

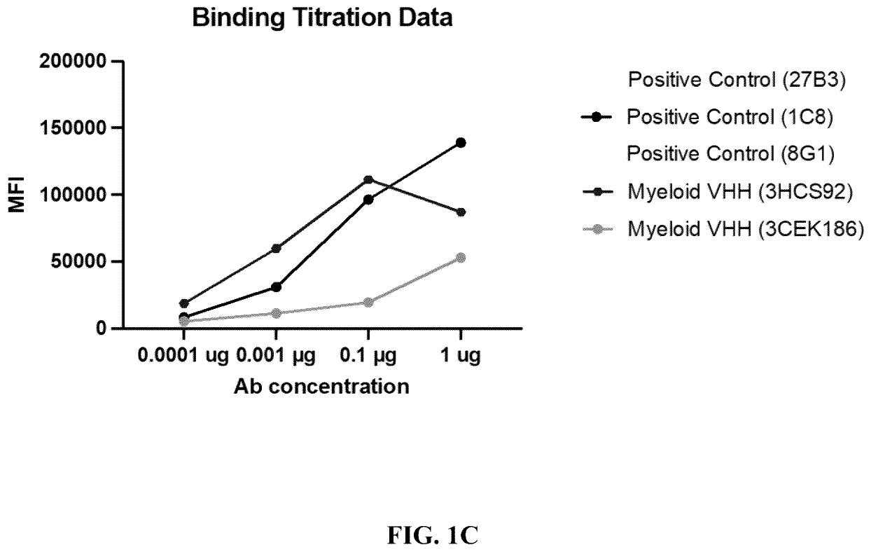

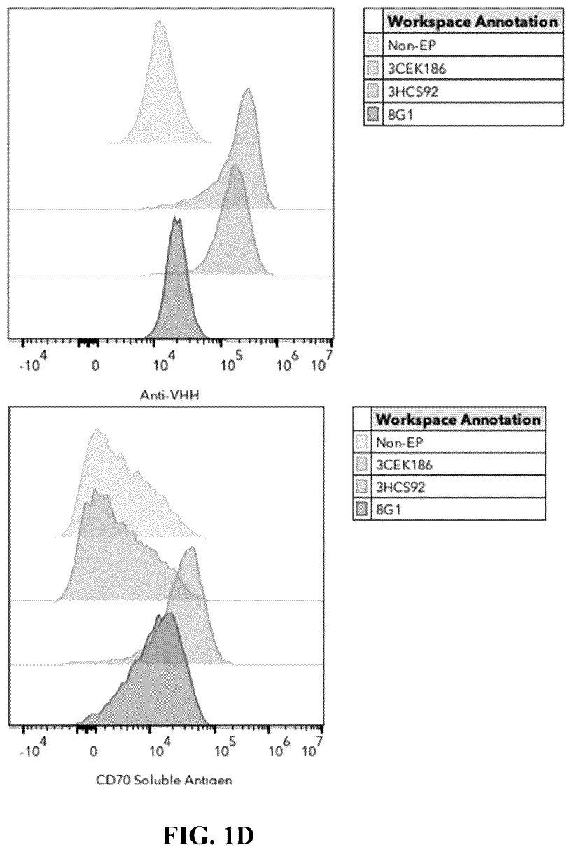

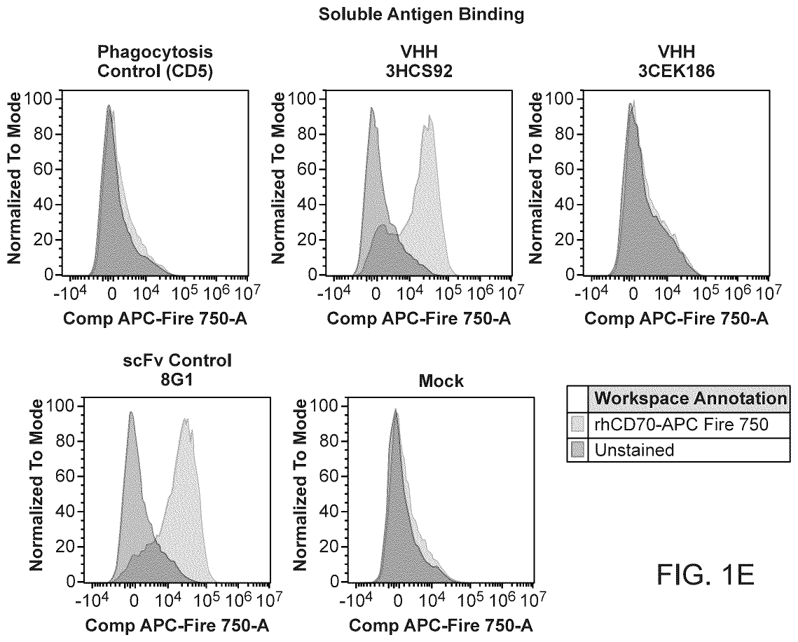

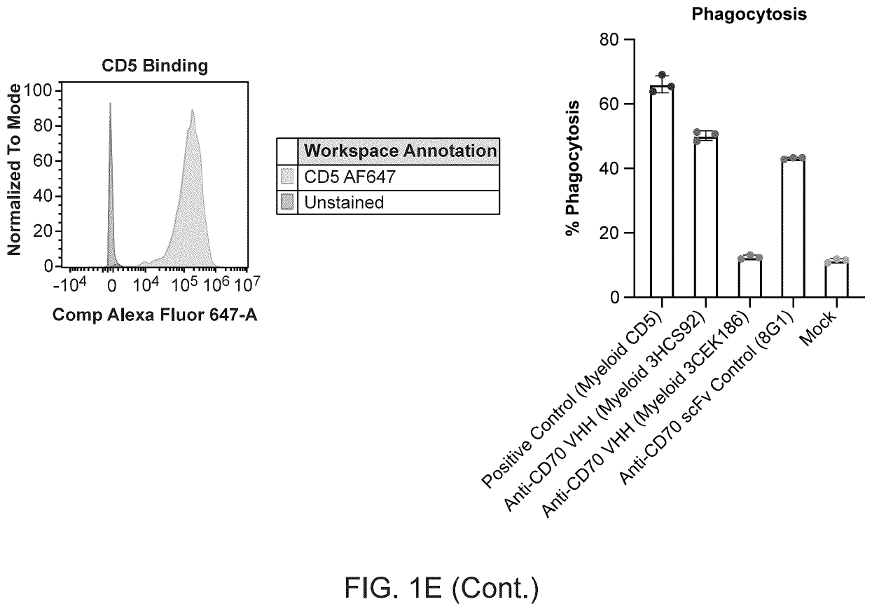

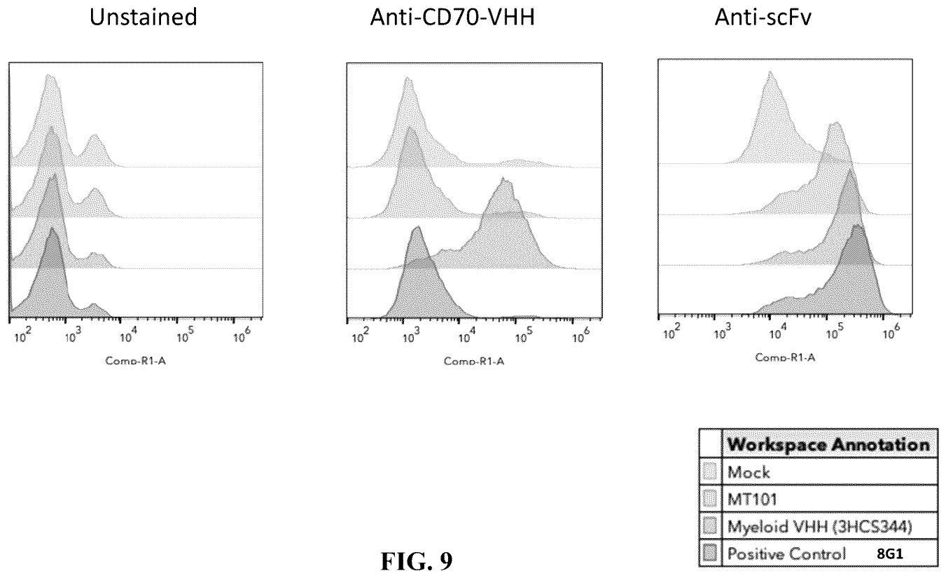

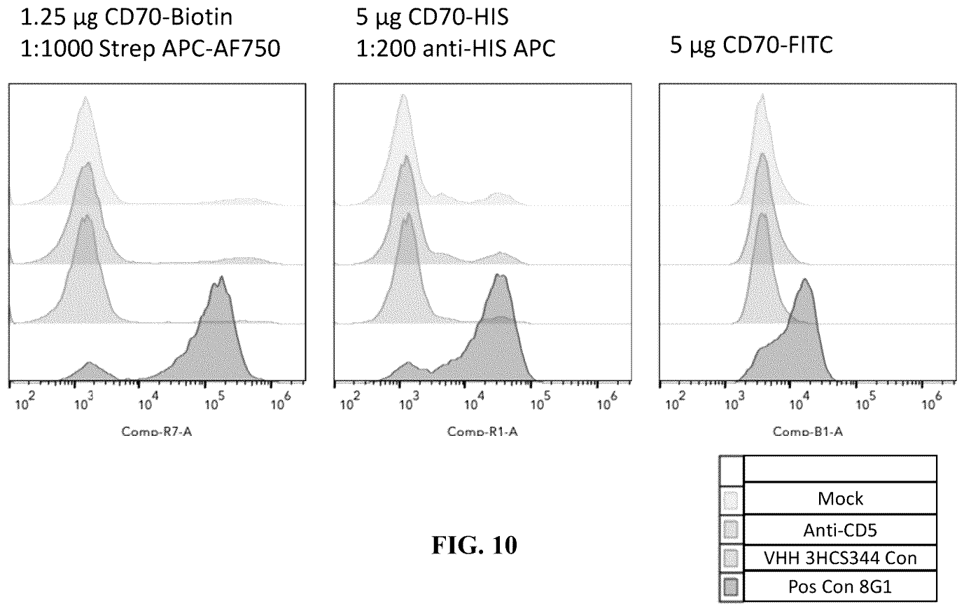

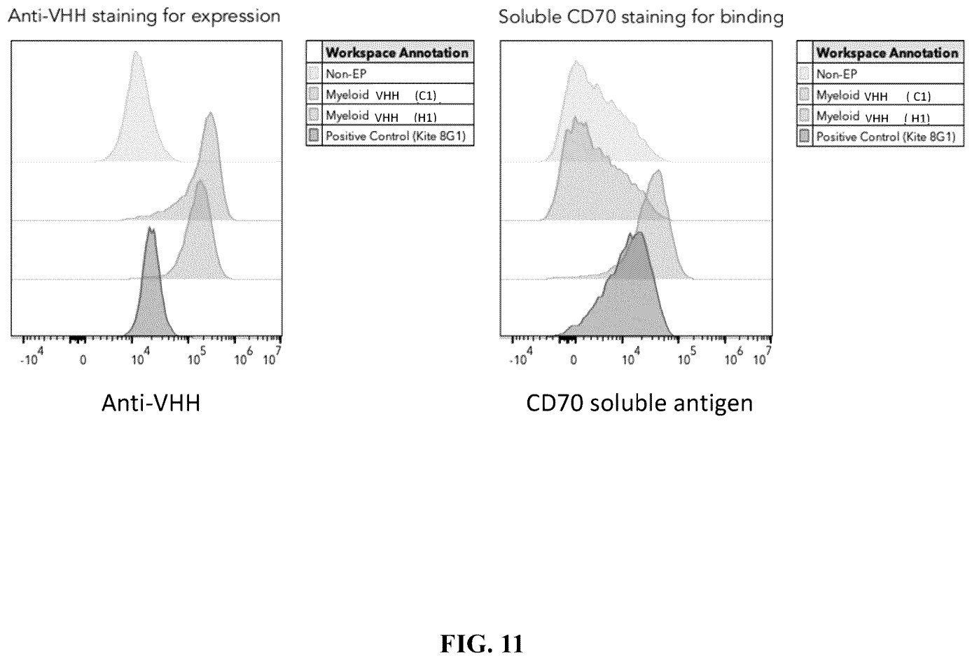

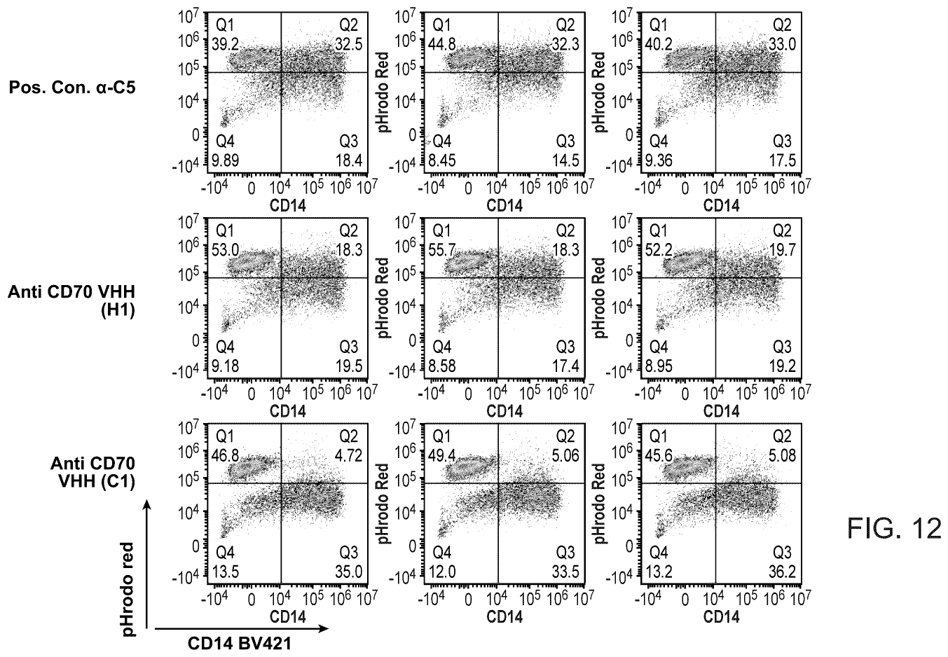

Commercial antibodies against VHH and scFv were tested for detection of VHH or ScFv without cross reactivity to the other (FIG. 9); it was found that anti-VHH antibody did not show any cross reactivity but the anti-scFv did. (FIG. 9). Anti-CD70 staining was tested (FIG. 10). Using anti-VHH staining, as well as with soluble CD70 staining, expression of H1 VHH and H1 CAR were readily detectable, whereas C1 constructs were poorly detected (FIG. 1D, FIG. 11). The anti-CD70 VHH construct 3HCS92 (henceforth H1) shows expression by anti-VHH and soluble antigen binding. The anti-CD70 3CEK 186 (henceforth C1) shows expression by anti-VHH but not with soluble antigen binding.

### Affinity Measurements

Next the polypeptides were characterized for CD70 binding kinetics, e.g. by affinity measurements. An affinity measurement method used in this study is illustrated in FIG. 1A, left bottom graphic image. Briefly, His-tagged VHH constructs were individually captured on HIS1K biosensors. VHH-captured biosensors were dipped in wells containing antigen and binding responses were measured. Biosensors were later dipped in buffer wells to observe the dissociation of bound antigen from the VHHs. Biosensors were regenerated and similar binding kinetic assays were performed for subsequent antigens. Exemplary data of the binding kinetics of anti-CD70 binding domains binding kinetics is shown in FIG. 1A. Single point K_{D} analysis using BioLayer interferometry with a soluble VHH format was undertaken. Exemplary results of the study are presented in the Table 6 below:

TABLE 6

| | | | Human CD70 mFc (1 uM) | | | | | | Mouse CD70 mFc (1 uM) | | | | | |
|---|---|---|---|---|---|---|---|---|---|---|---|---|---|---|
| | | mAb | Binding | | | | | | Binding | | | | | |
| | | Capture | Re- | | | | | | Re- | | | | | |
| | Clone | level | sponse | Kₐ | K_{d} | KD | t½ | | sponse | Kₐ | K_{d} | KD | t½ | |
| mAb# | ID | (nm^{s}) | (nm^{s}) | (1/Mₛ) | (1/s) * | (M) | (min) | R² | (nm^{s}) | (1/Ms) | (1/s) * | (M) | (min) | R² |
| HBS-BNT | buffer | 0.01 | 0.01^{$} | N.B. | N.B | N.B | N.B. | N.B. | −0.02^{$} | | | | | |
| mAb-01 | H1 | 0.28 | 0.87 | 7.13E+04 | 3.77E−04 | 5.29E−09 | 31 | 0.98 | 0.03^{$} | 1.28E+03 | 4.27E−04 | 3.34E−07 | 27 | .96 |
| | (WT) | | | | | | | | | | | | | |
| mAb-08 | C1 | 0.57 | 0.01^{$} | N.B. | N.B | N.B | N.B. | N.B. | 0.01^{$} | | | | | |
| | (WT) | | | | | | | | | | | | | |
| b- | GPC5 | 0.35 | 0.01^{$} | N.B. | N.B | N.B | N.B. | N.B. | 0.00^{$} | | | | | |
| 01MY2202 | CDR3 | | | | | | | | | | | | | |
| | f1 | | | | | | | | | | | | | |

Table cells marked with ^{$}indicated they have been fluffed for low binding. N.B=no binding. Carterra K_{D} analysis of the VHH-Fc fusion constructs are tabulated, shown in FIG. 1A (upper left). The 3HCS92 VHH (H1, binding domain of SEQ ID NO: 1) clone exhibits low cross-reactivity to mouse CD70. The affinity of 3CEK186 VHH monomer (C1, binding domain of SEQ ID NO: 11), could not be measured by single point K_{D} method. As Fc fusion, the affinity values were in the lower nanomolar range, as measured by Carterra, indicating high affinity.

### Example 3: Anti-CD70 Binding Domain Binding Data to H9 Parental Cells

Exemplary binding data of anti-CD70 binding domains to H9 parental is shown in FIG. 1B, FIGS. 6A and 6B. In this study, H9 cells were treated with 1 μg VHH-Fc (unconjugated), (Cells were resuspended in 100 μl of FACS buffer and 1 μg of unconjugated biologics was added to each well (wells A-E) or nothing was added (wells F-H). 1 mL of 1:1000 dilution of goat anti-human IgG AF647 was prepared. Wells A-E and G were resuspended in 100 μl in secondary staining reagent. Wells F and H were resuspended in 100 μl FACS buffer. Well H had 1 μl of anti-human CD70 APC added. The staining plate was incubated on ice for 20 minutes. 100 μl FACS buffer was added to each well and the plate was spun at 600×g for 2 minutes. The supernatant was discarded after spinning. Cells were resuspended in 200 μl of FACS buffer and run on Cytek instrument.

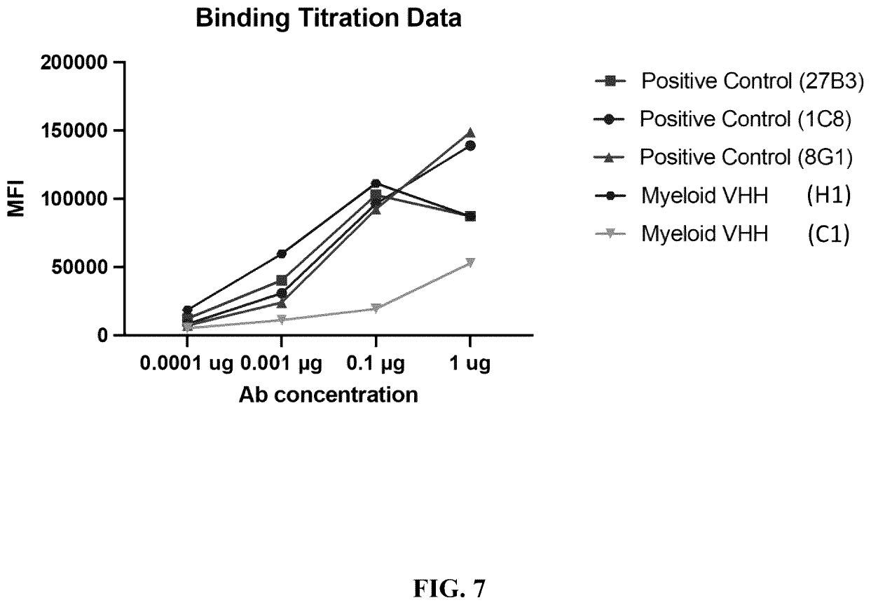

Exemplary titration data of anti-CD70 binding domains is shown in FIG. 1C and FIG. 7.

### Example 4: Phagocytosis of Cancer Cells and Cytokine Profile of the Anti-CD70 CAR Monocyte Constructs

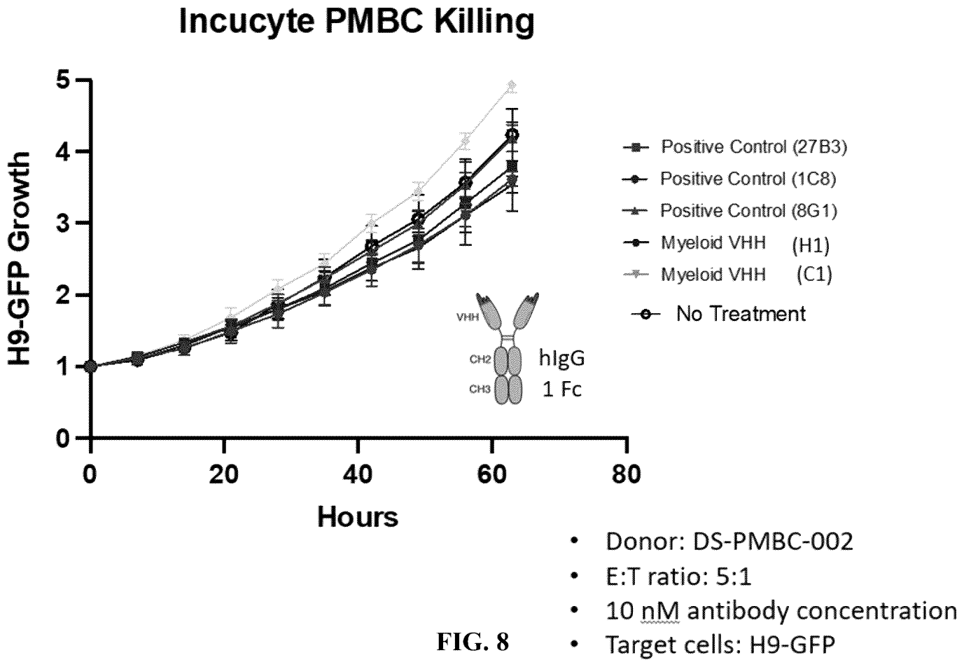

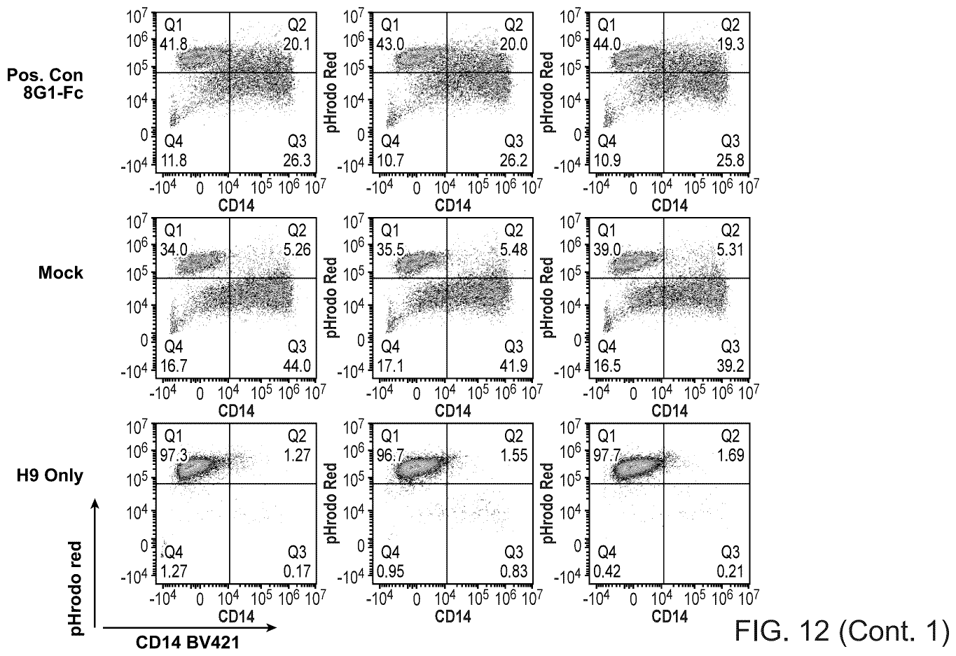

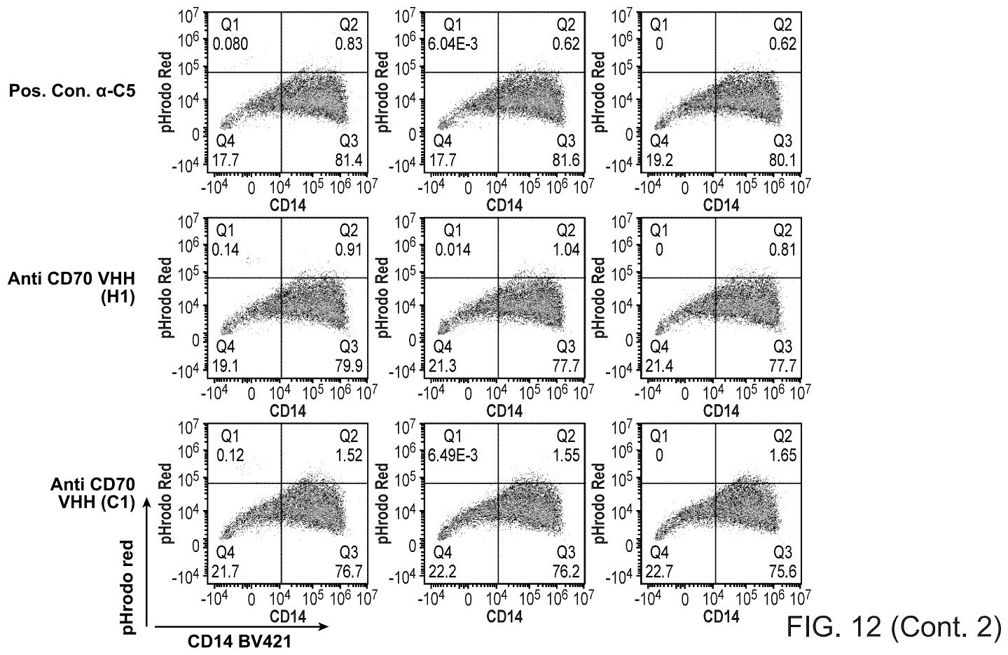

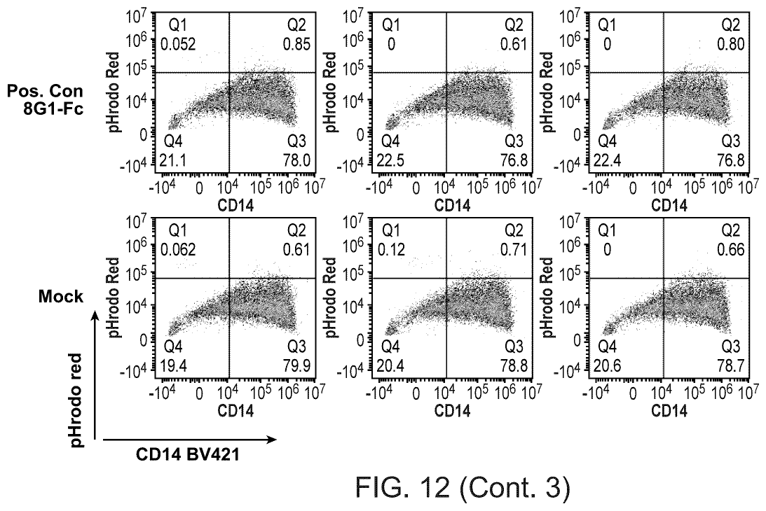

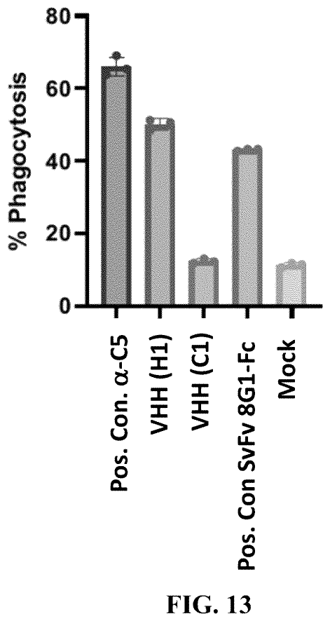

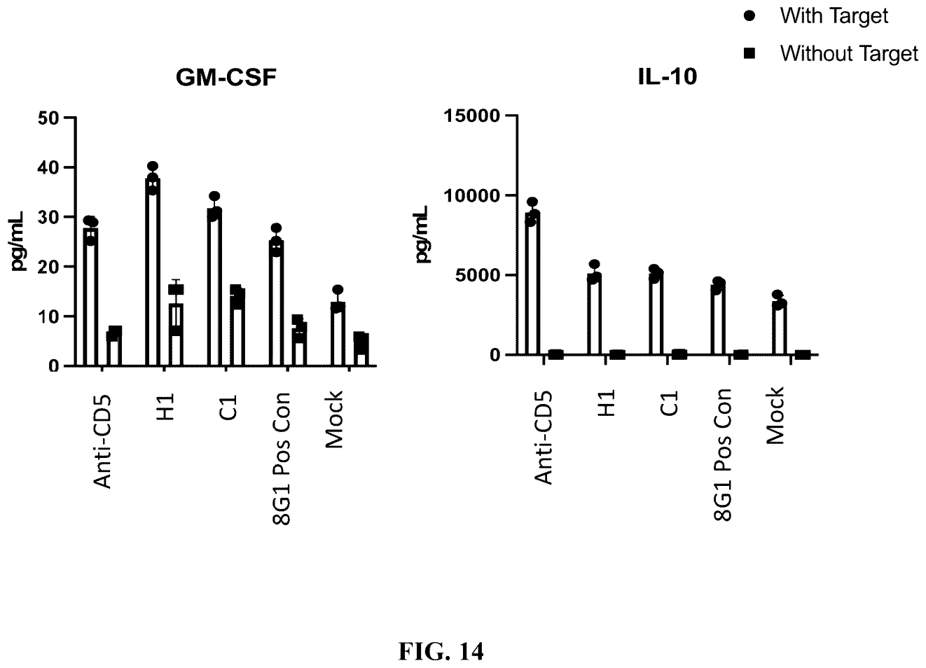

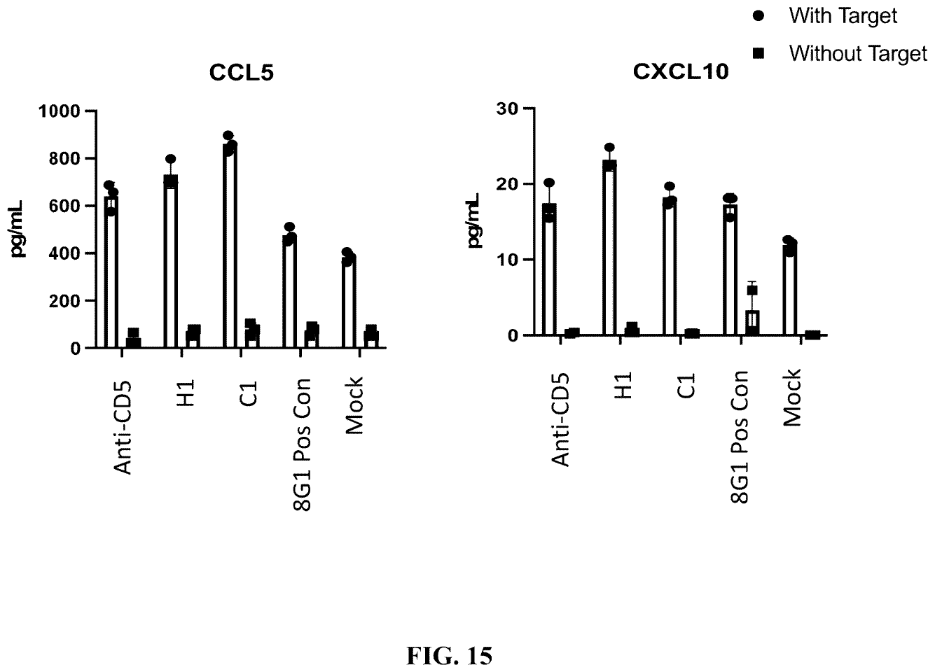

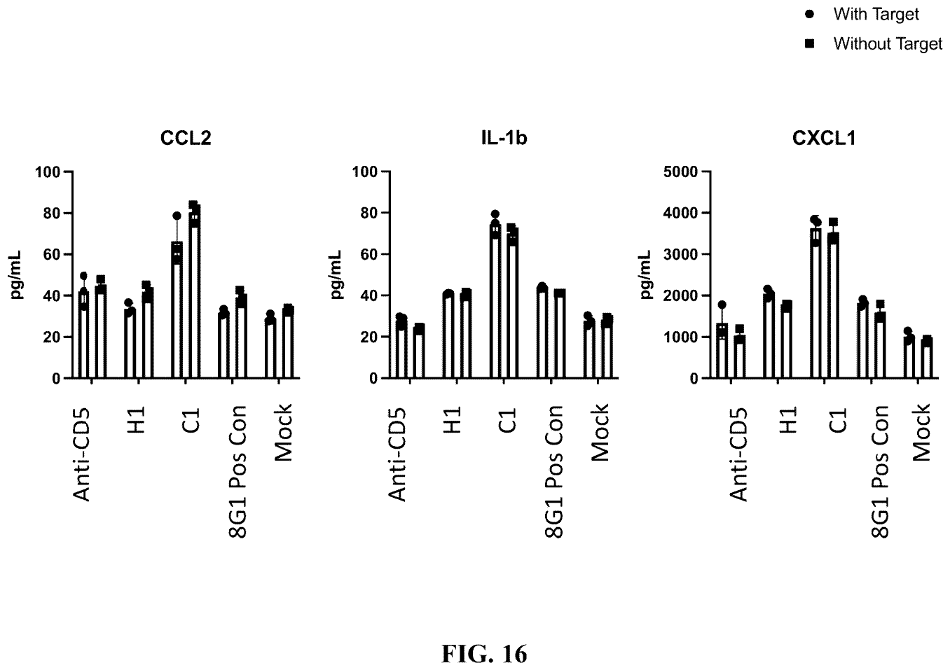

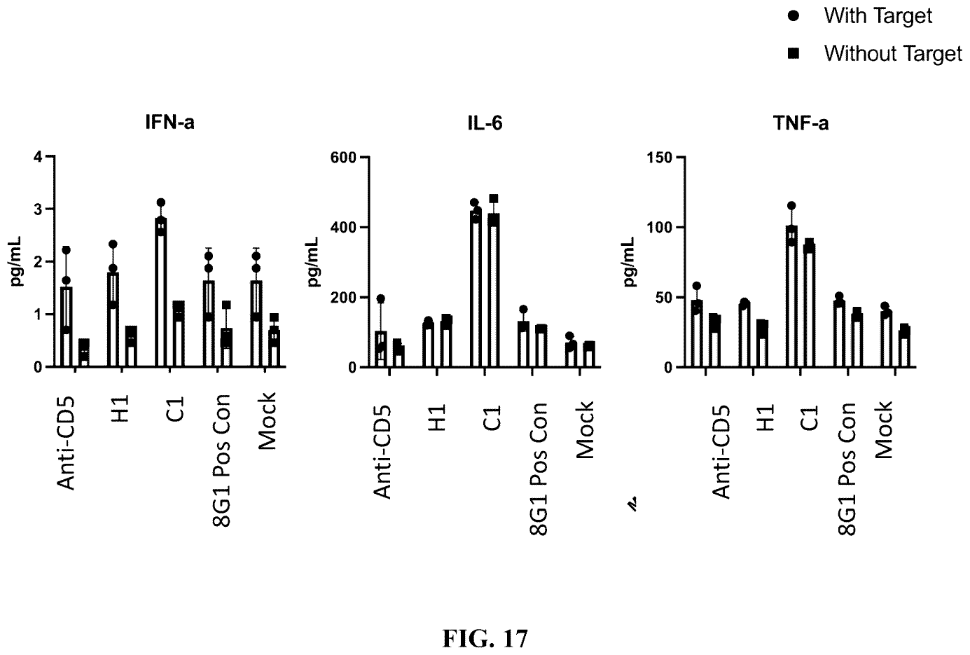

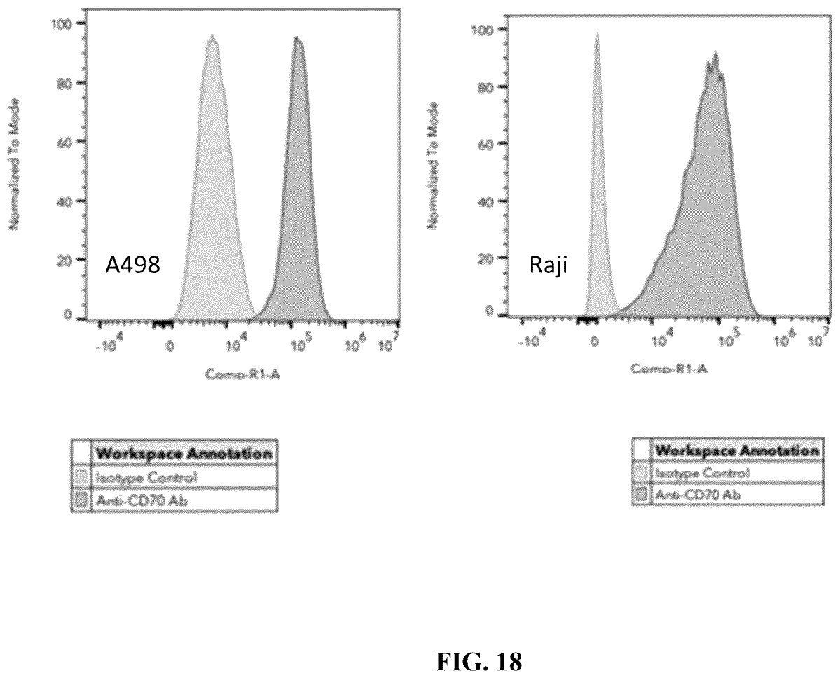

V_{H} sequences having SEQ ID NOs: 22-112 on Table 3 were obtained by performing immunization and screening as described in the previous section. Exemplary data showing phagocytosis of cancer cells with anti-CD70 CAR monocyte constructs is shown in FIG. 1E, FIG. 8 and FIG. 12. FIG. 8 depicts Incucyte PBMC killing using donor PBMCs. Target cells are H9-GFP+ cells. For in vitro killing assay, as a way to understand whether the binders (e.g. VHH-Fc fusion nanobodies) are capable of eliciting anti-cancer response, the test conditions were set to have effector to target cell ratio of 5:1, with an antibody concentration of 10 nM. Assaying was done at 72 hours post incubation and the readout is via Incucyte instrument. The flow cytometry data is summarized in FIG. 13. Surprisingly, the nanobodies induced enhanced killings, and H1 construct even showed higher phagocytosis than the positive control. Without wishing to be bound by any theories, it may be possible that VHHs have higher potency in activating myeloid cells even if they lack myeloid cell activating intracellular domains such as TLR, or TRIP of kinase recruiting domains. Cytokine and chemokine release assay results are shown in FIGS. 14-17, indicating cells are activated. Again H1 showed remarkable activity compared to the other and even compared to the commercial scFv 8C1 positive control. FIG. 18 shows expression of CD70 in different cancer cell lines indicating that different cancers could be the relevant indications for anti-CD70 therapeutics such as the biologics described herein.

### Example 5: Generation and Expression of GPC3 Binding VHH

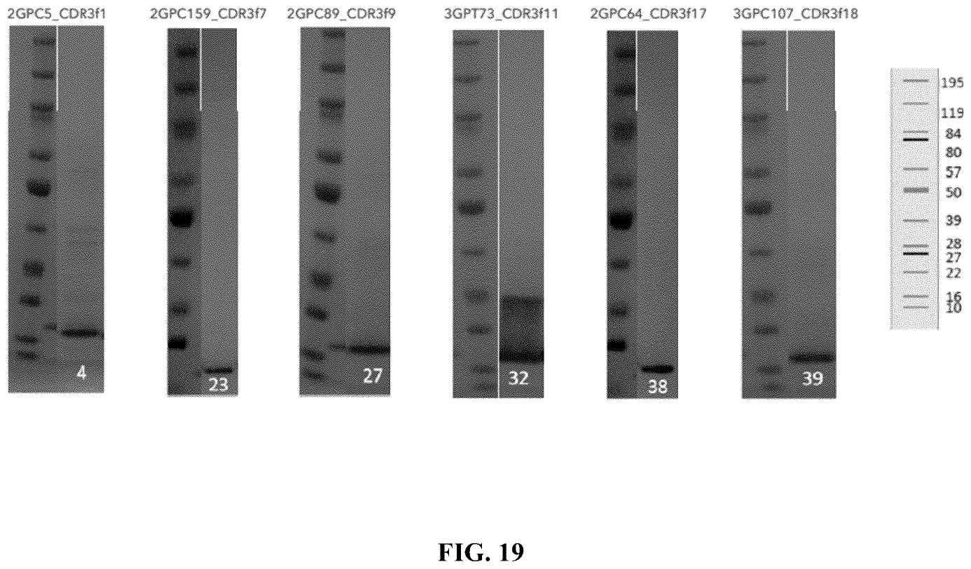

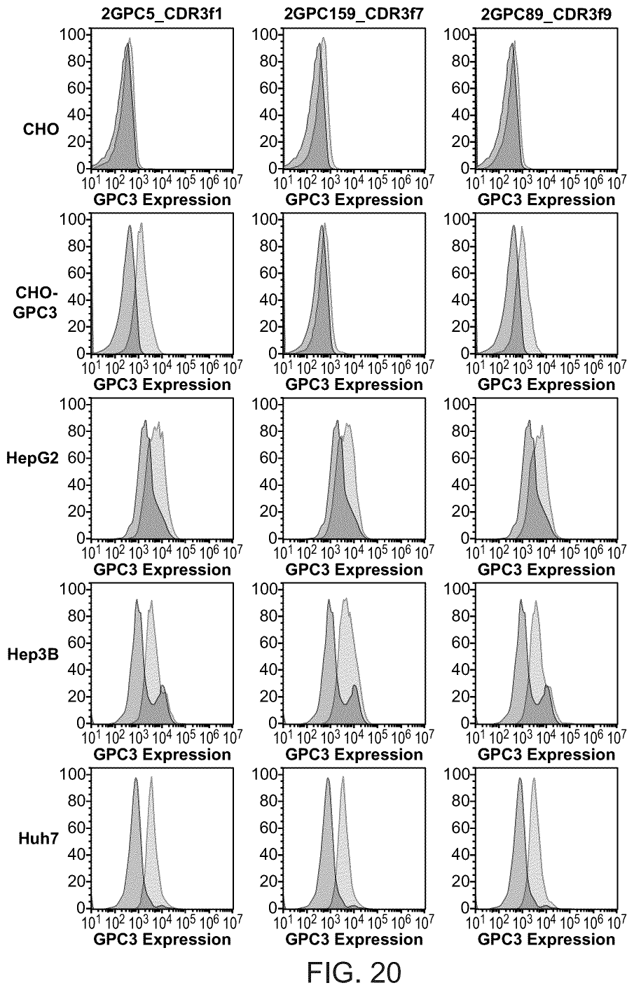

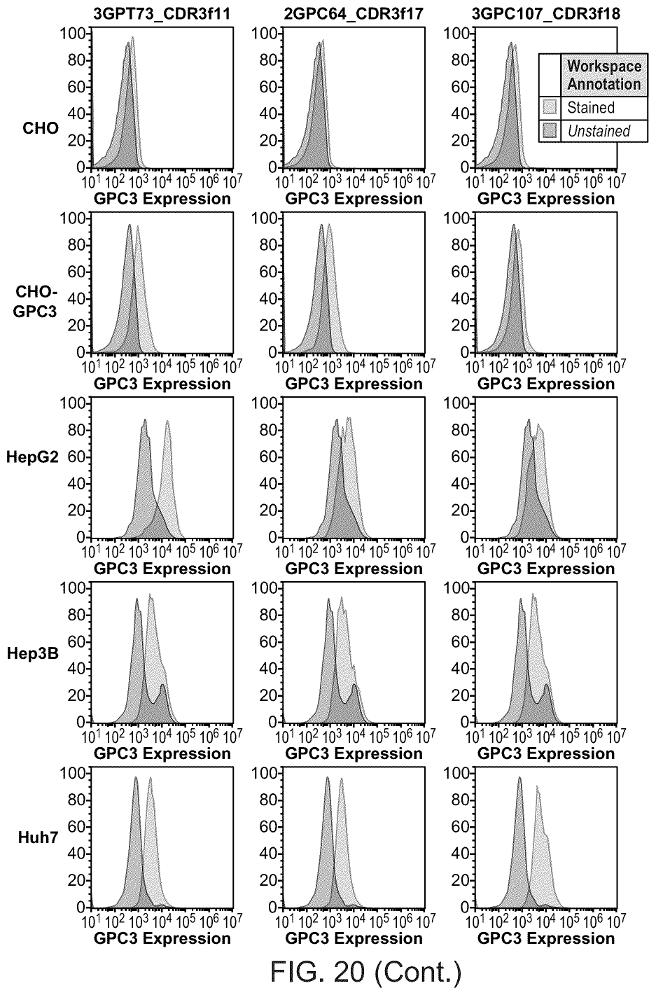

Generation of anti-GPC3 VHHs were as described for CD70 binders. Of the multitude clones the following were tested (Table 7) as representative species. Gel electrophoresis shows purified constructs are shown in FIG. 19. Binding to GPC3 on cells is shown in FIG. 20. Table 7 shows sequences of the 6 VHH constructs CDR1, 2, and 3 are underlined respectively from left to right. The designations are made following IMGT numbering scheme.

TABLE 7
Anti-GPC3 VHH domains further analyzed.

| | | SEQ ID |
|---|---|---|
| Name | Sequence | NO |
| G1f1 | QVQLQESGGGLVQPGGSLRLSCAASGFPLAYYAIGWFRQAPGK | 33 |
| | EREGVSCISASDGSTYYADSVKGRFTISRDNAKNTVYLQMNSLR | |
| | PEDTAVYYCATACADTTLYEYDYWGQGTQVTVSS | |
| G2f7 | QVQLQESGGGLVQPGGSLRLSCAASGFTLGYYAIGWFRQAPGK | 80 |
| | EREGVSCISSSDGSTYYADSVKGRFTISRDNAKNTVYLQMNSLK | |
| | PEDTAVYYCATACANWSSLGPYDYWGQGTQVTVSS | |
| G3f9 | QVQLQESGGGLVQPGGSLRLSCAASGFPLNYYAIGWFRQAPGK | 55 |
| | EREGVSCISASDGNTYYADSVKGRFTISRDNAKNTVYLQMNSLK | |
| | PEDTAVYYCATTCASPEKYEYDYWGQGTQVTVSS | |
| G4f11 | QVQLQESGGGVVQAGGSLKLSCAASGSDFRADAMGWYRQAPG | 108 |
| | KEREPVAIDSITSIYYVDSVEGRFTISRDNTKNTVYLQMTSLKPE | |
| | DTAVYYCYARYSGRTYWGRGTQVTVSS | |
| G5f17 | QVQLQESGGGLVQPGGSLRLSCAASGFSLAYYAIGWFRQAPGK | 58 |
| | EREGVSCIAASVGSTYYADSVKGRFTISRDDAKNTVYLQMNSLK | |
| | PEDTAVYYCATDCAGGVGHEYDYWGQGTQVTVSS | |
| G6f18 | QVQLQESGGGLVQPGGSLRLSCAASGFPLDYYAIGWFRQAPGK | 47 |
| | EREGVSCISSSDGSDGNTYYADSVKGRFTISRDNAKNTVYLQMN | |
| | SLKPEDTAVYYCATDCSLHGSDYPYDYWGQGTQVTVSS | |

### Example 6. GPC3 Binding Affinity and Binning Date

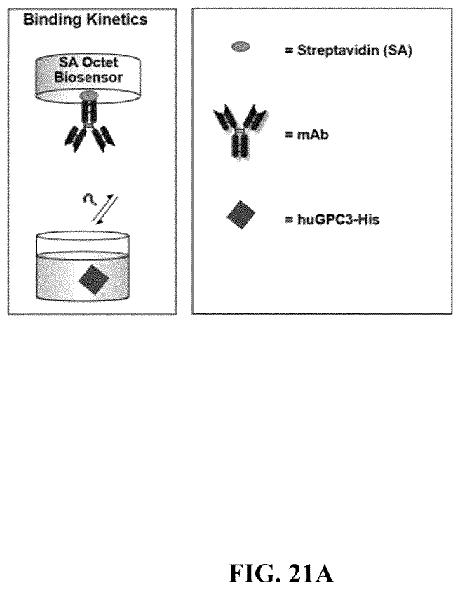

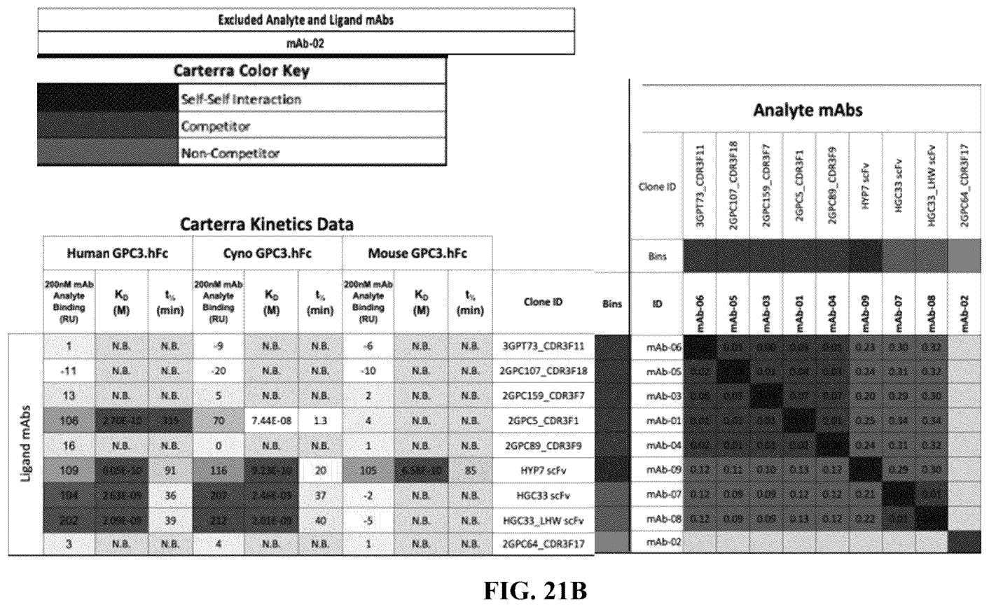

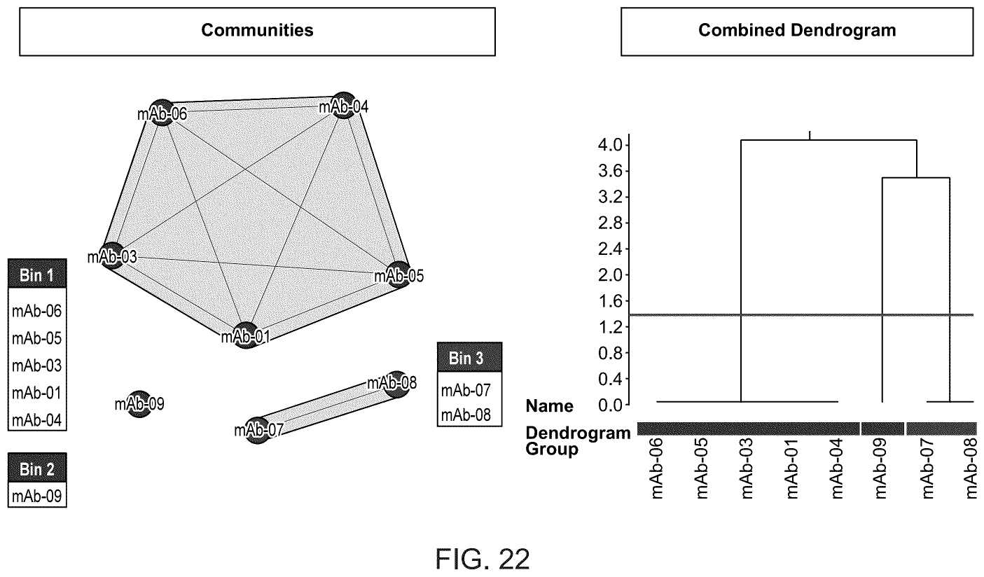

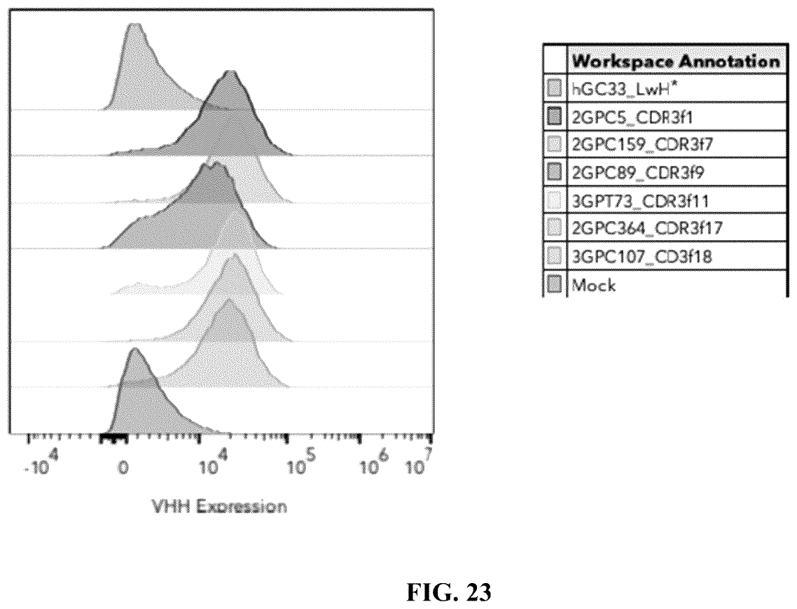

The assay method for affinity assay used is briefly: Biotinylated VHHs were individually captured on Streptavidin biosensors. VHH-captured biosensors were later dipped in wells containing antigen and the binding response was measured. Biosensors were later dipped in buffer wells to observe the dissociation of bound antigen from captured VHHs. Biosensors were regenerated and similar binding kinetic assays were performed for subsequent antigens. (FIG. 21A). Six GPC3 clones that were analzyed were binned as shown in FIGS. 21 and 22. Five of the six studied VHHs belong to the same bin and target the same epitope on GPC3. They do not share bin with the reference scFvs. FIG. 23 shows expression of the clones. All six VHH CARs can be detected on the surface of primary monocytes.

### Example 7. Analysis of IL13Ra2 Binders

Anti-IL13Rα2 V_{H} sequences having SEQ ID NOs:195-252 on Table 5 were obtained using the method described above. Of these, the following were analyzed as representative species, shown in Table 8 which shows sequences of the 6 VHH constructs CDR1, 2, and 3 are underlined respectively from left to right. The designations are made following IMGT numbering scheme.

TABLE 8
Anti-IL13Rα2 binding VHH domains further analyzed.

| | | SEQ |
|---|---|---|
| Name | Sequence | ID NO |
| VH3 | QVQLQESGGGSVQPGGSLRLSCAASGINISSDVMEWFRQGPGEQRE | 230 |
| 6f1 | LVAHITTNSRTEYADFVKGRFTISRANAKNTLYLQMNGLKPEDTAL | |
| | YYCALRRGGRILWGQGTQVTVSS | |
| VH4 | QVQLQESGGGLVQPGGSLRLSCTASGSIRGIGYMAWGRQGPGKPRELIA | 237 |
| 3f2 | TINSAGDTNYAESVKGRFTVSRDNAENTLYLQMNNLKSEDTAVYYCNA | |
| | RPGLOSYWGQGTQVTVSS | |
| VH5 | QVQLQESGGGLVQPGGSLRLSCEVSGSIFSINRMAWYRQVPEKEREMV | 246 |
| 2f3 | AQITYGDTIKYYGSAKGRFTISRDSAKNAVYLQMNSLKPEDTAVYRCNI | |
| | VGVLTTGHYEDSFWGQGTQVTVPS | |
| VH5 | QVQLQESGGGLVQPGGSLRLSCEVSGSISSINRMAWYRQVPGKEREMV | 247 |
| 3f3 | AQITYGDTIKYYGSAKGRFTISKDSAKNAVYLQMNSLKPEDTAVYRCNI | |
| | VGVLTTGHYENSFWGQGTQVTVSS | |
| VH5 | QVQLQESGGGLVQPGGSLRLSCAASGSIFNTNGMGWYRQAPGKQRERV | 251 |
| 7f5 | AGISSGGNTVYAASVEGRFTISRDNAKNTGYLQMNNLKPEDTAVYYCR | |
| | VAGRODDYWGQGTQVTVSS | |
| IL13Ra2_ | QVQLQESGGGLVQPGGSLRLSCAASGRTYGMGWFRQAPGKEREFVAV | 340 |
| 2TIG4_f3 | GVWSSGNTYYADFARGRFTISRDNAKNTVYLQMDSLKPEDTAVYYCA | |
| | APRYSSYTTYHAAYDYWGPGTQVTVSS | |

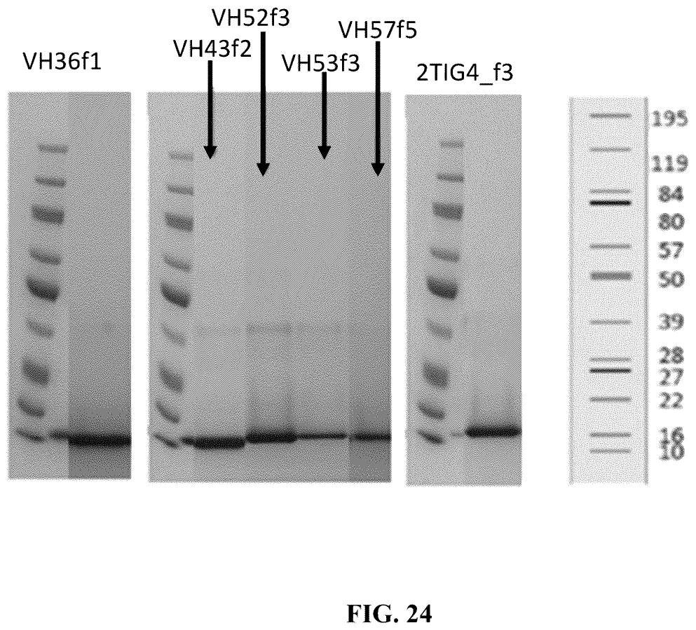

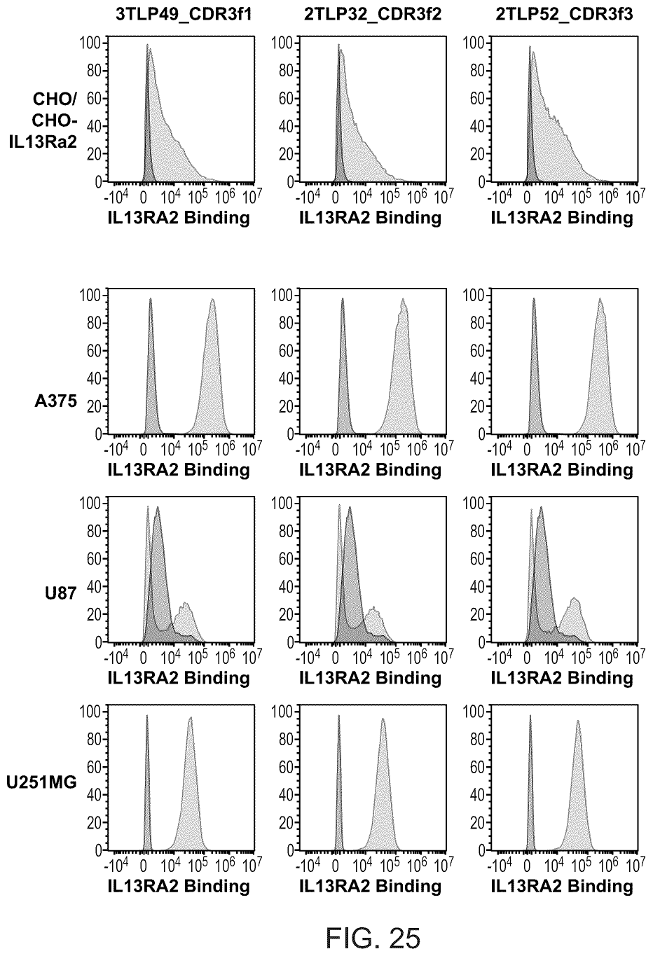

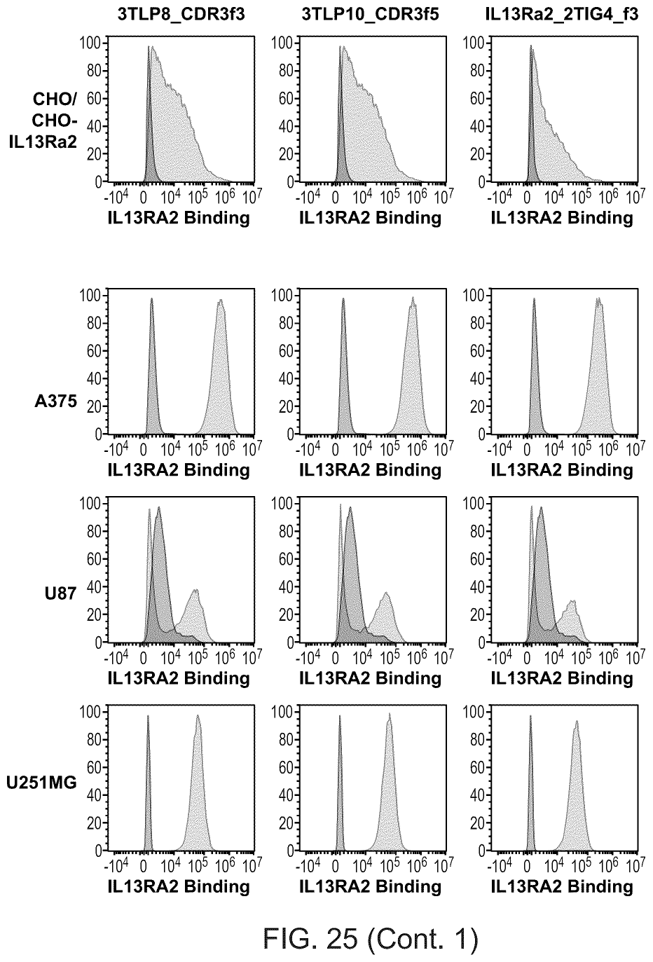

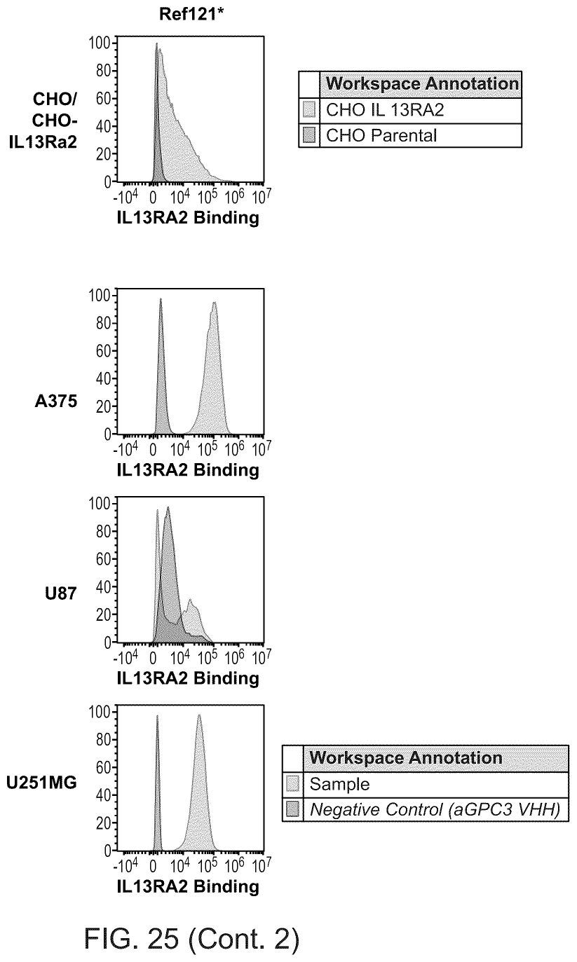

FIG. 24 shows resolution in SDS PAGE, showing expression and purity. FIG. 25 shows binding to cells expressing IL13Ra2, including 3 cancer cell lines.

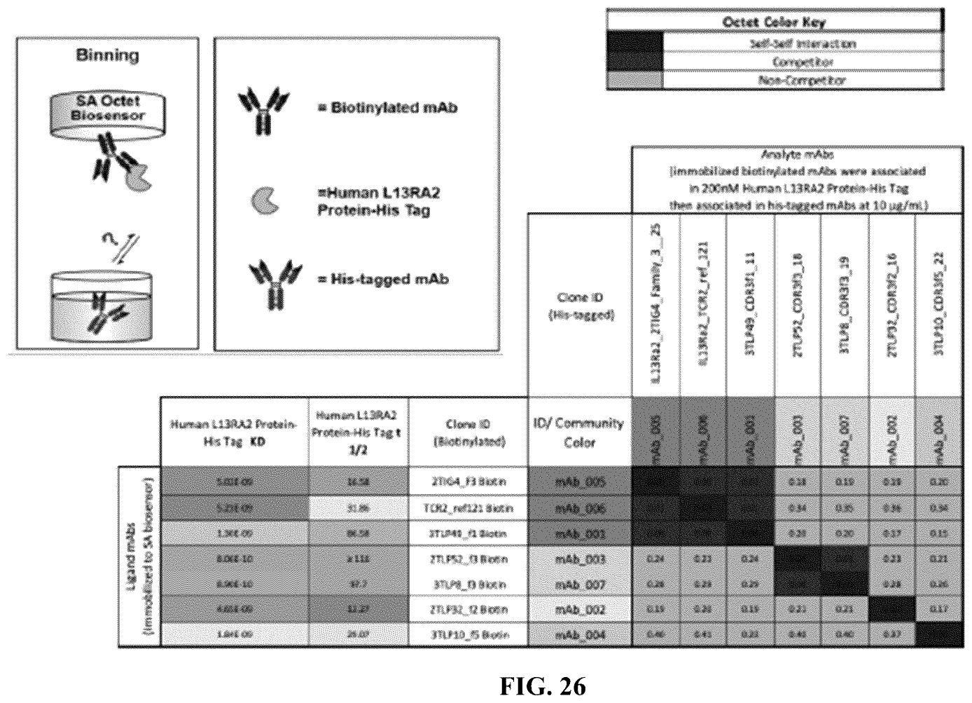

Binding Affinity for the V_{H} domains with IL13Rα2 are calculated as follows: Biotinylated VHHs were individually captured on streptavidin biosensors. VHH-captured biosensors were dipped in wells containing antigen and the binding response was measured. Biosensors were later dipped in buffer wells to observe the dissociation of bound antigen from captured VHH. (Schematic representation of the process depicted in FIG. 26, upper left corner). In this case, the IL13RA2 protein used was human recombinant protein extracellular domain; Rhesus macaque IL13R alpha 2 protein with His tag or mouse recombinant His tagged protein or irrelevant protein His tagged were used as controls. The Affinity data is shown in Table 9A-9C.

TABLE 9A

| | mAb | IL13RA2 Protein, Human, Recombinant (ECD)-His tag (1 uM) | | | | | |
|---|---|---|---|---|---|---|---|
| | Capture | Binding | | | | | |
| | level | Response | Kₐ | K_{d} | KD | t½ | |
| Name | (nm^{s}) | (nm^{s}) | (1/Mₛ) | (1/s)* | (M) | (min) | R² |
| IL13Ra2_2TIG4_f3- | 0.47 | 0.72 | 1.388E−05 | 6.967E−04 | 5.021E−09 | 17 | 0.94 |
| Biotin | | | | | | | |
| VH43f2-Biotin | 0.59 | 0.92 | 2.025E−05 | 9.412E−04 | 4.648E−09 | 12.27 | 0.96 |
| VH52f3 Biotin | 0.55 | 0.87 | 1.240E−05 | ≤1.00E−04 | 8.064E−10 | ≥116 | 0.94 |
| VH57f5 Biotin | 0.61 | 1.36 | 2.160E−05 | 3.973E−04 | 1.840E−09 | 29 | 0.96 |
| VH36f1 Biotin | 0.51 | 0.67 | 1.024E−05 | 1.334E−04 | 1.303E−09 | 87 | 0.95 |
| VH53f3 Biotin | 0.58 | 1.04 | 1.329E−05 | 1.182E−04 | 8.897E−10 | 98 | 0.93 |
| TCR2 ref121 Biotin | 0.97 | 1.18 | 6.937E−04 | 3.626E−04 | 5.226E−09 | 32 | 0.95 |
| mAb-01 biotin 2GPC5 | 0.65 | −0.01 | N.B. | N.B. | N.B. | N.B. | N.B. |

TABLE 9B

| | mAb | IL13RA2 Protein, Rhesus macaque, His tag (1 uM) | | | | | |
|---|---|---|---|---|---|---|---|
| | Capture | Binding | | | | | |
| | level | Response | Kₐ | K_{d} | KD | t½ | |
| Name | (nm^{s}) | (nm^{s}) | (1/Mₛ) | (1/s)* | (M) | (min) | R² |
| IL13Ra2_2TIG4_f3- | 0.47 | 1.17 | 1.072E05 | 3.882E−04 | 3.620E−09 | 30 | 0.95 |
| Biotin | | | | | | | |
| VH43f2-Biotin | 0.59 | 0.96 | 6.054E04 | 1.641E−03 | 2.711E−08 | 7.04 | 0.99 |
| VH52f3 Biotin | 0.55 | 1.13 | 6.705E04 | ≤1.004E−04 | 1.491E−09 | ≥116 | 0.99 |
| VH57f5 Biotin | 0.61 | 0.93 | 4.168E04 | 4.854E−03 | 1.124E−07 | 2.4 | 0.97 |
| VH36f1 Biotin | 0.51 | 1.02 | 6.933E04 | ≤1.004E−04 | 1.442E−09 | ≥116 | 0.98 |
| VH53f3 Biotin | 0.58 | 1.35 | 7.021E04 | ≤1.004E−04 | 1.424E−09 | ≥116 | 0.98 |
| TCR2 ref121 Biotin | 0.97 | 1.85 | 5.472E04 | 1.187E−04 | 2.170E−09 | 97 | 0.98 |
| mAb-01 biotin 2GPC5 | 0.65 | 0.0 | N.B. | N.B. | N.B. | N.B. | N.B. |

TABLE 9C

| | mAb | IL13RA2 Protein, mouse Recombinant- His tag (1 uM) | | | | | |
|---|---|---|---|---|---|---|---|
| | Capture | Binding | | | | | |
| | level | Response | Kₐ | K_{d} | KD | t½ | |
| Name | (nm^{s}) | (nm^{s}) | (1/Mₛ) | (1/s)* | (M) | (min) | R² |
| IL13Ra2_2TIG4_f3- | 0.47 | −0.02 | N.B. | N.B. | N.B. | N.B. | N.B. |
| Biotin | | | | | | | |
| VH43f2-Biotin | 0.59 | −0.04 | N.B. | N.B. | N.B. | N.B. | N.B. |
| VH52f3 Biotin | 0.55 | −0.02 | N.B. | N.B. | N.B. | N.B. | N.B. |
| VH57f5 Biotin | 0.61 | 1.17 | 1.397E05 | 4.854E−03 | 3.367E−08 | 2.46 | 0.94 |
| VH36f1 Biotin | 0.51 | −0.01 | N.B. | ≤1.004E−04 | 1.442E−09 | N.B. | N.B. |
| VH53f3 Biotin | 0.58 | −0.01 | N.B. | ≤1.004E−04 | 1.424E−09 | N.B. | N.B. |
| TCR2 ref121 Biotin | 0.97 | −0.03 | N.B. | 1.187E−04 | 2.170E−09 | N.B. | N.B. |
| mAb-01 biotin 2GPC5 | 0.65 | −0.01 | N.B. | N.B. | N.B. | N.B. | N.B. |

Four of the tested VHHs exhibit high affinity ranging in the low nM ranges (unit figures). Two of the VHHs demonstrate sub-nanomolar affinity. Four of six VHHs were highly cross-reactive to cynomolgous IL 13RA2, and one was cross reactive to both mouse and cynomolgous IL 13RA2 with decrease in affinity by about 2 folds compared to human counterpart.

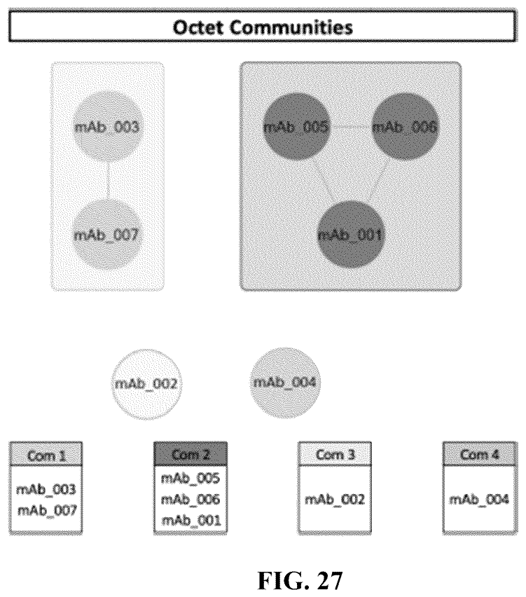

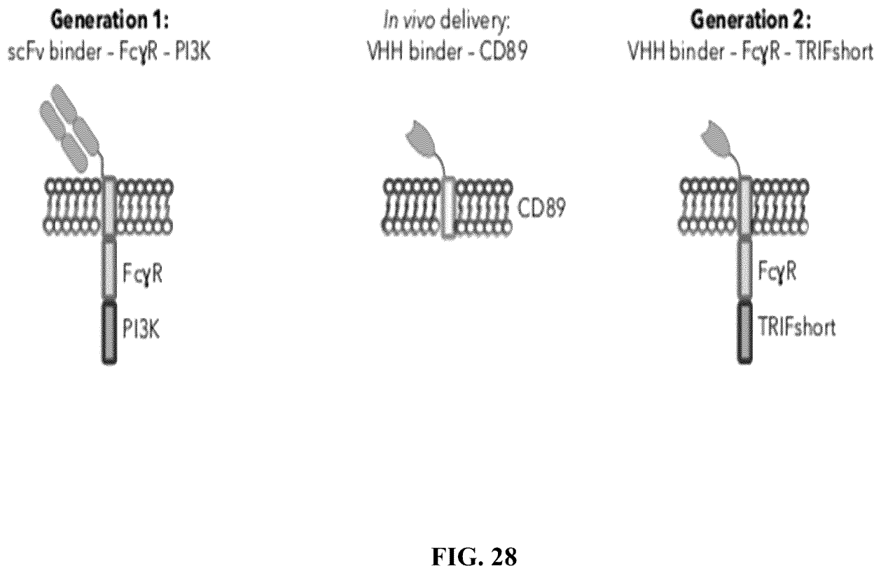

The binning assay in brief comprises Step 1: Capture biotinylated VHH (mAb-1) using streptavidin biosensor. Step 2: Dip in wells containing 20 nM human JL13Ra2-His6. “His6” is disclosed as (SEQ ID NO: 364). Step 3: Dip in wells containing nonbiotinylated His-tagged VHH (mAb-2). FIG. 26 shows binning method and data. FIG. 27 shows the different communities the binders are subdivided in.

1.-119. (canceled)

120. A composition comprising a polynucleic acid, wherein the polynucleic acid comprises a sequence encoding a chimeric fusion protein (CFP), the CFP comprising:
(a) an extracellular domain comprising an anti-GPC3 binding domain, wherein the anti-GPC3 binding domain comprises a variable heavy chain (VHH) domain comprising:
(i) a heavy chain (HC) complementarity determining region 3 (CDR3) comprising a sequence selected from the group consisting of
(SEQ ID NO: 114)
ATACADTTLYEYDY,
(SEQ ID NO: 136)
ATACANWSSLGPYDY,
(SEQ ID NO: 139)
ATTCASPEKYEYDY,
(SEQ ID NO: 142)
YARYSGRTY,
(SEQ ID NO: 148)
ATDCAGGVGHEYDY
and
(SEQ ID NO: 149)
ATDCSLHGSDYPYDY;
(ii) a HC CDR2 comprising a sequence selected from the group consisting of ISASDGST (SEQ ID NO: 168), ISSSDGST (SEQ ID NO: 169), ISASDGNT (SEQ ID NO: 190), DSITSI (SEQ ID NO: 191), IAASVGST (SEQ ID NO: 327), and ISSSDGSDGNT (SEQ ID NO: 193); and
(iii) a HC CDR1 comprising a sequence selected from the group consisting of GFPLAYYA (SEQ ID NO: 151), GFTLGYYA (SEQ ID NO: 157), GFPLNYYA (SEQ ID NO: 158), GSDFRADA (SEQ ID NO: 163), GFSLAYYA (SEQ ID NO: 165), and GFPLDYYA (SEQ ID NO: 153);
(b) a transmembrane domain; and
(c) an intracellular domain.

121. The composition of claim 120, wherein the variable heavy chain (VHH) domain comprises:
(i) the HC CDR1 of GFPLAYYA (SEQ ID NO: 151), the HC CDR2 of ISASDGST (SEQ ID NO: 168), and the HC CDR3 of ATACADTTLYEYDY (SEQ ID NO: 114);
(ii) the HC CDR1 of GFTLGYYA (SEQ ID NO: 157), the HC CDR2 of ISSSDGST (SEQ ID NO: 169), and the HC CDR3 of ATACANWSSLGPYDY (SEQ ID NO: 136);
(iii) the HC CDR1 of GFPLNYYA (SEQ ID NO: 158), the HC CDR2 of ISASDGNT (SEQ ID NO: 190), and the HC CDR3 of ATTCASPEKYEYDY (SEQ ID NO: 139);
(iv) the HC CDR1 of GSDFRADA (SEQ ID NO: 163), the HC CDR2 of DSITSI (SEQ ID NO: 163), and the HC CDR3 of YARYSGRTY (SEQ ID NO: 142);
(v) the HC CDR1 of GFSLAYYA (SEQ ID NO: 165), the HC CDR2 of IAASVGST (SEQ ID NO: 327), and the HC CDR3 of ATDCAGGVGHEYDY (SEQ ID NO: 148); or
(vi) the HC CDR1 of GFPLDYYA (SEQ ID NO: 153), the HC CDR2 of ISSSDGSDGNT (SEQ ID NO: 193), and the HC CDR3 of ATDCSLHGSDYPYDY(SEQ ID NO: 149).

122. The composition of claim 120, wherein the extracellular domain comprises a hinge, and wherein the hinge is operatively connected to the transmembrane domain.

123. The composition of claim 122, wherein the hinge is a CD8 alpha hinge.

124. The composition of claim 123, wherein the hinge has a sequence of
(SEQ ID NO: 307)
ALSNSIMYFSHFVPVFLPAKPTTTPAPRPPTPAPTIASQPLSLRPEACR
PAAGGAVHTRGLD.

125. The composition of claim 122, wherein the extracellular domain comprises a linker operatively linked to the VHH domain and the hinge, and wherein the linker has a sequence of SGGGSG (SEQ ID NO: 308).

126. The composition of claim 120, wherein the extracellular domain comprises a linker operatively linked to the VHH domain and the transmembrane domain, and wherein the linker has a sequence of SGGGSG (SEQ ID NO: 308).

127. The composition of claim 120, wherein the transmembrane domain is a CD8 transmembrane domain.

128. The composition of claim 127, wherein the transmembrane domain has a sequence of IYIWAPLAGTCGVLLLSLVITLYC (SEQ ID NO: 309).

129. The composition of claim 120, wherein the intracellular domain comprises an intracellular signaling domain from FcεR.

130. The composition of claim 129, wherein the intracellular domain comprises a sequence of RRLKIQVRKAAITSYEKSDGVYTGLSTRNQETYETLKHEKPPQ (SEQ ID NO: 310).

131. The composition of claim 120, wherein the intracellular domain comprises an intracellular signaling domain that comprises a PI3K recruitment domain.

132. The composition of claim 131, wherein the PI3K recruitment domain has a sequence of YEDMRGILYAAPQLRSIRGQPGPNHEEDADSYENM (SEQ ID NO: 314).

133. The composition of claim 120, wherein the intracellular domain comprises at least two intracellular signaling domains, and wherein the at least two intracellular signaling domains are separated by a linker.

134. The composition of claim 120, wherein the polynucleic acid is an mRNA.

135. The composition of claim 120, wherein the polynucleic acid is encapsulated in a lipid nanoparticle.

136. The composition of claim 120, wherein the VHH comprises a sequence with at least 80% identity to a sequence selected from the group consisting of SEQ ID NOs: 33, 47, 55, 58, 80 and 109.

137. A composition comprising a polynucleic acid, wherein the polynucleic acid comprises a sequence encoding a chimeric fusion protein (CFP), the CFP comprising:
(a) an extracellular domain comprising an anti-GPC3 binding domain, wherein the anti-GPC3 binding domain comprises a variable heavy chain (VHH) domain comprising a heavy chain (HC) complementarity determining region 1 (CDR1), HC CDR2, and a HC CDR3 as one of the following combinations:
CDR1: SEQ ID NO: 151, CDR2: SEQ ID NO: 170, CDR3: SEQ ID NO: 122;
CDR1: SEQ ID NO: 153, CDR2: SEQ ID NO: 171, CDR3: SEQ ID NO: 117;
CDR1: SEQ ID NO: 154, CDR2: SEQ ID NO: 182, CDR3: SEQ ID NO: 127;
CDR1: SEQ ID NO: 153, CDR2: SEQ ID NO: 171, CDR3: SEQ ID NO: 117;
CDR1: SEQ ID NO: 160, CDR2: SEQ ID NO: 186, CDR3: SEQ ID NO: 131;
CDR1: SEQ ID NO: 152, CDR2: SEQ ID NO: 184, CDR3: SEQ ID NO: 128;
CDR1: SEQ ID NO: 151, CDR2: SEQ ID NO: 168, CDR3: SEQ ID NO: 121;
CDR1: SEQ ID NO: 151, CDR2: SEQ ID NO: 169, CDR3: SEQ ID NO: 137;
CDR1: SEQ ID NO: 156, CDR2: SEQ ID NO: 170, CDR3: SEQ ID NO: 118;
CDR1: SEQ ID NO: 163, CDR2: SEQ ID NO: 191, CDR3: SEQ ID NO: 142;
CDR1: SEQ ID NO: 151, CDR2: SEQ ID NO: 170, CDR3: SEQ ID NO: 138;
CDR1: SEQ ID NO: 151, CDR2: SEQ ID NO: 170, CDR3: SEQ ID NO: 122;
CDR1: SEQ ID NO: 151, CDR2 SEQ ID NO: 170, CDR3 SEQ ID NO: 123;
CDR1 SEQ ID NO: 151, CDR2 SEQ ID NO: 170, CDR3 SEQ ID NO: 116;
CDR1 SEQ ID NO: 151, CDR2 SEQ ID NO: 169, CDR3 SEQ ID NO: 117;
CDR1 SEQ ID NO: 151, CDR2 SEQ ID NO: 169, CDR3 SEQ ID NO: 118;
CDR1 SEQ ID NO: 151, CDR2 SEQ ID NO: 169, CDR3 SEQ ID NO: 126;
CDR1 SEQ ID NO: 151, CDR2 SEQ ID NO: 170, CDR3 SEQ ID NO: 122;
CDR1 SEQ ID NO: 153, CDR2 SEQ ID NO: 179, CDR3 SEQ ID NO: 124;
CDR1 SEQ ID NO: 153, CDR2 SEQ ID NO: 171, CDR3 SEQ ID NO: 117;
CDR1 SEQ ID NO: 153, CDR2 SEQ ID NO: 171, CDR3 SEQ ID NO: 115;
CDR1 SEQ ID NO: 153, CDR2 SEQ ID NO: 177, CDR3 SEQ ID NO: 115;
CDR1 SEQ ID NO: 153, CDR2 SEQ ID NO: 169, CDR3 SEQ ID NO: 118;
CDR1 SEQ ID NO: 162, CDR2 SEQ ID NO: 169, CDR3 SEQ ID NO: 138;
CDR1 SEQ ID NO: 161, CDR2 SEQ ID NO: 175, CDR3 SEQ ID NO: 130;
CDR1 SEQ ID NO: 159, CDR2 SEQ ID NO: 169, CDR3 SEQ ID NO: 126;
CDR1 SEQ ID NO: 159, CDR2 SEQ ID NO: 169, CDR3 SEQ ID NO: 132;
CDR1 SEQ ID NO: 158, CDR2 SEQ ID NO: 169, CDR3 SEQ ID NO: 126;
CDR1 SEQ ID NO: 158, CDR2 SEQ ID NO: 169, CDR3 SEQ ID NO: 145;
CDR1 SEQ ID NO: 152, CDR2 SEQ ID NO: 169, CDR3 SEQ ID NO: 126;
CDR1 SEQ ID NO: 152, CDR2 SEQ ID NO: 169, CDR3 SEQ ID NO: 115;
CDR1 SEQ ID NO: 152, CDR2 SEQ ID NO: 169, CDR3 SEQ ID NO: 130;
CDR1 SEQ ID NO: 155, CDR2 SEQ ID NO: 174, CDR3 SEQ ID NO: 120;
CDR1 SEQ ID NO: 156, CDR2 SEQ ID NO: 169, CDR3 SEQ ID NO: 122;
CDR1 SEQ ID NO: 156, CDR2 SEQ ID NO: 169, CDR3 SEQ ID NO: 118;
CDR1 SEQ ID NO: 154, CDR2 SEQ ID NO: 169, CDR3 SEQ ID NO: 126;
CDR1 SEQ ID NO: 154, CDR2 SEQ ID NO: 169, CDR3 SEQ ID NO: 144;
CDR1 SEQ ID NO: 154, CDR2 SEQ ID NO: 190, CDR3 SEQ ID NO: 139;
CDR1 SEQ ID NO: 154, CDR2 SEQ ID NO: 188, CDR3 SEQ ID NO: 135;
CDR1 SEQ ID NO: 154, CDR2 SEQ ID NO: 181, CDR3 SEQ ID NO: 126;
CDR1 SEQ ID NO: 154, CDR2 SEQ ID NO: 176, CDR3 SEQ ID NO: 122;
CDR1 SEQ ID NO: 154, CDR2 SEQ ID NO: 183, CDR3 SEQ ID NO: 126;
CDR1 SEQ ID NO: 154, CDR2 SEQ ID NO: 180, CDR3 SEQ ID NO: 125;
CDR1 SEQ ID NO: 154, CDR2 SEQ ID NO: 169, CDR3 SEQ ID NO: 118;
CDR1 SEQ ID NO: 154, CDR2 SEQ ID NO: 169, CDR3 SEQ ID NO: 126;
CDR1 SEQ ID NO: 154, CDR2 SEQ ID NO: 172, CDR3 SEQ ID NO: 118;
CDR1 SEQ ID NO: 154, CDR2 SEQ ID NO: 172, CDR3 SEQ ID NO: 147;
CDR1 SEQ ID NO: 154, CDR2 SEQ ID NO: 172, CDR3 SEQ ID NO: 130;
CDR1 SEQ ID NO: 157, CDR2 SEQ ID NO: 169, CDR3 SEQ ID NO: 118;
CDR1 SEQ ID NO: 157, CDR2 SEQ ID NO: 169, CDR3 SEQ ID NO: 126;
CDR1 SEQ ID NO: 152, CDR2 SEQ ID NO: 181, CDR3 SEQ ID NO: 146;
CDR1 SEQ ID NO: 152, CDR2 SEQ ID NO: 184, CDR3 SEQ ID NO: 128;
CDR1 SEQ ID NO: 152, CDR2 SEQ ID NO: 178, CDR3 SEQ ID NO: 129;
CDR1 SEQ ID NO: 152, CDR2 SEQ ID NO: 178, CDR3 SEQ ID NO: 115;
CDR1 SEQ ID NO: 152, CDR2 SEQ ID NO: 178, CDR3 SEQ ID NO: 128;
CDR1 SEQ ID NO: 152, CDR2 SEQ ID NO: 185, CDR3 SEQ ID NO: 128;
CDR1 SEQ ID NO: 152, CDR2 SEQ ID NO: 184, CDR3 SEQ ID NO: 128;
CDR1 SEQ ID NO: 160, CDR2 SEQ ID NO: 184, CDR3 SEQ ID NO: 128;
CDR1 SEQ ID NO: 153, CDR2 SEQ ID NO: 169, CDR3 SEQ ID NO: 126;
CDR1 SEQ ID NO: 152, CDR2 SEQ ID NO: 167, CDR3 SEQ ID NO: 113;
CDR1 SEQ ID NO: 154, CDR2 SEQ ID NO: 189, CDR3 SEQ ID NO: 134;
CDR1 SEQ ID NO: 154, CDR2 SEQ ID NO: 175, CDR3 SEQ ID NO: 118;
CDR1 SEQ ID NO: 154, CDR2 SEQ ID NO: 187, CDR3 SEQ ID NO: 135;
CDR1 SEQ ID NO: 154, CDR2 SEQ ID NO: 176, CDR3 SEQ ID NO: 126;
CDR1 SEQ ID NO: 151, CDR2 SEQ ID NO: 168, CDR3 SEQ ID NO: 121;
CDR1 SEQ ID NO: 154, CDR2 SEQ ID NO: 169, CDR3 SEQ ID NO: 126;
CDR1 SEQ ID NO: 151, CDR2 SEQ ID NO: 170, CDR3 SEQ ID NO: 116;
CDR1 SEQ ID NO: 160, CDR2 SEQ ID NO: 184, CDR3 SEQ ID NO: 128;
CDR1 SEQ ID NO: 151, CDR2 SEQ ID NO: 170, CDR3 SEQ ID NO: 122;
CDR1 SEQ ID NO: 163, CDR2 SEQ ID NO: 191, CDR3 SEQ ID NO: 142;
CDR1 SEQ ID NO: 166, CDR2 SEQ ID NO: 194, CDR3 SEQ ID NO: 150;
CDR1 SEQ ID NO: 160, CDR2 SEQ ID NO: 184, CDR3 SEQ ID NO: 128;
CDR1 SEQ ID NO: 152, CDR2 SEQ ID NO: 184, CDR3 SEQ ID NO: 128;
(b) a transmembrane domain; and
(c) an intracellular domain.

138. The composition of claim 137, wherein the anti-GPC3 binding domain comprises an amino acid sequence with at least 80% sequence identity to any one of SEQ ID NOs: 22-112.

139. A method of treating a cancer in a subject in need thereof, the method comprising administering to the subject a composition comprising a polynucleic acid, wherein the polynucleic acid comprises a sequence encoding a chimeric fusion protein (CFP), the CFP comprising:
(a) an extracellular domain comprising an anti-GPC3 binding domain, wherein the anti-GPC3 binding domain comprises a variable heavy chain (VHH) domain comprising:
(i) a heavy chain (HC) complementarity determining region 3 (CDR3) sequence selected from the group consisting of ATACADTTLYEYDY (SEQ ID NO: 114), ATACANWSSLGPYDY (SEQ ID NO: 136), ATTCASPEKYEYDY (SEQ ID NO: 139), YARYSGRTY (SEQ ID NO: 142), ATDCAGGVGHEYDY (SEQ ID NO: 148) and ATDCSLHGSDYPYDY(SEQ ID NO: 149);
(ii) a HC CDR2 sequence selected from the group consisting of ISASDGST (SEQ ID NO: 168), ISSSDGST (SEQ ID NO: 169), ISASDGNT (SEQ ID NO: 190), DSITSI (SEQ ID NO: 191), IAASVGST (SEQ ID NO: 327), and ISSSDGSDGNT (SEQ ID NO: 193); and
(iii) a HC CDR1 sequence selected from the group consisting of
(SEQ ID NO: 151)
GFPLAYYA,
(SEQ ID NO: 157)
GFTLGYYA,
(SEQ ID NO: 158)
GFPLNYYA,
(SEQ ID NO: 163)
GSDFRADA,
(SEQ ID NO: 165)
GFSLAYYA,
and
(SEQ ID NO: 153)
GFPLDYYA;
(b) a transmembrane domain; and
(c) an intracellular domain.


SEQUENCE LISTING (364 sequences)

SEQ ID NO: 1 - protein, 119 aa, synthetic construct
QVQLQESGGGLVQAGGSLRLSCAAPRSIFSINAMGWYRQAPGKQRELVAAITSGGSPTYADSVKGRFTISRDNAKNTVYLQMNSLKAEDTAVYYCATGPYGLDNALDAWGQGTQVTVSS

SEQ ID NO: 2 - protein, 8 aa, synthetic construct
RSIFSINA

SEQ ID NO: 3 - protein, 7 aa, synthetic construct
ITSGGSP

SEQ ID NO: 4 - protein, 13 aa, synthetic construct
ATGPYGLDNALDA

SEQ ID NO: 5 - DNA, 357 bp, synthetic construct
  source 1..357: mol_type = other DNA
caagttcagctccaggaatcaggtgggggactcgtacaggctggaggaagcctgcgattgagctgtgccgctccacgatctatcttctcaataaatgctatgggttggtacaggcaggccccaggcaaacaaagagaacttgtagcagccattacttcaggggggagcccaacctatgctgactcagtaaaggggcggttcactattagtagggataacgcgaagaatacggtctacttgcagatgaacagtctgaaggccgaagatacggccgtgtattattgtgccacaggtccctatgggctggacaatgcgttggatgcgtggggtcagggcacgcaggtcactgtgagtagt

SEQ ID NO: 6 - protein, 119 aa, synthetic construct
EVQLLESGGGLVQPGGSLRLSCAAPRSIFSINAMGWYRQAPGKQRELVAAITSGGSPTYADSVKGRFTISRDNSKNTLYLQMNSLKAEDTAVYYCATGPYGLDNALDAWGQGTLVTVSS

SEQ ID NO: 7 - protein, 119 aa, synthetic construct
EVQLLESGGGLVQPGGSLRLSCAAPRSIFSINAMGWYRQAPGKQRELVAAITSGGSPTYADSVKGRFTISRDNSKNTLYLQMNSLRAEDTAVYYCATGPYGLDNALDAWGQGTLVTVSS

SEQ ID NO: 8 - protein, 119 aa, synthetic construct
EVQLLESGGGLVQPGGSLRLSCAAPRSIFSINAMGWYRQAPGKQRELVSAITSGGSPTYADSVKGRFTISRDNSKNTLYLQMNSLRAEDTAVYYCATGPYGLDNALDAWGQGTLVTVSS

SEQ ID NO: 9 - protein, 119 aa, synthetic construct
EVQLLESGGGLVQPGGSLRLSCAAPRSIFSINAMSWYRQAPGKQRELVSAITSGGSPTYADSVKGRFTISRDNSKNTLYLQMNSLRAEDTAVYYCATGPYGLDNALDAWGQGTLVTVSS

SEQ ID NO: 10 - protein, 119 aa, synthetic construct
EVQLLESGGGLVQPGGSLRLSCAASRSIFSINAMSWYRQAPGKQRELVSAITSGGSPTYADSVKGRFTISRDNSKNTLYLQMNSLRAEDTAVYYCATGPYGLDNALDAWGQGTLVTVSS

SEQ ID NO: 11 - protein, 122 aa, synthetic construct
QVQLQESGGGLVQTGGSLRLACTASGFTFDDYAIAWFRQAPGKEREFVAAISWSGGTTHYADSVKGRFTISRDNAKNTLYLQMSSLKPEDTAVYFCAKSLRSSPSSRWFGSRGQGTQVTVSS

SEQ ID NO: 12 - protein, 8 aa, synthetic construct
GFTFDDYA

SEQ ID NO: 13 - protein, 8 aa, synthetic construct
ISWSGGTT

SEQ ID NO: 14 - protein, 15 aa, synthetic construct
AKSLRSSPSSRWFGS

SEQ ID NO: 15 - DNA, 366 bp, synthetic construct
  source 1..366: mol_type = other DNA
caggtgcagttgcaggagagtggcggtggcctggtgcagaccggtggcagtctgaggctggcctgcactgccagcggcttcaccttcgacgactatgccatcgcgtggttcaggcaggcccctggcaaggagagggagtttgtcgcagccatcagctggagcggaggcaccacccactacgctgacagcgtgaagggcaggttcaccattagcagggacaatgccaagaacaccctgtacctgcagatgagcagtctcaagcccgaagataccgccgtgtacttctgcgccaagagcctgaggagcagccccagcagcaggtggttcggcagccgcggtcaaggcacccaggtgaccgtgagctct

SEQ ID NO: 16 - protein, 121 aa, synthetic construct
EVQLQESGGGLVQPGGSLRLSCAASGFTFDDYAIAWFRQAPGKEREFVSAISWSGGTTHYADSVKGRFTISRDNAKNTLYLQMSSLKPEDTAVYFCAKSLRSSPSSRWFGSRGQGTQVTVS

SEQ ID NO: 17 - protein, 121 aa, synthetic construct
EVQLQESGGGLVQPGGSLRLSCAASGFTFDDYAIAWFRQAPGKEREFVSAISWSGGTTHYADSVKGRFTISRDNAKNTLYLQMSSLKPEDTAVYFCAKSLRSSPSSRWFGSWGQGTQVTVS

SEQ ID NO: 18 - protein, 121 aa, synthetic construct
EVQLQESGGGLVQPGGSLRLSCAASGFTFDDYAIAWFRQAPGKEREFVSTISWSGGTTHYADSVKGRFTISRDNAKNTLYLQMSSLKPEDTAVYFCAKSLRSSPSSRWFGSWGQGTQVTVS

SEQ ID NO: 19 - protein, 121 aa, synthetic construct
EVQLQESGGGLVQPGGSLRLSCAASGFTFDDYAIAWFRQAPGKEREFVSTISWSGGTTHYADSVKGRFTISRDNAKNTLYLQMSSLKPEDTAVYYCAKSLRSSPSSRWFGSWGQGTQVTVS

SEQ ID NO: 20 - protein, 121 aa, synthetic construct
EVQLQESGGGLVQPGGSLRLSCAASGFTFDDYAITWFRQAPGKEREFVSTISWSGGTTHYADSVKGRFTISRDNAKNTLYLQMSSLKPEDTAVYYCAKSLRSSPSSRWFGSWGQGTQVTVS

SEQ ID NO: 21 - protein, 119 aa, synthetic construct; identical to SEQ ID NO: 10

SEQ ID NO: 22 - protein, 121 aa, synthetic construct
QVQLQESGGGLVHSGGSLRLSCAASGFPLAYYAIGWFRQAPGKEREGVSCISSSDGNTYYADAVKGRFTISRDNAKNTVYLQMNSLKPEDTAVYYCATACADTTQHEYDYWGQGTQVTVSS

SEQ ID NO: 23 - protein, 121 aa, synthetic construct
QVQLQESGGGLVHSGGSLRLSCAASGFPLDYYAIGWFRQAPGKEREGVSCISSADGSTYYADSVKGRFTISRDNAKNTVYLQMNSLGPEDTAVYYCATACADTTQYDYDYWGQGTQVTVSS

SEQ ID NO: 24 - protein, 120 aa, synthetic construct
QVQLQESGGGLVHSGGSLRLSCAASGFTLDYYAIGWFRRAPGKEREGVSCISSGDGKTYYADSVKGRFTISRDNAKNTVYLQMNSLKPEDTAVYYCATACAGAIGPYDYWGQGTQVTVSS

SEQ ID NO: 25 - protein, 121 aa, synthetic construct
QVQLQESGGGLVPPGGSLRLSCAASGFPLDYYAIGWFRQAPGKEREGVSCISSADGSTYYADSVKGRFTISRDNAKNTVYLQMNSLGPEDTAVYYCATACADTTQYDYDYWGQGTQVTVSS

SEQ ID NO: 26 - protein, 123 aa, synthetic construct
QVQLQESGGGLVQAGGSLRLSCAASGFSLGYYAIGWFRQAPGKEREGVSCISSSDGHSSTYYADSVKGRFTISRDNAKNTVYLQMNNLKPEDTAVYYCATDCAGGTATPYDYWGQGTQVTVSS

SEQ ID NO: 27 - protein, 124 aa, synthetic construct
QVQLQESGGGLVQAGGSLRLSCAASGRTFSSYGMGWFRQAPGKEREFVAAISWSGGSTYYADSVKGRFTISRDNAKNTVYLQMNSLKPEDTAVYYCASSAWPAGPKHQVEYDYWGQGTQVTVSS

SEQ ID NO: 28 - protein, 120 aa, synthetic construct
QVQLQESGGGLVQAGGSLRLSCTASGFSLDYYAIGWFRQAPGKEREGVACISSRTGSTYYADSVKGRFTISRDNAKNTVALQMNSLKPEDTAVYYCATACVVVGDQNDYWGQGTQVTVSS

SEQ ID NO: 29 - protein, 121 aa, synthetic construct
QVQLQESGGGLVQDGGSLRLSCAASGFPLAYYAIGWFRQAPGKEREGVSCISASDGSTYYADSVKGRFTISRDNAKNTVYLQMNSLKPEDTAVYYCATACAETTLYEYDYWGQGTQVTVSS

SEQ ID NO: 30 - protein, 122 aa, synthetic construct
QVQLQESGGGLVQPGESLRLSCAASGFPLAYYAIGWFRQAPGKEREGVSCISSSDGSTYYADSVKGRFTISRDNAKNTVYLQMNSLKPEDTAVYYCATACANWSTLGPYDYWGQGTQVTVSS

SEQ ID NO: 31 - protein, 121 aa, synthetic construct
QVQLQESGGGLVQPGESLRLSCAASGFTLAYYAIGWFRQAPGKEREGVSCISSSDGNTYYADSVKGRFTISRDNAKNTVYLQMNRLKPEDTAVYYCATACADTTQYEYDYWGQGTQVTVSS

SEQ ID NO: 32 - protein, 114 aa, synthetic construct
QVQLQESGGGLVQPGGSLKLSCAASGSDFRADAMGWYRQAPGKEREPVAIDSITSIYYVDSVEGRFTISRDNTKNTVYLQMTSLKPEDTAVYYCYARYSGRTYWGRGTQVTVSS

SEQ ID NO: 33 - protein, 121 aa, synthetic construct
QVQLQESGGGLVQPGGSLRLSCAASGFPLAYYAIGWFRQAPGKEREGVSCISASDGSTYYADSVKGRFTISRDNAKNTVYLQMNSLRPEDTAVYYCATACADTTLYEYDYWGQGTQVTVSS

SEQ ID NO: 34 - protein, 121 aa, synthetic construct
QVQLQESGGGLVQPGGSLRLSCAASGFPLAYYAIGWFRQAPGKEREGVSCISSSDGNTYYADAVKGRFAISRDNAKNTVYLQMNSLKPEDTAVYYCATACSDPRVYEYDYWGQGTQVTVSS

SEQ ID NO: 35 - protein, 121 aa, synthetic construct
QVQLQESGGGLVQPGGSLRLSCAASGFPLAYYAIGWFRQAPGKEREGVSCISSSDGNTYYADAVKGRFTISRDNAKNTVYLQMNSLKPEDTAVYYCATACADTTQHEYDYWGQGTQVTVSS

SEQ ID NO: 36 - protein, 121 aa, synthetic construct
QVQLQESGGGLVQPGGSLRLSCAASGFPLAYYAIGWFRQAPGKEREGVSCISSSDGNTYYADAVKGRFTISRDNAKNTVYLQMNSLKPEDTAVYYCATACVDTTHYEYDYWGQGTQVTVSS

SEQ ID NO: 37 - protein, 121 aa, synthetic construct
QVQLQESGGGLVQPGGSLRLSCAASGFPLAYYAIGWFRQAPGKEREGVSCISSSDGNTYYADSVKGRFTISRDNAKNTVYLQMNSLKPEDTAVYYCATACADATQHEYDYWGQGTQVTVSS

SEQ ID NO: 38 - protein, 121 aa, synthetic construct
QVQLQESGGGLVQPGGSLRLSCAASGFPLAYYAIGWFRQAPGKEREGVSCISSSDGSTYYADSVKGRFTISRDNAKNTVYLQMNSLGPEDTAVYYCATACADTTQYDYDYWGQGTQVTVSS

SEQ ID NO: 39 - protein, 121 aa, synthetic construct
QVQLQESGGGLVQPGGSLRLSCAASGFPLAYYAIGWFRQAPGKEREGVSCISSSDGSTYYADSVKGRFTISRDNAKNTVYLQMNSLKPEDTAVYYCATACADTTQYEYDYWGQGTQVTVSS

SEQ ID NO: 40 - protein, 120 aa, synthetic construct
QVQLQESGGGLVQPGGSLRLSCAASGFPLAYYAIGWFRQAPGKEREGVSCISSSDGSTYYADSVKGRFTISRDNAKNTVYLQMNSLKPEDTAVYYCATACGGATGPYDYWGQGTQVTVSS

SEQ ID NO: 41 - protein, 121 aa, synthetic construct
QVQLQESGGGLVQPGGSLRLSCAASGFPLAYYAIGWFRRAPGKEREGVSCISSSDGNTYYADAVKGRFTISRDNAKNTVYLQMNSLKPEDTAVYYCATACADTTQHEYDYWGQGTQVTVSS

SEQ ID NO: 42 - protein, 122 aa, synthetic construct
QVQLQESGGGLVQPGGSLRLSCAASGFPLDYYAIGWFRQAPGKEREGVSCISAGDGSSTYYADSVKGRFTISRDNAKNTVYLQMNSLKPEDTAVYYCATACASTTLYEYDYWGQGTQVTVSS

SEQ ID NO: 43 - protein, 121 aa, synthetic construct
QVQLQESGGGLVQPGGSLRLSCAASGFPLDYYAIGWFRQAPGKEREGVSCISSADGSTYYADSVKGRFTISRDNAKNAVYLQMNSLGPEDTAVYYCATACADTTQYDYDYWGQGTQVTVSS

SEQ ID NO: 44 - protein, 121 aa, synthetic construct
QVQLQESGGGLVQPGGSLRLSCAASGFPLDYYAIGWFRQAPGKEREGVSCISSADGSTYYADSVKGRFTISRDNAKNTVYLQMNSLGPEDTAVYYCATACADTTQYDYDYWGQGTQVTVSS

SEQ ID NO: 45 - protein, 121 aa, synthetic construct
QVQLQESGGGLVQPGGSLRLSCAASGFPLDYYAIGWFRQAPGKEREGVSCISSADGSTYYADSVKGRFTISRDNAKNTVYLQMNSLKPEDTAVYYCATACVDTTQYEYDYWGQGTQVTVSS

SEQ ID NO: 46 - protein, 121 aa, synthetic construct
QVQLQESGGGLVQPGGSLRLSCAASGFPLDYYAIGWFRQAPGKEREGVSCISSPDGSTYYADSVKGRFTISRDNAKNTVYLQMNSLKPEDTAVYYCATACVDTTQYEYDYWGQGTQVTVSS

SEQ ID NO: 47 - protein, 125 aa, synthetic construct
QVQLQESGGGLVQPGGSLRLSCAASGFPLDYYAIGWFRQAPGKEREGVSCISSSDGSDGNTYYADSVKGRFTISRDNAKNTVYLQMNSLKPEDTAVYYCATDCSLHGSDYPYDYWGQGTQVTVSS

SEQ ID NO: 48 - protein, 121 aa, synthetic construct
QVQLQESGGGLVQPGGSLRLSCAASGFPLDYYAIGWFRQAPGKEREGVSCISSSDGSTYYADSVKGRFTISRDNAKNTVYLQMNSLKPEDTAVYYCATACADTTQYEYDYWGQGTQVTVSS

SEQ ID NO: 49 - protein, 121 aa, synthetic construct
QVQLQESGGGLVQPGGSLRLSCAASGFPLEYYAIGWFRQAPGKEREGVSCISSSDGSTYYADSVKGRFTISRDNAKNTVYLQMNSLKPEDTAVYYCATACSDPRVYEYDYWGQGTQVTVSS

SEQ ID NO: 50 - protein, 121 aa, synthetic construct
QVQLQESGGGLVQPGGSLRLSCAASGFPLGYYAIGWFRQAPGKEREGVSCISSSDDSTYYADSVKGRFTISRDNDKNTVYLQMNSLKPEDTAVYYCATDCAGGTSTPYDYWGQGTQVTVSS

SEQ ID NO: 51 - protein, 121 aa, synthetic construct
QVQLQESGGGLVQPGGSLRLSCAASGFPLHYYAIGWFRQAPGKEREGVSCISSGDGSTYYADSVKGRFTISRDNAKNTVYLQMNSLKPEDTAVYYCATSCVVVTKNEYDYWGQGTQVTVSS

SEQ ID NO: 52 - protein, 120 aa, synthetic construct
QVQLQESGGGLVQPGGSLRLSCAASGFPLHYYAIGWFRQAPGKEREGVSCISSSDGSTYYADSVKGRFTISRDNAKNTVYLQMNSLKPEDTAVYYCATACGGATGPYDYWGQGTQVTVSS

SEQ ID NO: 53 - protein, 121 aa, synthetic construct
QVQLQESGGGLVQPGGSLRLSCAASGFPLHYYAIGWFRQAPGKEREGVSCISSSDGSTYYADSVKGRFTISRDNAKNTVYLQMNSLKPEDTAVYYCATACVVADRNEYDYWGQGTQVTVSS

SEQ ID NO: 54 - protein, 121 aa, synthetic construct
QVQLQESGGGLVQPGGSLRLSCAASGFPLHYYAIGWFRQAPGKEREGVSCISSSDGSTYYADSVKGRFTISRDNAKNTVYLQMNSLRPEDTAVYYCATACVVADRNEYDYWGQGTQVTVSS

SEQ ID NO: 55 - protein, 121 aa, synthetic construct
QVQLQESGGGLVQPGGSLRLSCAASGFPLNYYAIGWFRQAPGKEREGVSCISASDGNTYYADSVKGRFTISRDNAKNTVYLQMNSLKPEDTAVYYCATTCASPEKYEYDYWGQGTQVTVSS

SEQ ID NO: 56 - protein, 120 aa, synthetic construct
QVQLQESGGGLVQPGGSLRLSCAASGFPLNYYAIGWFRQAPGKEREGVSCISSSDGSTYYADSVKGRFIISRDNAKNTVYLQMNSLKPEDTAVYYCATACGGATGPYDYWGQGTQVTVSS

SEQ ID NO: 57 - protein, 120 aa, synthetic construct
QVQLQESGGGLVQPGGSLRLSCAASGFPLNYYAIGWFRQAPGKEREGVSCISSSDGSTYYADSVKGRFTISRDNAKNTVYLQMNSLKPEDTAVYYCATACGSAVHEYDYWGQGTQVTVSS

SEQ ID NO: 58 - protein, 121 aa, synthetic construct
QVQLQESGGGLVQPGGSLRLSCAASGFSLAYYAIGWFRQAPGKEREGVSCIAASVGSTYYADSVKGRFTISRDDAKNTVYLQMNSLKPEDTAVYYCATDCAGGVGHEYDYWGQGTQVTVSS

SEQ ID NO: 59 - protein, 120 aa, synthetic construct
QVQLQESGGGLVQPGGSLRLSCAASGFSLDYYAIGWFRQAPGKEREGVSCISSSDGSTYYADSVKGRFTISRDNAKNAVYLQMNSLKPEDTAVYYCATACGGATGPYDYWGQGTQVTVSS

SEQ ID NO: 60 - protein, 121 aa, synthetic construct
QVQLQESGGGLVQPGGSLRLSCAASGFSLDYYAIGWFRQAPGKEREGVSCISSSDGSTYYADSVKGRFTISRDNAKNAVYLQMNSLKPEDTAVYYCATACVDTTQYEYDYWGQGTQVTVSS

SEQ ID NO: 61 - protein, 121 aa, synthetic construct
QVQLQESGGGLVQPGGSLRLSCAASGFSLDYYAIGWFRQAPGKEREGVSCISSSDGSTYYADSVKGRFTISRDNAKNTVYLQMNSLKPEDTAVYYCATDCAGGTSTPYDYWGQGTQVTVSS

SEQ ID NO: 62 - protein, 121 aa, synthetic construct
QVQLQESGGGLVQPGGSLRLSCAASGFSLNYYAIGWFRQAPGKEREGVSCISAGDGNTYYADSVKGRFTISRDNAANTVSLQMDSLKPEDTAVYYCATACVITTLYEYDYWGQGTQVTVSS

SEQ ID NO: 63 - protein, 121 aa, synthetic construct
QVQLQESGGGLVQPGGSLRLSCAASGFTLAYYAIGWFRQAPGKEREGVSCISSSDGSTYYADSVKGRFTISRDNAKNTVYLQMNSLKPEDTAVYYCATACADTTQHEYDYWGQGTQVTVSS

SEQ ID NO: 64 - protein, 121 aa, synthetic construct
QVQLQESGGGLVQPGGSLRLSCAASGFTLAYYAIGWFRQAPGKEREGVSCISSSDGSTYYADSVKGRFTISRDNAKNTVYLQMNSLKPEDTAVYYCATACADTTQYEYDYWGQGTQVTVSS

SEQ ID NO: 65 - protein, 120 aa, synthetic construct
QVQLQESGGGLVQPGGSLRLSCAASGFTLDYYAIGWFRQAPGKEREGVACISSSDGSTYYADSVKGRFTISRDNAKNTVYLQMNSLKPEDTAVYYCATACGGATGPYDYWGQGTQVTVSS

SEQ ID NO: 66 - protein, 120 aa, synthetic construct
QVQLQESGGGLVQPGGSLRLSCAASGFTLDYYAIGWFRQAPGKEREGVACISSSDGSTYYADSVKGRFTISRDNAKNTVYLQMNSLKPQDTAVYYCATACGSLVGMYDYWGQGTQVTVSP

SEQ ID NO: 67 - protein, 121 aa, synthetic construct
QVQLQESGGGLVQPGGSLRLSCAASGFTLDYYAIGWFRQAPGKEREGVSCISASDGNTYYADSVKGRFTISRDNAKNTVYLQMNSLKPEDTAVYYCATTCASPEKYEYDYWGQGTQVTVSS

SEQ ID NO: 68 - protein, 120 aa, synthetic construct
QVQLQESGGGLVQPGGSLRLSCAASGFTLDYYAIGWFRQAPGKEREGVSCISASNGNTYYADSVKGRFTISRDSAKNTVYLQMNSLKPEDTAVYYCATTCSGLTHEYDYWGQGTQVTVSS

SEQ ID NO: 69 - protein, 120 aa, synthetic construct
QVQLQESGGGLVQPGGSLRLSCAASGFTLDYYAIGWFRQAPGKEREGVSCISSGDGNTYYADSVKGRFTISRDNAKNTVYLQMNSLKPEDTAVYYCATACGGATGPYDYWGQGTQVTVSS

SEQ ID NO: 70 - protein, 122 aa, synthetic construct
QVQLQESGGGLVQPGGSLRLSCAASGFTLDYYAIGWFRQAPGKEREGVSCISSGDGSTYYADSVKGRFTISRDNAKNTVYLQMNSLKPEDTAVYYCATHCGGSSWSNEYDYWGQGTQVTVSS

SEQ ID NO: 71 - protein, 121 aa, synthetic construct
QVQLQESGGGLVQPGGSLRLSCAASGFTLDYYAIGWFRQAPGKEREGVSCISSNDGSTYYADSVKGRFTISRDNAKNTVYLQMNSLKPEDTAVYYCATACADTTQHEYDYWGQGTQVTVSS

SEQ ID NO: 72 - protein, 120 aa, synthetic construct
QVQLQESGGGLVQPGGSLRLSCAASGFTLDYYAIGWFRQAPGKEREGVSCISSSDGGTYYADSVKGRFTISRDNAKNTVYLQMNSLKPEDTAVYYCATACGGATGPYDYWGQGTQVTVSS

SEQ ID NO: 73 - protein, 126 aa, synthetic construct
QVQLQESGGGLVQPGGSLRLSCAASGFTLDYYAIGWFRQAPGKEREGVSCISSSDGSSSDGNTYYADSVKGRFTISRDNAKNTVYLQMNNLKPEDTAVYYCATACVVTTLYEYDYWGQGTQVTVSS

SEQ ID NO: 74 - protein, 121 aa, synthetic construct
QVQLQESGGGLVQPGGSLRLSCAASGFTLDYYAIGWFRQAPGKEREGVSCISSSDGSTYYADSVKGRFTISRDNAKNTVYLQMNSLKPEDTAVYYCATACADTTQYEYDYWGQGTQVTVSP

SEQ ID NO: 75 - protein, 120 aa, synthetic construct
QVQLQESGGGLVQPGGSLRLSCAASGFTLDYYAIGWFRQAPGKEREGVSCISSSDGSTYYADSVKGRFTISRDNAKNTVYLQMNSLKPEDTAVYYCATACGGATGPYDYWGQGTQVTVSS

SEQ ID NO: 76 - protein, 121 aa, synthetic construct
QVQLQESGGGLVQPGGSLRLSCAASGFTLDYYAIGWFRQAPGKEREGVSCISSSGGSTYYADSVKGRFTISRDNAKNTVYLQMNMLKPEDTAVYYCATACADTTQYEYDYWGQGTQVTVSS

SEQ ID NO: 77 - protein, 121 aa, synthetic construct
QVQLQESGGGLVQPGGSLRLSCAASGFTLDYYAIGWFRQAPGKEREGVSCISSSGGSTYYADSVKGRFTISRDNAKNTVYLQMNSLKPEDTAVYYCATACASPVIYEYDYWGQGTQVTVSS

SEQ ID NO: 78 - protein, 121 aa, synthetic construct
QVQLQESGGGLVQPGGSLRLSCAASGFTLDYYAIGWFRQAPGKEREGVSCISSSGGSTYYADSVKGRFTISRDNAKNTVYLQMNSLKPEDTAVYYCATDCAGGTSTPYDYWGQGTQVTVSS

SEQ ID NO: 79 - protein, 121 aa, synthetic construct
QVQLQESGGGLVQPGGSLRLSCAASGFTLGYYAIGWFRQAPGKEREGVSCISSSDGSTYYADSVKGRFTISRDNAKNTVYLQMNSLKPEDTAVYYCATACADTTQYEYDYWGQGTQVTVSS

SEQ ID NO: 80 - protein, 122 aa, synthetic construct
QVQLQESGGGLVQPGGSLRLSCAASGFTLGYYAIGWFRQAPGKEREGVSCISSSDGSTYYADSVKGRFTISRDNAKNTVYLQMNSLKPEDTAVYYCATACANWSSLGPYDYWGQGTQVTVSS

SEQ ID NO: 81 - protein, 120 aa, synthetic construct
QVQLQESGGGLVQPGGSLRLSCAASGFTLGYYAIGWFRQAPGKEREGVSCISSSDGSTYYADSVKGRFTISRDNAKNTVYLQMNSLKPEDTGVYYCATACGGATGPYDYWGQGTQVTVSS

SEQ ID NO: 82 - protein, 121 aa, synthetic construct
QVQLQESGGGLVQPGGSLRLSCEGSGFSLDYYAIGWFRQAPGKEREGVSCISSGDGNTYYADSVKGRFTISRDNAKNTVYLQMNSLKPEDTAVYYCATDCVGFGSNWFDYWGQGTQVTVSS

SEQ ID NO: 83 - protein, 120 aa, synthetic construct
QVQLQESGGGLVQPGGSLRLSCTASGFSLDYYAIGWFRQAPGKEREGVACISSRTGSTYYADSVKGRFTISRDNAKNTVALQMNSLKPEDTAVYYCATACVVVGDQNDYWGQGTQVTVSS

SEQ ID NO: 84 - protein, 120 aa, synthetic construct
QVQLQESGGGLVQPGGSLRLSCTASGFSLDYYAIGWFRQAPGKEREGVSCISSRTGGTYYADSVKGRFTISRDDAKNTVYLQMNSLKPEDTAVYYCATACVVVGDRNDYWGQGTQVTVSS

SEQ ID NO: 85 - protein, 121 aa, synthetic construct
QVQLQESGGGLVQPGGSLRLSCTASGFSLDYYAIGWFRQAPGKEREGVSCISSRTGGTYYADSVKGRFTISRDNAKNTVYLQMNSLKPEDTAVYYCATACVDTTQYEYDYWGQGTQVTVSS

SEQ ID NO: 86 - protein, 120 aa, synthetic construct
QVQLQESGGGLVQPGGSLRLSCTASGFSLDYYAIGWFRQAPGKEREGVSCISSRTGGTYYADSVKGRFTISRDNAKNTVYLQMNSLKPEDTAVYYCATACVVVGDQNDYWGQGTQVTVSS

SEQ ID NO: 87 - protein, 120 aa, synthetic construct
QVQLQESGGGLVQPGGSLRLSCTASGFSLDYYAIGWFRQAPGKEREGVSCISSRTGNTYYADSVKGRFTISRDDAKNMVYLQMNSLKPEDTAVYYCATACVVVGDQNDYWGQGTQVTVSS

SEQ ID NO: 88 - protein, 120 aa, synthetic construct
QVQLQESGGGLVQPGGSLRLSCTASGFSLDYYAIGWFRQAPGKEREGVSCISSRTGSTYYADSVKGRFTISRDDAKNTVYLQMNSLKPEDTAVYYCATACVVVGDQNDYWGQGTQVTVSS

SEQ ID NO: 89 - protein, 120 aa, synthetic construct
QVQLQESGGGLVQPGGSLRLSCTASGFSLGYYAIGWFRQALGKEREGVSCISSRTGSTYYADSVKGRFTVSRDDAKNTVYLQMNSLKPEDTAVYYCATACVVVGDQNDYWGQGTQVTVSS

SEQ ID NO: 90 - protein, 120 aa, synthetic construct
QVQLQESGGGLVQPGGSLRLSCTASGFSLGYYAIGWFRQAPGKEREGVSCISSRTGSTYYADSVKGRFAISRDDAKNTVYLQMNSLKPEDTAVYYCATACVVVGDQNDYWGQGTQVTVSS

SEQ ID NO: 91 - protein, 120 aa, synthetic construct
QVQLQESGGGLVQPGGSLRLSCTASGFSLGYYAIGWFRQAPGKEREGVSCISSRTGSTYYADSVKGRFTISRDDAKNTVYLQMNSLKPEDTAVYYCATACVVVGDQNDYWGQGTQVTVSS

SEQ ID NO: 92 - protein, 120 aa, synthetic construct
QVQLQESGGGLVQPGGSLRLSCTASGFSLGYYAIGWFRQAPGKEREGVSCISSRTGSTYYADSVKGRFTVSRDDAKNTVYLQMNSLKPEDTAVYYCATACVVVGDQNDYWGQGTQVTVSS

SEQ ID NO: 93 - protein, 120 aa, synthetic construct
QVQLQESGGGLVQPGGSLRLSCVASGFPLDYYAIGWFRQAPGKEREGVSCISSSDGSTYYADSVKGRFTISRDNAKNTVYLQMNSLKPEDTAVYYCATACGGATGPYDYWGQGTQVTVSS

SEQ ID NO: 94 - protein, 121 aa, synthetic construct
QVQLQESGGGLVQPGGSLRLSCVASGFSLDYYAIGWFRQAPGKEREGVSCISNSDGSTYYADSVKGRFTISRDNAKNTVYLQMNSLKPEDTAVYYCATACADTTQYAYDYWGQGTQVTVSS

SEQ ID NO: 95 - protein, 121 aa, synthetic construct
QVQLQESGGGLVQPGGSLRLSCVASGFTLDYYAIGWFRQAPGKEREGVSCISSGSDGNTYYADSVKGRFTISRDNAKNTVYLQMNSLKPEDTAVYYCATACSGLTHEYDYWGQGTQVTVSS

SEQ ID NO: 96 - protein, 121 aa, synthetic construct
QVQLQESGGGLVQPGGSLRLSCVASGFTLDYYAIGWFRQAPGKEREGVSCISSSDDSTYYADSVKGRFTISRDNAKNTVYLQMNSLKPEDTAVYYCATACADTTQYEYDYWGQGTQVTVSS

SEQ ID NO: 97 - protein, 121 aa, synthetic construct
QVQLQESGGGLVQPGGSLRLSCVASGFTLDYYAIGWFRQAPGKEREGVSCISSSSDGNTYYADSVKGRFTISRDNAKNTVYLQMNSLKPEDTAVYYCATTCSGLTHEYDYWGQGTQVTVSS

SEQ ID NO: 98 - protein, 121 aa, synthetic construct
QVQLQESGGGLVQPGGSLRLSCVASGFTLGYYAIGWFRQAPGKEREGVSCISSSDGSTYYADSVKGRFTISRDNAKNTVYLQMNSLKPEDTAVYYCATACADTTQYDYDYWGQGTQVTVSS

SEQ ID NO: 99 - protein, 120 aa, synthetic construct
QVQLQESGGGLVQPGGSLRLSCVGSGFTLDYYAIGWFRQAPGKEREGVSCISSNDGSTYYADSVKGRFTISRDNAKNTVYLQMNSLKPEDTAVYYCATACGGATGPYDYWGQGTQVTVSS

SEQ ID NO: 100 - protein, 121 aa, synthetic construct
QVQLQESGGGLVQSGGSLRLSCAASGFPLAYYAIGWFRQAPGKEREGVSCISASDGSTYYADSVKGRFTISRDNAKNTVYLQMNSLKPEDTAVYYCATACAETTLYEYDYWGQGTQVTVSS

SEQ ID NO: 101 - protein, 120 aa, synthetic construct
QVQLQESGGGLVQTGGSLRLSCAASGFTLDYYAIGWFRQAPGKEREGVSCISSSDGSTYYADSVKGRFTISRDNAKNTVYLQMNSLKPEDTAVYYCATACGGATGPYDYWGQGTQVTVSS

SEQ ID NO: 102 - protein, 121 aa, synthetic construct
QVQLQESGGGMVQAGESLRLSCAASGFPLAYYAIGWFRQAPGKEREGVSCISSSDGNTYYADSVKGRFTISRDNAKNTVYLQMNSLKPEDTAVYYCATACADATQHEYDYWGQGTQVTVSS

SEQ ID NO: 103 - protein, 121 aa, synthetic construct
QVQLQESGGGSVQPGESLRLSCAASGFPLDYYAIGWFRQAPGKEREGVSCISASDGSTYYADSVKGRFTISRDNAKNTVYLQMNSLKPEDTAVYYCATACADTTLYEYDYWGQGTQVTVSS

SEQ ID NO: 104 - protein, 121 aa, synthetic construct
QVQLQESGGGSVQPGGSLRLSCAASGFTLDYYAIGWFRQAPGKEREGVSCISSGDGSTYYADSVKGRFTISRDNAKNTVYLQMNSLKPEDTAVYYCATACADTTHYEYDYWGQGTQVTVSS

SEQ ID NO: 105 - protein, 120 aa, synthetic construct
QVQLQESGGGSVQSGGSLRLSCTASGFSLGYYAIGWFRQAPGKEREGVSCISSRTGSTYYADSVKGRFTVSRDDAKNTVYLQMNSLKPEDTAVYYCATACVVVGDQNDYWGQGTQVTVSS

SEQ ID NO: 106 - protein, 121 aa, synthetic construct
QVQLQESGGGSVRPGGSLRLSCAASGFPLAYYAIGWFRQAPGKEREGVSCISSSDGNTYYADAVKGRFTISRDNAKNAVYLQMNSLKPEDTAVYYCATACADTTQHEYDYWGQGTQVTVSS

SEQ ID NO: 107 - protein, 121 aa, synthetic construct
QVQLQESGGGVAQPGGSLRLSCAASGFPLDYYAIGWFRQAPGKEREGVSCISASDGSTYYADSVKGRFTISRDNAKNTVYLQMNSLKPEDTAVYYCATACADTTLYEYDYWGQGTQVTVSS

SEQ ID NO: 108 - protein, 114 aa, synthetic construct
QVQLQESGGGVVQAGGSLKLSCAASGSDFRADAMGWYRQAPGKEREPVAIDSITSIYYVDSVEGRFTISRDNTKNTVYLQMTSLKPEDTAVYYCYARYSGRTYWGRGTQVTVSS

SEQ ID NO: 109 - protein, 122 aa, synthetic construct
QVQLQESGGGVVQPGGSLRLSCAASGFSLDYYAIGWFRQAPGKEREGVSCISSGDGSTYYADSVKGRFTISRDNAKNTVYLQMNSLKPEDTAVYYCATHCGGTSWGTSYDYWGQGTQVTVSS

SEQ ID NO: 110 - protein, 124 aa, synthetic construct
QVQLQESGGGVVQPGGSLRLSCAASGLTFRSVGMGWFRRAPGKEREFVATASPSGVITYYADSVKGRFTISRDNAKNTVYLEMNSLKPEDTAVYYCAVRIYSGSFDNTLAYDYWGQGTQVTVSS

SEQ ID NO: 111 - protein, 120 aa, synthetic construct
QVQLQESGGGVVQPGGSLRLSCTASGFSLGYYAIGWFRQAPGKEREGVSCISSRTGSTYYADSVKGRFTVSRDDAKNTVYLQMNSLKPEDTAVYYCATACVVVGDQNDYWGQGTQVTVSS

SEQ ID NO: 112 - protein, 120 aa, synthetic construct
QVQLQESGGGVVQSGGSLRLSCTASGFSLDYYAIGWFRQAPGKEREGVSCISSRTGSTYYADSVKGRFTISRDDAKNTVYLQMNSLKPEDTAVYYCATACVVVGDQNDYWGQGTQVTVSS

SEQ ID NO: 113 - protein, 14 aa, synthetic construct
ATACADTTQYAYDY

SEQ ID NO: 114 - protein, 14 aa, synthetic construct
ATACADTTLYEYDY

SEQ ID NO: 115 - protein, 14 aa, synthetic construct
ATACVDTTQYEYDY

SEQ ID NO: 116 - protein, 14 aa, synthetic construct
ATACADATQHEYDY

SEQ ID NO: 117 - protein, 14 aa, synthetic construct
ATACADTTQYDYDY

SEQ ID NO: 118 - protein, 14 aa, synthetic construct
ATACADTTQYEYDY

SEQ ID NO: 119 - protein, 14 aa, synthetic construct
ATACADTTHYEYDY

SEQ ID NO: 120 - protein, 14 aa, synthetic construct
ATACVITTLYEYDY

SEQ ID NO: 121 - protein, 14 aa, synthetic construct
ATACAETTLYEYDY

SEQ ID NO: 122 - protein, 14 aa, synthetic construct
ATACADTTQHEYDY

SEQ ID NO: 123 - protein, 14 aa, synthetic construct
ATACVDTTHYEYDY

SEQ ID NO: 124 - protein, 14 aa, synthetic construct
ATACASTTLYEYDY

SEQ ID NO: 125 - protein, 14 aa, synthetic construct
ATACVVTTLYEYDY

SEQ ID NO: 126 - protein, 13 aa, synthetic construct
ATACGGATGPYDY

SEQ ID NO: 127 - protein, 13 aa, synthetic construct
ATACAGAIGPYDY

SEQ ID NO: 128 - protein, 13 aa, synthetic construct
ATACVVVGDQNDY

SEQ ID NO: 129 - protein, 13 aa, synthetic construct
ATACVVVGDRNDY

SEQ ID NO: 130 - protein, 14 aa, synthetic construct
ATDCAGGTSTPYDY

SEQ ID NO: 131 - protein, 14 aa, synthetic construct
ATDCAGGTATPYDY

SEQ ID NO: 132 - protein, 14 aa, synthetic construct
ATACVVADRNEYDY

SEQ ID NO: 133 - protein, 14 aa, synthetic construct
ATSCVVVTKNEYDY

SEQ ID NO: 134 - protein, 13 aa, synthetic construct
ATACSGLTHEYDY

SEQ ID NO: 135 - protein, 13 aa, synthetic construct
ATTCSGLTHEYDY

SEQ ID NO: 136 - protein, 15 aa, synthetic construct
ATACANWSSLGPYDY

SEQ ID NO: 137 - protein, 15 aa, synthetic construct
ATACANWSTLGPYDY

SEQ ID NO: 138 - protein, 14 aa, synthetic construct
ATACSDPRVYEYDY

SEQ ID NO: 139 - protein, 14 aa, synthetic construct
ATTCASPEKYEYDY

SEQ ID NO: 140 - protein, 15 aa, synthetic construct
ATHCGGTSWGTSYDY

SEQ ID NO: 141 - protein, 15 aa, synthetic construct
ATHCGGSSWSNEYDY

SEQ ID NO: 142 - protein, 9 aa, synthetic construct
YARYSGRTY

SEQ ID NO: 143 - protein, 17 aa, synthetic construct
ASSAWPAGPKHQVEYDY

SEQ ID NO: 144 - protein, 13 aa, synthetic construct
ATACGSLVGMYDY

SEQ ID NO: 145 - protein, 13 aa, synthetic construct
ATACGSAVHEYDY

SEQ ID NO: 146 - protein, 14 aa, synthetic construct
ATDCVGFGSNWFDY

SEQ ID NO: 147 - protein, 14 aa, synthetic construct
ATACASPVIYEYDY

SEQ ID NO: 148 - protein, 14 aa, synthetic construct
ATDCAGGVGHEYDY

SEQ ID NO: 149 - protein, 15 aa, synthetic construct
ATDCSLHGSDYPYDY

SEQ ID NO: 150 - protein, 17 aa, synthetic construct
AVRIYSGSFDNTLAYDY

SEQ ID NO: 151 - protein, 8 aa, synthetic construct
GFPLAYYA

SEQ ID NO: 152 - protein, 8 aa, synthetic construct
GFSLDYYA

SEQ ID NO: 153 - protein, 8 aa, synthetic construct
GFPLDYYA

SEQ ID NO: 154 - protein, 8 aa, synthetic construct
GFTLDYYA

SEQ ID NO: 155 - protein, 8 aa, synthetic construct
GFSLNYYA

SEQ ID NO: 156 - protein, 8 aa, synthetic construct
GFTLAYYA

SEQ ID NO: 157 - protein, 8 aa, synthetic construct
GFTLGYYA

SEQ ID NO: 158 - protein, 8 aa, synthetic construct
GFPLNYYA

SEQ ID NO: 159 - protein, 8 aa, synthetic construct
GFPLHYYA

SEQ ID NO: 160 - protein, 8 aa, synthetic construct
GFSLGYYA

SEQ ID NO: 161 - protein, 8 aa, synthetic construct
GFPLGYYA

SEQ ID NO: 162 - protein, 8 aa, synthetic construct
GFPLEYYA

SEQ ID NO: 163 - protein, 8 aa, synthetic construct
GSDFRADA

SEQ ID NO: 164 - protein, 8 aa, synthetic construct
GRTFSSYG

SEQ ID NO: 165 - protein, 8 aa, synthetic construct
GFSLAYYA

SEQ ID NO: 166 - protein, 8 aa, synthetic construct
GLTFRSVG

SEQ ID NO: 167 - protein, 8 aa, synthetic construct
ISNSDGST

SEQ ID NO: 168 - protein, 8 aa, synthetic construct
ISASDGST

SEQ ID NO: 169 - protein, 8 aa, synthetic construct
ISSSDGST

SEQ ID NO: 170 - protein, 8 aa, synthetic construct
ISSSDGNT

SEQ ID NO: 171 - protein, 8 aa, synthetic construct
ISSADGST

SEQ ID NO: 172 - protein, 8 aa, synthetic construct
ISSSGGST

SEQ ID NO: 173 - protein, 8 aa, synthetic construct
ISSGDGST

SEQ ID NO: 174 - protein, 8 aa, synthetic construct
ISAGDGNT

SEQ ID NO: 175 - protein, 8 aa, synthetic construct
ISSSDDST

SEQ ID NO: 176 - protein, 8 aa, synthetic construct
ISSNDGST

SEQ ID NO: 177 - protein, 8 aa, synthetic construct
ISSPDGST

SEQ ID NO: 178 - protein, 8 aa, synthetic construct
ISSRTGGT

SEQ ID NO: 179 - protein, 9 aa, synthetic construct
ISAGDGSST

SEQ ID NO: 180 - protein, 13 aa, synthetic construct
ISSSDGSSSDGNT

SEQ ID NO: 181 - protein, 8 aa, synthetic construct
ISSGDGNT

SEQ ID NO: 182 - protein, 8 aa, synthetic construct
ISSGDGKT

SEQ ID NO: 183 - protein, 8 aa, synthetic construct
ISSSDGGT

SEQ ID NO: 184 - protein, 8 aa, synthetic construct
ISSRTGST

SEQ ID NO: 185 - protein, 8 aa, synthetic construct
ISSRTGNT

SEQ ID NO: 186 - protein, 10 aa, synthetic construct
ISSSDGHSST

SEQ ID NO: 187 - protein, 9 aa, synthetic construct
ISSSSDGNT

SEQ ID NO: 188 - protein, 8 aa, synthetic construct
ISASNGNT

SEQ ID NO: 189 - protein, 9 aa, synthetic construct
ISSGSDGNT

SEQ ID NO: 190 - protein, 8 aa, synthetic construct
ISASDGNT

SEQ ID NO: 191 - protein, 7 aa, synthetic construct
IDSITSI

SEQ ID NO: 192 - protein, 16 aa, synthetic construct
ISWSGGSTIAASVGST

SEQ ID NO: 193 - protein, 11 aa, synthetic construct
ISSSDGSDGNT

SEQ ID NO: 194 - protein, 8 aa, synthetic construct
ASPSGVIT

SEQ ID NO: 195 - protein, 115 aa, synthetic construct
QVQLQESGGGLVQAGGSLRLSCAASGINISSDVMGWYRQGPDNERELVAHVTTDSHTEYADSVKGRFTISRDNAKNTVYLQMNDLKPEDTAPYYCALRRGARILWGQGTQVTVSS

SEQ ID NO: 196 - protein, 115 aa, synthetic construct
QVQLQESGGGLVQPGESLRLSCAASGINISSDVMGWYRQGPDNERELVAHVTTDSHTEYADSVKGRFTISRDNAKNTVYLQMNDLKPEDTAPYYCALRRGARILWGQGTQVTVSS

SEQ ID NO: 197 - protein, 115 aa, synthetic construct
QVQLQESGGGSVQPGGSLRLSCAASGINISSDVMGWYRQGPGNERELVAHVTTDSHTEYADSVKGRFTISRDNAKNTVYLQMNDLKPEDTAPYYCALRRGARILWGQGTQVTVSS

SEQ ID NO: 198 - protein, 115 aa, synthetic construct
QVQLQESGGGSVQPGESLRLSCAASGINISSDVMGWYRQGPDNERELVAHVTTDSHTEYADSVKGRFTISRDNAKNTVYLQMNDLKPEDTAPYYCALRRGARILWGQGTQVTVSS

SEQ ID NO: 199 - protein, 115 aa, synthetic construct
QVQLQESGGGLVQPGGSLRLSCATSGSSYSTSDMGWYRQAPGKQRELVAAISPAGATNYADFAQGRFAASRDNAKNTVYLQMNDLKPEDTAPYYCALRRGARILWGQGTQVTVSS

SEQ ID NO: 200 - protein, 115 aa, synthetic construct
QVQLQESGGGSVRPGGSLRLSCAASGINASGDVMGWYRQAPGNERELVAHITTDSRTNYADSVKGRFTISRDNAKNTVYLQMNDLKPEDTAPYYCALRRGARILWGQGTQVTVSS

SEQ ID NO: 201 - protein, 115 aa, synthetic construct
QVQLQESGGGSVQHGGSLRLSCTASGLTFSNYDMGWYRQGPDNERELVAHVTTDSHTEYADSVKGRFTISRDNAKNTVYLEMNDLKPEDTAPYYCALRRGARILWAQGTQVTVSS

SEQ ID NO: 202 - protein, 115 aa, synthetic construct
QVQLQESGGGSVQPGESLRLSCAASGINISSDVMGWYRQGPDNERELVAHVTTDSHTEYADSVKGRFTISRDNAKNMVYLQMNDLKPEDTAPYYCALRRGARILWGQGTQVTVSS

SEQ ID NO: 203 - protein, 115 aa, synthetic construct
QVQLQESGGGLVQAGESLRLSCAASGINISSDVMGWYRQGPDNERELVAHVTTDSHTEYADSVKGRFTISRDNAKNTVYLQMNDLKPEDTAPYYCALRRGARILWGQGTQVTVSS

SEQ ID NO: 204 - protein, 115 aa, synthetic construct
QVQLQESGGGLVQPGGSLRLSCAASGIFVSANSMGWYRQGPEDARELVASITSSGDTMYGDSWKGRFAISRDNAKNTLYLQMNLLKSDDTAVYYCNARRGARVLWGKGTQVTVSS

SEQ ID NO: 205 - protein, 115 aa, synthetic construct
QVQLQESGGGSVQPGGSLRLSCAASGINISSDVMGWYRQGPDNERELVAHVTTDSHTEYADSVKGRFTISRDNAKNTVYLQMNDLKPEDTAPYYCALRRGARILWGQGTQVTVSS

SEQ ID NO: 206 - protein, 115 aa, synthetic construct
QVQLQESGGGLVQPGGSLRLSCAASGINISSDVMGWYRQGPDNERELVAHVTTDSHTEYADSVKGRFTISRDNAKNTVYLQMNDLKPEDTAPYYCALRRGARILWGQGTQVTVSS

SEQ ID NO: 207 - protein, 115 aa, synthetic construct
QVQLQESGGGLVHSGGSLRLSCAASGINISSDVMGWYRQGPDNERELVAHVTTDSHTEYADSVKGRFTISRDNAKNTVYLQMNDLKPEDTAPYYCALRRGARILWGQGTQVTVSS

SEQ ID NO: 208 - protein, 115 aa, synthetic construct
QVQLQESGGGSVQPGESLRLSCAASGINISSDVMGWYRQGPDNERELVAHVTTDSHTEYADSVKGRFTISRDNAKNTVYLEMNDLKPEDTAPYYCALRRGARILWGQGTQVTVSS

SEQ ID NO: 209 - protein, 113 aa, synthetic construct
QVQLQESGGGVAQPGESLRLSCKVSGIALDYYAIGWLRQAPGKEREGVAMLGSTTDYSASVKGRFTISRGDAKNTVYLDMNNLKPEDTATYYCALRRGARILWGQGTQVTVSS

SEQ ID NO: 210 - protein, 115 aa, synthetic construct
QVQLQESGGGSVQPGGSLRLSCVASGINISSDVMEWYRQAPGEERELVAHITTYSRTDYSDSVKGRFTISRANAKNTLYLQMNGLKPEDTALYYCALRRGARILWGQGTQVTVSS

SEQ ID NO: 211 - protein, 115 aa, synthetic construct
QVQLQESGGGSVQSGESLKLSCVGSGDIYRINTMAWYRQTPGKEREMVAVIRRGGNTNYADSVKGRFTISRDDSKNTMSLQMNSLRPEDTATYYCALRRGARILWGQGTQVTVSS

SEQ ID NO: 212 - protein, 115 aa, synthetic construct
QVQLQESGGGSVQPGESLRLSCAASGINISSDVMGWYRQGPDNERELVAHVTTDSHTEYADSVKGRFTISRDNAKNTVYLQMNDLKPEDTAPYYCALRRGARILWGPGTQVTVSS

SEQ ID NO: 213 - protein, 115 aa, synthetic construct
QVQLQESGGGSVQPGESLRLSCAASGINISSDVMGWYRQGPDNERELVAHVTTDSHTEYADSVKGRFTISRDNAKNTVYLQMNDLKPEDTAPYYCALRRGARILWSQGTQVTVSS

SEQ ID NO: 214 - protein, 115 aa, synthetic construct
QVQLQESGGGLVEPGGSLRLSCQISGGIFTVNDMGWYRRSPGTDRELVAHVTTDSHTEYADSVKGRFTISRDNAKNTVYLQMNDLKPEDTAPYYCALRRGARILWGQGTQVTVSS

SEQ ID NO: 215 - protein, 115 aa, synthetic construct
QVQLQESGGGLVKPGGSLRLSCAASGIFVSANSMGWYRQGPEDARELVASITSSGDTMYGDSWKGRFAISRDNAKNTLYLQMNLLKSDDTAVYYCNARRGARVLWGKGTQVTVSS

SEQ ID NO: 216 - protein, 115 aa, synthetic construct
QVQLQESGGGSVQPGGSLRLSCAASGINISRDVMGWYRQGPVNERELVAHVTTDSHTEYADSVKGRFTISRDNAKNTVYLEMNDLKPEDTAPYYCALRRGARILWGQGTQVTVSS

SEQ ID NO: 217 - protein, 115 aa, synthetic construct
QVQLQESGGGLVQPGGSLRLSCAASGINISSDVMGWYRQGPDNERELVAHVTTDSHTEYADSVKGRFTISRDNAKNTVYLQMNDLRPEDTAPYYCALRRGARILWGQGTQVTVSS

SEQ ID NO: 218 - protein, 115 aa, synthetic construct
QVQLQESGGGLVQPGESLRLSCAASGINISSDVMGWYRQGPDNERELVAHVSTDSHTEYADSVKGRFTISRDNAKNTVYLQMNDLKPEDTAPYYCALRRGARILWGQGTQVTVSS

SEQ ID NO: 219 - protein, 115 aa, synthetic construct
QVQLQESGGGLVHSGESLRLSCAASGINISSDVMGWYRQGPDNERELVAHVTTDSHTEYADSVKGRFTISRDNAKNTVYLQMNDLKPEDTAPYYCALRRGARILWGQGTQVTVSS

SEQ ID NO: 220 - protein, 115 aa, synthetic construct
QVQLQESGGGSVQPGGSLRLSCAASGINISRDVMGWYRQGPGNERELVAHVTTDSHTEYADSVKGRFTISRDNAKNTVYLEMNDLKPEDTAPYYCALRRGARILWGQGTQVTVSS

SEQ ID NO: 221 - protein, 115 aa, synthetic construct
QVQLQESGGGSVQPGESLRLSCAASGINISSDVMGWYRQGPDNEHELVAHVTTDSHTEYADSVKGRFTISRDNAKNTVYLQMNDLKPEDTAPYYCALRRGARILWGQGTQVTVSS

SEQ ID NO: 222 - protein, 115 aa, synthetic construct
QVQLQESGGGSVQPGESLRLSCAASGINISSDVMGWYRQGPDNERELVAHVTTDSHTEYADSVKGRFTISRDNAKNTVYLQTNDLKPEDTAPYYCALRRGARILWGQGTQVTVSS

SEQ ID NO: 223 - protein, 115 aa, synthetic construct
QVQLQESGGGSVQPGGSLRLSCAASGIRISSDVMGWYRQGPGNDRELVAHVTADSHTEYADSAKGRFTISRDNAKNTVYLQMNDLKPEDTAPYYCALRRGARILWGQGTQVTVSS

SEQ ID NO: 224 - protein, 115 aa, synthetic construct
QVQLQESGGGSVQPGESLRLSCAAPGINISSDVMGWYRQGPDNERELVAHVTTDSHTEYADSVKGRFTISRDNAKNTVYLQMNDLKPEDTAPYYCALRRGARILWGQGTQVTVSS

SEQ ID NO: 225 - protein, 115 aa, synthetic construct
QVQLQESGGGSVQPGESLRLSCAASGINISSDVMGWYRQGPGNERELVAHVTTDSHTEYADSVKGRFTISRDNAKNTVYLQMNDLKPEDTAPYYCALRRGARILWGQGTQVTVSS

SEQ ID NO: 226 - protein, 115 aa, synthetic construct
QVQLQESGGGSVQPGESLRLSCAASGINISSDVMGWYRQGPDNERELVAHVTTDSHTEYADSVKGRFTISRDNASNTVYLQMNDLKPEDTAPYYCALRRGARILWGQGTQVTVSS

SEQ ID NO: 227 - protein, 115 aa, synthetic construct
QVQLQESGGGLVQPGGSLRLSCAASGIFVSANTMGWYRQGPEDARELVASITSSGDTTYADSWKGRFAISRDNAKNTLYLQMNLLKSDDTAVYYCNARRGARVLWGKGTQVTVSS

SEQ ID NO: 228 - protein, 115 aa, synthetic construct
QVQLQESGGGLVQPGGSLRLSCAASGINISSDVMGWYRQGPDDERELVAHVTTDSHTEYADSVKGRFTISRDNAKNTVYLQMNDLKPEDTAPYYCALRRGARILWGQGTQVTVSS

SEQ ID NO: 229 - protein, 115 aa, synthetic construct
QVQLQESGGGMVQPGESLRLSCVASGVTGEIEAMGWYRQGPEDARELVASITSSGDTTYGDSWKGRFAISRDNAKNTLYLQMNLLKSDDTAVYYCNARRGARVLWGKGTQVTVSS

SEQ ID NO: 230 - protein, 115 aa, synthetic construct
QVQLQESGGGSVQPGGSLRLSCAASGINISSDVMEWFRQGPGEQRELVAHITTNSRTEYADFVKGRFTISRANAKNTLYLQMNGLKPEDTALYYCALRRGGRILWGQGTQVTVSS

SEQ ID NO: 231 - protein, 115 aa, synthetic construct
QVQLQESGGGLVQPGESLRLSCAASGINISSDVMGWYRQGPDNERELVAHVTTDSHTEYADSVKGRLTISRDNAKNTVYLQMNDLKPEDTAPYYCALRRGARILWGQGTQVTVSS

SEQ ID NO: 232 - protein, 115 aa, synthetic construct
QVQLQESGGGSVQPGESLRLSCAASGTNISSDVMGWYRQGPDNERELVAHVTTDSHTEYADSVKGRFTISRDNAKNTVYLQMNDLKPEDTAPYYCALRRGARILWGQGTQVTVSS

SEQ ID NO: 233 - protein, 115 aa, synthetic construct
QVQLQESGGGLVTPGDSLRLSCAASGINISSDVMGWYRQGPDNERELVAHVTTDSHTEYADSVKGRFTISRDNAKNTVYLQMNDLKPEDTAPYYCALRRGARILWGQGTQVTVSS

SEQ ID NO: 234 - protein, 115 aa, synthetic construct
QVQLQESGGGLVQPGGSLSVSCAASGINISSDVMGWYRQGPDNERELVAHVTTDSHTEYADSVKGRFTISRDNAKNTVYLQMNDLKPEDTAPYYCALRRGARILWGQGTQVTVSS

SEQ ID NO: 235 - protein, 115 aa, synthetic construct
QVQLQESGGGSVQPGESLRLSCAASGINISNDVMGWYRQGPDNERELVAHVTTDSHTEYADSVKGRFTISRDNAKNTVYLQMNDLKPEDTAPYYCALRRGARILWGQGTQVTVSS

SEQ ID NO: 236 - protein, 115 aa, synthetic construct
QVQLQESGGGSVQPGESLRLSCAASGINISSDVMGWYRQGPDNERELVAHVTTDSHTEYADSVKGRFTISRDNAKNTVYLQMNDLKPEDAAPYYCALRRGARILWGQGTQVTVSS

SEQ ID NO: 237 - protein, 115 aa, synthetic construct
QVQLQESGGGLVQPGGSLRLSCTASGSIRGIGYMAWGRQGPGKPRELIATINSAGDTNYAESVKGRFTVSRDNAENTLYLQMNNLKSEDTAVYYCNARPGLQSYWGQGTQVTVSS

SEQ ID NO: 238 - protein, 115 aa, synthetic construct
QVQLQESGGGLVQPGGSLRLSCTASGSIRDIGYMAWGRQAPGKQRELIATINSAGNTNYAESVKGRFTVSKDNAENTLYLQMNNLKSEDTAVYYCNARPGLRSYWGQGTQVTVSS

SEQ ID NO: 239 - protein, 115 aa, synthetic construct
QVQLQESGGGSAQSGGSLRLSCTASGSIRGIGYMAWGRQAPGKQRELIATINSAGDTNYAESVKGRFTVSRDNAENTLYLQMNNLKSEDTAVYYCNARPGLRSYWGQGTQVTVSS

SEQ ID NO: 240 - protein, 115 aa, synthetic construct
QVQLQESGGGLVQPGGSLRLSCTASGSIRGIGYMAWGRQAPDKPRELIATINSAGATNYAESVKGRFTVSRDNAANTLYLQMNNLKSEDTAVYYCNARPGLQSFWGQGTQVTVSS

SEQ ID NO: 241 - protein, 115 aa, synthetic construct
QVQLQESGGGLVQPGGSLRLSCTASGSIRGIGYMAWGRQAPGKQRELIATINSAGDTNYAESVKGRFTVSRDNAENTLYLQMNNLKSEDTAVYYCNARPGLRSYWGQGTQVTVSS

SEQ ID NO: 242 - protein, 115 aa, synthetic construct
QVQLQESGGGSVQAGDSLRLSCTASGSIRGIGYMAWGRQAPGKQRELIATINSAGDTNYAESVKGRFTVSRDNAENTLYLQMNNLKSEDTAVYYCNARPGLRSYWGQGTQVTVSS

SEQ ID NO: 243 - protein, 115 aa, synthetic construct
QVQLQESGGGLVHSGGSLTLSCTASGSIRDIGYMAWGRQAPGKQRELIATINSAGNTNYAESVKGRFTVSKDNAENTLYLQMNNLKSEDTAVYYCNARPGLRSYWGQGTQVTVSS

SEQ ID NO: 244 - protein, 115 aa, synthetic construct
QVQLQESGGGLVQPGGSLRLSCTASGSIRDIGYMAWGRQAPGEPRELIATLNSAGNTNYAESVKGRFTVSKDNAENTLYLQMNNLKSEDTAVYYCNARPGLRSYWGQGTQVTVSS

SEQ ID NO: 245 - protein, 121 aa, synthetic construct
QVQLQESGGGLVQPGGSLRLSCEVSGSIFSINRMAWYRQVPEKQREMVAQITYGDTIKYYGSAKGRFTISRDSAKNAVYLQMNSLKPEDTAVYRCNIVGVLTTGHYEDSFWGQGTQVTVPS

SEQ ID NO: 246 - protein, 121 aa, synthetic construct
QVQLQESGGGLVQPGGSLRLSCEVSGSIFSINRMAWYRQVPEKEREMVAQITYGDTIKYYGSAKGRFTISRDSAKNAVYLQMNSLKPEDTAVYRCNIVGVLTTGHYEDSFWGQGTQVTVPS

SEQ ID NO: 247 - protein, 121 aa, synthetic construct
QVQLQESGGGLVQPGGSLRLSCEVSGSISSINRMAWYRQVPGKEREMVAQITYGDTIKYYGSAKGRFTISKDSAKNAVYLQMNSLKPEDTAVYRCNIVGVLTTGHYENSFWGQGTQVTVSS

SEQ ID NO: 248 - protein, 123 aa, synthetic construct
QVQLQESGGGSVQPGESLRLSCAASGINISSDVMGWYRQGPDNERELVAHVTTDSHTEYADSVKGRFTISRPSARNTVWLQMNSLKPEDTAVYYCAARDRLLNTVTNIDYDYWGQGTQVTVSS

SEQ ID NO: 249 - protein, 121 aa, synthetic construct
QVQLQESGGGLVQPGGSLRLSCEVSGSIFSINRMAWYRQVPGKQREMVAQITYGDTIKYYGSAKGRFTISRDSAKNAVYLQMNSLKPEDTAVYRCNVVGVLHTGHYENSFWGQGTQVTVSS

SEQ ID NO: 250 - protein, 115 aa, synthetic construct
QVQLQESGGGLVQPGGSLRLSCATSASIFSISAMGWYRQTSGSERELVALMDTAYITDYADTVKGRFTISRDNAKNTVNLQMNSLKPEDTAVYYCAARRASTIAWGQGTQVTVSS

SEQ ID NO: 251 - protein, 115 aa, synthetic construct
QVQLQESGGGLVQPGGSLRLSCAASGSIFNTNGMGWYRQAPGKQRERVAGISSGGNTVYAASVEGRFTISRDNAKNTGYLQMNNLKPEDTAVYYCRVAGRQDDYWGQGTQVTVSS

SEQ ID NO: 252 - protein, 121 aa, synthetic construct
QVQLQESGGGSVQAGGSLRLSCAASQTITRLSTIGWYRQSAGKGRDLVASMTGNGDTMYADSVRGRFTISRDNAKNTVYLQMDSLKPEDTAVYYCGADAVFYSGGYYSDSWGQGTQVTVSS

SEQ ID NO: 253 - protein, 9 aa, synthetic construct
ALRRGARIL

SEQ ID NO: 254 - protein, 17 aa, synthetic construct
AARDRLLNTVTNIDYDY

SEQ ID NO: 255 - protein, 15 aa, synthetic construct
GADAVFYSGGYYSDS

SEQ ID NO: 256 - protein, 9 aa, synthetic construct
RVAGRQDDY

SEQ ID NO: 257 - protein, 9 aa, synthetic construct
AARRASTIA

SEQ ID NO: 258 - protein, 15 aa, synthetic construct
NVVGVLHTGHYENSF

SEQ ID NO: 259 - protein, 9 aa, synthetic construct
NARRGARVL

SEQ ID NO: 260 - protein, 9 aa, synthetic construct
NARPGLRSY

SEQ ID NO: 261 - protein, 15 aa, synthetic construct
NIVGVLTTGHYEDSF

SEQ ID NO: 262 - protein, 15 aa, synthetic construct
NIVGVLTTGHYENSF

SEQ ID NO: 263 - protein, 9 aa, synthetic construct
ALRRGGRIL

SEQ ID NO: 264 - protein, 9 aa, synthetic construct
NARPGLQSY

SEQ ID NO: 265 - protein, 9 aa, synthetic construct
NARPGLQSF

SEQ ID NO: 266 - protein, 8 aa, synthetic construct
GINISSDV

SEQ ID NO: 267 - protein, 8 aa, synthetic construct
GSSYSTSD

SEQ ID NO: 268 - protein, 8 aa, synthetic construct
GSIFSINR

SEQ ID NO: 269 - protein, 8 aa, synthetic construct
ASIFSISA

SEQ ID NO: 270 - protein, 8 aa, synthetic construct
QTITRLST

SEQ ID NO: 271 - protein, 8 aa, synthetic construct
GSIFNTNG

SEQ ID NO: 272 - protein, 8 aa, synthetic construct
GSISSINR

SEQ ID NO: 273 - protein, 8 aa, synthetic construct
GSIRDIGY

SEQ ID NO: 274 - protein, 8 aa, synthetic construct
GSIRGIGY

SEQ ID NO: 275 - protein, 8 aa, synthetic construct
GIFVSANS

SEQ ID NO: 276 - protein, 8 aa, synthetic construct
GINASGDV

SEQ ID NO: 277 - protein, 8 aa, synthetic construct
GLTFSNYD

SEQ ID NO: 278 - protein, 8 aa, synthetic construct
GGIFTVND

SEQ ID NO: 279 - protein, 8 aa, synthetic construct
GINISRDV

SEQ ID NO: 280 - protein, 8 aa, synthetic construct
GIRISSDV

SEQ ID NO: 281 - protein, 8 aa, synthetic construct
GIFVSANT

SEQ ID NO: 282 - protein, 8 aa, synthetic construct
GVTGEIEA

SEQ ID NO: 283 - protein, 8 aa, synthetic construct
GINISNDV

SEQ ID NO: 284 - protein, 8 aa, synthetic construct
GTNISSDV

SEQ ID NO: 285 - protein, 8 aa, synthetic construct
GDIYRINT

SEQ ID NO: 286 - protein, 8 aa, synthetic construct
GIALDYYA

SEQ ID NO: 287 - protein, 9 aa, synthetic construct
HVTTDSHTE

SEQ ID NO: 288 - protein, 9 aa, synthetic construct
AISPAGATN

SEQ ID NO: 289 - protein, 9 aa, synthetic construct
HITTDSRTN

SEQ ID NO: 290 - protein, 9 aa, synthetic construct
SITSSGDTM

SEQ ID NO: 291 - protein, 7 aa, synthetic construct
MLGSTTD

SEQ ID NO: 292 - protein, 9 aa, synthetic construct
HITTYSRTD

SEQ ID NO: 293 - protein, 9 aa, synthetic construct
VIRRGGNTN

SEQ ID NO: 294 - protein, 9 aa, synthetic construct
HVSTDSHTE

SEQ ID NO: 295 - protein, 9 aa, synthetic construct
HVTADSHTE

SEQ ID NO: 296 - protein, 9 aa, synthetic construct
SITSSGDTT

SEQ ID NO: 297 - protein, 9 aa, synthetic construct
HITTNSRTE

SEQ ID NO: 298 - protein, 9 aa, synthetic construct
TINSAGDTN

SEQ ID NO: 299 - protein, 9 aa, synthetic construct
TINSAGNTN

SEQ ID NO: 300 - protein, 9 aa, synthetic construct
TINSAGATN

SEQ ID NO: 301 - protein, 9 aa, synthetic construct
TLNSAGNTN

SEQ ID NO: 302 - protein, 9 aa, synthetic construct
QITYGDTIK

SEQ ID NO: 303 - protein, 9 aa, synthetic construct
LMDTAYITD

SEQ ID NO: 304 - protein, 9 aa, synthetic construct
GISSGGNTV

SEQ ID NO: 305 - protein, 9 aa, synthetic construct
SMTGNGDTM

SEQ ID NO: 306 - protein, 17 aa, unidentified
MWLQSLLLLGTVACSIS

SEQ ID NO: 307 - protein, 62 aa, unidentified
ALSNSIMYFSHFVPVFLPAKPTTTPAPRPPTPAPTIASQPLSLRPEACRPAAGGAVHTRGLD

SEQ ID NO: 308 - protein, 6 aa, synthetic construct
SGGGSG

SEQ ID NO: 309 - protein, 24 aa, unidentified
IYIWAPLAGTCGVLLLSLVITLYC

SEQ ID NO: 310 - protein, 43 aa, unidentified
RRLKIQVRKAAITSYEKSDGVYTGLSTRNQETYETLKHEKPPQ

SEQ ID NO: 311 - protein, 4 aa, synthetic construct
GSGS

SEQ ID NO: 312 - protein, 37 aa, Homo sapiens
GLAVSTISSFFPPGYQVSFCLVMVLLFAVDTGLYFSV

SEQ ID NO: 313 - protein, 28 aa, Homo sapiens
IHQDYTTQNLIRMAVAGLVLVALLAILV

SEQ ID NO: 314 - protein, 35 aa, unidentified
YEDMRGILYAAPQLRSIRGQPGPNHEEDADSYENM

SEQ ID NO: 315 - protein, 294 aa, unidentified
QVQLQESGGGLVQAGGSLRLSCAAPRSIFSINAMGWYRQAPGKQRELVAAITSGGSPTYADSVKGRFTISRDNAKNTVYLQMNSLKAEDTAVYYCATGPYGLDNALDAWGQGTQVTVSSSGGGGSGALSNSIMYFSHFVPVFLPAKPTTTPAPRPPTPAPTIASQPLSLRPEACRPAAGGAVHTRGLDIYIWAPLAGTCGVLLLSLVITLYCRRLKIQVRKAAITSYEKSDGVYTGLSTRNQETYETLKHEKPPQGSGSYEDMRGILYAAPQLRSIRGQPGPNHEEDADSYENM

SEQ ID NO: 316 - protein, 311 aa, unidentified
MWLQSLLLLGTVACSISQVQLQESGGGLVQAGGSLRLSCAAPRSIFSINAMGWYRQAPGKQRELVAAITSGGSPTYADSVKGRFTISRDNAKNTVYLQMNSLKAEDTAVYYCATGPYGLDNALDAWGQGTQVTVSSSGGGGSGALSNSIMYFSHFVPVFLPAKPTTTPAPRPPTPAPTIASQPLSLRPEACRPAAGGAVHTRGLDIYIWAPLAGTCGVLLLSLVITLYCRRLKIQVRKAAITSYEKSDGVYTGLSTRNQETYETLKHEKPPQGSGSYEDMRGILYAAPQLRSIRGQPGPNHEEDADSYENM

SEQ ID NO: 317 - protein, 14 aa, unidentified
ATACADTTLYEYDY

SEQ ID NO: 318 - protein, 8 aa, synthetic construct
ISASDGST

SEQ ID NO: 319 - protein, 13 aa, synthetic construct
ACANWSSLGPYDY

SEQ ID NO: 320 - protein, 8 aa, synthetic construct
GFTLGYYA

SEQ ID NO: 321 - protein, 8 aa, synthetic construct
ISSSDGST

SEQ ID NO: 322 - protein, 12 aa, synthetic construct
TCASPEKYEYDY

SEQ ID NO: 323 - protein, 8 aa, synthetic construct
ISASDGNT

SEQ ID NO: 324 - protein, 7 aa, synthetic construct
RYSGRTY

SEQ ID NO: 325 - protein, 6 aa, synthetic construct
DSITSI

SEQ ID NO: 326 - protein, 12 aa, synthetic construct
DCAGGVGHEYDY

SEQ ID NO: 327 - protein, 8 aa, synthetic construct
IAASVGST

SEQ ID NO: 328 - protein, 12 aa, synthetic construct
DCSLHGSDYPYD

SEQ ID NO: 329 - protein, 11 aa, synthetic construct
ISSSDGSDGNT

SEQ ID NO: 330 - protein, 7 aa, synthetic construct
RRGGRIL

SEQ ID NO: 331 - protein, 8 aa, synthetic construct
GINISSDV

SEQ ID NO: 332 - protein, 8 aa, synthetic construct
ITTNSRTE

SEQ ID NO: 333 - protein, 7 aa, synthetic construct
RPGLQSY

SEQ ID NO: 334 - protein, 8 aa, synthetic construct
GSIRGIGY

SEQ ID NO: 335 - protein, 7 aa, synthetic construct
INSAGDT

SEQ ID NO: 336 - protein, 13 aa, synthetic construct
VGVLTTGHYENSF

SEQ ID NO: 337 - protein, 7 aa, synthetic construct
ITYGDTI

SEQ ID NO: 338 - protein, 7 aa, synthetic construct
AGRQDDY

SEQ ID NO: 339 - protein, 7 aa, synthetic construct
ISSGGNT

SEQ ID NO: 340 - protein, 121 aa, synthetic construct
QVQLQESGGGLVQPGGSLRLSCAASGRTYGMGWFRQAPGKEREFVAVGVWSSGNTYYADFARGRFTISRDNAKNTVYLQMDSLKPEDTAVYYCAAPRYSSYTTYHAAYDYWGPGTQVTVSS

SEQ ID NO: 341 - protein, 13 aa, synthetic construct
VGVLTTGHYEDSF

SEQ ID NO: 342 - protein, 122 aa, synthetic construct
EVQLQESGGGLVQPGGSLRLSCAASGFTFDDYAMTWFRQAPGKEREFVSTISWSGGTTHYADSVKGRFTISRDNAKNTLYLQMSSLKPEDTAVYYCAKSLRSSPSSRWFGSWGQGTQVTVSS

SEQ ID NO: 343 - protein, 12 aa, synthetic construct
ACADTTLYEYDY

SEQ ID NO: 344 - protein, 8 aa, synthetic construct
QITYGDTI

SEQ ID NO: 345 - protein, 7 aa, synthetic construct
SGGGGSG

SEQ ID NO: 346 - protein, 314 aa, unidentified
MWLQSLLLLGTVACSISQVQLQESGGGLVQTGGSLRLACTASGFTFDDYAIAWFRQAPGKEREFVAAISWSGGTTHYADSVKGRFTISRDNAKNTLYLQMSSLKPEDTAVYFCAKSLRSSPSSRWFGSRGQGTQVTVSSSGGGGSGALSNSIMYFSHFVPVFLPAKPTTTPAPRPPTPAPTIASQPLSLRPEACRPAAGGAVHTRGLDIYIWAPLAGTCGVLLLSLVITLYCRRLKIQVRKAAITSYEKSDGVYTGLSTRNQETYETLKHEKPPQGSGSYEDMRGILYAAPQLRSIRGQPGPNHEEDADSYENM

SEQ ID NO: 347 - protein, 297 aa, unidentified
QVQLQESGGGLVQTGGSLRLACTASGFTFDDYAIAWFRQAPGKEREFVAAISWSGGTTHYADSVKGRFTISRDNAKNTLYLQMSSLKPEDTAVYFCAKSLRSSPSSRWFGSRGQGTQVTVSSSGGGGSGALSNSIMYFSHFVPVFLPAKPTTTPAPRPPTPAPTIASQPLSLRPEACRPAAGGAVHTRGLDIYIWAPLAGTCGVLLLSLVITLYCRRLKIQVRKAAITSYEKSDGVYTGLSTRNQETYETLKHEKPPQGSGSYEDMRGILYAAPQLRSIRGQPGPNHEEDADSYENM

SEQ ID NO: 348 - DNA, 941 bp, unidentified
  source 1..941: mol_type = unassigned DNA
atgtggctgcagtctctgctgctgctgggaacagtggcctgcagcatcagccaggtgcagttgcaggagagtggcggtggcctggtgcagaccggtggcagtctgaggctggcctgcactgccagcggcttcaccttcgacgactatgccatcgcgtggttcaggcaggcccctggcaaggagagggagtttgtcgcagccatcagctggagcggaggcaccacccactacgctgacagcgtgaagggcaggttcaccattagcagggacaatgccaagaacaccctgtacctgcagatgagcagtctcaagcccgaagataccgccgtgtacttctgcgccaagagcctgaggagcagccccagcagcaggtggttcggcagccgcggtcaaggcacccaggtgaccgtgagctctagcggaggcggaggatccggagccctgagcaatagcatcatgtacttcagccacttcgtgcccgtgtttctgcccgccaagcctacaacaacacccgctcctagaccacctacaccagctcctacaatcgccagccagcctctgtctctcagacctgaagcctgtagacctgcagctggcggagctgtgcataccagaggcctggatatctacatttgggcccctctggctggcacatgtggcgttctgctgctctctctggtcatcaccctgtactgcagacggctgaagatccaagtgcggaaggccgccatcaccagctacgagaaatctgatggcgtgtacaccggcctgagcacccggaatcaagaaacctacgagacactgaagcacgagaagcctccacaagcagcggctcctatgaggacatgagaggcattctgtacgccgctccacagctgagatctatcagaggccagcctggacctaaccacgaggaagatgccgactcctacgagaacatg

SEQ ID NO: 349 - DNA, 890 bp, unidentified
  source 1..890: mol_type = unassigned DNA
caggtgcagttgcaggagagtggcggtggcctggtgcagaccggtggcagtctgaggctggcctgcactgccagcggcttcaccttcgacgactatgccatcgcgtggttcaggcaggcccctggcaaggagagggagtttgtcgcagccatcagctggagcggaggcaccacccactacgctgacagcgtgaagggcaggttcaccattagcagggacaatgccaagaacaccctgtacctgcagatgagcagtctcaagcccgaagataccgccgtgtacttctgcgccaagagcctgaggagcagccccagcagcaggtggttcggcagccgcggtcaaggcacccaggtgaccgtgagctctagcggaggcggaggatccggagccctgagcaatagcatcatgtacttcagccacttcgtgcccgtgtttctgcccgccaagcctacaacaacacccgctcctagaccacctacaccagctcctacaatcgccagccagcctctgtctctcagacctgaagcctgtagacctgcagctggcggagctgtgcataccagaggcctggatatctacatttgggcccctctggctggcacatgtggcgttctgctgctctctctggtcatcaccctgtactgcagacggctgaagatccaagtgcggaaggccgccatcaccagctacgagaaatctgatggcgtgtacaccggcctgagcacccggaatcaagaaacctacgagacactgaagcacgagaagcctccacaagcagcggctcctatgaggacatgagaggcattctgtacgccgctccacagctgagatctatcagaggccagcctggacctaaccacgaggaagatgccgactcctacgagaacatg

SEQ ID NO: 350 - DNA, 932 bp, unidentified
  source 1..932: mol_type = unassigned DNA
atgtggctgcagtctctgctgctgctgggaacagtggcctgcagcatcagccaagttcagctccaggaatcaggtgggggactcgtacaggctggaggaagcctgcgattgagctgtgccgctccacgatctatcttctcaataaatgctatgggttggtacaggcaggccccaggcaaacaaagagaacttgtagcagccattacttcaggggggagcccaacctatgctgactcagtaaaggggcggttcactattagtagggataacgcgaagaatacggtctacttgcagatgaacagtctgaaggccgaagatacggccgtgtattattgtgccacaggtccctatgggctggacaatgcgttggatgcgtggggtcagggcacgcaggtcactgtgagtagtagcggaggcggaggatccggagccctgagcaatagcatcatgtacttcagccacttcgtgcccgtgtttctgcccgccaagcctacaacaacacccgctcctagaccacctacaccagctcctacaatcgccagccagcctctgtctctcagacctgaagcctgtagacctgcagctggcggagctgtgcataccagaggcctggatatctacatttgggcccctctggctggcacatgtggcgttctgctgctctctctggtcatcaccctgtactgcagacggctgaagatccaagtgcggaaggccgccatcaccagctacgagaaatctgatggcgtgtacaccggcctgagcacccggaatcaagaaacctacgagacactgaagcacgagaagcctccacaagcagcggctcctatgaggacatgagaggcattctgtacgccgctccacagctgagatctatcagaggccagcctggacctaaccacgaggaagatgccgactcctacgagaacatg

SEQ ID NO: 351 - DNA, 881 bp, unidentified
  source 1..881: mol_type = unassigned DNA
caagttcagctccaggaatcaggtgggggactcgtacaggctggaggaagcctgcgattgagctgtgccgctccacgatctatcttctcaataaatgctatgggttggtacaggcaggccccaggcaaacaaagagaacttgtagcagccattacttcaggggggagcccaacctatgctgactcagtaaaggggcggttcactattagtagggataacgcgaagaatacggtctacttgcagatgaacagtctgaaggccgaagatacggccgtgtattattgtgccacaggtccctatgggctggacaatgcgttggatgcgtggggtcagggcacgcaggtcactgtgagtagtagcggaggcggaggatccggagccctgagcaatagcatcatgtacttcagccacttcgtgcccgtgtttctgcccgccaagcctacaacaacacccgctcctagaccacctacaccagctcctacaatcgccagccagcctctgtctctcagacctgaagcctgtagacctgcagctggcggagctgtgcataccagaggcctggatatctacatttgggcccctctggctggcacatgtggcgttctgctgctctctctggtcatcaccctgtactgcagacggctgaagatccaagtgcggaaggccgccatcaccagctacgagaaatctgatggcgtgtacaccggcctgagcacccggaatcaagaaacctacgagacactgaagcacgagaagcctccacaagcagcggctcctatgaggacatgagaggcattctgtacgccgctccacagctgagatctatcagaggccagcctggacctaaccacgaggaagatgccgactcctacgagaacatg

SEQ ID NO: 352 - DNA, 942 bp, unidentified
  source 1..942: mol_type = unassigned DNA
atgtggctgcagtctctgctgctgctgggaacagtggcctgcagcatcagccaggtgcagttgcaggagagtggcggtggcctggtgcagaccggtggcagtctgaggctggcctgcactgccagcggcttcaccttcgacgactatgccatcgcgtggttcaggcaggcccctggcaaggagagggagtttgtcgcagccatcagctggagcggaggcaccacccactacgctgacagcgtgaagggcaggttcaccattagcagggacaatgccaagaacaccctgtacctgcagatgagcagtctcaagcccgaagataccgccgtgtacttctgcgccaagagcctgaggagcagccccagcagcaggtggttcggcagccgcggtcaaggcacccaggtgaccgtgagctctagcggaggcggaggatccggagccctgagcaatagcatcatgtacttcagccacttcgtgcccgtgtttctgcccgccaagcctacaacaacacccgctcctagaccacctacaccagctcctacaatcgccagccagcctctgtctctcagacctgaagcctgtagacctgcagctggcggagctgtgcataccagaggcctggatatctacatttgggcccctctggctggcacatgtggcgttctgctgctctctctggtcatcaccctgtactgcagacggctgaagatccaagtgcggaaggccgccatcaccagctacgagaaatctgatggcgtgtacaccggcctgagcacccggaatcaagaaacctacgagacactgaagcacgagaagcctccacaaggcagcggctcctatgaggacatgagaggcattctgtacgccgctccacagctgagatctatcagaggccagcctggacctaaccacgaggaagatgccgactcctacgagaacatg

SEQ ID NO: 353 - DNA, 891 bp, unidentified
  source 1..891: mol_type = unassigned DNA
caggtgcagttgcaggagagtggcggtggcctggtgcagaccggtggcagtctgaggctggcctgcactgccagcggcttcaccttcgacgactatgccatcgcgtggttcaggcaggcccctggcaaggagagggagtttgtcgcagccatcagctggagcggaggcaccacccactacgctgacagcgtgaagggcaggttcaccattagcagggacaatgccaagaacaccctgtacctgcagatgagcagtctcaagcccgaagataccgccgtgtacttctgcgccaagagcctgaggagcagccccagcagcaggtggttcggcagccgcggtcaaggcacccaggtgaccgtgagctctagcggaggcggaggatccggagccctgagcaatagcatcatgtacttcagccacttcgtgcccgtgtttctgcccgccaagcctacaacaacacccgctcctagaccacctacaccagctcctacaatcgccagccagcctctgtctctcagacctgaagcctgtagacctgcagctggcggagctgtgcataccagaggcctggatatctacatttgggcccctctggctggcacatgtggcgttctgctgctctctctggtcatcaccctgtactgcagacggctgaagatccaagtgcggaaggccgccatcaccagctacgagaaatctgatggcgtgtacaccggcctgagcacccggaatcaagaaacctacgagacactgaagcacgagaagcctccacaaggcagcggctcctatgaggacatgagaggcattctgtacgccgctccacagctgagatctatcagaggccagcctggacctaaccacgaggaagatgccgactcctacgagaacatg

SEQ ID NO: 354 - DNA, 933 bp, unidentified
  source 1..933: mol_type = unassigned DNA
atgtggctgcagtctctgctgctgctgggaacagtggcctgcagcatcagccaagttcagctccaggaatcaggtgggggactcgtacaggctggaggaagcctgcgattgagctgtgccgctccacgatctatcttctcaataaatgctatgggttggtacaggcaggccccaggcaaacaaagagaacttgtagcagccattacttcaggggggagcccaacctatgctgactcagtaaaggggcggttcactattagtagggataacgcgaagaatacggtctacttgcagatgaacagtctgaaggccgaagatacggccgtgtattattgtgccacaggtccctatgggctggacaatgcgttggatgcgtggggtcagggcacgcaggtcactgtgagtagtagcggaggcggaggatccggagccctgagcaatagcatcatgtacttcagccacttcgtgcccgtgtttctgcccgccaagcctacaacaacacccgctcctagaccacctacaccagctcctacaatcgccagccagcctctgtctctcagacctgaagcctgtagacctgcagctggcggagctgtgcataccagaggcctggatatctacatttgggcccctctggctggcacatgtggcgttctgctgctctctctggtcatcaccctgtactgcagacggctgaagatccaagtgcggaaggccgccatcaccagctacgagaaatctgatggcgtgtacaccggcctgagcacccggaatcaagaaacctacgagacactgaagcacgagaagcctccacaaggcagcggctcctatgaggacatgagaggcattctgtacgccgctccacagctgagatctatcagaggccagcctggacctaaccacgaggaagatgccgactcctacgagaacatg

SEQ ID NO: 355 - DNA, 882 bp, unidentified
  source 1..882: mol_type = unassigned DNA
caagttcagctccaggaatcaggtgggggactcgtacaggctggaggaagcctgcgattgagctgtgccgctccacgatctatcttctcaataaatgctatgggttggtacaggcaggccccaggcaaacaaagagaacttgtagcagccattacttcaggggggagcccaacctatgctgactcagtaaaggggcggttcactattagtagggataacgcgaagaatacggtctacttgcagatgaacagtctgaaggccgaagatacggccgtgtattattgtgccacaggtccctatgggctggacaatgcgttggatgcgtggggtcagggcacgcaggtcactgtgagtagtagcggaggcggaggatccggagccctgagcaatagcatcatgtacttcagccacttcgtgcccgtgtttctgcccgccaagcctacaacaacacccgctcctagaccacctacaccagctcctacaatcgccagccagcctctgtctctcagacctgaagcctgtagacctgcagctggcggagctgtgcataccagaggcctggatatctacatttgggcccctctggctggcacatgtggcgttctgctgctctctctggtcatcaccctgtactgcagacggctgaagatccaagtgcggaaggccgccatcaccagctacgagaaatctgatggcgtgtacaccggcctgagcacccggaatcaagaaacctacgagacactgaagcacgagaagcctccacaaggcagcggctcctatgaggacatgagaggcattctgtacgccgctccacagctgagatctatcagaggccagcctggacctaaccacgaggaagatgccgactcctacgagaacatg

SEQ ID NO: 356 - protein, 20 aa, synthetic construct
  VARIANT 1..20: note = This sequence may encompass 1-4 GxS repeating units, wherein x is 1-4
GGGGSGGGGSGGGGSGGGGS

SEQ ID NO: 357 - protein, 22 aa, unidentified
QVQLVESGGGLVQPGGSLRLSC

SEQ ID NO: 358 - protein, 13 aa, unidentified
WFRQAPGQGLEAV

SEQ ID NO: 359 - protein, 30 aa, unidentified
RFTISRDNSKNTLYLQMNSLRAEDTAVYYC

SEQ ID NO: 360 - protein, 12 aa, synthetic construct
YWGQGTLVTVSS

SEQ ID NO: 361 - protein, 22 aa, synthetic construct
EVQLVESGGGLVQPGGSLRLSC

SEQ ID NO: 362 - protein, 13 aa, synthetic construct
WVRQAPGKGLEWV

SEQ ID NO: 363 - protein, 29 aa, synthetic construct
FTISRDNSKNTLYLQMNSLRAEDTAVYYC

SEQ ID NO: 364 - protein, 6 aa, synthetic construct
HHHHHH